SEQUENCE LISTING (467 sequences)

SEQ ID NO: 1 - protein, 1080 aa, unidentified
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPNGSMPIVTKFETDDLISDNQRKAMISNFTKNAAAKAAKKPIPYLDRLKEHMVSLCDEYNVYAWAAAITNSNADVTARNTRNLTFIGEQNSRRKELSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 2 - protein, 1061 aa, unidentified
MSSDVVRPYNTKLLPDNRKYNMFLQTFKRLNLISSNHFDLLVCLYAAITNKKAEEYKSEKEDHVTADSLCAINWFRPMSKRYIKYATTTFKMLELFKEYSGHEPDTYSKNYLMSNIVSDRFVWVDCRKFAKDFANQMELSFHEFTTLSETLLANSILVLNESTKANWAWGAVSALYGGGDKEDSTLKSKILLAFVDALNNPELKTRREILNHVCESLKYQSYQDMYVDFRSVVDDKGNKKSPNGSMPIVTKFESDDLIGDNQRKTMISSFTKNAAAKASKKPIPYLDILKDHMISLCEEYNVYAWAAAITNSNADVTARNTRNLTFIGEQNTRRKELSVLQTSTNEKAKDILNKINDNLIPEVRYTPAPKHLGRDLANLFEMFKEKDINQIGNEEEKQNVINDCIEQYVDDCRSLNRNPVAALLKHISGYYEDFSAKNFLDGAKLNVLTEVVNRQKAHPTICSEKAYTWISKIDKNRRQANSSLVGWVVPPEEVHKEKIAGQQSMMWVTLTLLDDGKWVKHHIPFADSRYYSEVYAYNPNLPYLEGGIPRQSKFGNKPTTNLTAESQALLANSKHKKANKTFLRAKENITHNVRVSPNTSLCIRPLKDSAGNQMFDNIGNMLFGMQINHRITVGKPNYKIEVGDRFLGFDQNQSENHTYAVLQRVSESSHGTHHFNGWDVKVIEKGKVTSDVVVRDEVYDQLSYEGVPYDSPKFTEWREKRRKFVLENMSIQIEEGKTFLTEFDKLNKDSLYRWNMNYMKLLRKAIRAGGKEFAKITKAEIFELGVMRFGPMNLGSLSQVSLKMIAAFKGVIQSYFSVSGCIDDASKKAHDSMLFAFLCSADEKRTNKREEKTNRAASFILQKAYSHGCKMIVCEDDLPIADGKVGKAQNADRMDWCARSLAKKVNDGCVAMSICYRAIPAYMSSHQDPFTHMQDKKTSVLRPRFMEVGKDSIRDHHVAGLRRMLNSKGNTGTSVYYREAALRFCEALGVLPELVKNKKTHASELGKHMGSAMLMPWRGGRIYVASKKLTSDAKSIKYCGEDMWQYHADEIAAINIAMYEV

SEQ ID NO: 3 - protein, 1075 aa, unidentified
MGISISRPYGTKLRPDARKKEMLDKFFTTLAKGQRVFADLGLCIYGSLTLEMVKRLEPESDSELVCAIGWFRLVDKVTWSENEIKQENLVRQYETYSGKEASEVIKTYLSSPSSDKYVWIDCRQKFLRFQRDLGTRNLSEDFECMLFEQYLRLTKGELDGHTAMSNMFGTKTKEDRATKLRYAARMKEWLEANEEITWEQYHQALQDKLDANTLEEAVDNYKGKAGGSNPFFSYTLLNRGQIDKKTHEQQLKKFNKVLKTKSKNLNFPNKEKLKQYLETAIGIPVDAQVYGQMFNNGVSEVQPKTTRNMSFSMEKLELLNELKSLNKTDGFERANEVLNGFFDSELHTTEDKFNITSRYLGGDRNNRLPKLYELWKKEGVDREEGIQQFSQAIQDKMGQIPVKNVLRYIWEFRETVSAEDFEAAAKANQLEEKITRTKAHPVVISNRYWTFGSSALVGNIMPADKMHKDQYAGQSFKMWLEAELHYDGKKVKHHLPFYNARFFEEVYCYHPSVAEVTPFKTKQFGYAIGKDIPADVSVVLKDNPYKKATKRFLRAISNPVANTVDVNKPTVCSFMIKRENDEYKLVINRKIGVDRPKRIKVGRKVMGYDRNQTASDTYWIGELVPHGTTGAYRIGEWSVQYIKSGPVLSSTQGVNDSTTDQLIYNGMPSSSERFKAWKKSRMSFIRKLIRQLNAEGLESKGQDYVPENPSSFDVRGETLYVFNSNYMKALVSKHRKAKKPVEGILEEIEALTSKAKDSCSLMRLSSLSDAAMQGIASLKSLINSYFNKNGCKTIEDKEKFNPDLYVKLVEVEQKRTNKRKEKVGRIAGSLEQLALLNGVDVVIGEADLGEVKKGKSKKQNSRNMDWCAKQVAERLEYKLTFHCIGYFGVNPMYTSHQDPFEHRRVADHLVMRARFEEVNVSNVSEWHMRNFSNYLRADSGTGLYYKQATLDFLKHYDLEEHADDLEKQNIKFYDFRKILEDKQLTSVIVPKRGGRIYMATNPVTSDSTPVTYAGKTYNRCNADEVAAANIAISVLAPHSKKEEKEDKIPIISKKPKSKNTPKARKNLKTSQLPQK

SEQ ID NO: 4 - protein, 1003 aa, unidentified
MASKHVVRPFNGKVTATGKRLAYLEETFHYLEKAAGGVSTLFAALGSYLDATTISNLINKNQDLAVVIFRYHVVPKGEAHTLPVGTDMVSRFVADYGMEPNEFQRAYLDSPIDQEKYCWQDNRDVGCWLGEQLGVSEADMRAIAVTFYNNQMLYDCVKGTGSGNAVSLLFGSGKKSDYSMKGVIAGKAASVLAKYRPATYQDARKMILEANGFTSVKDLVTSYGITGRSSALQIFMEGIESGPISSKTLDARIKKFTEDSERNGRKNLVPHAGAIRNWLIEQAGSSVENYQMAWCEVYGNVSADWNAKVESNFNFVAEKVKALTELSNIQKSTPDLGKALKLFEEYLTTCQDEFAIAPYHFSVMEEVRMEMATGREFNDAYDDALNSLDMESKQPIQPLCKFLIERGGSISFDTFKSAAKYLKTQSKIAGRYPHPFVKGNQGFTFGSKNIWAAINDPMMEYADGRIAGGSAMMWVTATLLDGKKWVRHHIPFANTRYFEEVYASKKGLPVLPCARDGKHSFKLGNNLSVERVEKVKEGGRTKATKAQERILSNLTHNVQFDSSTTFIIRRQEESFVICVNHRHPAPLMKKEMEVGDKIIGIDQNVTAPTTYAIVERVASGGIERNGKQYKVTAMGAISSVQKTRGGEVDVLSYMGVELSDSKNGFQSLWNKCLDFVTKHGTENDVKYYNNTAVWANKLYVWHKMYFRLLKQLMRRAKDLKPFRDHLQHLLFHPNLSPLQRHSLSLTSLEATKIVRNCIHSYFSLLGLKTLDERKAADINLLEVLEKLYAGLVERRKERTKLTAGLLVRLCNEHGISFAAIEGDLPVVGEGKSKAANNTQQDWTARELEKRLSEMAEVVGIKVIAVLPHYTSHQDPFVYSKNTKKMRCRWNWRTTKTFTDRDALSIRRILSKPETGTNLYYQKGLKAFAEKHGLDLAEMKKRKDAQWYLERIQDKNFLVPMNGGRVYLSSVKLAGKETIDMGGEILYLNDADQVAALNVLLVKI

SEQ ID NO: 5 - protein, 1018 aa, unidentified
MAKKEHIIRPFKGTLPLRGDRLRYLQDTMKYMKKVEDTITELCAAVIAYAKPTIIQQILGEEIETTSTFCSFRLVGIHENFTMPLTTNMIKHFQKTFNINPSEKQAIYLSSGFDSDKYRWQDTSEVSRNFANKCRLTNQEFQEFAEQALLNMCFIGCSGSPGATNAVSQIFGTGEKSDYQRKSQIAKIAADTLENHKPSTYESARLMVLNTLGHKTIEDCVNDYGAIGAKSAFRLFMESKEIGPITSEQLTTKIKKFREDHKKNSIKKQLPHVEKVRNALLSQFKEQYLPSAWAEAWCNIMGEFNSKLSNNNNFIDQKTKMVNDCDNIKKSNPQLDKAVNMLDEWKYKNWDDNSAIHPYHIGDLKKLMAIFNINNEGTFDERFSASWEQFSTSLEYGEKPPVRDLLAHIIKNMNDLTYTDVINAAKFLKLQDNIRNKYPHPFVMPNKGCTFGKDNLWGEINDPTAKIKSTEEVAGQRPMMWLTAKLLDNGKWVEHHIPFASSRYFAEVYYTNPALPTLPIARDGKHSYKLTKTIDANTAKTLVNNPRDKAAKLIARTKANTTHNVKWIKPTYRIQKENNQFVITINHRHPCITPPKEIILGDRILSFDQNETAPTAFSILEKTTKGTEFCGHHIKVLKTGMLEAKIKTSKKSIDAFTYMGPMEDDHASGFPTLLNICEKFISENGDEKDKSFSSRKLPFKRSLYFFHGSHFDLLKKMIRKAKNDPKKLKLVRIHINEILFNSNLSPIKLHSLSIHSMENTKKVIAAISCYMNVHEWKTIDEQKNADITLYNAKEKLYNNLVNRRKERVKVTAGMLIRLARENNCRFMVGEAELPTQQQGKSKKNNNSKQDWCARDIAQRCEDMCEVVGIKWNGVTPHNTSHQNPFIYKNTSGQQMRCRYSLVKKSEMTDKMAEKIRNILHAEPVGTTAYYREGILEFAKHHGLDLGMMKKRRDAKYYDNLPDEFLLPTRGGRIYLSENQLGGNETIVINGKKYFVNQADQVAAVNIGLLYLLPKKNQS

SEQ ID NO: 6 - protein, 1047 aa, unidentified
MSEKKFHIRPYRCSISPNARKADMLKATISYLDSLTSVFRSGFTALLAGIDPSTVSRLAPSGAVGSPDLWSAVNWFRIVPLAEAGDARVGQASLKNLFRGYAGHEPDEEASIYMESRVDDKRHAWVDCRAMFRAMALECGLEEAQLASDVFALASREVIVFKDGEINGWGIASLLFGEGEKADSQKKVALLRSVRLALEGDYATYEELSGLMLAKTGASSGSDLLDEYKRSEKGGSSGGRHPFFDEVFRRGGRVKQEERERLLKSCDTAIQKQGQALPLSHVASWRQWFLRRVTLLRNRRQESFAVCITNALMDLQPKNLRNVHYVTNPKSEKDKGVLELRVDVKNNEGPDVAGAQAVFDAYMARLAPDLRFSVMPRHLGSLKDLYALWAKLGRDEAIEEYLEGYEGPFSKRPIAGILQIIHAHRGKVGHDSLLRAARLNRAMDRLERKRAHACAAGNKGYVYGKSSMVGRINPQSLEVGGRKSGRSPMMWVTLDLVDGDRFAQHHLPFQSARFFSEVYCHGDGLPATRVPGMVRNRRNGLAIGNGLGEGGLSALRAGSDRRKRANKRTLRALENITHNVEIDPSTSFTLREDGIIISHRIEKIEPKLVAFGDRALGFDLNQTGAHTFAVLQKVDSGGLDVGHSRVSIVLTGTVRSICKGNQASGGRDYDLLSYDGPERDDGAFTAWRSDRQAFLMSAIRELPTPAEGEKDYKADLLSQMASLDHYRRLYAYNRKCLGIYIGALRRATRRQAVAAFKDEILSIANHRCGPLMRGSLSVNGMESLANLKGLATAYLSKFKDSKSEDLLSKDEEMADLYRACARRMTGKRKERYRRAASEIVRLANEHGCLFVFGEKELPTTSKGNKSKQNQRNTDWSARAIVKAVKEACEGCGLGFKPVWKEYSSLTDPFERDGDGRPALRCRFAKVAAPDSELPPRLTKAVGSYVKNALKADKAEKKQTCYQRGAIEFCSRHGIDVRKATDKAIRKAVRGSSDLLVPFDGGRTFLLSTRLSPESRKVEWAGRTLYEFPSDMVAAINIACRGLEPRKA

SEQ ID NO: 7 - protein, 1029 aa, unidentified
MDEQAVVSSGSDKTLKIVRPYRAKVTATGIRLEGIKNTLNYLKRTEICLSRLNAACGAFLTPAIVEQICKDDPALVCAIARFQLVPVGSEATLSDSGLMRHFKAALGELTPLQEAYLNSSYNDELYAWQDTLVLARQIIAETGLTEDQFRAFAHACFKNGNIIGCAGGPGASNAISGIFGEGIKSDYSLRSEMTAAVAKVFEEKRPITYEEARALALEATGHASVQSFVEAFGKQGRKGTLILFMEDTKTGAFPSNEFDYKLKKLKEDAERVGRKGIIPHRDVIASYLRNQTGADIEYNSKAWCESYCCAVSEYNSKMSNNVRFATEKSLDLTKLDETIRETPKISEAMLVFENYMARIDADLRFIVSKHHLGNLAKFRQTMMHVSASEFEEAFKAMWADYLAGLEYGEKPAICELVRYVLTHGNDLPVEAFYAACKFLSLDDKIKNRYPHPFVPGNKGYTFGAKNLWAEINDPFKPIRQGNPEVAGQRPMMWATADLLDNNKWVLHHIPFASSRYFEEVYYTDPSLPTAQKARDGKHGYRLGKVLDEAARERLKANNRQRKAAKAIERIKANCEHNVAWDPTTTFMLQLDSEGNVKMTINHRHIAYRAPKEIGVGDRVIGIDQNETAPTTYAILERTENPRDLEYNGKYYRVVKMGSVTSPNVSKYRTVDALTYDGVSLSDDASGAVNFVVLCREFFAAHGDDEGRKYLERTLGWSSSLYSFHGNYFKCLTQMMRRSARSGGDLTVYRAHLQQILFQHNLSPLRMHSLSLRSMESTMKVISCMKSYMSLCGWKTDADRIANDRSLFEAARKLYTSLVNRRTERVRVTAGILMRLCLEHNVRFIHMEDELPVAETGKSKKSNGAKMHWCARELAVRLSQMAEVTSVKFTGVSPHYTSHQDPFVHSKTSKVMRARWSWRNRADFTDKDAERIRTILGGDDAGTKAYYRSALAEFASRYGLDMEQMRKRRDAQWYQERLPETFIIPQRGGRVYLSSHDLGSGQKVDGIYGGRAFVNHADEVAALNVALVRL

SEQ ID NO: 8 - protein, 1029 aa, unidentified
MKTETLIRPYPGKLNLQPRRAQFLEDSIQYHQKMTEFFYQFLQAVGGATTHQNISDFIDNKATDEHQATLLFQVVSKDSTTPECPAEELLARFAQYTGKQPNEAVTHYLTSRINTDKYRWQDNRLLAQNIASQLNISETQFQEIAHAILSNNLYIGQTASNAAANFISQVTGTGQKAPKAARLDVLFQTNQALAKTQPTTFGQLQQIIVQACGESTTDAVLAKFGNKGAATSLQLALKTDPNTTLDQKKYEALQKKFAEDETKYRNKVDIPHKTQLRNLILNTSNQFCNWHTKPAIEAFKCAIADIQSKVSNNLRIMQEKAKLYEAFRNVDPQVQIAVQALENHMNTLEEPYAPYAHSFGSVKDFYEDLNNGSNLDEAIQTIVHDSDNFNRKPDPNWLRIIAPLHSSHSASQIMEAVKYLSSKQDYELRKPFPFVATNLPATYGKFNIPGTLNPPTDSLHGRLNGSHSNMWLTALLLDGRDWKNHHLCFASSRYFEEVYFTNPSLPTTDKVRSPKCGFTLKSVLDSEAKDRIRNAPKSRTKAVKAIERIKANSTHNVAWNPETSFQMQKRNDEFYITINHRIEMEKIPGQKKTDDGFTIHPKGLFAILKEGDRILSQDLNQTAATHCAVYEVAKPDQNTFNHHGIHLKLIATEELKMPLKTKKSTIPDALSYQGIHAHDRENGLQQLKDACGAFISPRLDPKQKATWDNSVSKKENLYPFITAYMKLLKKVMKAGRQELKLFRTHLDHILFKHNLSPLKLHGVSMIGLESSRATKSVINSFFNLQNAKTEQQQIALDRPLFEAGKTLINNQTRRRQERVRLETSLTMRLAHKYNAKAIIIEGELPHSSTGTSQYQNNVRLDWSAKKSAKLKTESANCAGIAICQIDPCHTSHQNPFRHTPTNPDLRPRFAQVKKGKMFQYQLNGLQRLLNPRSKSSTAIYYRQAVQSFCAHHNLTERDITSAKFPSDLEKKIKDDTYLIPQRGGRIYISSFPVTSCARPCTSNHYFGGGQFECNADAVAAVNIMLKVHP

SEQ ID NO: 9 - protein, 1011 aa, unidentified
MPIRGYKCTVVPNVRKKKLLEKTYSYLQEGSDVFFDLFLSLYGGIAPKMIPQDLGINEQVICAANWFKIVEKTKDCIADDALLNQFAQYYGEKPNEKVVQFLTASYNKDKYVWVDCRQKFYTLQKDLGVQNLENDLECLIREDLLPVGSDKEVNGWHSISKLFGCGEKEDRTIKAKILNGLWERIEKEDILTEEDARNELLHSAGVLTPKEFRKVYKGAAGGRDCYHTLLVDGRNFTFNLKTLIKQTKDKLKEKSVDVEIPNKEALRLYLEKRIGRSFEQKPWSEMYKTALSAVMPKNTLNYCFAIDRHAQYTKIQTLKQPYDSAITALNGFFESECFTGSDVFVISPSHLGKTLKKLYNYKDVESGISEIVEDEDNSLRSGVNVNLLRYIFTLKDMFSAEDFIKAAEYNVVFERYNRQKVHPTVKGNQSFTFGNSALSGKVIPPSKCLSNLPGQMWLAINLLDQGEWKEHHIPFHSARFYEEIYATSDNQNNPVDLRTKRFGCSLNKTFSAADIEKVKESAKKKHGKAAKRILRAKNTNTAVNWVDCGFMLEKTEVNFKITVNYKLPDQKLGKFEPIVGTKILAYDQNQTAPDAYAILEICDDSEAFDYKGYKIKCLSTGDLASKSLTKQTEVDQLAYKGVDKTSNFYKKWKQQRRLFVKSLNIPDALKSFENINKEYLYGFNNSYLKLLKQILRGKFGPILVDIRPELIEMCQGIGSIMRLSSLNHDSLDAIQSLKSLLHSYFDLKVKEEIKTEELREKADKEVFKLLQQVIQKQKNKRKEKVNRTVDAILTLAADEQVQVIVGEGDLCVSTKGTKKRQNNRTIDWCARAVVEKLEKACKLHGLHFKEIPPHYTSHQDCFEHNKDIENPKEVMKCRFNSSENVAPWMIKKFANYLKCETKYYVQGMQDFLEHYGLVEYKDHIKKGKISIGDFQKLIKLALEKVGEKEIVFPCKGGRIYLSTYCLTNESKPIVFNGRRCYVNNADHVAAINVGICLLNFNARAKVAEKTP

SEQ ID NO: 10 - protein, 1093 aa, unidentified
MAKKDFIARPYNSFLLPNDRKLAYLEETWTAYKSIKTVLHRFLIAAYGAIPFQTFAKTIENTQEDELQLAYAVRMFRLVPKDFSKNENNIPPDMLISKLASYTNINQSPTNVLSYVNSNYDPEKYKWIDSRNEAISLSKEIGIKLDELADYATTMLWEDWLPLNKDTVNGWGTTSGLFGAGKKEDRTQKVQMLNALLLGLKNNPPKDYKQYSTILLKAFDAKSWEEAVKIYKGECSGRTSSYLTEKHGDISPETLEKLIQSIQRDIADKQHPINLPKREEIKAYLEKQSGTPYNLNLWSQALHNAMSSIKKTDTRNFNSTLEKYEKEIQLKECLQDGDDVELLGNKFFSSPYHKTNDVFVICSEHIGTNRKYNVVEQMYQLASEHADFETVFTLLKDEYEEKGIKTPIKNILEYIWNNKNVPVGTWGRIAKYNQLKDRLAGIKANPTVECNRGMTFGNSAMVGEVMRSNRISTSTKNKGQILAQMHNDRPVGSNNMIWLEMTLLNNGKWQKHHIPTHNNKFFEEVHAFNPELKQSVNVRNRMYRSQNYSQLPTSLTDGLQGNPKAKIFKRQYRALNNMTANVIDPKLSFIVNKKDGRFEISIIHNVEVIRARRDVLVGDYLVGMDQNQTASNTYAVMQVVQPNTPDSHEFRNQWVKFIESGKIESSTLNSRGEYIDQLSHDGVDLQEIKDSEWIPAAEKFLNKLGAINKDGTPISISNTSKRAYTFNSIYFKILLNYLRANDVDLNLVREEILRIANGRFSPMRLGSLSWTTLKMLGNFRNLIHSYFDHCGFKEMPERESKDKTMYDLLMHTITKLTNKRAERTSRIAGSLMNVAHKYKIGTSVVHVVVEGSLSKTDKSSSKGNNRNTTDWCSRAVVKKLEDMCVFYGFNLKAVSAHYTSHQDPLVHRADYDDPKLALRCRYSSYSRADFEKWGEKSFAAVIRWATDKKSNTCYKVGAVEFFKNYKIPEDKITKKLTIKEFLEIMCAESHYPNEYDDILIPRRGGRIYLTTKKLLSDSTHQRESVHSHTAVVKMNGKEYYSSDADEVAAINICLHDWVVPLNWTNHCLPAGWCSDHLKECVQCHTPDPVRISM

SEQ ID NO: 11 - RNA, 36 nt, unidentified
  source 1..36: mol_type = other RNA
ctagcaatgactcagaaatgtgtccccagttgacac

SEQ ID NO: 12 - RNA, 36 nt, unidentified
  source 1..36: mol_type = other RNA
atcgcaacatcttagaaatccgtccttagttgacgg

SEQ ID NO: 13 - RNA, 36 nt, unidentified
  source 1..36: mol_type = other RNA
tctcaacgatagtcagacatgtgtccccagtgacac

SEQ ID NO: 14 - RNA, 36 nt, unidentified
  source 1..36: mol_type = other RNA
ctcaaagtgtcaaaagaatgtccctgctaatgggac

SEQ ID NO: 15 - RNA, 37 nt, unidentified
  source 1..37: mol_type = other RNA
tcccaaagtggcaaaagaatctccctgttaatgggag

SEQ ID NO: 16 - RNA, 36 nt, unidentified
  source 1..36: mol_type = other RNA
gtctaactgccatagaatcgtgcctgcaattggcac

SEQ ID NO: 17 - RNA, 35 nt, unidentified
  source 1..35: mol_type = other RNA
tcggggcaccaaaataatctccttggtaatgggag

SEQ ID NO: 18 - RNA, 36 nt, unidentified
  source 1..36: mol_type = other RNA
ccacaacaaccaaaagaatgtccctgaaagtgggac

SEQ ID NO: 19 - RNA, 36 nt, unidentified
  source 1..36: mol_type = other RNA
gtaacagtggctaagtaatgtgtcttccaatgacac

SEQ ID NO: 20 - RNA, 24 nt, unidentified
  source 1..24: mol_type = other RNA
gagagaatgtgtgcaaagtcacac

SEQ ID NO: 21 - DNA, 3243 bp, unidentified
  source 1..3243: mol_type = other DNA
atgtctagtgatgtcgttcgtccatataacaccaaactgcttccagataatcgcaaacacaatatgtttttgcaaactttcaagcgacttaattctatttctcttaatcattttgatctcttaatttgtctttatgctgccattaccaacaagaaggcagaagaatataagtctgaaaaagaagctcatgtaaccgctgatagcctttgtgctatcaattggttccgtcctatgtccaagcgttacagcaaatacgcaactacaactttcaatatgcttgaattgttcaaagaatactctgggcatgaaccagatgcttattccaagaattatcttatgtccaatattgactcagacaggtttgtctgggttgattgccgtaaatttgccaaagattttgcgtatcaaatggaacttggtttccatgaatttacagtcttggcagaaaccttgttggcaaatagtattcttgtactcaacgaatcaactaaggcaaattgggcatggggcaccgtttctgcactttacggtggaggcgataaggaagattctacgctgaagtcgaaaatccttttggcttttgttgatgcactcaataaccacgaacttaaaactaagcgtgaaattctcaatcaagtttgtgaatcactaaaatatcaatcataccaagacatgtatgttgatttccgttctgttgttgacgaaaatggaaacaagaagtctcccaatggctcaatgccaatcgtcaccaagtttgaaacagatgatttgatttctgataatcaacgcaaagcaatgatttctaatttcacaaagaatgctgctgctaaagcggctaaaaaacctattccctacctagacagactcaaggaacatatggtttccttgtgcgatgaatataatgtttatgcttgggcagcagctatcactaactctaatgccgatgtaacagctaggaatactcgcaatttaacattcatcggggaacaaaattctcgaaggaaagaactatcggttttacaaactacaacaaacgaaaaagcaaaagatatcttgaataagattaatgacaatcttattcaagaagtaaggtatacccctgcccccaagcacttggggcgtgatcttgccaatctttttgatactctgaaagaaaaagatatcaataatattgaaaacgaagaagagaagcagaatgtaattaatgattgcattgagcaatatgttgatgattgccgttcactgaaccgcaatcccattgctgctttgctcaagcacattagccgatactatgaagatttttcagccaagaatttcttggatggtgccaagttgaatgtcttgactgaagttgtaaatcgtcaaaaggcacatccaactatttggtctgaaaaggcttatacttggatttccaagtttgacaagaataggcgacaagcaaactcttctttggttggatgggttgttccaccagaagaagtccataaagagaagattgctggtcaacaaagcatgatgtgggtcactttgactctgcttgatgatggcaagtgggtaaagcaccatattcctttttcagattccagatattattctgaagtctatgcctacaatccaaatttgccatatcttgatggtggtattccacgccagtcaaagtttggcaataaaccaaccactaatctgactgctgaaagtcaagcgttacttgcaaacagcaagtataaaaaggcaaataagtcatttctccgtgccaaggaaaatgctactcacaatgtccgtgttagtccaaacacttccttgtgcattcgtttgctcaaggatagtgctggtaatcaaatgtttgataagattggcaatgttctgtttggaatgcagatcaaccataaaatcaccgttggcaagcccaactacaagatcgaagttggtgataggttccttggtttcgaccagaaccaaagtgaaaaccacacttatgctgtcttgcaacgagtctctgaaagctctcatgacactcatcattttaatggatgggatgtcaaggttcttgaaaagggcaaagtaacaagtgatgtcatcgttagagatgaggtctatgaccaacttagctatgagggcgttccttatgattcttcaaagtttgcagaatggagagacaagaggagaaggtttgttttggaaaacttgtctatccagttggaagaaggcaaaacattcttgactgaattcgacaaattaaataaagattctctttatcgttggaatatgaattatctgaaactgctcaggaaagctattcgtgccggtggcaaggaatttgccaagattgctaagactgagatttttgaattggcagttgaaaggtttggaccaatcaaccttggtagtttgtcacaaattagcttgaagatgattgcatctttcaagggagtggttcagtcttacttttctgtatctggttgtgttgatgacgcatccaagaaggcacatgattccatgctcttcactttcatgtgtgcagcagaagaaaaaaggacaaacaaaagagaagaaaagactaatcgtgcagcatcttttatcttgcagaaagcatatttgcatggctgcaagatgattgtttgcgaagacgatcttcctgttgctgatggaaaaacaggcaaggcacaaaatgcggatcgtatggactggtgtgcccgtgctttggcaaagaaagtcaacgatggttgtgtggcaatgtctatctgctatcgtgccattccagcttatatgtctagccaccaagatccatttgttcacatgcaagacaaaaagacttctgttttgcgtccaaggttcatggaagttaacaaggatagcatcagggattatcatgttgctggtttgcggagaatgctgaacagcaagagtgatgcaggcacttccgtttactatcgtcaggcagctttgcatttctgcgaagcgttgggcgtgtctccagaattagtcaagaacaaaaagactcatgctgccgaattaggaaagcatatgggttctgccatgttgatgccttggcggggtggcagggtttatattgccagcaagaagttgacttcggatgctaaaagtgtaaaatactgtggagaagatatgtggcagtatcatgctgatgagattgctgctgtcaatatcgcaatgtatgaagtttgctgccagacaggtgcgtttggcaagaagcaaaagaagagtgatgaactaccgggataa

SEQ ID NO: 22 - DNA, 3244 bp, unidentified
  source 1..3244: mol_type = other DNA
catgtctagtgatgttgttcgtccatataacactaagctgcttcctgataatcgcaaatacaatatgtttttgcaaactttcaaaagactcaatttgatttcatcaaatcattttgatctcttggtttgtctttatgctgctatcaccaacaaaaaagctgaagaatataagtcagaaaaagaagatcatgtaaccgctgatagcctttgcgccatcaattggttccgtcctatgtccaagcgttatatcaaatacgcaaccactacttttaagatgcttgaattgtttaaggagtactctggtcatgaaccagatacttattccaagaattatctcatgtccaatatcgtctcagataggtttgtttgggttgattgccgcaaatttgccaaagattttgccaatcaaatggaacttagtttccacgaatttaccactttgtcagagactttgttggcaaatagtatccttgtactcaatgagtcaaccaaggcaaattgggcatggggtgctgtttcagcactttatggtggaggcgacaaagaagattctacgctgaagtccaaaatccttttggcttttgttgatgctctcaataatcctgaacttaaaactaggcgggaaattctcaatcatgtttgtgaatcactaaaatatcaatcataccaagatatgtatgttgattttcgatctgtcgttgatgataagggaaacaagaagtctcccaatggctcaatgccaatcgtcactaagtttgaatcagatgatttgattggtgacaatcaacgcaaaactatgatttctagtttcacaaaaaacgccgctgccaaagcgtctaagaagcccattccatatctagacattctaaaagaccacatgatttccttgtgcgaggaatacaatgtctatgcttgggcagcagctattaccaattccaatgctgatgtaactgctagaaacactcgcaatctgacattcatcggggaacaaaatacccgaaggaaagaactatcggttttacaaacttctacaaacgaaaaagcaaaagatatcttaaataagattaacgacaatcttattccagaagtaaggtacacccctgctcccaagcacttggggcgtgatcttgccaatctttttgaaatgttcaaagaaaaagatataaatcagattggaaatgaagaagaaaagcaaaatgtgatcaatgattgcattgagcaatatgtcgatgattgccgttcattgaaccgcaatcctgttgcagctttgctcaagcatattagcggatattatgaagatttttcagccaagaatttcttggatggtgccaagttgaatgtcttgacggaagttgtcaatcgtcaaaaggcacatccaactatttgttctgaaaaggcttatacttggatttccaagattgacaagaataggcgacaagcaaactcttctttggttggatgggttgttccaccggaggaagtccataaggaaaaaattgccggtcaacaaagcatgatgtgggtcactttgactttgcttgatgacggcaagtgggtaaagcatcatattccttttgcagactcaagatattattctgaagtctatgcctataatccaaatttgccatatcttgaaggtggtattccacgacaatcaaagtttggcaataaaccaacaactaatttgaccgctgaaagccaagcattacttgccaacagtaagcacaagaaagccaacaagacatttctccgtgccaaggagaatatcactcacaatgttcgtgttagtccaaatacttcattgtgcattcgtcccctcaaggatagtgctggtaatcaaatgtttgacaacattggtaatatgttgtttggaatgcagatcaatcacagaattactgtcggcaagccaaactacaagatcgaagttggtgatcggttccttggttttgaccagaaccaaagcgaaaaccacacctatgcagttcttcaacgagtatccgaaagctctcatggcactcatcatttcaatggttgggatgtcaaagtgattgagaagggcaaggtgacaagtgatgtcgtcgtcagagatgaagtctatgatcaattaagctacgagggtgtcccttacgattctccaaagtttacagaatggagagagaagaggcgaaagtttgtcttggaaaatatgtcaatccagattgaagaaggcaaaacattcttgactgaatttgacaagttaaacaaagactctttgtatcgttggaacatgaattacatgaaattgcttaggaaggcaattcgtgctggtggcaaggaatttgccaagattacaaaggctgagatttttgaactaggagttatgagatttggaccaatgaacttgggcagcttgtcgcaagtcagcttgaagatgattgctgcttttaagggagttattcagtcttacttttccgtatctggttgcattgatgacgcatccaagaaagctcatgattcgatgttattcgctttcttgtgttcagcagatgagaaaaggacaaacaagagggaagaaaagacaaatcgtgcagcatctttcatattgcagaaagcatactcgcatggttgcaagatgattgtttgcgaggatgatcttcccattgccgatggcaaggtgggcaaggcacaaaatgcggatcgcatggactggtgcgcccgttcattggcaaagaaagtcaacgatggttgtgtggctatgtccatatgttatcgtgccattccagcatatatgtcaagccatcaagatccatttactcatatgcaagataaaaagacttctgttttgcgtccaaggttcatggaagtcggcaaggatagcattagggatcatcatgttgctggtctgcggagaatgctgaacagtaaaggtaatactggcacttctgtttactatcgtgaggcagctttgcgtttctgcgaagcgttgggtgtgcttcccgaattagtcaagaacaaaaagactcatgcttcggaattaggaaagcatatgggttctgccatgttgatgccttggcggggtggcaggatctatgtcgccagcaagaaattgacttcggatgccaagagtataaaatattgtggagaagatatgtggcaatatcatgctgatgagattgctgctatcaatatcgcaatgtatgaggtctgctgtcagacaggtgcttttggcaaaaaacaaaagaagagtgatgaactaccgggataa

SEQ ID NO: 23 - DNA, 3225 bp, unidentified
  source 1..3225: mol_type = other DNA
atgggtattagcatttcacgtccgtacggtacaaagttgcgtcctgatgctcgtaagaaggaaatgttggataagtttttcaccacgctagcaaaaggtcagcgtgtttttgcggatctgggactgtgcatttacggcagccttactttagaaatggtaaagcggcttgagccagaatccgattctgaacttgtctgtgcaattggttggtttcgtcttgtagataaggtaacttggtctgagaatgaaattaaacaagagaacctggttagacaatatgagacgtattcaggaaaagaagcgtctgaggttatcaagacttacctaagctctccaagttcagacaagtatgtgtggatagactgccgacaaaagtttcttaggtttcaaagggatctgggaacacgtaatctgtctgaagactttgagtgcatgctttttgaacagtacctcagactcacaaagggagagcttgatgggcataccgctatgtccaacatgtttggaacaaaaacaaaagaagatcgcgccacaaaactgagatatgccgcaaggatgaaagaatggctcgaggctaacgaagaaattacttgggaacaatatcaccaagcgttgcaagataaattagacgccaatactttagaggaggctgttgataattacaaaggcaaagcgggaggctctaatccattttttagttacacgcttttaaacagaggtcagattgataaaaaaactcacgagcagcaattaaagaaattcaacaaagttctaaaaaccaaatccaaaaatttaaattttccaaacaaagagaagttaaaacaatatttagaaacagcaattggtattcctgttgatgctcaggtctacggtcagatgtttaataacggcgtttctgaagttcaaccaaagacaacgcgcaacatgtctttttctatggagaagcttgagcttttaaacgagttgaaaagtctcaacaagactgacggttttgaacgcgctaatgaagtcttgaatggtttctttgattctgaacttcacactactgaagacaagttcaacatcacttccaggtatttgggtggagacagaaacaatcggctaccaaagctgtacgagctttggaaaaaggaaggagtagatcgtgaggaaggtatccagcaattcagccaagcaatccaagataagatgggtcagatacctgttaagaatgtccttaggtatatttgggaatttcgtgagactgtttctgccgaagactttgaagcggcagcgaaagcgaatcagttggaagaaaaaatcacgcgtaccaaagcgcaccccgttgttatatctaacaggtattggacatttggctcttcggctcttgttggtaatatcatgccagcagacaagatgcacaaagaccagtacgcaggtcaaagtttcaagatgtggcttgaagccgaactgcactacgacggtaagaaagtcaaacatcacttgccgttctacaacgccaggttctttgaagaggtctactgctatcacccgagcgtagctgaagttacaccattcaaaaccaagcagtttggttatgcaattggaaaagatattccagctgacgtttcggttgtactgaaagacaatccttataaaaaggcaaccaagcgcttccttcgggctatcagcaatccagtcgccaacacagtggatgtaaacaagcctacagtttgctcattcatgattaaacgagaaaatgacgaatacaaactagtcattaatcgaaagatcggtgttgatcgcccaaagcgtattaaagtaggtaggaaggtcatgggctatgaccgtaaccaaactgcttctgatacttactggattggagagcttgttccacatggaacaaccggagcgtaccgtattggagaatggagcgtccagtatatcaagagcggtcccgtgttgtcttctacgcaaggcgtaaatgacagtactacggatcaacttatatacaacggaatgccgagctccagcgaacgttttaaagcttggaagaaatctaggatgtctttcattcgtaagttgatacgccaactgaacgccgaaggcttggaaagtaaaggacaggactatgttcctgaaaatccaagtagctttgatgttaggggcgaaacactttacgtattcaacagcaactatatgaaagctttggtgtctaagcatcgaaaagccaagaaacctgttgaaggtattcttgaagaaatagaagccttgacaagcaaagctaaagattcttgttcgttgatgcgtttgagttctttgtctgatgcggctatgcaaggtattgcttcgttgaagagtttgatcaactcatacttcaacaagaatggttgcaaaacaattgaagacaaagaaaagtttaacccagatctgtatgtgaaacttgttgaagttgagcaaaagagaactaacaagagaaaagaaaaagttggtcgaatcgccggttctcttgaacagttagctttgcttaacggtgttgacgttgttatcggtgaagctgatcttggcgaagtcaagaaaggcaaatccaaaaaacaaaatagtcgaaacatggactggtgtgccaagcaagtcgctgagcggcttgagtacaagctgaccttccattgtattggttattttggtgtcaacccgatgtatacgtctcatcaagatccatttgaacatcgtcgcgttgctgaccacctagtaatgcgtgcgaggtttgaagaagtgaatgtaagtaatgtttcggaatggcacatgcgaaacttctcaaactatctgcgtgcggactcaggtactggtttgtattacaaacaagctaccttggatttcctcaagcattatgatttggaagagcacgccgatgatttggaaaagcagaatatcaaattctatgacttcaggaaaattcttgaagacaaacaattgacttctgttattgttccaaaacgtggcggtcgcatttacatggcgactaacccggtaacttccgatagtacgcctgtcacttatgccggtaaaacttacaaccggtgtaatgctgacgaagtggctgcggctaacatcgctatcagcgtcttagctcctcactctaagaaagaagaaaaggaagataagatcccgattatttctaagaagcctaagtctaagaatactcccaaggcccggaagaatttaaagacttctcaacttcctcagaaa

SEQ ID NO: 24 - DNA, 3012 bp, unidentified
  source 1..3012: mol_type = other DNA
atggctagcaaacatgtagtgcgtccctttaatggcaaagtaacagctactggcaagcgtttggcatacttggaagaaacttttcattatttggaaaaagctgctggtggtgttagtactttgtttgctgcccttggttcttatcttgatgcaaccacaataagcaatttaattaataaaaatcaagatttagccgttgtaatatttcgttatcatgtggttcccaaaggtgaggctcatactttacctgtaggtacagacatggttagtcgttttgttgccgactatggtatggagccgaatgagtttcagagagcttatttggacagtccgattgaccaagaaaagtattgttggcaggataatagggatgttggttgttggttgggtgagcaattgggtgttagcgaagcggacatgcgggcaatagcagtaactttttataacaatcagatgctttatgattgtgtaaaaggtactgggagtggtaatgctgtgagtcttttgtttggcagtggtaaaaagtctgattacagtatgaagggcgttatagcaggtaaggctgcttcagtactggcaaaatatcgcccagctacctatcaagatgcccgaaagatgattttggaagctaatggtttcacctcagtaaaagatttggttacttcttatggaataactggaaggtctagtgctttgcagatatttatggaagggattgaaagtggtcctattagcagcaagacattagatgctcgtattaagaagttcacagaggattcggagcgcaatggcaggaagaatctagtccctcatgctggggctatacgaaattggctgattgagcaagctggtagtagtgtagaaaactatcagatggcatggtgcgaggtttacggtaatgtgtctgccgactggaatgccaaagtagaaagtaatttcaatttcgtagcggagaaagtaaaggcattaacagaattatccaacattcagaaatcgactcctgatttgggtaaggctttgaaattatttgaagaatatttgactacttgtcaggatgaatttgctattgcgccttatcattttagcgtcatggaagaggtgcgaatggaaatggcaacaggcagggaattcaatgatgcttatgatgacgccctaaatagcttggacatggagtctaagcagcccattcagcctttgtgtaagtttttgattgagcgtggaggtagtatcagttttgatactttcaagagtgcagccaagtatttgaaaacacagagcaagattgctggtcgatatccacatccatttgtaaaaggtaatcagggatttacttttggttccaaaaacatttgggcagccatcaacgatcctatgatggagtatgcagatggtcgtattgctggtggttctgcaatgatgtgggtgacggctacattgttggatgggaaaaagtgggttcgccatcatatcccatttgccaatactcgatactttgaggaggtttatgctagcaagaaagggttgcctgtattgccttgtgctagagatggcaaacactcatttaaattgggcaataatttgagtgtagagagagttgaaaaggtcaaagaaggcggtagaactaaagcaaccaaggcacaagagcgtattttaagcaacttgactcacaatgtgcagtttgacagttcgacaacttttattattcgtcgtcaggaagaaagttttgtaatttgcgtgaatcatcgacatccagctccgctcatgaagaaggagatggaagttggcgacaaaatcattggtatcgaccagaatgtgacggcacccacaacctatgccatagttgagcgtgtggcttctggcggcattgagcgtaacggcaagcagtacaaagtgacggcgatgggagccatttccagcgttcagaagaccagaggcggtgaggtggatgttttgagttatatgggggttgaactttctgacagcaaaaatggatttcaaagcttgtggaataaatgtttggactttgttaccaaacatggcactgaaaatgatgttaaatattataacaacactgctgtctgggccaacaagctgtatgtgtggcacaagatgtatttccggcttttgaagcagttgatgcgtcgggcaaaggacttgaaacctttcagggaccatttacagcatctattattccatcctaatcttagtcccttgcaacgccatagcttgtccttaacaagtctggaagcaactaagatagtgcggaattgcattcattcgtatttcagtctattggggttgaagaccttggatgaacgcaaagccgctgacatcaatttattggaagttttggaaaagctgtatgctggtttggttgagaggcgaaaagaaagaaccaaactaaccgctgggctattggttcgcttatgtaatgagcatgggatttcttttgcagctattgagggtgatttgccggtcgttggagagggcaaatctaaagctgccaacaatacacaacaggattggacagccagagagttagagaagcgattatctgagatggcggaggtggttggcatcaaggtaatagctgttttgccccactataccagtcatcaggacccatttgtttatagtaaaaataccaagaaaatgagatgtcgttggaactggaggaccaccaagaccttcactgatcgtgatgctttgagtatacgcaggatattaagcaagcctgagacgggtacaaatttgtattatcagaagggcttgaaagcatttgctgaaaagcatggtctggatttggcagagatgaagaagcgcaaggatgctcaatggtatcttgagcgcattcaagacaagaattttttggtgccaatgaatggtggtagagtttatttgagttctgtcaaattagccgggaaagaaacaattgacatgggtggcgaaattttatatcttaacgatgccgatcaagtcgcagcgttgaatgttttgttagtgaagatttga

SEQ ID NO: 25 - DNA, 3058 bp, unidentified
  source 1..3058: mol_type = other DNA
atggctaagaaagaacatattataagaccattcaaaggaacactaccacttcgtggtgatagactaaggtatcttcaagataccatgaaatatatgaaaaaggttgaagatactatcacagaactctgcgccgctgttatcgcctatgccaaacccaccatcattcaacaaatacttggcgaagaaattgaaaccaccagcacattttgtagcttccgcttagtaggcattcatgaaaactttaccatgccactaaccacaaatatgataaaacacttccagaaaacctttaacataaacccatcagaaaaacaagcaatctatctctccagtggattcgattcagataaatatcgctggcaagatacttccgaagtatccagaaacttcgccaacaaatgccgacttactaatcaagaattccaagaatttgccgaacaagcactactcaatatgtgcttcataggttgctctggtagccccggtgcaactaatgccgtctcacaaatctttggcacaggcgaaaaaagcgattaccaacgcaaaagccaaatcgctaaaattgctgctgataccctcgaaaaccacaaacctagcacctatgagtctgctagattaatggttcttaatacacttggacacaaaacaatagaagattgtgtcaatgactatggcgcaataggagccaaatccgccttccgactattcatggaatcaaaagaaataggaccaattacatctgaacaactcacaaccaaaattaagaagttcagagaagatcataaaaagaactccatcaagaaacaacttccacatgtagaaaaagttcgtaacgctttgctatcacaattcaaagaacaatacctgccctcagcatgggcagaagcatggtgcaatatcatgggcgaatttaactccaaattatcaaataataataacttcatcgaccaaaaaacaaaaatggtcaatgactgcgataatattaaaaaatctaatccacaactagacaaagctgttaatatgctcgatgaatggaaatataaaaactgggatgataattctgctatacacccatatcatattggcgatcttaaaaaactcatggcaatattcaatatcaataacgaaggaaccttcgacgaaagattttcagctagctgggaacaattctccacatcactagaatacggggagaaaccacccgttcgtgatctactagcccatatcatcaaaaatatgaatgacctcacctacacagacgtaatcaacgccgcaaaatttctcaaacttcaagataatataagaaataaatacccacaccctttcgttatgccaaataaaggatgtacctttggtaaagataacctttggggcgaaattaatgaccccacagccaaaatcaaatcaacagaagaagttgctggacaaagacctatgatgtggctgacagccaaacttctcgataatggaaaatgggtagaacaccacatccctttcgcctccagtagatactttgccgaagtttattataccaatccagcactccccactctaccaatagctagagatggaaaacattcatacaaattaacaaaaactatagatgccaatactgcaaaaactctagtaaataatcctagagataaagcagctaaactaatcgcacgaactaaagccaatactacacacaatgtaaaatggattaaacctacatacagaatccaaaaagaaaataaccaattcgttattactatcaatcatcgacacccatgcataacaccaccaaaggaaatcatactcggagatcgtatcctatccttcgaccaaaacgaaacagcccccacagcattctccattctcgaaaaaacaaccaaaggtacagaattctgtggccaccacattaaagtgctaaagactggtatgctagaagctaaaattaaaaccagtaagaaatcaatagatgcattcacatacatgggaccaatggaagatgatcatgcgtctggcttcccaacactactcaacatatgtgaaaaattcatatcagagaatggagatgaaaaagacaaaagtttctcttctcgtaaattgccctttaaaaggtctttgtacttctttcatggctcacacttcgatttactaaagaaaatgatcagaaaggccaaaaatgaccccaagaaattgaagttagtaagaattcatatcaatgaaattctattcaattccaatttgtcaccaataaaactacacagtctgtctattcacagcatggaaaataccaaaaaagttatagctgctattagctgctatatgaatgttcatgaatggaaaactatcgatgaacaaaagaatgctgatataacattgtataatgctaaagaaaaactatacaacaaccttgttaaccgccgtaaagaaagagtaaaagtaactgcaggtatgttgattcgattagctagagaaaacaattgcagattcatggtcggggaagcagaattacccacccaacaacaaggcaaatcaaaaaagaacaataactccaaacaggattggtgcgccagagatatagcacaacgatgtgaagatatgtgcgaagtcgtaggtataaaatggaatggcgttactccgcataataccagccatcaaaacccattcatctataaaaatactagtggacaacaaatgcgatgccgttatagtctcgtaaagaagtcagaaatgacagacaagatggcagaaaaaattagaaatattttacacgctgaacctgtaggcactacagcatactaccgtgaaggcattttggaattcgccaaacatcatggattagatctgggaatgatgaaaaaacgaagagatgctaagtattatgataatcttccagatgagtttctgcttcctactagaggtggtagaatctatctgtccgaaaatcaactaggcggaaacgaaaccattgttattaatgggaaaaaatattttgtcaatcaggcagatcaagtcgctgccgtaaatattggcctgctttatcttctgccgaagaaaaaccagagttaag

SEQ ID NO: 26 - DNA, 3144 bp, unidentified
  source 1..3144: mol_type = other DNA
atgtccgagaagaagttccacatcaggccctaccgctgctcgataagcccgaacgcccgcaaggccgatatgctcaaggcgacgatctcctaccttgactccctgacctccgtgttcaggtcgggattcaccgcactacttgcgggcatagacccgtcgacggtgagccgcctggcgccttcgggggccgtcggcagcccggacctgtggagcgccgtcaactggttccgcatcgtgccgctcgcagaggccggcgacgcccgagtcggccaggcatcgctcaagaacctcttccgtggctacgcaggccacgagcccgacgaagaggcgtcgatctatatggagtcgagagtggacgataagaggcacgcgtgggtggactgccgtgccatgttcagggcgatggcgctcgagtgcgggctggaggaggcccagctcgcctccgacgtgttcgccctcgcctcaagggaggtcatagtcttcaaggacggcgagatcaacggctggggcatagcctccctgctgttcggcgagggcgagaaggccgactcgcaaaagaaggtcgccctgctccgctccgtgaggctggcccttgagggggactacgcgacctacgaggaactctccgggctcatgctggccaagaccggagcctccagcggctccgacctccttgacgagtacaagaggagcgagaagggcggcagcagcggcggcaggcaccccttcttcgacgaggtcttccggaggggcggcagggtcaagcaggaggagcgcgagaggctgctgaagagctgcgacacagcgatccagaagcaggggcaggcgctgccgctgtcgcacgtcgcatcttggaggcaatggttcctgcgcagggtcacgctgctgcgcaaccgcaggcaagagtcgttcgcagtctgcatcaccaacgccctcatggacctacagcccaagaacctacgcaacgtccactacgtgacgaaccccaagagcgagaaggacaagggcgtgctcgagctgcgcgtcgacgtcaagaacaacgaggggccggacgtggcgggcgcgcaggcggtcttcgacgcctacatggcgaggctggcacccgacctgcgcttctccgtgatgccacggcacctcggctccctcaaggacctctacgccctttgggccaagctcgggcgggacgaggccatcgaggagtacctcgagggctacgagggaccattcagcaagaggcccatcgcaggcattctacaaatcatccacgcacaccgtggcaaggtgggctacgatagcctgttgcgtgcggcgaggctcaacagggcgatggacaggctggagaggaagagggcccacgcctgcgcagccggcaacaagggttacgtctacggcaagagctcgatggtcggccgcatcaacccgcagagcctcgaggtcggcggccgcaagtcgggccgaagcccgatgatgtgggtgaccctcgacctggtggacggcgacaggttcgcgcagcaccaccttcccttccagagcgcccgcttcttctccgaggtctactgccacggcgacgggctcccggccacccgtgtccccggcatggtcaggaaccgtcgcaacgggctggcgatagggaacgggctcggggagggtggactctcagcgctgcgcgcaggcagcgacaggaggaagagggccaacaagaggacgctgcgcgccctcgagaacatcacgcacaacgtggagatcgaccccagcacctccttcacgctgcgggaggacgggataatcatttcgcacaggatcgagaagattgagccgaagcttgtcgccttcggggacagggcgctcggcttcgacctcaaccagacaggggctcatacgtttgcggtgctccagaaggtggactcgggcggcctagacgtcggccactctcgcgtgtcgatcgtgctcaccggcactgttcgcagcatctgcaagggcaaccaggcgagcggcggacgggactacgacctgctttcctacgacggccccgagcgcgacgacggggcgttcacggcatggaggtcggacaggcaggccttcctgatgtctgccatacgggagctgcccacgcccgccgagggggaaaaggactacaaggcagacctcctctcccagatggcgagccttgaccactacaggcgactgtacgcgtacaacaggaagtgcctcggcatctacatcggggccttgagacgcgcgaccaggaggcaggccgtggccgcattcaaggacgagatactctcgatcgcgaatcaccgctgcgggcctctcatgcgtgggagcctttcggtgaacggcatggagtccctcgcgaacctcaagggcctagccacggcatacctgagcaagttcaaggacagcaagtccgaggacctgctgtcgaaggacgaggagatggccgacctgtacagggcttgcgcgcgcagaatgactggcaagcgcaaggagaggtacaggagggcggctagcgagatcgtccggctggccaacgagcacggctgcctgttcgtcttcggcgagaaagagctgcccaccaccagcaagggcaacaagagcaagcagaaccagaggaacaccgactggtcggcccgtgccatagtgaaggcggtcaaggaggcctgcgagggctgcggtctcggcttcaagcccgtgtggaaggagtactcgagcctcacggacccgttcgagagggacggggacggaaggcctgccctccgctgccggttcgccaaggtggccgcacccgactccgaactcccgcctcgcctgacgaaggccgtcggctcctatgtgaagaacgccctcaaggccgacaaggcggagaagaagcagacctgctaccagcgtggcgccatcgagttctgctcaaggcacggcatcgacgtccggaaggcgaccgacaaggccattcgcaaggcagtccgtggctcctccgacctgcttgtgccgttcgacggggggaggaccttcctgctctcgacgaggctgtccccggagtcgcgaaaggtggagtgggccgggcgcaccctgtacgagttccccagcgacatggtcgccgcaatcaacatcgcctgcaggggcctagagccacgcaaggcctag

SEQ ID NO: 27 - DNA, 3090 bp, unidentified
  source 1..3090: mol_type = other DNA
atggacgagcaagctgttgtttcctctggttccgacaagaccctcaagatcgtacgcccttacagggcaaaagtaaccgctactggaattcgccttgagggaattaaaaataccctgaattacctgaagcgtacagaaatttgtctgtcacgcctgaatgcagcttgtggagcttttctcactcctgccatcgtggagcagatctgtaaggacgatcctgccctagtttgtgccattgctcgctttcaattggttccggttggtagtgaagccactttgtccgacagtgggctaatgcgtcattttaaggctgctctcggtgaattgaccccgctacaagaagcctacctgaatagcagctataacgacgaattgtacgcatggcaggatactcttgtcttagcgcgacagattattgctgaaaccggattgactgaagatcaattccgcgcctttgctcatgcctgtttcaagaacggcaatattatcgggtgcgctggtggtcccggtgccagcaacgccatctctggcatttttggcgagggaattaaatccgattattcactccgaagtgaaatgaccgctgccgttgcaaaggtgtttgaagagaaacgtcctatcacttacgaagaagctcgggctctcgctctggaagcaactggacacgccagcgttcagtctttcgtggaagcatttggtaaacaggggcgtaaaggcactctgattcttttcatggaagataccaagacaggcgcattcccaagcaatgaattcgattacaagctcaagaaactgaaggaggatgcagagcgtgtcgggcgtaagggtatcatcccgcaccgcgatgtgattgcttcttatctccgcaatcagactggtgctgatattgaatacaactccaaggcatggtgcgagtcctactgttgtgccgtgagcgaatacaactcaaagatgagcaacaatgttcgatttgccacggaaaaaagtcttgatttgaccaagcttgatgaaacgatcagggaaacgcccaagatcagtgaagccatgcttgtttttgaaaactacatggcgcgaattgatgccgatctccggttcattgtgagcaagcatcatctcggcaatctcgccaaattccgtcagaccatgatgcatgtctctgcatcagaatttgaagaggcttttaaggcgatgtgggctgattacttggctggtctggaatacggtgaaaaacccgcgatctgtgaactggtgcggtatgtcctgacccatggcaacgatttgcctgtcgaagcgttttacgctgcgtgcaagttccttagcttggatgacaagatcaagaatcgttaccctcacccatttgttccgggtaacaaaggctacacctttggcgcgaaaaacttgtgggcagaaatcaatgatcccttcaagcccatccgtcaaggcaacccagaggttgctggtcaacgccccatgatgtgggctaccgccgaccttctggacaacaacaaatgggtcttgcatcacatcccctttgcctccagcaggtatttcgaggaagtgtactacaccgatccctcgcttcctacggctcaaaaggcgcgagacggcaagcatggctatcggttgggcaaagtgctggatgaggctgctcgggagcgtttaaaagcaaataatcgccagcgcaaggcagctaaagccatcgagcggatcaaagccaactgtgagcacaatgtggcttgggatccgaccaccaccttcatgcttcagttggattctgagggtaatgtgaaaatgacgatcaatcatcgtcacattgcctatcgcgcacccaaggaaattggtgttggggacagggtgattggcatcgaccaaaacgagactgctcctacaacctacgccattcttgagcgcacggaaaatcctcgcgatcttgaatacaacggcaagtattaccgtgtagtcaagatgggtagtgtgacttcaccgaatgtcagcaagtatcgcacggtggacgctttgacttacgatggcgtgtccttgtcggatgatgcttctggtgctgtgaactttgtggtattgtgtcgcgagttttttgcagcacatggcgacgatgagggtcgcaagtaccttgagaggactttggggtggagttcaagcctgtattccttccatggaaactatttcaagtgccttacgcagatgatgcgtcgatccgctcgttctggtggtgatttgacggtctatcgcgcccatttgcagcagatcctgttccaacacaatctgtcgcccttgaggatgcacagcttgtctttaaggagcatggaatcgacgatgaaggtcatcagttgcatgaagagctacatgtctctttgtggctggaagaccgacgcggatcggattgccaatgataggtcgctgtttgaggctgctcgtaagctttacaccagtttggtaaatcgtcggacggagcgggttcgtgtgactgctggcattctgatgcgtctgtgcttggagcacaacgttaggtttattcacatggaggatgaacttcctgtggctgaaacgggcaaaagcaagaaaagcaatggcgcgaagatgcattggtgtgcccgggagcttgccgttcgtttgtcccagatggcagaggtgacgagcgtcaagttcacaggtgtgtcaccgcattacactagccatcaagacccatttgtgcattccaagactagtaaggtaatgcgtgcccgttggagttggcggaatcgtgccgatttcacggacaaggatgcggagcgtattcggacgattctgggtggtgatgacgcagggacgaaggcttattatcgctcggcgttggctgaatttgcctcgcgctatggtctggacatggagcagatgcggaagaggcgcgatgctcagtggtatcaagagagactgccagaaacctttattattcctcagcggggtggtagagtgtacttgtcttctcacgatctgggatcaggtcaaaaagttgacgggatttatggtggtcgtgctttcgtgaatcacgctgacgaggttgctgcgctgaatgtggcgttggtcaggctgtga

SEQ ID NO: 28 - DNA, 3090 bp, unidentified
  source 1..3090: mol_type = other DNA
atgaagactgaaactcttatccgtccctaccccggcaaactcaacctccaaccccgtcgagcacaattcctcgaagactccattcaatatcaccagaaaatgacggaatttttctaccaattcctccaagcagtcggcggtgccaccacgcaccaaaacatcagcgatttcatcgacaataaagccaccgatgaacaccaagccactctcctcttccaagtagtctccaaagacagcacaacaccagaatgccccgcagaagaactcctagcccgatttgcccaatacaccggcaaacaacccaatgaggctgtcacccactacctgaccagcagaatcaatacagataaataccgctggcaggacaatcgactcctcgcccaaaacatcgcttcacaactgaacatctccgaaactcaattccaagagatcgctcacgcaatcctgtccaacaacctatacatcggtcaaactgcatccaacgcagcagccaacttcatcagccaagtcacaggcacaggccagaaagcccccaaggcagcacggctcgatgtcctgttccagaccaaccaagccctcgccaaaacacaacccacaaccttcggccaactccaacagatcatcgtacaagcctgcggtgaatccaccaccgatgcagtcctcgccaaattcggcaacaaaggcgctgcaaccagccttcaactggcccttaaaaccgaccccaacacaacgctggatcagaagaagtacgaagccctgcaaaagaaatttgcagaggacgaaaccaaatatcgcaacaaggtcgatatcccccacaagacccaactgcgcaacctcatcctcaacacctcaaaccaattctgcaactggcacaccaagccagccatcgaagcctttaagtgcgccatcgctgacatccagtccaaagtcagcaacaacctccgcatcatgcaggaaaaggccaaactctacgaggcattcagaaatgtcgatccacaagtccagatcgccgtccaagctcttgaaaaccacatgaacacacttgaggaaccctacgcaccctacgcccactcgttcggcagcgtcaaagacttctacgaagacctcaacaacggctccaacttagatgaggccattcaaaccatcgtccacgattccgacaacttcaacaggaagccagaccccaactggctccgcatcatcgcacctctccactcatcccattccgcaagccaaatcatggaggcagtaaaatacctgtccagcaaacaggattacgaactccgtaaacccttcccattcgtcgccactaacctgccagcaacctacgggaaatttaacattcccggcaccctcaacccacccaccgacagccttcacggcagactgaacggtagccactccaatatgtggctcacagccctgctcctcgacggcagggattggaaaaaccaccacctttgcttcgcctcaagccgctacttcgaggaggtctacttcacaaaccccagcctgcccactacagacaaagtccgtagccccaaatgcggcttcacactcaagagcgtgctcgactccgaagccaaagacaggattcgcaacgctcccaaatcccgcaccaaggccgtgaaagccatcgaacgcatcaaggccaactccacccacaatgtggcgtggaaccccgaaacctctttccagatgcagaaaagaaacgatgagttctacatcaccatcaaccaccgcatcgaaatggaaaaaatccccggtcagaaaaagaccgatgacggtttcacaatccaccccaaaggtctcttcgccatcctcaaggaaggcgacagaatcctgtcacaagacctcaaccagaccgcagccacacattgcgccgtctatgaagtcgccaaacccgaccagaacaccttcaaccaccacggcattcacctcaagctgattgccacagaagaactcaaaatgcccctcaagaccaaaaagtccacaatcccagatgccctctcctaccaaggcatccacgcccacgaccgtgaaaacggcttacaacaactcaaagatgcctgcggagctttcatcagccccagactcgatcccaaacaaaaggctacttgggacaactccgtctccaagaaggagaatctctatccattcatcaccgcctacatgaaactcctcaagaaggtcatgaaggcaggtcgtcaagaactgaaacttttcaggacacaccttgaccacatcctctttaaacacaacctcagccccctcaagctgcacggtgtgtccatgatcggtctggaatcatccagagcaaccaaatccgtcatcaacagcttcttcaaccttcagaacgccaagacggaacagcagcagatcgccctcgaccgacccctgtttgaggccggtaaaaccctcatcaacaaccaaacccgccgacgacaggaaagggtcaggttagaaaccagtctcaccatgagactggcacacaaatacaacgccaaggcaatcatcatcgagggtgaactgccacactccagcaccggaacctcgcagtaccagaacaatgtccgtctggactggtctgccaagaaatccgcaaagctgaaaaccgaatcagccaactgtgcaggcattgccatatgccagatcgatccgtgccacacaagccaccaaaatcccttccggcacactccaactaacccagacctcagaccacgatttgcgcaagtcaaaaagggcaaaatgttccagtatcaactcaatggactacagaggctgctcaaccccagaagcaaatcctcaactgccatctactacaggcaggcagtccaaagtttctgcgcccaccacaacctgacggagagggacatcacctctgccaaattccccagcgatctggagaaaaaaatcaaggatgacacctatctgattccccagagaggtggtagaatatacatcagcagcttccccgtcactagctgcgcccgtccctgcaccagcaaccattatttcgggggtggacaattcgagtgcaatgctgacgctgtcgcagccgtcaacatcatgctgaaggttcacccgtaa

SEQ ID NO: 29 - DNA, 3036 bp, unidentified
  source 1..3036: mol_type = other DNA
atgcccattcgcggatataaatgcactgttgtcccaaacgtacgcaaaaagaaactcttggaaaaaacctatagctacttacaagagggttctgatgtattttttgatcttttcttgagtctgtatggtgggatcgccccaaaaatgattccacaagacctggggatcaatgaacaagtaatttgtgctgccaattggttcaaaattgttgaaaaaacgaaagattgcatcgctgatgatgcgttgttgaatcaatttgctcaatattatggggaaaaacccaatgaaaaggttgttcaatttttgacggcatcttacaataaagacaaatatgtttgggtcgattgtcgtcaaaaattttacactctgcaaaaggatttgggagtccaaaacctagaaaacgacctggagtgtttgattcgagaagatttgttgcccgtaggaagcgacaaagaagttaatggatggcactcgatatcaaaattgtttggttgtggagaaaaagaagacagaacaattaaggctaaaattctgaatggcctatgggaaagaattgagaaagaagatattctaacagaagaagacgcaagaaatgaactattgcactctgctggggtgttgactccaaaagaatttagaaaagtatataaaggggctgctggtgggcgtgattgttatcacacgttgctggtagatgggagaaacttcacttttaaccttaaaacactcattaagcagaccaaggataaattaaaagaaaagtctgttgatgttgaaatccccaataaagaagcattgcgtctatatctcgaaaaacgaattggacggtctttcgagcaaaagccatggagcgaaatgtataaaacggccctctcagccgttatgccaaaaaatacgctaaattattgtttcgccattgataggcacgcccaatatacaaaaattcaaacactaaagcagccatatgattcggcaattactgccctaaatgggttttttgagtctgaatgctttacaggctcagatgtttttgttatttctccctcccatttggggaaaactcttaaaaaactttataattacaaagatgttgaatctggcattagcgaaattgttgaagatgaagacaatagtttgcgatctggggtaaatgtaaatttacttagatatatttttactcttaaagatatgttttctgctgaggatttcatcaaagcggcagaatataatgttgtatttgaacgctacaacaggcaaaaagtccaccctacagtaaaagggaatcaatcgttcactttcggcaattccgcattgagcggtaaagttattcctccatcaaaatgcttgtccaatttgcctggacaaatgtggctggccattaatctacttgaccagggcgaatggaaagaacatcacattccttttcacagtgcaagattctatgaagaaatctatgcaacaagtgacaatcaaaataatcccgtagatttgcgaactaaacgttttggctgctctcttaacaagactttttctgctgctgacatcgaaaaggtgaaagaaagtgccaagaaaaaacatggcaaagcagctaaacgtattttgagagccaaaaacaccaatacagccgtaaattgggttgattgcggttttatgttggaaaaaacagaggttaactttaaaattactgttaactacaaacttccagaccaaaagttgggaaaatttgaaccaattgttgggacgaagattttggcttatgaccaaaatcaaaccgctcctgatgcttatgcgattcttgaaatttgcgatgatagcgaagcttttgattacaagggatataaaatcaaatgtttgtctactggtgatttggcttcaaagtcattgaccaaacaaacagaagttgatcagctagcttataagggtgtggacaaaactagcaatttttacaaaaagtggaaacagcaacgaaggctttttgtcaaaagtcttaacattccagatgccctaaagagttttgaaaacatcaataaagaatatctttatgggttcaacaattcgtatctgaagttgcttaaacaaattttacggggcaaatttggaccaattcttgttgatattcgaccagaacttattgaaatgtgtcagggaattggctctatcatgcgattgtctagtctaaaccatgatagtttggacgcaattcaatctctcaaatccttgcttcactcctattttgatctcaaagtaaaggaagaaatcaaaacagaagaattgagagaaaaagcagataaagaggtttttaagttgcttcaacaagtgattcaaaaacaaaagaataaacgcaaagaaaaagttaatagaactgttgatgccattttgactttggcggctgatgagcaagtacaagtcattgtaggagagggagatctttgtgtttccaccaaaggaacaaaaaagagacaaaacaacagaaccattgattggtgtgccagagcagttgtggaaaaactagaaaaagcatgcaaactacatgggttgcattttaaggaaattccaccacattacacttcacatcaagattgttttgaacacaacaaggatattgaaaatccaaaagaagtcatgaagtgtcgtttcaatagcagcgaaaatgtagctccttggatgatcaagaaattcgcaaattatcttaaatgcgaaacaaaatattatgttcaaggaatgcaagattttctagagcattatggtctagtagaatacaaagatcacatcaaaaagggaaaaatctcaattggggattttcaaaaacttatcaaacttgctcttgagaaagttggagaaaaagagattgtttttccatgtaaaggtggtagaatctatttgtcaacctattgcttaacaaatgagtctaaacccattgttttcaatggcagaagatgctatgttaataatgcagaccatgttgctgcgattaatgttggcatttgtcttttgaattttaatgcgagagccaaggtggcggaaaaaaccccttga

SEQ ID NO: 30 - DNA, 3282 bp, unidentified
  source 1..3282: mol_type = other DNA
atggctaagaaggattttatcgctcgtccctacaattcattcctgctccccaacgacagaaagcttgcttatctggaagaaacttggactgcctacaagtcaatcaaaacagtactgcaccgtttcctcatcgcagcatacggcgctattcccttccagacctttgcaaaaaccatcgaaaacacacaagaagacgaattgcaattggcatatgccgttagaatgttcagactagttccaaaagacttctccaagaatgaaaacaacataccccccgatatgctcattagcaagcttgctagctatacaaatataaatcaatcaccaaccaatgtcttgagctatgtaaacagcaactacgatccagaaaagtataagtggatcgactcacgcaacgaagccatctcattgtccaaagaaatcggcatcaaactcgatgagttggcagactacgctaccaccatgctttgggaggactggcttccacttaacaaagacacagtcaacggttggggcaccactagcggcctattcggcgcaggaaaaaaagaggatcgtacccaaaaggtacaaatgctcaacgcattgcttttggggcttaaaaacaaccctcccaaggactacaaacagtattcgaccatccttctcaaggcatttgatgccaaatcatgggaagaggctgttaaaatttataaaggcgaatgctcaggtagaaccagtagctacctgacagaaaagcatggagacatttccccagaaactttggaaaaactaattcaaagtattcagagagatattgctgacaaacaacaccccatcaatctacctaaaagagaagaaattaaggcatacttggaaaagcagagtggtactccatacaatctcaatctctggtcacaagccctacacaacgctatgtcttctatcaagaagacagatactcgcaatttcaatagcacactagaaaaatatgaaaaagaaattcaactcaaggagtgcttgcaagatggtgatgatgtagaattacttggcaacaaattcttttcatctccatatcataagaccaacgatgtctttgtcatttgctctgagcatatcggcaccaatcgcaaatacaatgtcgttgagcagatgtaccaactcgctagcgaacatgccgattttgaaacagtgttcactctcctcaaagatgaatacgaagaaaaaggtatcaaaaccccaatcaaaaacattcttgaatacatttggaacaacaagaatgtgcctgtaggcacttggggtagaattgccaaatacaatcagctgaaagatagattggctggaatcaaagccaatcctaccgttgaatgcaaccgtggcatgacatttggcaattctgcgatggttggcgaagttatgcgatccaatcgcatttcgaccagcacgaagaataaaggccagattttggcccaaatgcacaacgataggcccgttgggtcaaacaacatgatctggctggaaatgacgcttttaaacaacgggaaatggcaaaaacaccacatcccgacccacaataataagttctttgaagaagtccatgctttcaatccagaactgaagcaatccgtgaatgtgcgaaatagaatgtatcgttctcaaaactattcgcaacttccaacatctctgaccgatgggctgcaaggcaacccaaaagccaagattttcaagcgtcaatatcgtgcgctcaataacatgacggcaaacgtgattgatccaaagttgagttttattgttaacaaaaaggatggcagattcgaaattagcatcattcacaatgttgaagtgatcagggccagacgagatgttctggtcggggattacttggtcggcatggatcaaaaccagactgccagcaacacttacgctgtcatgcaggtggttcagccaaacactcctgactcccatgaatttcgcaaccaatgggtgaagtttattgagagtggcaagattgaatcttctactctcaattctagaggcgaatacattgaccagttgagtcatgatggcgtggatttgcaagaaatcaaggattctgaatggattccagctgctgagaaattcttaaacaagttgggagcaatcaacaaggacggcactccaatcagcatctctaatacttcaaagagggcttacaccttcaactccatatatttcaaaatcttattgaattatcttcgtgctaatgatgttgatctgaatttggtgagagaggagattctgcgtattgccaacggcaggttttcgcccatgcgtctgggtagtctgtcgtggactactcttaagatgttgggcaactttagaaatttgattcatagttatttcgatcactgtggtttcaaggaaatgcctgaaagggaatctaaagacaaaaccatgtacgatctgttgatgcataccatcacaaagctgacaaacaagcgtgccgaaagaacgagtaggattgctggttctttgatgaatgtagcccataagtataaaattggcacaagcgttgtgcatgttgtcgttgaaggcagtctaagcaagaccgacaaatccagcagcaagggtaataaccgaaataccactgattggtgctcaagggctgtagtcaaaaagctggaagacatgtgcgtcttttatgggttcaatttgaaagcagtttcggcgcattacactagtcaccaagacccattggttcatcgggctgattatgatgatcccaagcttgctttgcggtgtcgatattcgtcgtatagtcgggctgattttgaaaagtggggtgagaagtcgtttgctgctgtgattcgttgggctaccgacaaaaagagcaatacttgttacaaggttggggctgtggagttctttaaaaattataaaatcccagaggacaagatcaccaagaagctgaccataaaggaattccttgagataatgtgtgcagagtcacactatccgaatgagtatgacgatattttgattcctcgccgtggaggcaggatttatctgacaacgaagaagttgctaagtgattcgacccaccaaagagaaagtgtgcatagtcacacggctgttgtcaaaatgaacgggaaagagtattattcctcagatgcagatgaggtggctgcgatcaacatctgcctacatgactgggttgtcccactgaattggaccaatcactgcctacctgctggctggtgctctgaccacctgaaagaatgtgtgcaatgtcacactccagacccagtacgaatatccatgtaa

SEQ ID NO: 31 - DNA, 3240 bp, synthetic construct
  source 1..3240: mol_type = other DNA
atgagttctgatgtggtgcggccttataacacaaagctgctcccagataacagaaagcacaatatgttcctgcagaccttcaagcggctgaacagcatctctctgaaccacttcgacctgctgatctgcctgtacgctgcaatcaccaacaagaaggccgaggaatacaagtctgaaaaggaagcccacgtgaccgccgatagcctgtgtgccatcaattggttcagacccatgagcaagagatacagcaaatacgccaccaccaccttcaacatgttagaactgtttaaggagtacagcggccacgagcctgatgcctattccaagaactacctgatgagcaatatcgacagcgacagattcgtgtgggtggattgtaggaagttcgctaaggactttgcctatcagatggaactgggtttccacgagttcaccgtgttggccgaaaccctgctggctaattctatcctggtgctgaacgagagcaccaaggccaattgggcttggggaaccgtgtctgccctgtacggcggcggagataaggaggacagcacactgaagagcaagattctgctggccttcgtggacgccctgaacaaccacgagctgaaaacaaagagagaaatcttgaatcaagtgtgtgaatctctgaaataccagagctaccaggacatgtacgtggattttagaagcgtggttgacgaaaacggcaacaagaagtctcctaacggctctatgcctatcgtgaccaagttcgagacagacgacctgatcagcgacaaccaaagaaaggccatgatcagcaacttcactaagaacgccgctgccaaggcagctaagaaacctatcccttacttggaccgcctgaaggagcacatggtgtccctgtgcgacgagtacaatgtgtatgcctgggccgcggccatcacaaacagcaacgccgacgtgaccgcccggaataccagaaacctgacattcatcggcgaacagaacagcagacgaaaggaactgagcgtgctgcagacaacaaccaacgagaaggctaaggacatcctgaacaagatcaacgacaacctgattcaggaggtgcggtacacccctgcccctaagcacctgggcagagatctggccaacctgtttgatacactgaaggaaaaggacatcaacaacatcgagaacgaagaagagaaacagaacgtgatcaatgactgtatcgagcagtacgtggacgattgcagaagcctcaaccggaaccccatcgcagccctcctgaagcacatctctaggtactacgaggatttcagcgccaagaatttcctggacggcgccaagctgaacgtgctgactgaggtggtgaaccggcagaaggcccaccccaccatctggagcgagaaggcttacacctggatcagcaagttcgacaagaaccggagacaggccaacagcagcctggtcggatgggttgtgccccccgaggaggtgcacaaggagaaaatcgccggacagcagagcatgatgtgggtgaccctcaccctgctggacgacggcaagtgggtcaaacatcacatccccttcagcgacagcagatactacagcgaagtgtacgcctacaaccctaatctgccttatctggacggaggcatcccaagacagagcaagttcggcaacaaaccaacaaccaacctgacagccgagtcccaggccctcctggctaattctaagtacaagaaagccaacaagagcttcctgcgggctaaagagaatgccacacacaacgtgcgggtgtcccctaacacctctctgtgcattagactgctgaaggacagcgccggaaaccagatgttcgacaaaatcggcaacgtgctcttcggcatgcagatcaaccacaagatcaccgtgggaaaacctaactacaagatcgaggtgggcgacagattcctgggcttcgatcagaaccagagcgagaaccacacctacgccgtgctgcagagagtgtccgagagcagtcacgacacccaccactttaacggctgggacgtgaaggtgctggaaaagggcaaagtgaccagcgatgtgatcgtgcgggacgaggtctacgaccaactgtcttacgagggcgtcccctacgatagcagcaagttcgccgagtggcgggacaagcgcagaagatttgtgcttgagaacctgagcatccagctggaagagggcaagaccttcctgacagagttcgacaagctgaataaggacagcctgtaccgctggaacatgaactacctgaaactgctgagaaaggccatccgggccggaggcaaagagttcgccaagatcgctaagacagagatcttcgagctggcggtggaaagattcggccctattaacctgggcagcctgtcccagatcagccttaagatgattgcctcctttaagggcgtggtccagtcctacttctccgtgagcggctgcgtggatgatgcctccaaaaaggcccatgattctatgctgttcacatttatgtgcgccgccgaagaaaagcggaccaacaagagagaagaaaagaccaacagagccgccagctttatcctgcaaaaagcctacctgcatggctgcaagatgatcgtgtgcgaggacgaccttcctgtggccgacggcaagacaggcaaagcccagaatgccgaccggatggactggtgcgccagagccctggccaagaaggtgaacgacggctgtgttgccatgagcatctgctacagagctatccctgcctacatgagcagccaccaggacccctttgtgcacatgcaggataagaaaaccagcgtgctgcggcctagattcatggaagttaataaggatagcatcagagactaccacgtggcgggcctgagaagaatgctgaacagcaagagtgacgctggcaccagtgtttattaccggcaagctgccctgcatttctgcgaagccctgggcgtgagccctgaactggtgaaaaacaagaaaacccacgccgccgaactgggcaagcacatgggcagcgctatgctgatgccctggagaggcggtagagtgtacatcgccagcaaaaagctgacctccgatgccaaatcagtgaagtactgcggcgaggatatgtggcagtaccacgccgatgagatcgccgctgttaacatcgccatgtatgaggtgtgctgccagaccggcgctttcggaaagaaacagaaaaaatcggacgagctgcctgga

SEQ ID NO: 32 - DNA, 3183 bp, synthetic construct
  source 1..3183: mol_type = other DNA
atgagctctgacgtggtgcggccttacaataccaagctgctgccagacaaccggaagtacaacatgtttctgcagaccttcaagagactgaacctgatctccagcaaccacttcgacctgctggtgtgcctgtacgccgctatcaccaacaagaaagctgaggaatacaagagcgaaaaagaggatcacgttacagccgacagcctgtgtgccatcaactggttccggcctatgtctaagcggtacatcaagtacgctacaaccacctttaagatgctggaactgttcaaggagtacagcggccacgagcctgacacctacagcaagaactacctgatgtctaatatcgtgagcgataggttcgtgtgggtggactgccggaaattcgctaaggacttcgccaatcaaatggaactgtccttccacgagttcaccaccctgagtgaaaccctgctggctaacagcatcctggtgctaaatgagtctacaaaggccaactgggcctggggcgccgtgagtgctctgtacggcggcggcgacaaagaggactctacactgaaaagcaagatccttctggcctttgtggacgccctgaacaaccctgaactgaaaacacgtagagaaattctgaaccacgtgtgcgaatctctgaagtatcagagctaccaggacatgtacgtcgatttcagaagcgtggtcgatgataagggcaacaagaagagcccaaacggcagcatgcctatcgtgaccaagttcgagagcgatgatctgatcggcgataaccagagaaagacaatgatctctagctttacgaagaacgccgccgccaaggccagcaagaagcccatcccatacctggacatcctcaaggaccacatgatcagcctgtgtgaagagtacaacgtgtatgcctgggccgctgccatcaccaacagcaacgccgacgtgacagcccgcaacaccagaaacctgacattcatcggagaacagaacacccggaggaaggaactgagcgtgctgcagacaagcaccaacgagaaggctaaagacatcctgaacaaaatcaacgacaacctgatccctgaggtgcggtacacacctgcccctaagcacctgggtcgggacctggccaatctgttcgagatgttcaaggaaaaggacatcaaccagatcggcaacgaggaggagaagcagaacgtgatcaacgactgcatcgaacagtacgtggacgactgtagaagcctgaacagaaacccagtggccgccctgctaaagcacatcagcggatactacgaggatttcagcgccaaaaatttcctggacggcgccaagctgaatgtgctgaccgaagtggtcaacagacagaaggctcatcctacaatctgcagcgaaaaggcctacacctggattagcaagatcgataagaaccggcggcaggccaattcctccctggtcggatgggtggtgccccccgaggaagtgcacaaggaaaagattgccggccagcagagcatgatgtgggtgacactgacactgctggacgacggcaagtgggttaagcaccacatccccttcgccgattctagatactacagcgaggtgtatgcctataatcctaacctgccttatctcgagggcggcatccccagacagtctaagtttggcaacaaacctaccaccaacctgaccgccgaatctcaggccctgttggccaactccaagcacaaaaaagccaacaagaccttcctgagggccaaagagaacatcacccacaacgtgagagtgtctcctaataccagcctgtgcatcagaccactgaaggactctgctggcaatcaaatgttcgacaacatcggcaacatgctgttcggtatgcagatcaaccatagaatcaccgtaggaaaacccaactacaagatagaggtgggcgatagatttctcggattcgaccagaatcagagcgagaaccacacctacgcagtgctgcaaagagtatctgagagcagccacggcacacaccactttaacggctgggacgtgaaagtgatcgagaagggcaaggtgaccagcgacgtggtggtgcgggacgaggtgtacgatcagctgtcctacgaaggcgttccttacgactcccctaagtttaccgaatggcgggaaaaacggagaaagttcgtgctggaaaacatgagcatccagatcgaggagggcaagacttttctgaccgagttcgataagctgaataaagacagcctgtatagatggaacatgaactacatgaaactgctgaggaaggccatcagagccggcggaaaagagttcgccaagatcaccaaggccgagatcttcgaactgggcgtgatgagattcgggcctatgaacctgggcagcctgagccaagtgagtctcaagatgatcgccgccttcaagggagtgatccagagctacttctctgtgtctggctgcatcgatgatgcttccaagaaggcccacgacagcatgctgttcgccttcctgtgtagcgccgatgaaaagcggaccaacaagcgggaagaaaagaccaatcgggccgccagcttcatccttcaaaaggcctactcccacggctgtaaaatgattgtgtgcgaggacgaccttcctatcgccgatggcaaagtgggaaaggcccagaacgccgacagaatggactggtgcgcccggagcctggctaagaaagtgaacgatggctgcgtggccatgtccatctgctacagagccatccccgcctacatgagctcccaccaggaccccttcacccatatgcaggataagaaaaccagcgtgctgcggcctagatttatggaagttggcaaggacagcatccgggaccaccacgtggctggcctgagacggatgctgaatagcaagggcaacacaggcaccagcgtgtactacagagaggccgcactgcgcttctgcgaggccctgggcgtgctgcctgagctggtgaagaataagaaaacacacgccagcgagctgggaaagcatatgggcagcgcaatgctgatgccttggagaggcggcagaatctacgtggccagcaagaaactgacaagcgacgccaaatctatcaagtactgcggcgaggatatgtggcagtaccacgccgacgagatcgctgctatcaacatcgccatgtacgaggtc

SEQ ID NO: 33 - DNA, 3225 bp, synthetic construct
  source 1..3225: mol_type = other DNA
atgggcatctctatcagcagaccttacggcaccaaactgcggcctgatgccagaaagaaagaaatgctggataaattcttcaccaccctggccaaaggccagagagtgttcgccgacctgggcctgtgcatctacggcagcctgacactggagatggtgaaaagactggagcctgagagcgacagcgagctggtgtgcgccatcggctggttccggctggtggataaagtgacctggagcgaaaacgagatcaagcaggaaaacctggtgcggcagtacgaaacctactctggcaaggaagccagcgaggtgatcaagacctatctgagcagtccctcttctgataagtacgtgtggatagattgcagacagaagtttctgcggttccagcgggacctgggcacaagaaacctgtccgaggatttcgagtgcatgctgttcgagcagtatctgagactgactaagggcgagctggatggacacaccgccatgagcaatatgttcggcaccaagacaaaggaggatagagccaccaagctgcgatacgccgccagaatgaaggagtggctggaagctaatgaggagatcacctgggaacagtaccaccaggccctgcaggataagctcgacgcgaacactctggaggaagccgtggataactacaagggcaaggctggcggaagcaaccctttctttagctacaccctgctgaaccgaggacagatcgacaagaaaacccacgagcagcagctgaagaagttcaacaaggtgctgaaaaccaagtctaagaacctgaacttccctaacaaagagaagctaaagcagtacctcgagacagcgatcggaatccccgtggacgctcaggtgtacggccagatgtttaacaacggcgtgtctgaggttcaacctaagacaaccagaaacatgtcctttagcatggaaaagctggagctcctgaacgaactgaagagcctgaacaagaccgacggattcgagagagccaacgaggtgctcaatggcttcttcgacagcgaactgcacacaacagaggacaaattcaatatcacaagcagatacctgggcggcgacagaaacaaccggctccctaagctgtatgagttgtggaagaaggagggcgtggacagagaggagggcatccagcaattttcccaagccatccaggacaagatgggccaaatccctgttaagaacgtgctccgctacatctgggagttccgggaaaccgtgagcgcagaagatttcgaggctgctgccaaggccaaccagctggaggaaaagatcacccggaccaaagcccaccccgtcgtgatcagcaacagatactggaccttcgggtccagcgccctggtgggcaacatcatgcctgccgacaagatgcacaaggaccagtacgccggccagagctttaagatgtggctggaagctgagctgcactacgacggcaagaaggtgaagcaccacctgcccttctacaatgccagattcttcgaggaggtgtactgctaccacccatcagtggccgaagtgaccccttttaagaccaagcagttcggatatgccatcggcaaggacatcccagctgacgtgtctgtggtgctgaaagataacccctacaagaaggccaccaagagatttctgagggccatcagcaatccagtcgccaacactgtggacgtgaacaagcctacagtgtgtagcttcatgatcaagcgggaaaacgacgagtacaagctggtgatcaacagaaagatcggagtggacagacccaagagaatcaaggtgggcagaaaagtgatgggctacgacagaaaccagaccgccagcgacacatattggatcggcgagctggttcctcatgggaccacaggcgcctacagaatcggagaatggagcgtgcaatacattaaaagcggccctgtgctttcttctacacagggcgtgaacgattctaccaccgatcagctgatctacaacggaatgcccagcagcagcgagcggttcaaggcctggaagaagtccagaatgagcttcatccggaagctgatcagacagctgaatgccgaaggcctggaaagcaaaggacaggactacgtgcccgagaaccctagcagcttcgacgtcagaggagaaacactgtacgtgtttaacagcaactacatgaaagccctggtgtccaagcacaggaaggccaagaagcccgtggaaggcatcctggaagaaatcgaggctctgacctccaaagccaaggacagctgcagcctgatgcgcctgagctctctgagcgacgccgccatgcagggcatcgccagcctgaagtccctgatcaactcttatttcaacaagaatggctgtaaaaccatcgaggacaaggaaaagttcaaccccgacctgtacgtgaagctggtcgaggtcgaacagaaaagaaccaacaagcggaaggagaaggtgggccggatcgccggcagcctggaacagctcgccctgctgaatggtgttgacgtggtgatcggcgaggccgatctgggggaagtcaagaaaggcaagtctaagaagcagaatagcagaaacatggactggtgcgccaagcaggtcgctgagcgcctggaatacaaactgaccttccactgtatcggctacttcggcgtgaaccctatgtacacaagccaccaagatccttttgaacaccggagagtggccgaccacctggtgatgagagctaggttcgaagaggtgaacgttagcaacgtaagcgaatggcacatgagaaacttcagcaattacctgcgggccgacagcggcacaggtctgtactacaagcaagccaccctggactttctgaaacattacgacctggaggagcacgccgacgacctggagaaacagaatatcaagttctacgatttcagaaagatcctggaggacaagcagctgacatctgttatagtgcctaagcggggcggcagaatctacatggccacaaaccccgtgacatcagacagcacccctgtgacctacgccggcaagacctacaatagatgcaacgccgatgaggtggctgccgctaatatcgctatttctgtgctggcccctcacagcaagaaggaagagaaagaggataagatccctatcatcagcaagaagcctaagtccaagaacaccccaaaggctagaaagaacctgaaaacaagccagctgcctcagaag

SEQ ID NO: 34 - DNA, 3009 bp, synthetic construct
  source 1..3009: mol_type = other DNA
atggccagcaaacacgtggtgcggccttttaacggcaaagtgaccgctaccggcaagcggctggcctacctggaggaaacctttcattacctggagaaggccgccggcggcgtgtctaccctgttcgccgctctgggcagctacctcgacgccacaaccatcagcaacctgatcaacaagaaccaggacttggctgtcgtgatcttccggtaccacgtggtgcctaagggcgaagcccacacactgcccgtgggcaccgacatggtgtcaaggttcgtggccgactacggcatggagcctaatgagttccaaagagcctacctggatagccccatcgatcaggagaagtactgctggcaggacaatcgggacgtgggatgttggctgggcgaacagctgggtgtttctgaggccgacatgcgggctatcgccgtgactttttacaacaaccagatgctgtacgactgtgtgaagggaactggcagcggcaatgccgtctctctgctgtttggcagcggcaagaagtccgactacagcatgaagggagtcattgccggcaaggctgcctcagtgctggctaagtatagacctgccacctaccaggatgccagaaagatgatcctggaagctaatggcttcaccagcgtgaaagatctggtcacatcttacggcatcaccggcagaagcagcgccctgcaaatcttcatggaaggcattgaaagcggacctatctcctccaaaacattggacgccagaatcaagaagttcacggaagatagtgagcggaacggccgcaagaacctggtcccccacgccggcgccattagaaattggctgatcgagcaggccggttcttctgtggaaaactaccaaatggcctggtgcgaggtttacggcaacgtgagcgctgactggaacgccaaggtggaaagcaacttcaacttcgtggccgagaaggtgaaagccctgaccgagctgagcaatatccagaagagcacccctgatctgggcaaggctctgaaactgtttgaggagtacctgaccacatgccaggacgagttcgccatcgccccataccacttcagcgtgatggaagaggtgcggatggaaatggccacaggcagagagtttaacgatgcatacgacgacgctctgaacagcctggacatggaaagcaagcagcctatccagcctctgtgtaaattcctgatcgagcggggcggaagcatcagcttcgacaccttcaagagcgccgccaaatacctgaaaacccagagcaagattgccggcagataccctcatccattcgtgaagggaaaccagggcttcacattcggctccaagaacatctgggccgccataaacgaccccatgatggagtacgccgacggccggatcgccggcggctctgccatgatgtgggtcaccgctaccctgctggacggcaagaagtgggtgagacaccacatccccttcgccaacacaagatacttcgaggaggtttacgccagcaagaagggcctgcctgtcctgccgtgcgccagagatggcaagcacagctttaagctgggtaacaacctgagcgtggagagagtggaaaaggtgaaggaaggcggcagaacaaaggccacaaaggctcaggagagaatcctgagcaacctgacacacaacgtgcagttcgacagcagcaccaccttcatcatccggagacaggaggaatcctttgtgatctgcgtgaaccacagacaccccgcccctctgatgaagaaggagatggaagtgggcgacaagatcatcggcatcgaccagaacgtgaccgcccctaccacctacgccatcgtggagagggtggccagcggaggcatcgagcggaacggcaaacagtacaaggtgacagccatgggcgccatctcctctgtgcagaaaaccagaggcggagaggtggacgtgctgagctacatgggtgtggagctgtccgactcgaagaacggattccagagcctgtggaacaagtgtctggacttcgtgaccaagcacggcacagagaacgacgtgaagtactacaacaacacagccgtgtgggccaacaagctttacgtgtggcacaagatgtacttcagactgctcaagcaactgatgagaagagccaaggacctgaagcctttcagagatcacctgcaacacctgctgttccaccctaacctgtctcctctgcagcggcatagcctgtctcttacaagcctggaggctaccaagatcgtgcgcaattgcatccacagctatttcagccttctcgggctgaaaaccctggatgagagaaaggcagccgacatcaacctgctcgaggtgctggaaaagctgtatgccggccttgtggaaagaaggaaggagagaaccaagctgacagccggcctgctggtcagactgtgcaacgagcacggaattagctttgccgccatcgaaggcgacctgcctgtggtgggcgaaggcaagagcaaggccgctaacaacacccagcaggactggaccgcccgggaactggagaagagactgagcgaaatggctgaggtggtgggcatcaaggtgatcgctgttctaccacactacaccagccaccaggaccctttcgtttactccaagaataccaagaaaatgcggtgcagatggaattggcggaccaccaagaccttcaccgatagagatgccctgagcatccggagaatcctgagcaagcccgaaaccggaaccaacctgtattaccagaagggactgaaggccttcgccgagaagcacggcctggatctggccgaaatgaagaagcggaaggacgcccagtggtacctggaaagaatccaggataagaacttcctggtgcccatgaacggcggaagagtgtacctgagcagcgtgaagctggccggcaaagagacaatcgacatgggcggcgagattctgtacctgaacgacgccgatcaggtggccgccctcaacgtgctgctggtgaagatc

SEQ ID NO: 35 - DNA, 3054 bp, synthetic construct
  source 1..3054: mol_type = other DNA
atggccaaaaaggaacacattatcagacctttcaagggcaccctgccactgcggggggacagactgagatacctgcaggacaccatgaagtacatgaagaaggttgaggacaccatcaccgagctgtgcgccgccgtgatcgcctacgccaagcctacaatcatccagcagattctgggagaagaaatcgagactacctccaccttctgcagcttcagactggttgggattcatgagaacttcactatgcccctgacaaccaatatgatcaagcacttccagaaaaccttcaacatcaatccttctgagaagcaggccatctatctgagcagcggatttgatagcgacaaatacagatggcaggatacaagcgaggtgtctagaaatttcgctaataagtgccgcctgaccaaccaggagttccaggagttcgccgagcaagctctgttaaacatgtgctttatcggctgtagcggatctcctggcgccacaaacgccgtgtcccagatcttcggcaccggcgaaaagtctgattaccagcggaagtctcagatcgccaagatcgccgctgataccctcgagaaccacaaacctagcacatacgagtctgctaggctgatggtgctgaacacactgggacacaagacgatcgaagattgcgtgaacgactacggcgctattggagccaagtccgccttccggctgtttatggaaagtaaagaaatcggcccaatcaccagcgaacaactgaccacaaaaatcaagaaattcagagaggaccacaagaagaacagcatcaagaagcagctgcctcatgtggaaaaggtgcggaacgcactactgagccagttcaaggagcagtacctgccaagcgcctgggccgaggcctggtgtaacatcatgggagagttcaatagcaagctgtccaacaacaacaatttcatcgaccaaaaaaccaagatggtcaacgactgcgacaacatcaaaaaatctaacccccagctggataaggccgtgaatatgctggacgaatggaagtacaagaattgggacgacaattctgccatccacccctaccacatcggcgatctgaaaaagctgatggccatcttcaacatcaacaatgagggcaccttcgacgagagattcagcgccagctgggagcagttttctaccagcctggagtacggcgagaagccccccgtgcgggacctgctggcccacatcatcaagaacatgaacgacctgacttacaccgacgtgatcaatgccgctaagttcctgaagctgcaagataatatcagaaacaagtatcctcacccttttgtgatgcctaacaagggatgtaccttcggcaaggataacctgtggggcgagatcaatgatcctacagctaagatcaagtccacagaggaagtggccggccagcggcctatgatgtggctgaccgccaagctcctggacaacggcaaatgggtcgagcaccatatccccttcgcctctagcagatacttcgccgaagtgtactacaccaaccccgccctgcctaccttacccatcgcccgcgacggcaagcacagctacaagctgaccaagaccatcgacgccaacaccgccaaaaccctggtgaacaaccctagagacaaggccgccaagctcattgccagaacaaaggcgaacaccacccacaacgtgaagtggatcaaacctacatacagaatccagaaagagaacaaccagttcgtgatcaccatcaatcacagacacccatgtatcacccctcctaaggaaatcatcttgggcgatagaatcctgtcattcgaccaaaacgagacagcccctaccgcctttagcatcctggaaaagaccaccaagggcacagagttctgcggccaccacatcaaagtgctgaaaaccggcatgctggaagccaagatcaagacatcgaagaaatccatcgacgccttcacctacatgggccctatggaggacgaccacgccagcggtttccccaccctgctgaacatctgtgaaaagttcatcagcgagaacggcgacgagaaggacaagagcttcagcagcagaaagctgccttttaagagaagcctgtattttttccacggcagccacttcgacctgctgaagaagatgatccggaaggctaaaaatgaccctaagaaactgaagctggtgagaatccacatcaacgagatcctattcaacagcaacctgtcccctatcaagctgcacagcctgagcatccactctatggagaacacaaaaaaggtgatcgctgccatctcttgctacatgaacgtacacgagtggaaaaccatcgatgagcaaaaaaacgccgacatcacactgtacaacgccaaggaaaagctgtacaacaacctggttaatagaagaaaggaaagagtgaaggtgaccgctggcatgctgatccggctggcccgggaaaacaactgcagattcatggtgggcgaagccgaactgccaacacagcagcagggcaagagcaagaagaacaacaacagcaagcaggactggtgcgccagagacatcgcacagagatgcgaggatatgtgcgaggtggtgggcatcaaatggaacggcgtgacacctcacaacaccagccaccagaatccattcatctacaagaacacctccggccagcagatgcggtgcagatacagcctggtcaaaaagtctgagatgaccgataagatggctgagaagatccggaacattctgcacgccgagcctgtgggcacaaccgcttattacagagagggcatcctggagtttgccaagcaccacggactggacctgggcatgatgaagaaaagaagagatgccaagtattacgacaacctgcccgacgaatttctgctgccgacaagaggcggaagaatatacctgtcggaaaaccagctgggcggcaacgagacaatcgtgatcaacggcaagaaatacttcgtgaatcaggccgaccaggtggccgccgtgaacatagggctgctgtacctgctgcctaagaagaaccagagc

SEQ ID NO: 36 - DNA, 3141 bp, synthetic construct
  source 1..3141: mol_type = other DNA
atgagcgagaagaaattccacatcagaccctacagatgcagcatctcccctaacgcccggaaggccgacatgctgaaggctaccatctcctacctggacagcctgacctctgtgttcagaagcgggtttaccgccctgctggctggaatcgatcctagcaccgtgtccaggctggctcctagcggcgccgtgggcagccccgacctgtggagcgccgtgaactggttcagaatcgtgcccctggccgaagccggcgatgccagagtcggccaggcaagcctgaaaaacctgtttagaggctacgccgggcacgaacctgacgaggaagccagcatctacatggaaagcagagtggacgacaaacggcacgcctgggtcgactgcagggccatgttcagagctatggccctcgagtgcggcctggaggaagcccagctggcttccgacgtgttcgccctggccagcagagaggtgatcgtgttcaaggacggcgaaatcaacggctggggcatcgccagtctgctgttcggcgaaggagagaaggctgattctcagaaaaaggtggccctgctgagaagcgtgagactggccctcgagggcgattacgctacctacgaggagctgtctggcctgatgctggccaagaccggcgccagctctggctccgatctgctggacgagtacaaacggtccgaaaaaggtggctcttctggaggcagacatcctttctttgacgaggtgtttcggagaggcggcagagttaaacaggaggaaagagagagactcctgaaaagctgcgacaccgcaatccagaagcagggacaggccctgcctctgtctcacgtggccagctggcggcagtggttcctgagaagagtgaccctgctgaggaatagacggcaggagagcttcgctgtgtgcatcacaaacgccctgatggacctgcaacccaagaacctgagaaatgtgcactacgtgaccaaccccaagagcgagaaggataagggggttctggaactgcgggtggacgtcaaaaacaacgagggccctgatgtggctggcgcccaagccgtgtttgacgcctacatggccagacttgccccagatctgagattcagcgtgatgcctagacatctgggctcactgaaggacctgtacgccttgtgggccaagctgggaagagatgaggcgatcgaggagtacctggaaggctatgagggccctttcagcaaaagaccaatcgccggcatcctgcagatcatccacgcccatcggggcaaggtggggcacgacagcctgttgagagccgccagacttaacagagctatggatagactggagagaaaaagagcccacgcctgtgccgccggcaacaagggatatgtgtacggcaagagcagcatggtgggccggatcaaccctcagagccttgaagtgggcggacggaagtctggccggagccccatgatgtgggtgacactggacctggtcgacggcgacagattcgcccagcaccacctgccctttcaatctgcccggttcttcagcgaagtgtactgccacggagacggcctgcccgccaccagagtgccaggcatggtcagaaaccggagaaatggcctggccatcggaaatggcctgggcgagggaggactgagtgctctgagagccggaagcgaccggagaaagcgggctaacaagagaacactgagagccctggagaatatcacccacaacgtggaaatcgatcctagcacatccttcacactgagagaggacggcatcatcatcagccacagaatcgagaagatcgagcctaagctggtggcttttggagacagagctctgggcttcgacctgaaccagaccggcgcccacacctttgccgtgctgcagaaggtggacagcggcgggctggatgtgggtcacagccgggtcagcattgtgctgaccggcaccgtgcggagcatctgcaagggcaatcaggccagcgggggccgggactacgacctgctgtcttacgacggccccgagagagatgatggcgcttttaccgcctggaggtctgacagacaggcctttctgatgagcgccattcgggaactgcctacccctgccgagggcgagaaagattacaaggccgacctgctgtcccagatggccagcctggaccactaccggaggctgtacgcctacaacagaaagtgcctgggcatctacatcggtgccctgcggcgcgccacaagacggcaggccgttgccgccttcaaggacgagattctgtccatcgccaaccacagatgcggccccctgatgagaggctccctgagcgtcaacggcatggaaagcctggccaacctgaagggcctggcaaccgcttatctgtctaagttcaaggacagcaagtccgaggacctgctgagtaaggacgaagaaatggccgacctgtacagagcttgcgccagacgcatgaccggaaaaagaaaggaacggtaccggcgtgctgccagcgaaatcgtgagactggctaacgagcacggctgtctgttcgtgttcggcgagaaggaactgcctacaaccagcaagggcaacaagtctaaacagaaccagcggaacaccgactggtcggcccgggccatcgtgaaggccgtgaaggaggcctgcgagggatgtggcctgggcttcaagccggtgtggaaggaatactctagcttgaccgaccccttcgagagggacggcgatggccggcctgctctgagatgtagattcgccaaggtggctgctcccgacagcgagctcccacctagactgacaaaggccgtgggaagctatgtgaagaacgccctaaaggccgataaggccgagaagaaacaaacatgttaccagagaggagccatcgagttctgcagcaggcacggcatcgacgtccggaaagctacagataaggccattcggaaagctgtgcggggtagcagtgacctattagtgcctttcgatggaggcagaaccttcctgctatcaacaagactgagccctgagagcagaaaggtggaatgggccggaagaacactgtacgagttcccttctgatatggtggccgccatcaacatcgcctgccggggcctggaacctagaaaggca

SEQ ID NO: 37 - DNA, 3087 bp, synthetic construct
  source 1..3087: mol_type = other DNA
atggacgagcaggccgtggtgagcagcggctctgataagaccctgaagatcgtgaggccctacagagctaaggtgaccgctactggaatcagattggaagggatcaaaaacaccctgaattacctgaagagaacagagatttgtctgtccagactgaacgccgcttgcggcgcctttctgacccctgccatcgtggagcagatctgtaaagacgatcccgccctggtgtgcgccatagctagattccagctggtgcctgtgggcagcgaagctaccctgagcgatagcggactgatgcggcacttcaaggcggcgctgggcgaactgacccctctgcaggaagcctacctgaacagcagttataacgatgagctgtacgcctggcaggataccctggtgctggccagacagatcatcgcggaaaccggcctgaccgaggaccagttccgggcatttgcccacgcctgcttcaagaacggtaatatcatcggttgtgccggaggccctggcgcaagcaatgccattagcggcatcttcggcgagggaatcaagagcgactacagcctccgcagcgagatgacagccgctgtggctaaggtgttcgaggaaaagcggcccatcacatacgaggaagccagagccctggccctcgaagccaccggccacgcctctgtgcagagctttgtcgaggcctttggcaaacagggcagaaagggcaccctgatcctgttcatggaggacaccaaaacaggcgccttcccctccaacgagttcgactataagctgaagaagctgaaggaggacgcagagcgggtgggcagaaagggcatcatcccacatcgggacgtgatcgcctcttacctccggaaccagaccggagccgacatcgagtacaacagcaaggcctggtgcgaaagctactgctgcgccgtttctgaatacaacagcaagatgagcaacaacgtgcggttcgctacagagaagagcctggacctgactaagctggacgagacaatcagggaaaccccaaagatcagcgaggccatgctggtgttcgagaactacatggccagaatcgatgccgacctgaggttcatcgtgtcgaagcaccacctgggaaacctggccaagttccggcaaacaatgatgcacgtgtccgccagcgagttcgaggaagccttcaaggccatgtgggccgattacctggctggcttggagtatggcgagaaacctgctatctgcgagctggttagatacgtgctgacccacggcaatgacctgcctgtggaagccttttacgccgcctgcaagtttctgtccctggacgacaagatcaagaacagataccctcatcctttcgtgcccggcaacaagggctatacattcggcgcaaagaacctctgggccgagatcaacgaccctttcaagcctatcagacagggcaatcctgaggtagccggccaaagacccatgatgtgggccacagctgatctgctggacaacaacaagtgggtgctgcaccatattccttttgcctcgagcagatactttgaggaagtgtactacacagacccatctctcccaaccgcccagaaggccagagacggcaagcacggctacagactgggaaaggtgctggatgaggccgccagagaaagactgaaggccaacaacagacaaagaaaggccgccaaggccatcgagcggatcaaggccaattgcgagcacaatgtggcctgggaccctaccaccaccttcatgctgcaactggacagcgagggcaacgtgaagatgaccatcaaccacagacacatcgcctaccgggctcctaaggaaatcggcgtgggcgaccgggttatcggcatcgaccagaacgaaaccgcccctacaacatacgccatcttggaaagaacggaaaacccccgggacctggaatataacggcaagtactacagagtggtgaagatgggcagcgtgacctctcctaacgtgtccaaatacagaaccgtggacgccctgacttacgacggcgtgtctctgagcgacgacgccagcggagccgtgaacttcgtcgtgctgtgcagagagttcttcgccgctcatggcgacgacgagggccggaaatacctggagagaaccctgggctggagctccagcctgtatagcttccacggcaactacttcaagtgcctgacccagatgatgcggagaagcgcccgctctggcggcgatctgaccgtgtaccgcgctcacctgcagcagatcctgtttcagcacaacctgtcccctctgagaatgcacagcctgagcctgcggagcatggaatctaccatgaaggtgatcagctgcatgaagtcttacatgagcctgtgcggctggaaaaccgatgctgacagaatcgccaacgaccggagcctgttcgaagccgccagaaagctgtacacatctctggtcaatcggcggaccgaaagagtgcgggtgacagcaggcatccttatgagactgtgtctggagcacaatgtgcggtttatccacatggaggacgagctgcctgtggctgaaaccggcaaaagcaaaaaaagcaacggcgccaagatgcactggtgtgcccgggagctggcagttagactgtctcagatggccgaagtgaccagcgttaagttcaccggagtgagcccccactacactagtcaccaggaccccttcgtgcactctaaaaccagcaaagtgatgcgcgccagatggtcctggcggaaccgggccgacttcacagataaggacgccgagagaatccggactatcctgggcggcgatgacgccgggaccaaagcttactacagaagcgccctggccgagttcgccagcagatacggcctggatatggagcaaatgagaaagagacgggatgcccagtggtaccaggagagactgcctgaaaccttcatcatcccccagagaggcgggagagtgtacctgagctcccacgacctgggcagcggccagaaagtggacggcatctacggcggaagggccttcgtgaatcacgctgatgaggtggccgcccttaacgtggctctggtccgcctc

SEQ ID NO: 38 - DNA, 3087 bp, synthetic construct
  source 1..3087: mol_type = other DNA
atgaaaacagagacactgatccgcccttaccccggcaagctgaacctgcagcctcggcgggcccaattcctggaggattcaatccagtaccaccagaaaatgaccgagttcttctaccagttcctgcaggccgtaggcggcgcgaccacacatcagaacatcagcgatttcattgacaacaaggccactgatgagcaccaggccacccttctcttccaggtcgtgtccaaggacagcaccacccctgagtgccctgccgaggaactgctggccagattcgcccagtacaccggcaaacagcccaacgaggccgtgacccactacctgaccagcagaatcaacaccgacaagtacagatggcaggacaatagactactggcccagaacatcgccagccaacttaacatctccgagacacaattccaggaaatcgcgcacgctatcctcagcaacaacctgtacatcggacagaccgccagcaacgctgccgccaacttcatctctcaggtgaccggcaccggccagaaagccccaaaggctgccagactggacgtgctgttccagacgaaccaagccctggccaaaacccagcctacaacctttggccagctccagcagattatcgtgcaggcttgtggagaaagcaccaccgacgccgtgctggccaagttcggcaacaaaggtgccgccacctcgctgcagctggctctgaaaaccgaccccaacaccaccctggatcagaaaaagtatgaggccctgcaaaagaaattcgccgaggacgaaacaaagtaccggaacaaggttgacattccccacaaaacgcagctgagaaatctgatcctgaacacaagcaatcaattttgcaactggcacacaaagcctgccatcgaggcttttaagtgcgccatcgccgacatccagagcaaggtgtccaacaacctgaggatcatgcaggagaaggccaagctgtacgaggccttcagaaacgtggacccccaggtgcagatcgctgtccaagccctggagaatcacatgaacaccctcgaagaaccctacgccccttacgcccacagcttcggcagcgtgaaggacttctatgaggacctgaacaacggcagcaatctggacgaggcaattcagaccatcgtgcacgattctgataacttcaaccggaagcctgatcctaactggctgagaatcatcgccccactgcactctagccacagcgcctctcagatcatggaagctgtgaaatacctgagcagcaagcaggactacgaactgaggaagcccttcccattcgtggccaccaacctgcctgccacatacggcaagttcaatatccccggcaccctgaaccctcctacagactctctgcacggcagactgaacggctctcacagcaacatgtggctgacagccctgctgctggacggcagagactggaagaaccaccacctgtgcttcgccagcagcagatacttcgaagaagtctacttcaccaaccctagcctgcccaccaccgataaagtgcggtccccaaagtgcggctttaccctgaagagcgtgctggacagcgaggctaaggatagaatccgtaatgcccctaagagcagaaccaaggccgtgaaggccatcgagagaattaaggctaattctacccacaacgtggcctggaaccccgagacaagcttccagatgcagaagagaaacgacgagttctacatcacaatcaaccacaggatcgagatggaaaagatccccggccaaaagaaaacagacgacggcttcaccatccaccccaagggcctgtttgctatcctgaaggaaggagatagaatcctgagccaggatctgaatcagacagccgctacacactgcgccgtgtacgaggtggccaagcctgaccagaacaccttcaaccaccatggcatccacctgaagctgatcgccaccgaagaactgaagatgcctctgaaaaccaagaagtctaccatcccagatgccctgtcataccagggcatccacgcccacgaccgggaaaacggcctgcagcagctgaaggacgcttgcggagccttcatctcacctagactggaccccaagcagaaggccacctgggacaacagcgtgtccaagaaagaaaacctgtaccctttcatcaccgcctacatgaagctgctgaagaaggtgatgaaggcgggccggcaggagctgaagctgtttcggactcatctggatcacatcctgttcaaacacaatctcagccctctgaaactgcacggcgtgagcatgatcggcctggagagcagcagagctacaaaaagcgtgatcaacagcttcttcaacctgcagaacgctaagactgagcagcagcagatcgccttagacagacccctgttcgaggccggcaagacactgatcaataatcagaccagaagaaggcaggaaagagtgcggctggaaacatctctgaccatgagactggcccataagtataacgctaaagccatcatcattgagggagagctgcctcacagctccaccggcacatctcagtaccagaacaacgtgcggctggattggagtgccaagaagagcgccaagctgaaaaccgaaagcgccaactgcgctggaatcgccatctgccagatcgacccttgtcacacctcccaccagaacccttttcggcacacccctacaaaccctgacctgcggccacggttcgcccaggtgaagaaaggcaagatgttccagtaccagcttaatggcctccagcggctgctgaatcctagatcaaagtctagcacagcaatctactaccggcaggccgtgcaaagcttttgtgcccaccacaacctgaccgagagagacatcacctctgccaaatttcccagcgacctggaaaagaagatcaaggacgacacctacctgatccctcagagaggcggccggatctacatcagtagcttccctgttacaagctgcgccagaccttgcacaagcaaccattatttcggcggaggccagttcgagtgtaatgctgatgccgtggccgccgtgaacatcatgctgaaggtccaccct

SEQ ID NO: 39 - DNA, 3033 bp, synthetic construct
  source 1..3033: mol_type = other DNA
atgcctatccggggctataagtgcaccgtggtgcctaatgtgcggaaaaagaaactgctggagaaaacatacagctacctgcaggagggcagcgacgtgtttttcgatctgttcctgtcactgtatggcggcatcgcccctaagatgatccctcaggatctgggcatcaacgagcaagtgatctgtgccgcaaactggttcaagatcgtggaaaagaccaaggactgcatcgccgacgacgccctgctgaaccagtttgcccagtactacggcgagaagcctaacgagaaggttgtgcagtttctgacagcttcttataacaaagataagtacgtgtgggtcgactgccgtcaaaagttctacaccctgcagaaagacctgggagtgcagaacctcgagaacgacctggagtgcctgatccgcgaggacctgctgcctgtgggatctgataaggaagtgaatggatggcacagcatcagcaaactcttcggctgcggcgagaaggaggacagaaccatcaaggccaagattctgaacggcctgtgggagcggatcgagaaggaagatattctgaccgaggaggacgccagaaacgagctgctgcatagcgctggcgtgctgacccctaaggagttcagaaaggtgtacaagggcgccgccggcggacgggactgctaccacaccctgctggttgacggcagaaacttcaccttcaacctgaaaaccctgatcaagcagaccaaggacaagctcaaggaaaagtccgtggatgtggaaatccccaacaaggaggccctgaggctgtacctggaaaagcgaatcggaagatctttcgagcagaagccttggtccgagatgtacaaaaccgccctgagcgctgttatgcccaagaacaccctgaattactgctttgccatcgatagacacgcccagtacacgaagatccagaccctgaagcaaccttacgactctgccatcaccgccctgaacggcttcttcgagagcgaatgcttcaccgggagcgacgtgttcgtgatcagccctagccacctgggaaaaaccctgaagaagctgtacaactacaaggacgttgagagcggaatcagcgagatcgtcgaggacgaggataatagcctgcggagcggcgtgaacgtgaatctgcttcggtacatcttcacactgaaggatatgttcagcgccgaggacttcatcaaggccgccgagtacaacgtagtgtttgagagatacaatagacagaaagtccaccctacagtgaagggcaatcaaagcttcacatttggcaacagcgctctgtctggcaaggtgatccctccatctaagtgtctgagcaacctgcctggacagatgtggctggccatcaatctgctggaccagggcgagtggaaggagcaccacattcccttccacagcgccagattctacgaggaaatctacgctacatctgataaccagaacaaccccgtggacctgcggaccaagagattcggctgttctctgaacaagaccttcagcgccgctgacatcgagaaggtgaaggagtctgccaagaaaaagcacggaaaggccgctaagagaatcctgcgtgccaagaacacaaacaccgccgtgaactgggtggattgcggcttcatgctggaaaagaccgaagtgaacttcaaaatcaccgtcaattacaaactgcccgatcagaagctgggcaagttcgagcctatcgtgggcacaaaaatcctggcttatgaccagaatcagaccgccccagatgcctacgccatcctggaaatttgcgacgattctgaagccttcgactacaagggctacaaaatcaaatgtctgagcaccggggacctggccagcaagtccctgacaaagcagacagaagtggaccagctggcatataagggcgtagacaaaaccagcaacttctacaagaagtggaagcagcagcggagactttttgtgaagagcctgaatatcccagacgccctgaaatcttttgaaaacatcaacaaggagtacctgtacggctttaacaatagttacctgaagctactgaagcaaattctgagaggcaaattcggacctatcctggtggacatcagacctgagctgatcgagatgtgccagggcatcggcagcatcatgcggctgtccagcttgaaccacgacagcctggacgccattcagtccctgaagagcctgctgcactcttacttcgacctgaaggtgaaggaagaaatcaagaccgaagagctgagagagaaggccgataaggaagtgtttaagctgctgcaacaggtgatccagaagcagaagaataagagaaaggaaaaggtgaacagaacagtggatgctatcctgacactggccgccgacgagcaagtgcaggtgatcgtgggcgaaggcgacctgtgcgtgtccaccaagggcaccaaaaagagacagaacaaccggacaatcgactggtgcgcgagagccgtggtcgagaaactggaaaaagcctgcaagctgcacggcctgcacttcaaggaaatccccccccactacaccagccaccaggactgtttcgagcacaacaaggacatcgagaatcctaaggaagtgatgaagtgtagattcaacagcagcgagaacgtggccccttggatgattaagaagttcgccaactaccttaaatgcgagacaaaatactacgtgcagggcatgcaggacttcctggaacattacggcctggtggaatacaaggaccatatcaagaagggaaagatcagtatcggcgattttcagaaactgatcaagctggccctggaaaaagtaggcgagaaggaaatcgtgtttccttgcaaaggcggcagaatctacctgagcacctactgtctgaccaacgagtccaaacccatcgtgttcaacggcagacggtgctatgtgaacaacgccgaccacgtggccgctatcaacgtgggcatctgcctgttgaatttcaacgccagagctaaggtggctgaaaagacacca

SEQ ID NO: 40 - DNA, 3279 bp, synthetic construct
  source 1..3279: mol_type = other DNA
atggccaagaaggacttcatcgccagaccttacaacagctttctgctgcctaacgacagaaagctggcttacctggaagaaacatggaccgcctacaagagcatcaagaccgtgctgcacagatttctgatcgcggcctatggcgccatccccttccagacattcgccaaaaccattgaaaacacccaagaggacgagctgcaactggcctatgccgtgcggatgttcagactggtgcccaaggacttcagcaagaacgagaacaacattccacctgacatgctgatcagcaagctggccagctacaccaatatcaaccagtccccaacaaacgttctcagctacgtgaatagcaactacgacccagagaaatacaagtggatcgattctagaaacgaggccatcagcctgagcaaggagatcggcatcaagctggacgagctcgctgattacgccaccaccatgctgtgggaggattggctgcccctgaacaaggacacagtgaacggctggggaaccacctctggcctgttcggcgccggcaaaaaagaggataggacccaaaaggtgcagatgctgaacgccctgctgctgggcctgaaaaacaacccccccaaggattacaagcagtacagcaccatcctactgaaggcatttgatgccaagagctgggaagaggccgtgaagatttacaaaggcgagtgttctggccgaacaagtagttacctgactgagaagcacggtgacatcagccctgagacactggaaaagctgatccagagcatccagcgggacatcgccgacaaacagcacccaatcaacctgccaaagagagaagaaatcaaagcctacctggagaaacagtctggcaccccatacaacctgaacctgtggagccaggccctgcacaacgccatgagctctatcaagaaaaccgacaccagaaatttcaactctaccctggagaagtacgagaaggaaatccagctgaaggagtgccttcaagatggcgacgatgtggagctgctggggaacaagtttttctcttctccttaccacaagacaaatgatgtgttcgtgatctgctctgaacacatcggaacaaatagaaagtacaacgtggtcgagcagatgtatcagctggccagcgagcacgccgacttcgagacagttttcaccctgctgaaggacgagtatgaggaaaagggcatcaagacacccatcaaaaacatcctggagtacatctggaacaacaagaacgtccctgtgggcacatggggccggatcgctaaatacaaccagctgaaggacagattagcagggatcaaggccaatcccacagtggaatgcaacagaggcatgacatttggcaacagcgccatggtgggcgaagtgatgcgctccaaccggatcagcaccagcaccaagaacaagggccagatcttggcccagatgcacaacgaccggcctgtgggcagcaacaacatgatttggctggaaatgaccctcctgaacaacggcaagtggcagaagcaccacatccccacacacaacaacaaatttttcgaggaagtgcacgccttcaaccctgaactgaagcagagcgtgaacgtgagaaacagaatgtacagaagccagaactactcacagctgcctaccagcctgaccgacggcctgcagggaaatcctaaggccaagatcttcaagagacagtacagagccctgaacaacatgaccgctaatgtgatcgaccctaagctgtccttcatcgtgaacaagaaagatggaagattcgagatcagcatcatccacaacgtggaagtgatccgagccagacgggacgtgctggtcggcgactacctggtgggcatggaccaaaaccagacggcttctaatacctacgccgtcatgcaggtggtgcagcctaacacccccgacagccatgagttcagaaaccagtgggtcaagttcatcgagagcggcaagatcgagagctcaacactgaactcccggggtgagtacatcgaccagctgagccacgatggcgtcgacctgcaggagattaaggattctgagtggattcctgccgccgaaaaattcctgaacaagctaggagctatcaacaaagacggcacccccatcagcatctccaacaccagcaaacgggcctacacattcaatagcatctatttcaaaatcctgctgaattatctgagagccaacgacgtggacctgaatctggtgcgggaagagatcctgcggatcgccaacggcagattcagccctatgcggctgggatctctgtcctggaccacactaaaaatgctgggcaatttccggaacctaattcacagctacttcgaccactgtggctttaaggaaatgcctgagagagaaagcaaggacaagaccatgtacgatctgctgatgcacaccatcaccaagctgaccaacaagcgggccgagcgcaccagcagaatcgctggaagcctgatgaacgtggctcacaagtacaagatcggcacaagcgtggtccacgtggtggtggaaggctctctgagcaaaaccgacaagagcagctccaagggcaacaatcggaataccacagactggtgcagccgggccgtggtgaagaagcttgaagatatgtgcgtgttctacggcttcaacctgaaagccgtgagcgcccactacaccagccaccaggaccctctggttcatagagccgattacgatgatcctaagttggccctgagatgcagatactcttcttacagcagagctgattttgagaagtggggcgaaaaatctttcgccgccgtgatcagatgggccacagacaagaagagcaacacctgctacaaggtgggagccgtagagttcttcaagaactacaaaatccctgaggacaagatcaccaaaaagctgaccatcaaagagttcctggaaattatgtgcgctgagagccactaccctaatgagtacgacgacattctgatccctagaaggggcggcagaatctacctcacaactaagaagctgctgtccgatagcacccaccagagagagtctgtgcatagccataccgccgtggtgaagatgaacggcaaggaatactatagcagcgacgccgatgaggtggctgctatcaatatctgcctgcacgactgggtggtccccctgaattggacaaatcactgcctgcctgccggatggtgtagcgaccacctgaaggaatgcgtgcaatgtcacacccctgatcctgtgagaatcagcatg

SEQ ID NO: 41 - DNA, 1979 bp, synthetic construct
  source 1..1979: mol_type = other DNA
atgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatgaattcgagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccggatccgtgtctttcccattacagtaggagcatacgggagacaagctttggccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 42 - DNA, 50 bp, synthetic construct
  source 1..50: mol_type = other DNA
ggatccgtgtctttcccattacagtaggagcatacgggagacaagctttg

SEQ ID NO: 43 - DNA, 20 bp, synthetic construct
  source 1..20: mol_type = other DNA
ccattacagtaggagcatac

SEQ ID NO: 44 - DNA, 20 bp, synthetic construct
  source 1..20: mol_type = other DNA
gtatgctcctactgtaatgg

SEQ ID NO: 45 - RNA, 20 nt, synthetic construct
  source 1..20: mol_type = other RNA
ccattacagtaggagcatac

SEQ ID NO: 46 - RNA, 20 nt, synthetic construct
  source 1..20: mol_type = other RNA
ggtcttcgataagaagacct

SEQ ID NO: 47 - protein, 1368 aa, Streptococcus pyogenes [sequence omitted]

SEQ ID NO: 48 - RNA, 96 nt, synthetic construct
  source 1..96: mol_type = other RNA
ccattacagtaggagcatacgttttagagctagaaatagcaagttaaaataaggctagtccgttatcaacttgaaaaagtggcaccgagtcggtgc

SEQ ID NO: 49 - protein, 1209 aa, unidentified
  source 1..1209: note = Lachnospiraceae bacterium MA2020
MAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 50 - RNA, 41 nt, synthetic construct
  source 1..41: mol_type = other RNA
taatttctactaagtgtagatccattacagtaggagcatac

SEQ ID NO: 51 - RNA, 56 nt, synthetic construct
  source 1..56: mol_type = other RNA
ctagcaatgactcagaaatgtgtccccagttgacacccattacagtaggagcatac

SEQ ID NO: 52 - RNA, 56 nt, synthetic construct
  source 1..56: mol_type = other RNA
atcgcaacatcttagaaatccgtccttagttgacggccattacagtaggagcatac

SEQ ID NO: 53 - RNA, 56 nt, synthetic construct
  source 1..56: mol_type = other RNA
tctcaacgatagtcagacatgtgtccccagtgacacccattacagtaggagcatac

SEQ ID NO: 54 - RNA, 56 nt, synthetic construct
  source 1..56: mol_type = other RNA
ctcaaagtgtcaaaagaatgtccctgctaatgggacccattacagtaggagcatac

SEQ ID NO: 55 - RNA, 57 nt, synthetic construct
  source 1..57: mol_type = other RNA
tcccaaagtggcaaaagaatctccctgttaatgggagccattacagtaggagcatac

SEQ ID NO: 56 - RNA, 56 nt, synthetic construct
  source 1..56: mol_type = other RNA
gtctaactgccatagaatcgtgcctgcaattggcacccattacagtaggagcatac

SEQ ID NO: 57 - RNA, 55 nt, synthetic construct
  source 1..55: mol_type = other RNA
tcggggcaccaaaataatctccttggtaatgggagccattacagtaggagcatac

SEQ ID NO: 58 - RNA, 56 nt, synthetic construct
  source 1..56: mol_type = other RNA
ccacaacaaccaaaagaatgtccctgaaagtgggacccattacagtaggagcatac

SEQ ID NO: 59 - RNA, 56 nt, synthetic construct
  source 1..56: mol_type = other RNA
gtaacagtggctaagtaatgtgtcttccaatgacacccattacagtaggagcatac

SEQ ID NO: 60 - RNA, 44 nt, synthetic construct
  source 1..44: mol_type = other RNA
gagagaatgtgtgcaaagtcacacccattacagtaggagcatac

SEQ ID NO: 61 - RNA, 10 nt, synthetic construct
  source 1..10: mol_type = other RNA
ccattacagt

SEQ ID NO: 62 - RNA, 12 nt, synthetic construct
  source 1..12: mol_type = other RNA
ccattacagtag

SEQ ID NO: 63 - RNA, 14 nt, synthetic construct
  source 1..14: mol_type = other RNA
ccattacagtagga

SEQ ID NO: 64 - RNA, 15 nt, synthetic construct
  source 1..15: mol_type = other RNA
ccattacagtaggag

SEQ ID NO: 65 - RNA, 16 nt, synthetic construct
  source 1..16: mol_type = other RNA
ccattacagtaggagc

SEQ ID NO: 66 - RNA, 17 nt, synthetic construct
  source 1..17: mol_type = other RNA
ccattacagtaggagca

SEQ ID NO: 67 - RNA, 18 nt, synthetic construct
  source 1..18: mol_type = other RNA
ccattacagtaggagcat

SEQ ID NO: 68 - RNA, 19 nt, synthetic construct
  source 1..19: mol_type = other RNA
ccattacagtaggagcata

SEQ ID NO: 69 - RNA, 21 nt, synthetic construct
  source 1..21: mol_type = other RNA
ccattacagtaggagcatacg

SEQ ID NO: 70 - RNA, 22 nt, synthetic construct
  source 1..22: mol_type = other RNA
ccattacagtaggagcatacgg

SEQ ID NO: 71 - RNA, 23 nt, synthetic construct
  source 1..23: mol_type = other RNA
ccattacagtaggagcatacggg

SEQ ID NO: 72 - RNA, 24 nt, synthetic construct
  source 1..24: mol_type = other RNA
ccattacagtaggagcatacggga

SEQ ID NO: 73 - RNA, 26 nt, synthetic construct
  source 1..26: mol_type = other RNA
ccattacagtaggagcatacgggaga

SEQ ID NO: 74 - RNA, 27 nt, synthetic construct
  source 1..27: mol_type = other RNA
ccattacagtaggagcatacgggagac

SEQ ID NO: 75 - RNA, 28 nt, synthetic construct
  source 1..28: mol_type = other RNA
ccattacagtaggagcatacgggagaca

SEQ ID NO: 76 - RNA, 30 nt, synthetic construct
  source 1..30: mol_type = other RNA
ccattacagtaggagcatacgggagacaag

SEQ ID NO: 77 - RNA, 32 nt, synthetic construct
  source 1..32: mol_type = other RNA
ccattacagtaggagcatacgggagacaagct

SEQ ID NO: 78 - RNA, 35 nt, synthetic construct
  source 1..35: mol_type = other RNA
ccattacagtaggagcatacgggagacaagctttg

SEQ ID NO: 79 - RNA, 40 nt, synthetic construct
  source 1..40: mol_type = other RNA
ccattacagtaggagcatacgggagacaagctttggccac

SEQ ID NO: 80 - RNA, 45 nt, synthetic construct
  source 1..45: mol_type = other RNA
ccattacagtaggagcatacgggagacaagctttggccacctacg

SEQ ID NO: 81 - RNA, 50 nt, synthetic construct
  source 1..50: mol_type = other RNA
ccattacagtaggagcatacgggagacaagctttggccacctacggcaag

SEQ ID NO: 82 - DNA, 20 bp, synthetic construct
  source 1..20: mol_type = other DNA
caaaaacccaaaatatttta

SEQ ID NO: 83 - DNA, 20 bp, synthetic construct
  source 1..20: mol_type = other DNA
gctcctactcagactgttac

SEQ ID NO: 84 - DNA, 19 bp, synthetic construct
  source 1..19: mol_type = other DNA
tgtcaccagagtaacagtc

SEQ ID NO: 85 - DNA, 20 bp, synthetic construct
  source 1..20: mol_type = other DNA
cctcgctggactggtatttg

SEQ ID NO: 86 - DNA, 20 bp, synthetic construct
  source 1..20: mol_type = other DNA
tgtctgaagctggccccgcg

SEQ ID NO: 87 - DNA, 21 bp, synthetic construct
  source 1..21: mol_type = other DNA
ccttcgactcttcctcctttg

SEQ ID NO: 88 - DNA, 21 bp, synthetic construct
  source 1..21: mol_type = other DNA
ctcctttgcctcgctggactg

SEQ ID NO: 89 - DNA, 20 bp, synthetic construct
  source 1..20: mol_type = other DNA
accatcagaggacatttgga

SEQ ID NO: 90 - DNA, 20 bp, synthetic construct
  source 1..20: mol_type = other DNA
gattctccagcaccctgggc

SEQ ID NO: 91 - DNA, 20 bp, synthetic construct
  source 1..20: mol_type = other DNA
cagccacgtctacagcaggg

SEQ ID NO: 92 - DNA, 20 bp, synthetic construct
  source 1..20: mol_type = other DNA
acagccacgtctacagcagg

SEQ ID NO: 93 - DNA, 20 bp, synthetic construct
  source 1..20: mol_type = other DNA
gaacacttttacagccacgt

SEQ ID NO: 94 - DNA, 20 bp, synthetic construct
  source 1..20: mol_type = other DNA
aaaaagacctctgagggatc

SEQ ID NO: 95 - DNA, 20 bp, synthetic construct
  source 1..20: mol_type = other DNA
aacacttttacagccacgtc

SEQ ID NO: 96 - DNA, 20 bp, synthetic construct
  source 1..20: mol_type = other DNA
tagaaggagtgtacagagta

SEQ ID NO: 97 - DNA, 20 bp, synthetic construct
  source 1..20: mol_type = other DNA
gcatttccccgttccatgaa

SEQ ID NO: 98 - DNA, 20 bp, synthetic construct
  source 1..20: mol_type = other DNA
tcatctgtggtgagcccgtg

SEQ ID NO: 99 - DNA, 20 bp, synthetic construct
  source 1..20: mol_type = other DNA
gtgtccagttctactctgta

SEQ ID NO: 100 - DNA, 20 bp, synthetic construct
  source 1..20: mol_type = other DNA
cagtacgatttggtgtccag

SEQ ID NO: 101 - DNA, 20 bp, synthetic construct
  source 1..20: mol_type = other DNA
tacaaacttctcatctgtgg

SEQ ID NO: 102 - DNA, 20 bp, synthetic construct
  source 1..20: mol_type = other DNA
cacagccaacgactctggcc

SEQ ID NO: 103 - DNA, 20 bp, synthetic construct
  source 1..20: mol_type = other DNA
acagccaacgactctggcca

SEQ ID NO: 104 - DNA, 22 bp, synthetic construct
  source 1..22: mol_type = other DNA
ctgacgacagccgtggtgctgt

SEQ ID NO: 105 - DNA, 22 bp, synthetic construct
  source 1..22: mol_type = other DNA
aaaaagacctctgagggatcct

SEQ ID NO: 106 - DNA, 22 bp, synthetic construct
  source 1..22: mol_type = other DNA
agaaaggctgctgatgacacct

SEQ ID NO: 107 - DNA, 22 bp, synthetic construct
  source 1..22: mol_type = other DNA
tagaagggatatacaaagtgga

SEQ ID NO: 108 - DNA, 22 bp, synthetic construct
  source 1..22: mol_type = other DNA
caccacggctgtcgtcaccaat

SEQ ID NO: 109 - DNA, 22 bp, synthetic construct
  source 1..22: mol_type = other DNA
aatccaagtgtcctctgatggt

SEQ ID NO: 110 - DNA, 22 bp, synthetic construct
  source 1..22: mol_type = other DNA
aatgtggccgtgcatgtgttca

SEQ ID NO: 111 - DNA, 22 bp, synthetic construct
  source 1..22: mol_type = other DNA
tagatgctgtccgaggcagtcc

SEQ ID NO: 112 - DNA, 22 bp, synthetic construct
  source 1..22: mol_type = other DNA
gcatgggctcacaactgaggag

SEQ ID NO: 113 - DNA, 22 bp, synthetic construct
  source 1..22: mol_type = other DNA
tatacaaagtggaaatagacac

SEQ ID NO: 114 - DNA, 22 bp, synthetic construct
  source 1..22: mol_type = other DNA
ctggaaggcacttggcatctcc

SEQ ID NO: 115 - DNA, 22 bp, synthetic construct
  source 1..22: mol_type = other DNA
gcatctccccattccatgagca

SEQ ID NO: 116 - DNA, 22 bp, synthetic construct
  source 1..22: mol_type = other DNA
acagccaacgactccggccccc

SEQ ID NO: 117 - DNA, 19 bp, synthetic construct
  source 1..19: mol_type = other DNA
cctggcacctacgtggtgg

SEQ ID NO: 118 - DNA, 18 bp, synthetic construct
  source 1..18: mol_type = other DNA
catggccttcttcctggc

SEQ ID NO: 119 - DNA, 20 bp, synthetic construct
  source 1..20: mol_type = other DNA
ttcctggcttcctggtgaag

SEQ ID NO: 120 - skipped

SEQ ID NO: 121 - DNA, 20 bp, synthetic construct
  source 1..20: mol_type = other DNA
aagttgccccatgtcgacta

SEQ ID NO: 122 - DNA, 20 bp, synthetic construct
  source 1..20: mol_type = other DNA
cccagagcatcccgtggaac

SEQ ID NO: 123 - DNA, 20 bp, synthetic construct
  source 1..20: mol_type = other DNA
cagatacgaacctaaacttt

SEQ ID NO: 124 - DNA, 20 bp, synthetic construct
  source 1..20: mol_type = other DNA
gagtctctcagctggtacac

SEQ ID NO: 125 - DNA, 20 bp, synthetic construct
  source 1..20: mol_type = other DNA
tctgtgatatacacatcaga

SEQ ID NO: 126 - skipped

SEQ ID NO: 127 - DNA, 22 bp, synthetic construct
  source 1..22: mol_type = other DNA
cagcaggcttctacaaagtgga

SEQ ID NO: 128 - DNA, 22 bp, synthetic construct
  source 1..22: mol_type = other DNA
taaaagggatatacaatatgta

SEQ ID NO: 129 - DNA, 22 bp, synthetic construct
  source 1..22: mol_type = other DNA
tagaagggatatagaaagtatc

SEQ ID NO: 130 - DNA, 22 bp, synthetic construct
  source 1..22: mol_type = other DNA
tagaagggatatacaaagtgga

SEQ ID NO: 131 - DNA, 23 bp, synthetic construct
  source 1..23: mol_type = other DNA
tgtctgaggctggccctacggtg

SEQ ID NO: 132 - DNA, 23 bp, synthetic construct
  source 1..23: mol_type = other DNA
accatcagaggacacttggattc

SEQ ID NO: 133 - DNA, 23 bp, synthetic construct
  source 1..23: mol_type = other DNA
tgaacacatgcacggccacattg

SEQ ID NO: 134 - DNA, 23 bp, synthetic construct
  source 1..23: mol_type = other DNA
cctctgggtaagttgccaaagaa

SEQ ID NO: 135 - DNA, 23 bp, synthetic construct
  source 1..23: mol_type = other DNA
gcaacttacccagaggcaaatgg

SEQ ID NO: 136 - DNA, 23 bp, synthetic construct
  source 1..23: mol_type = other DNA
acaccttataggaaaaccagtga

SEQ ID NO: 137 - DNA, 23 bp, synthetic construct
  source 1..23: mol_type = other DNA
ctataaggtgtgaaagtctggat

SEQ ID NO: 138 - DNA, 23 bp, synthetic construct
  source 1..23: mol_type = other DNA
cctataaggtgtgaaagtctgga

SEQ ID NO: 139 - DNA, 23 bp, synthetic construct
  source 1..23: mol_type = other DNA
tagaagggatatacaaagtggaa

SEQ ID NO: 140 - DNA, 23 bp, synthetic construct
  source 1..23: mol_type = other DNA
tatatcccttctacaaattcctc

SEQ ID NO: 141 - DNA, 23 bp, synthetic construct
  source 1..23: mol_type = other DNA
cactttgtatatcccttctacaa

SEQ ID NO: 142 - DNA, 23 bp, synthetic construct
  source 1..23: mol_type = other DNA
gtgtctatttccactttgtatat

SEQ ID NO: 143 - DNA, 23 bp, synthetic construct
  source 1..23: mol_type = other DNA
tgtctgaggctggccctacggtg

SEQ ID NO: 144 - DNA, 23 bp, synthetic construct
  source 1..23: mol_type = other DNA
accatcagaggacacttggattc

SEQ ID NO: 145 - DNA, 23 bp, synthetic construct
  source 1..23: mol_type = other DNA
tgaacacatgcacggccacattg

SEQ ID NO: 146 - DNA, 23 bp, synthetic construct
  source 1..23: mol_type = other DNA
cctctgggtaagttgccaaagaa

SEQ ID NO: 147 - DNA, 23 bp, synthetic construct
  source 1..23: mol_type = other DNA
gcaacttacccagaggcaaatgg

SEQ ID NO: 148 - DNA, 23 bp, synthetic construct
  source 1..23: mol_type = other DNA
acaccttataggaaaaccagtga

SEQ ID NO: 149 - DNA, 23 bp, synthetic construct
  source 1..23: mol_type = other DNA
ctataaggtgtgaaagtctggat

SEQ ID NO: 150 - DNA, 23 bp, synthetic construct
  source 1..23: mol_type = other DNA
cctataaggtgtgaaagtctgga

SEQ ID NO: 151 - DNA, 23 bp, synthetic construct
  source 1..23: mol_type = other DNA
tagaagggatatacaaagtggaa

SEQ ID NO: 152 - DNA, 23 bp, synthetic construct
  source 1..23: mol_type = other DNA
tatatcccttctacaaattcctc

SEQ ID NO: 153 - DNA, 23 bp, synthetic construct
  source 1..23: mol_type = other DNA
cactttgtatatcccttctacaa

SEQ ID NO: 154 - DNA, 23 bp, synthetic construct
  source 1..23: mol_type = other DNA
gtgtctatttccactttgtatat

SEQ ID NO: 155 - DNA, 20 bp, synthetic construct
  source 1..20: mol_type = other DNA
ccacctatgagagaagacag

SEQ ID NO: 156 - DNA, 20 bp, synthetic construct
  source 1..20: mol_type = other DNA
ggctgtcgtcaccaatccca

SEQ ID NO: 157 - DNA, 20 bp, synthetic construct
  source 1..20: mol_type = other DNA
gacgacagccgtggtggaat

SEQ ID NO: 158 - DNA, 20 bp, synthetic construct
  source 1..20: mol_type = other DNA
ctgaacacatgcacggccac

SEQ ID NO: 159 - DNA, 20 bp, synthetic construct
  source 1..20: mol_type = other DNA
cacccagggcaccggtgaat

SEQ ID NO: 160 - DNA, 20 bp, synthetic construct
  source 1..20: mol_type = other DNA
aatggtgtagcggcgggggc

SEQ ID NO: 161 - DNA, 20 bp, synthetic construct
  source 1..20: mol_type = other DNA
ctttggcaacttacccagag

SEQ ID NO: 162 - DNA, 20 bp, synthetic construct
  source 1..20: mol_type = other DNA
tgtctgaggctggccctacg

SEQ ID NO: 163 - DNA, 20 bp, synthetic construct
  source 1..20: mol_type = other DNA
tttgtgtctgaggctggccc

SEQ ID NO: 164 - DNA, 20 bp, synthetic construct
  source 1..20: mol_type = other DNA
attggtgacgacagccgtgg

SEQ ID NO: 165 - DNA, 20 bp, synthetic construct
  source 1..20: mol_type = other DNA
aggagaagtccctcattcct

SEQ ID NO: 166 - DNA, 20 bp, synthetic construct
  source 1..20: mol_type = other DNA
ccaagtgccttccagtaaga

SEQ ID NO: 167 - DNA, 20 bp, synthetic construct
  source 1..20: mol_type = other DNA
acacaaataccagtccagca

SEQ ID NO: 168 - DNA, 20 bp, synthetic construct
  source 1..20: mol_type = other DNA
ccagtccagcaaggcagagg

SEQ ID NO: 169 - DNA, 20 bp, synthetic construct
  source 1..20: mol_type = other DNA
gaagtccactcattcttggc

SEQ ID NO: 170 - DNA, 20 bp, synthetic construct
  source 1..20: mol_type = other DNA
aaagttctagatgctgtccg

SEQ ID NO: 171 - DNA, 20 bp, synthetic construct
  source 1..20: mol_type = other DNA
cccagaggcaaatggctccc

SEQ ID NO: 172 - DNA, 20 bp, synthetic construct
  source 1..20: mol_type = other DNA
ttctttggcaacttacccag

SEQ ID NO: 173 - DNA, 20 bp, synthetic construct
  source 1..20: mol_type = other DNA
actgaggaggaatttgtaga

SEQ ID NO: 174 - DNA, 20 bp, synthetic construct
  source 1..20: mol_type = other DNA
cccattccatgagcatgcag

SEQ ID NO: 175 - DNA, 20 bp, synthetic construct
  source 1..20: mol_type = other DNA
gcatgggctcacaactgagg

SEQ ID NO: 176 - DNA, 20 bp, synthetic construct
  source 1..20: mol_type = other DNA
aataggagtaggggctcagc

SEQ ID NO: 177 - DNA, 20 bp, synthetic construct
  source 1..20: mol_type = other DNA
gacgacagccgtggtggaat

SEQ ID NO: 178 - DNA, 20 bp, synthetic construct
  source 1..20: mol_type = other DNA
ggctgtcgtcaccaatccca

SEQ ID NO: 179 - DNA, 20 bp, synthetic construct
  source 1..20: mol_type = other DNA
gtcaccaatcccaaggaatg

SEQ ID NO: 180 - DNA, 20 bp, synthetic construct
  source 1..20: mol_type = other DNA
tgtgtctgaggctggcccta

SEQ ID NO: 181 - DNA, 20 bp, synthetic construct
  source 1..20: mol_type = other DNA
agcctttctgaacacatgca

SEQ ID NO: 182 - DNA, 20 bp, synthetic construct
  source 1..20: mol_type = other DNA
cagaggacacttggattcac

SEQ ID NO: 183 - DNA, 20 bp, synthetic construct
  source 1..20: mol_type = other DNA
cattgatggcaggactgcct

SEQ ID NO: 184 - DNA, 20 bp, synthetic construct
  source 1..20: mol_type = other DNA
cttctctacacccagggcac

SEQ ID NO: 185 - DNA, 20 bp, synthetic construct
  source 1..20: mol_type = other DNA
aatggtgtagcggcgggggc

SEQ ID NO: 186 - DNA, 20 bp, synthetic construct
  source 1..20: mol_type = other DNA
cccctactcctattccacca

SEQ ID NO: 187 - DNA, 20 bp, synthetic construct
  source 1..20: mol_type = other DNA
gcagggcggcaatggtgtag

SEQ ID NO: 188 - DNA, 20 bp, synthetic construct
  source 1..20: mol_type = other DNA
ggagtaggggctcagcaggg

SEQ ID NO: 189 - DNA, 20 bp, synthetic construct
  source 1..20: mol_type = other DNA
gtattcacagccaacgactc

SEQ ID NO: 190 - DNA, 20 bp, synthetic construct
  source 1..20: mol_type = other DNA
tcacagaaacactcaccgta

SEQ ID NO: 191 - DNA, 20 bp, synthetic construct
  source 1..20: mol_type = other DNA
aaaggctgctgatgacacct

SEQ ID NO: 192 - DNA, 20 bp, synthetic construct
  source 1..20: mol_type = other DNA
cttggattcaccggtgccct

SEQ ID NO: 193 - DNA, 20 bp, synthetic construct
  source 1..20: mol_type = other DNA
gccgtggtggaataggagta

SEQ ID NO: 194 - DNA, 20 bp, synthetic construct
  source 1..20: mol_type = other DNA
gcggcaatggtgtagcggcg

SEQ ID NO: 195 - DNA, 20 bp, synthetic construct
  source 1..20: mol_type = other DNA
ggagaagtccctcattcctt

SEQ ID NO: 196 - DNA, 20 bp, synthetic construct
  source 1..20: mol_type = other DNA
ggcggcaatggtgtagcggc

SEQ ID NO: 197 - DNA, 20 bp, synthetic construct
  source 1..20: mol_type = other DNA
tcaccaatcccaaggaatga

SEQ ID NO: 198 - DNA, 20 bp, synthetic construct
  source 1..20: mol_type = other DNA
gcaacttacccagaggcaaa

SEQ ID NO: 199 - DNA, 20 bp, synthetic construct
  source 1..20: mol_type = other DNA
aagtgccttccagtaagatt

SEQ ID NO: 200 - DNA, 20 bp, synthetic construct
  source 1..20: mol_type = other DNA
acctctgcatgctcatggaa

SEQ ID NO: 201 - DNA, 20 bp, synthetic construct
  source 1..20: mol_type = other DNA
tactcacctctgcatgctca

SEQ ID NO: 202 - DNA, 20 bp, synthetic construct
  source 1..20: mol_type = other DNA
tgtagaagggatatacaaag

SEQ ID NO: 203 - DNA, 20 bp, synthetic construct
  source 1..20: mol_type = other DNA
aggagaagtccctcattcct

SEQ ID NO: 204 - DNA, 20 bp, synthetic construct
  source 1..20: mol_type = other DNA
attggtgacgacagccgtgg

SEQ ID NO: 205 - DNA, 20 bp, synthetic construct
  source 1..20: mol_type = other DNA
gcggcgggggccggagtcgt

SEQ ID NO: 206 - DNA, 20 bp, synthetic construct
  source 1..20: mol_type = other DNA
gggattggtgacgacagccg

SEQ ID NO: 207 - DNA, 20 bp, synthetic construct
  source 1..20: mol_type = other DNA
ggggctcagcagggcggcaa

SEQ ID NO: 208 - DNA, 23 bp, synthetic construct
  source 1..23: mol_type = other DNA
tgtctgaggctggccctacggtg

SEQ ID NO: 209 - DNA, 23 bp, synthetic construct
  source 1..23: mol_type = other DNA
accatcagaggacacttggattc

SEQ ID NO: 210 - DNA, 23 bp, synthetic construct
  source 1..23: mol_type = other DNA
tgaacacatgcacggccacattg

SEQ ID NO: 211 - DNA, 23 bp, synthetic construct
  source 1..23: mol_type = other DNA
cctctgggtaagttgccaaagaa

SEQ ID NO: 212 - DNA, 23 bp, synthetic construct
  source 1..23: mol_type = other DNA
gcaacttacccagaggcaaatgg

SEQ ID NO: 213 - DNA, 23 bp, synthetic construct
  source 1..23: mol_type = other DNA
acaccttataggaaaaccagtga

SEQ ID NO: 214 - DNA, 23 bp, synthetic construct
  source 1..23: mol_type = other DNA
ctataaggtgtgaaagtctggat

SEQ ID NO: 215 - DNA, 23 bp, synthetic construct
  source 1..23: mol_type = other DNA
cctataaggtgtgaaagtctgga

SEQ ID NO: 216 - DNA, 23 bp, synthetic construct
  source 1..23: mol_type = other DNA
tagaagggatatacaaagtggaa

SEQ ID NO: 217 - DNA, 23 bp, synthetic construct
  source 1..23: mol_type = other DNA
tatatcccttctacaaattcctc

SEQ ID NO: 218 - DNA, 23 bp, synthetic construct
  source 1..23: mol_type = other DNA
cactttgtatatcccttctacaa

SEQ ID NO: 219 - DNA, 23 bp, synthetic construct
  source 1..23: mol_type = other DNA
gtgtctatttccactttgtatat

SEQ ID NO: 220 - DNA, 20 bp, synthetic construct
  source 1..20: mol_type = other DNA
ctggaaggcacttggcatct

SEQ ID NO: 221 - DNA, 20 bp, synthetic construct
  source 1..20: mol_type = other DNA
taggaaaaccagtgagtctg

SEQ ID NO: 222 - DNA, 20 bp, synthetic construct
  source 1..20: mol_type = other DNA
tcatcgtctgctcctcctct

SEQ ID NO: 223 - DNA, 20 bp, synthetic construct
  source 1..20: mol_type = other DNA
ttggcaggatggcttctcat

SEQ ID NO: 224 - DNA, 20 bp, synthetic construct
  source 1..20: mol_type = other DNA
accggtgccctgggtgtaga

SEQ ID NO: 225 - DNA, 20 bp, synthetic construct
  source 1..20: mol_type = other DNA
agaaaggctgctgatgacac

SEQ ID NO: 226 - DNA, 20 bp, synthetic construct
  source 1..20: mol_type = other DNA
tagatgctgtccgaggcagt

SEQ ID NO: 227 - DNA, 20 bp, synthetic construct
  source 1..20: mol_type = other DNA
tctacacccagggcaccggt

SEQ ID NO: 228 - DNA, 20 bp, synthetic construct
  source 1..20: mol_type = other DNA
tttggcaacttacccagagg

SEQ ID NO: 229 - DNA, 20 bp, synthetic construct
  source 1..20: mol_type = other DNA
cagtaagatttggtgtctat

SEQ ID NO: 230 - DNA, 20 bp, synthetic construct
  source 1..20: mol_type = other DNA
catgagcatgcagaggtgag

SEQ ID NO: 231 - DNA, 20 bp, synthetic construct
  source 1..20: mol_type = other DNA
ctcctcagttgtgagcccat

SEQ ID NO: 232 - DNA, 20 bp, synthetic construct
  source 1..20: mol_type = other DNA
tacaaattcctcctcagttg

SEQ ID NO: 233 - DNA, 20 bp, synthetic construct
  source 1..20: mol_type = other DNA
acagccaacgactccggccc

SEQ ID NO: 234 - DNA, 20 bp, synthetic construct
  source 1..20: mol_type = other DNA
caccacggctgtcgtcacca

SEQ ID NO: 235 - DNA, 20 bp, synthetic construct
  source 1..20: mol_type = other DNA
cttgggattggtgacgacag

SEQ ID NO: 236 - DNA, 20 bp, synthetic construct
  source 1..20: mol_type = other DNA
tctcataggtggtattcaca

SEQ ID NO: 237 - DNA, 20 bp, synthetic construct
  source 1..20: mol_type = other DNA
ctggactggtatttgtgtct

SEQ ID NO: 238 - DNA, 20 bp, synthetic construct
  source 1..20: mol_type = other DNA
gcaggatggcttctcatcgt

SEQ ID NO: 239 - DNA, 20 bp, synthetic construct
  source 1..20: mol_type = other DNA
atggcaggactgcctcggac

SEQ ID NO: 240 - DNA, 20 bp, synthetic construct
  source 1..20: mol_type = other DNA
gattcaccggtgccctgggt

SEQ ID NO: 241 - DNA, 20 bp, synthetic construct
  source 1..20: mol_type = other DNA
gcatctccccattccatgag

SEQ ID NO: 242 - DNA, 20 bp, synthetic construct
  source 1..20: mol_type = other DNA
tgagcccatgcagctctcca

SEQ ID NO: 243 - DNA, 20 bp, synthetic construct
  source 1..20: mol_type = other DNA
ccgccctgctgagcccctac

SEQ ID NO: 244 - DNA, 20 bp, synthetic construct
  source 1..20: mol_type = other DNA
gctgtgaataccacctatga

SEQ ID NO: 245 - DNA, 20 bp, synthetic construct
  source 1..20: mol_type = other DNA
ggattggtgacgacagccgt

SEQ ID NO: 246 - DNA, 20 bp, synthetic construct
  source 1..20: mol_type = other DNA
gtgacgacagccgtggtgga

SEQ ID NO: 247 - DNA, 20 bp, synthetic construct
  source 1..20: mol_type = other DNA
gtgtctgaggctggccctac

SEQ ID NO: 248 - DNA, 20 bp, synthetic construct
  source 1..20: mol_type = other DNA
gaccatcagaggacacttgg

SEQ ID NO: 249 - DNA, 20 bp, synthetic construct
  source 1..20: mol_type = other DNA
gcctctgggtaagttgccaa

SEQ ID NO: 250 - DNA, 20 bp, synthetic construct
  source 1..20: mol_type = other DNA
ggcaacttacccagaggcaa

SEQ ID NO: 251 - DNA, 20 bp, synthetic construct
  source 1..20: mol_type = other DNA
ggtgtctatttccactttgt

SEQ ID NO: 252 - DNA, 20 bp, synthetic construct
  source 1..20: mol_type = other DNA
gtatatcccttctacaaatt

SEQ ID NO: 253 - DNA, 23 bp, synthetic construct
  source 1..23: mol_type = other DNA
tgtctgaggctggccctacggtg

SEQ ID NO: 254 - DNA, 23 bp, synthetic construct
  source 1..23: mol_type = other DNA
accatcagaggacacttggattc

SEQ ID NO: 255 - DNA, 23 bp, synthetic construct
  source 1..23: mol_type = other DNA
tgaacacatgcacggccacattg

SEQ ID NO: 256 - DNA, 23 bp, synthetic construct
  source 1..23: mol_type = other DNA
cctctgggtaagttgccaaagaa

SEQ ID NO: 257 - DNA, 23 bp, synthetic construct
  source 1..23: mol_type = other DNA
acaccttataggaaaaccagtga

SEQ ID NO: 258 - DNA, 23 bp, synthetic construct
  source 1..23: mol_type = other DNA
ctataaggtgtgaaagtctggat

SEQ ID NO: 259 - DNA, 23 bp, synthetic construct
  source 1..23: mol_type = other DNA
cctataaggtgtgaaagtctgga

SEQ ID NO: 260 - DNA, 23 bp, synthetic construct
  source 1..23: mol_type = other DNA
tagaagggatatacaaagtggaa

SEQ ID NO: 261 - DNA, 23 bp, synthetic construct
  source 1..23: mol_type = other DNA
tatatcccttctacaaattcctc

SEQ ID NO: 262 - DNA, 23 bp, synthetic construct
  source 1..23: mol_type = other DNA
cactttgtatatcccttctacaa

SEQ ID NO: 263 - DNA, 23 bp, synthetic construct
  source 1..23: mol_type = other DNA
gtgtctatttccactttgtatat

SEQ ID NO: 264 - DNA, 20 bp, synthetic construct
  source 1..20: mol_type = other DNA
ctggaaggcacttggcatct

SEQ ID NO: 265 - DNA, 20 bp, synthetic construct
  source 1..20: mol_type = other DNA
taggaaaaccagtgagtctg

SEQ ID NO: 266 - DNA, 20 bp, synthetic construct
  source 1..20: mol_type = other DNA
tcatcgtctgctcctcctct

SEQ ID NO: 267 - DNA, 20 bp, synthetic construct
  source 1..20: mol_type = other DNA
ttggcaggatggcttctcat

SEQ ID NO: 268 - DNA, 20 bp, synthetic construct
  source 1..20: mol_type = other DNA
accggtgccctgggtgtaga

SEQ ID NO: 269 - DNA, 20 bp, synthetic construct
  source 1..20: mol_type = other DNA
agaaaggctgctgatgacac

SEQ ID NO: 270 - DNA, 20 bp, synthetic construct
  source 1..20: mol_type = other DNA
tagatgctgtccgaggcagt

SEQ ID NO: 271 - DNA, 20 bp, synthetic construct
  source 1..20: mol_type = other DNA
tttggcaacttacccagagg

SEQ ID NO: 272 - DNA, 20 bp, synthetic construct
  source 1..20: mol_type = other DNA
cagtaagatttggtgtctat

SEQ ID NO: 273 - DNA, 20 bp, synthetic construct
  source 1..20: mol_type = other DNA
catgagcatgcagaggtgag

SEQ ID NO: 274 - DNA, 20 bp, synthetic construct
  source 1..20: mol_type = other DNA
ctcctcagttgtgagcccat

SEQ ID NO: 275 - DNA, 20 bp, synthetic construct
  source 1..20: mol_type = other DNA
tacaaattcctcctcagttg

SEQ ID NO: 276 - DNA, 20 bp, synthetic construct
  source 1..20: mol_type = other DNA
acagccaacgactccggccc

SEQ ID NO: 277 - DNA, 20 bp, synthetic construct
  source 1..20: mol_type = other DNA
caccacggctgtcgtcacca

SEQ ID NO: 278 - DNA, 20 bp, synthetic construct
  source 1..20: mol_type = other DNA
cttgggattggtgacgacag

SEQ ID NO: 279 - DNA, 20 bp, synthetic construct
  source 1..20: mol_type = other DNA
tctcataggtggtattcaca

SEQ ID NO: 280 - DNA, 20 bp, synthetic construct
  source 1..20: mol_type = other DNA
ctggactggtatttgtgtct

SEQ ID NO: 281 - DNA, 20 bp, synthetic construct
  source 1..20: mol_type = other DNA
gcaggatggcttctcatcgt

SEQ ID NO: 282 - DNA, 20 bp, synthetic construct
  source 1..20: mol_type = other DNA
atggcaggactgcctcggac

SEQ ID NO: 283 - DNA, 20 bp, synthetic construct
  source 1..20: mol_type = other DNA
gattcaccggtgccctgggt

SEQ ID NO: 284 - DNA, 20 bp, synthetic construct
  source 1..20: mol_type = other DNA
gcatctccccattccatgag

SEQ ID NO: 285 - DNA, 20 bp, synthetic construct
  source 1..20: mol_type = other DNA
tgagcccatgcagctctcca

SEQ ID NO: 286 - DNA, 20 bp, synthetic construct
  source 1..20: mol_type = other DNA
ccgccctgctgagcccctac

SEQ ID NO: 287 - DNA, 20 bp, synthetic construct
  source 1..20: mol_type = other DNA
gctgtgaataccacctatga

SEQ ID NO: 288 - DNA, 20 bp, synthetic construct
  source 1..20: mol_type = other DNA
ggattggtgacgacagccgt

SEQ ID NO: 289 - DNA, 20 bp, synthetic construct
  source 1..20: mol_type = other DNA
gtgacgacagccgtggtgga

SEQ ID NO: 290 - DNA, 20 bp, synthetic construct
  source 1..20: mol_type = other DNA
gtgtctgaggctggccctac

SEQ ID NO: 291 - DNA, 20 bp, synthetic construct
  source 1..20: mol_type = other DNA
gaccatcagaggacacttgg

SEQ ID NO: 292 - DNA, 20 bp, synthetic construct
  source 1..20: mol_type = other DNA
gcctctgggtaagttgccaa

SEQ ID NO: 293 - DNA, 20 bp, synthetic construct
  source 1..20: mol_type = other DNA
ggcaacttacccagaggcaa

SEQ ID NO: 294 - DNA, 20 bp, synthetic construct
  source 1..20: mol_type = other DNA
ggtgtctatttccactttgt

SEQ ID NO: 295 - DNA, 20 bp, synthetic construct
  source 1..20: mol_type = other DNA
gtatatcccttctacaaatt

SEQ ID NO: 296 - DNA, 23 bp, synthetic construct
  source 1..23: mol_type = other DNA
tgtctgaggctggccctacggtg

SEQ ID NO: 297 - DNA, 23 bp, synthetic construct
  source 1..23: mol_type = other DNA
accatcagaggacacttggattc

SEQ ID NO: 298 - DNA, 23 bp, synthetic construct
  source 1..23: mol_type = other DNA
tgaacacatgcacggccacattg

SEQ ID NO: 299 - DNA, 23 bp, synthetic construct
  source 1..23: mol_type = other DNA
cctctgggtaagttgccaaagaa

SEQ ID NO: 300 - DNA, 23 bp, synthetic construct
  source 1..23: mol_type = other DNA
acaccttataggaaaaccagtga

SEQ ID NO: 301 - DNA, 23 bp, synthetic construct
  source 1..23: mol_type = other DNA
ctataaggtgtgaaagtctggat

SEQ ID NO: 302 - DNA, 23 bp, synthetic construct
  source 1..23: mol_type = other DNA
cctataaggtgtgaaagtctgga

SEQ ID NO: 303 - DNA, 23 bp, synthetic construct
  source 1..23: mol_type = other DNA
tagaagggatatacaaagtggaa

SEQ ID NO: 304 - DNA, 23 bp, synthetic construct
  source 1..23: mol_type = other DNA
tatatcccttctacaaattcctc

SEQ ID NO: 305 - DNA, 23 bp, synthetic construct
  source 1..23: mol_type = other DNA
cactttgtatatcccttctacaa

SEQ ID NO: 306 - DNA, 23 bp, synthetic construct
  source 1..23: mol_type = other DNA
gtgtctatttccactttgtatat

SEQ ID NO: 307 - DNA, 20 bp, synthetic construct
  source 1..20: mol_type = other DNA
ctggaaggcacttggcatct

SEQ ID NO: 308 - DNA, 20 bp, synthetic construct
  source 1..20: mol_type = other DNA
taggaaaaccagtgagtctg

SEQ ID NO: 309 - DNA, 20 bp, synthetic construct
  source 1..20: mol_type = other DNA
tcatcgtctgctcctcctct

SEQ ID NO: 310 - DNA, 20 bp, synthetic construct
  source 1..20: mol_type = other DNA
ttggcaggatggcttctcat

SEQ ID NO: 311 - DNA, 20 bp, synthetic construct
  source 1..20: mol_type = other DNA
accggtgccctgggtgtaga

SEQ ID NO: 312 - DNA, 20 bp, synthetic construct
  source 1..20: mol_type = other DNA
agaaaggctgctgatgacac

SEQ ID NO: 313 - DNA, 20 bp, synthetic construct
  source 1..20: mol_type = other DNA
tagatgctgtccgaggcagt

SEQ ID NO: 314 - DNA, 20 bp, synthetic construct
  source 1..20: mol_type = other DNA
tttggcaacttacccagagg

SEQ ID NO: 315 - DNA, 20 bp, synthetic construct
  source 1..20: mol_type = other DNA
cagtaagatttggtgtctat

SEQ ID NO: 316 - DNA, 20 bp, synthetic construct
  source 1..20: mol_type = other DNA
catgagcatgcagaggtgag

SEQ ID NO: 317 - DNA, 20 bp, synthetic construct
  source 1..20: mol_type = other DNA
ctcctcagttgtgagcccat

SEQ ID NO: 318 - DNA, 20 bp, synthetic construct
  source 1..20: mol_type = other DNA
tacaaattcctcctcagttg

SEQ ID NO: 319 - DNA, 20 bp, synthetic construct
  source 1..20: mol_type = other DNA
acagccaacgactccggccc

SEQ ID NO: 320 - DNA, 20 bp, synthetic construct
  source 1..20: mol_type = other DNA
caccacggctgtcgtcacca

SEQ ID NO: 321 - DNA, 20 bp, synthetic construct
  source 1..20: mol_type = other DNA
cttgggattggtgacgacag

SEQ ID NO: 322 - DNA, 20 bp, synthetic construct
  source 1..20: mol_type = other DNA
tctcataggtggtattcaca

SEQ ID NO: 323 - DNA, 20 bp, synthetic construct
  source 1..20: mol_type = other DNA
ctggactggtatttgtgtct

SEQ ID NO: 324 - DNA, 20 bp, synthetic construct
  source 1..20: mol_type = other DNA
gcaggatggcttctcatcgt

SEQ ID NO: 325 - DNA, 20 bp, synthetic construct
  source 1..20: mol_type = other DNA
atggcaggactgcctcggac

SEQ ID NO: 326 - DNA, 20 bp, synthetic construct
  source 1..20: mol_type = other DNA
gattcaccggtgccctgggt

SEQ ID NO: 327 - DNA, 20 bp, synthetic construct
  source 1..20: mol_type = other DNA
gcatctccccattccatgag

SEQ ID NO: 328 - DNA, 20 bp, synthetic construct
  source 1..20: mol_type = other DNA
tgagcccatgcagctctcca

SEQ ID NO: 329 - DNA, 20 bp, synthetic construct
  source 1..20: mol_type = other DNA
ccgccctgctgagcccctac

SEQ ID NO: 330 - DNA, 20 bp, synthetic construct
  source 1..20: mol_type = other DNA
gctgtgaataccacctatga

SEQ ID NO: 331 - DNA, 20 bp, synthetic construct
  source 1..20: mol_type = other DNA
ggattggtgacgacagccgt

SEQ ID NO: 332 - DNA, 20 bp, synthetic construct
  source 1..20: mol_type = other DNA
gtgacgacagccgtggtgga

SEQ ID NO: 333 - DNA, 20 bp, synthetic construct
  source 1..20: mol_type = other DNA
gtgtctgaggctggccctac

SEQ ID NO: 334 - DNA, 20 bp, synthetic construct
  source 1..20: mol_type = other DNA
gaccatcagaggacacttgg

SEQ ID NO: 335 - DNA, 20 bp, synthetic construct
  source 1..20: mol_type = other DNA
gcctctgggtaagttgccaa

SEQ ID NO: 336 - DNA, 20 bp, synthetic construct
  source 1..20: mol_type = other DNA
ggcaacttacccagaggcaa

SEQ ID NO: 337 - DNA, 20 bp, synthetic construct
  source 1..20: mol_type = other DNA
ggtgtctatttccactttgt

SEQ ID NO: 338 - DNA, 20 bp, synthetic construct
  source 1..20: mol_type = other DNA
gtatatcccttctacaaatt

SEQ ID NO: 339 - DNA, 23 bp, synthetic construct
  source 1..23: mol_type = other DNA
tgtctgaggctggccctacggtg

SEQ ID NO: 340 - DNA, 23 bp, synthetic construct
  source 1..23: mol_type = other DNA
accatcagaggacacttggattc

SEQ ID NO: 341 - DNA, 23 bp, synthetic construct
  source 1..23: mol_type = other DNA
tgaacacatgcacggccacattg

SEQ ID NO: 342 - DNA, 23 bp, synthetic construct
  source 1..23: mol_type = other DNA
cctctgggtaagttgccaaagaa

SEQ ID NO: 343 - DNA, 23 bp, synthetic construct
  source 1..23: mol_type = other DNA
acaccttataggaaaaccagtga

SEQ ID NO: 344 - DNA, 23 bp, synthetic construct
  source 1..23: mol_type = other DNA
ctataaggtgtgaaagtctggat

SEQ ID NO: 345 - DNA, 23 bp, synthetic construct
  source 1..23: mol_type = other DNA
cctataaggtgtgaaagtctgga

SEQ ID NO: 346 - DNA, 23 bp, synthetic construct
  source 1..23: mol_type = other DNA
tagaagggatatacaaagtggaa

SEQ ID NO: 347 - DNA, 23 bp, synthetic construct
  source 1..23: mol_type = other DNA
tatatcccttctacaaattcctc

SEQ ID NO: 348 - DNA, 23 bp, synthetic construct
  source 1..23: mol_type = other DNA
cactttgtatatcccttctacaa

SEQ ID NO: 349 - DNA, 23 bp, synthetic construct
  source 1..23: mol_type = other DNA
gtgtctatttccactttgtatat

SEQ ID NO: 350 - DNA, 20 bp, synthetic construct
  source 1..20: mol_type = other DNA
ctggaaggcacttggcatct

SEQ ID NO: 351 - DNA, 20 bp, synthetic construct
  source 1..20: mol_type = other DNA
taggaaaaccagtgagtctg

SEQ ID NO: 352 - DNA, 20 bp, synthetic construct
  source 1..20: mol_type = other DNA
tcatcgtctgctcctcctct

SEQ ID NO: 353 - DNA, 20 bp, synthetic construct
  source 1..20: mol_type = other DNA
ttggcaggatggcttctcat

SEQ ID NO: 354 - DNA, 20 bp, synthetic construct
  source 1..20: mol_type = other DNA
accggtgccctgggtgtaga

SEQ ID NO: 355 - DNA, 20 bp, synthetic construct
  source 1..20: mol_type = other DNA
agaaaggctgctgatgacac

SEQ ID NO: 356 - DNA, 20 bp, synthetic construct
  source 1..20: mol_type = other DNA
tagatgctgtccgaggcagt

SEQ ID NO: 357 - DNA, 20 bp, synthetic construct
  source 1..20: mol_type = other DNA
tttggcaacttacccagagg

SEQ ID NO: 358 - DNA, 20 bp, synthetic construct
  source 1..20: mol_type = other DNA
cagtaagatttggtgtctat

SEQ ID NO: 359 - DNA, 20 bp, synthetic construct
  source 1..20: mol_type = other DNA
catgagcatgcagaggtgag

SEQ ID NO: 360 - DNA, 20 bp, synthetic construct
  source 1..20: mol_type = other DNA
ctcctcagttgtgagcccat

SEQ ID NO: 361 - DNA, 20 bp, synthetic construct
  source 1..20: mol_type = other DNA
tacaaattcctcctcagttg

SEQ ID NO: 362 - DNA, 20 bp, synthetic construct
  source 1..20: mol_type = other DNA
acagccaacgactccggccc

SEQ ID NO: 363 - DNA, 20 bp, synthetic construct
  source 1..20: mol_type = other DNA
caccacggctgtcgtcacca

SEQ ID NO: 364 - DNA, 20 bp, synthetic construct
  source 1..20: mol_type = other DNA
cttgggattggtgacgacag

SEQ ID NO: 365 - DNA, 20 bp, synthetic construct
  source 1..20: mol_type = other DNA
tctcataggtggtattcaca

SEQ ID NO: 366 - DNA, 20 bp, synthetic construct
  source 1..20: mol_type = other DNA
ctggactggtatttgtgtct

SEQ ID NO: 367 - DNA, 20 bp, synthetic construct
  source 1..20: mol_type = other DNA
gcaggatggcttctcatcgt

SEQ ID NO: 368 - DNA, 20 bp, synthetic construct
  source 1..20: mol_type = other DNA
atggcaggactgcctcggac

SEQ ID NO: 369 - DNA, 20 bp, synthetic construct
  source 1..20: mol_type = other DNA
gattcaccggtgccctgggt

SEQ ID NO: 370 - DNA, 20 bp, synthetic construct
  source 1..20: mol_type = other DNA
gcatctccccattccatgag

SEQ ID NO: 371 - DNA, 20 bp, synthetic construct
  source 1..20: mol_type = other DNA
tgagcccatgcagctctcca

SEQ ID NO: 372 - DNA, 20 bp, synthetic construct
  source 1..20: mol_type = other DNA
ccgccctgctgagcccctac

SEQ ID NO: 373 - DNA, 20 bp, synthetic construct
  source 1..20: mol_type = other DNA
gctgtgaataccacctatga

SEQ ID NO: 374 - DNA, 20 bp, synthetic construct
  source 1..20: mol_type = other DNA
ggattggtgacgacagccgt

SEQ ID NO: 375 - DNA, 20 bp, synthetic construct
  source 1..20: mol_type = other DNA
gtgacgacagccgtggtgga

SEQ ID NO: 376 - DNA, 20 bp, synthetic construct
  source 1..20: mol_type = other DNA
gtgtctgaggctggccctac

SEQ ID NO: 377 - DNA, 20 bp, synthetic construct
  source 1..20: mol_type = other DNA
gaccatcagaggacacttgg

SEQ ID NO: 378 - DNA, 20 bp, synthetic construct
  source 1..20: mol_type = other DNA
gcctctgggtaagttgccaa

SEQ ID NO: 379 - DNA, 20 bp, synthetic construct
  source 1..20: mol_type = other DNA
ggcaacttacccagaggcaa

SEQ ID NO: 380 - DNA, 20 bp, synthetic construct
  source 1..20: mol_type = other DNA
ggtgtctatttccactttgt

SEQ ID NO: 381 - DNA, 20 bp, synthetic construct
  source 1..20: mol_type = other DNA
gtatatcccttctacaaatt

SEQ ID NO: 382 - DNA, 20 bp, synthetic construct
  source 1..20: mol_type = other DNA
cacataggccattcagaaac

SEQ ID NO: 383 - DNA, 20 bp, synthetic construct
  source 1..20: mol_type = other DNA
catatgggtcattcagaaac

SEQ ID NO: 384 - DNA, 20 bp, synthetic construct
  source 1..20: mol_type = other DNA
cacacaggcctttcagcaac

SEQ ID NO: 385 - DNA, 20 bp, synthetic construct
  source 1..20: mol_type = other DNA
cacagatgccattcagatac

SEQ ID NO: 386 - DNA, 20 bp, synthetic construct
  source 1..20: mol_type = other DNA
cacataggtcattcacaaat

SEQ ID NO: 387 - DNA, 20 bp, synthetic construct
  source 1..20: mol_type = other DNA
cagatagtcctttcagaaac

SEQ ID NO: 388 - DNA, 20 bp, synthetic construct
  source 1..20: mol_type = other DNA
cacataggctattcaaaatc

SEQ ID NO: 389 - DNA, 20 bp, synthetic construct
  source 1..20: mol_type = other DNA
cacatggcccatacagaaac

SEQ ID NO: 390 - DNA, 20 bp, synthetic construct
  source 1..20: mol_type = other DNA
ctcaaaggccattcaggaac

SEQ ID NO: 391 - DNA, 20 bp, synthetic construct
  source 1..20: mol_type = other DNA
cacataggccattcagaaac

SEQ ID NO: 392 - DNA, 20 bp, synthetic construct
  source 1..20: mol_type = other DNA
ccctggctccctcacccacc

SEQ ID NO: 393 - DNA, 20 bp, synthetic construct
  source 1..20: mol_type = other DNA
catatgggtcattcagaaac

SEQ ID NO: 394 - DNA, 20 bp, synthetic construct
  source 1..20: mol_type = other DNA
cacacaggcctttcagcaac

SEQ ID NO: 395 - DNA, 20 bp, synthetic construct
  source 1..20: mol_type = other DNA
cacagatgccattcagatac

SEQ ID NO: 396 - DNA, 20 bp, synthetic construct
  source 1..20: mol_type = other DNA
cacataggtcattcacaaat

SEQ ID NO: 397 - DNA, 20 bp, synthetic construct
  source 1..20: mol_type = other DNA
cagatagtcctttcagaaac

SEQ ID NO: 398 - DNA, 20 bp, synthetic construct
  source 1..20: mol_type = other DNA
agcaccaccacgtaggtgcc

SEQ ID NO: 399 - DNA, 20 bp, synthetic construct
  source 1..20: mol_type = other DNA
ctggtgaagatgagtggcga

SEQ ID NO: 400 - DNA, 20 bp, synthetic construct
  source 1..20: mol_type = other DNA
aagttgccccatgtcgacta

SEQ ID NO: 401 - DNA, 20 bp, synthetic construct
  source 1..20: mol_type = other DNA
ccccatgtcgactacatcga

SEQ ID NO: 402 - DNA, 20 bp, synthetic construct
  source 1..20: mol_type = other DNA
cccagagcatcccgtggaac

SEQ ID NO: 403 - DNA, 20 bp, synthetic construct
  source 1..20: mol_type = other DNA
ccggtggtcactctgtatgc

SEQ ID NO: 404 - DNA, 20 bp, synthetic construct
  source 1..20: mol_type = other DNA
agcacgcgcaggctgcgcat

SEQ ID NO: 405 - DNA, 20 bp, synthetic construct
  source 1..20: mol_type = other DNA
gcggcaccctcataggtgag

SEQ ID NO: 406 - DNA, 20 bp, synthetic construct
  source 1..20: mol_type = other DNA
gggccaccaatgcccaggac

SEQ ID NO: 407 - DNA, 20 bp, synthetic construct
  source 1..20: mol_type = other DNA
gtggccccaactgtgatgac

SEQ ID NO: 408 - DNA, 20 bp, synthetic construct
  source 1..20: mol_type = other DNA
gtgcctccagcgactgcagc

SEQ ID NO: 409 - DNA, 20 bp, synthetic construct
  source 1..20: mol_type = other DNA
acccctgcaccaggcattgc

SEQ ID NO: 410 - DNA, 20 bp, synthetic construct
  source 1..20: mol_type = other DNA
cctgaggaccagcgggtact

SEQ ID NO: 411 - DNA, 20 bp, synthetic construct
  source 1..20: mol_type = other DNA
gtggcagtggacacgggtcc

SEQ ID NO: 412 - DNA, 20 bp, synthetic construct
  source 1..20: mol_type = other DNA
tctacggcgtaggcccccag

SEQ ID NO: 413 - DNA, 20 bp, synthetic construct
  source 1..20: mol_type = other DNA
caagtgtcctctgatggtca

SEQ ID NO: 414 - DNA, 20 bp, synthetic construct
  source 1..20: mol_type = other DNA
gtcaaagttctagatgctgt

SEQ ID NO: 415 - DNA, 20 bp, synthetic construct
  source 1..20: mol_type = other DNA
ctgtccgaggcagtcctgcc

SEQ ID NO: 416 - DNA, 20 bp, synthetic construct
  source 1..20: mol_type = other DNA
tggccgtgcatgtgttcaga

SEQ ID NO: 417 - DNA, 20 bp, synthetic construct
  source 1..20: mol_type = other DNA
tgttcagaaaggctgctgat

SEQ ID NO: 418 - DNA, 20 bp, synthetic construct
  source 1..20: mol_type = other DNA
acacctgggagccatttgcc

SEQ ID NO: 419 - DNA, 20 bp, synthetic construct
  source 1..20: mol_type = other DNA
caccggtgccctgggtgtag

SEQ ID NO: 420 - DNA, 20 bp, synthetic construct
  source 1..20: mol_type = other DNA
agaggacacttggattcacc

SEQ ID NO: 421 - DNA, 20 bp, synthetic construct
  source 1..20: mol_type = other DNA
tagaactttgaccatcagag

SEQ ID NO: 422 - DNA, 20 bp, synthetic construct
  source 1..20: mol_type = other DNA
gcaggactgcctcggacagc

SEQ ID NO: 423 - DNA, 20 bp, synthetic construct
  source 1..20: mol_type = other DNA
gatggcaggactgcctcgga

SEQ ID NO: 424 - DNA, 20 bp, synthetic construct
  source 1..20: mol_type = other DNA
cacggccacattgatggcag

SEQ ID NO: 425 - DNA, 20 bp, synthetic construct
  source 1..20: mol_type = other DNA
agcagcctttctgaacacat

SEQ ID NO: 426 - DNA, 20 bp, synthetic construct
  source 1..20: mol_type = other DNA
tgatggtcaaagttctagat

SEQ ID NO: 427 - DNA, 20 bp, synthetic construct
  source 1..20: mol_type = other DNA
atggtcaaagttctagatgc

SEQ ID NO: 428 - DNA, 20 bp, synthetic construct
  source 1..20: mol_type = other DNA
tccgaggcagtcctgccatc

SEQ ID NO: 429 - DNA, 20 bp, synthetic construct
  source 1..20: mol_type = other DNA
atgacacctgggagccattt

SEQ ID NO: 430 - DNA, 20 bp, synthetic construct
  source 1..20: mol_type = other DNA
ggagccatttgcctctgggt

SEQ ID NO: 431 - DNA, 20 bp, synthetic construct
  source 1..20: mol_type = other DNA
tgggtaagttgccaaagaac

SEQ ID NO: 432 - DNA, 20 bp, synthetic construct
  source 1..20: mol_type = other DNA
ggattcaccggtgccctggg

SEQ ID NO: 433 - DNA, 20 bp, synthetic construct
  source 1..20: mol_type = other DNA
tgaccatcagaggacacttg

SEQ ID NO: 434 - DNA, 20 bp, synthetic construct
  source 1..20: mol_type = other DNA
gaactttgaccatcagagga

SEQ ID NO: 435 - DNA, 20 bp, synthetic construct
  source 1..20: mol_type = other DNA
ggacagcatctagaactttg

SEQ ID NO: 436 - DNA, 20 bp, synthetic construct
  source 1..20: mol_type = other DNA
cctcggacagcatctagaac

SEQ ID NO: 437 - DNA, 20 bp, synthetic construct
  source 1..20: mol_type = other DNA
ccaggtgtcatcagcagcct

SEQ ID NO: 438 - DNA, 20 bp, synthetic construct
  source 1..20: mol_type = other DNA
acccagaggcaaatggctcc

SEQ ID NO: 439 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 440 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 441 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 442 - protein, 16 aa, synthetic construct
SGSETPGTSESATPES

SEQ ID NO: 443 - protein, 18 aa, synthetic construct
KRTADGSEFESPKKKRKV

SEQ ID NO: 444 - protein, 7 aa, synthetic construct
PKKKRKV

SEQ ID NO: 445 - protein, 16 aa, synthetic construct
KRPAATKKAGQAKKKK

SEQ ID NO: 446 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 447 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 448 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 449 - protein, 291 aa, synthetic construct
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 450 - DNA, 794 bp, synthetic construct
  source 1..794: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 451 - RNA, 30 nt, synthetic construct
  source 1..30: mol_type = other RNA
atgactcagaaatgtgtccccagttgacac

SEQ ID NO: 452 - RNA, 23 nt, synthetic construct
  source 1..23: mol_type = other RNA
agaaatgtgtccccagttgacac

SEQ ID NO: 453 - RNA, 23 nt, synthetic construct
  source 1..23: mol_type = other RNA
agaaatccgtccttagttgacgg

SEQ ID NO: 454 - RNA, 22 nt, synthetic construct
  source 1..22: mol_type = other RNA
agacatgtgtccccagtgacac

SEQ ID NO: 455 - RNA, 23 nt, synthetic construct
  source 1..23: mol_type = other RNA
agaaatgtttccccagttgaaac

SEQ ID NO: 456 - RNA, 23 nt, synthetic construct
  source 1..23: mol_type = other RNA
agaaatgtgttcccagttaacac

SEQ ID NO: 457 - RNA, 23 nt, synthetic construct
  source 1..23: mol_type = other RNA
agaaatttgtccccagttgacaa

SEQ ID NO: 458 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKELSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 459 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNARRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 460 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADRKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 461 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 462 - protein, 17 aa, synthetic construct
KRTADGSESEPKKKRKV

SEQ ID NO: 463 - protein, 1315 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSKRTADGSEFESPKKKRKVSGGSSGGSMSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPGKRPAATKKAGQAKKKK

SEQ ID NO: 464 - protein, 1453 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGGSSGGSKRTADGSEFESPKKKRKVSGGSSGGSMSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPGGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSSGGSKRTADGSESEPKKKRKV

SEQ ID NO: 465 - protein, 34 aa, synthetic construct
SGGSSGGSKRTADGSEFESPKKKRKVSGGSSGGS

SEQ ID NO: 466 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPG

SEQ ID NO: 467 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

(19) United States
(12) Patent Application Publication
(10) Pub. No.: US 2025/0101394 A1
(43) Pub. Date: Mar. 27, 2025

(54) NOVEL CRISPR-CAS12I SYSTEMS AND USES THEREOF

(71) Applicant: Huidagene Therapeutics (Singapore) PTE. LTD., Singapore (SG)

(72) Inventors: Hainan ZHANG, Shanghai (CN); Jingxing ZHOU, Shanghai (CN); Haoqiang WANG, Shanghai (CN); Weihong ZHANG, Shanghai (CN)

(21) Appl. No.: 18/832,823

(22) PCT Filed: Jan. 20, 2023
(86) PCT No.: PCT/CN2023/073420

§ 371 Date: Jul. 24, 2024

(30) Foreign Application Priority Data
Jan. 24, 2022 (CN) 202210081981.1
Apr. 25, 2022 (WO) PCT/CN2022/089074
Nov. 2, 2022 (WO) PCT/CN2022/129376

(51) Int. Cl. C12N 9/22; C12N 15/10; C12N 15/11

(52) U.S. Cl. CPC C12N 9/22; C12N 15/102; C12N 15/111; C12N 2310/20

(57) ABSTRACT

Provided are Cas12i polypeptides, fusion proteins comprising such Cas12i polypeptides, CRISPR-Cas12i systems comprising such Cas12i polypeptides or fusion proteins, and methods of using the same.

### CROSS-REFERENCE TO RELATED APPLICATIONS

This application claims the benefits of and priorities to CN patent application No. 202210081981.1, filed on Jan. 24, 2022, entitled “NOVEL CRISPR-CAS12I SYSTEMS”; PCT Patent Application No. PCT/CN2022/089074, filed on Apr. 25, 2022, entitled “NOVEL CRISPR-CAS12I SYSTEMS”, and PCT Patent Application No. PCT/CN2022/129376, filed on Nov. 2, 2022, entitled “NOVEL CRISPR-CAS12I SYSTEMS AND USES THEREOF”, the entire contents of which, including any sequence listing and drawings, are incorporated herein by reference in its entirety.

### REFERENCE TO AN ELECTRONIC SEQUENCE LISTING

The contents of the electronic sequence listing (“HEP001PCT2.xml”; Size is 499,182 bytes and it was created on Jan. 20, 2023) is incorporated herein by reference in its entirety. Wherever a sequence is an RNA sequence, the T in the sequence shall be deemed as U.

### TECHNICAL FIELD

The disclosure is generally directed to Cas12i polypeptides, fusion proteins comprising such Cas12i polypeptides, CRISPR-Cas12i systems comprising such Cas12i polypeptides or fusion proteins, and methods of using the same.

### BACKGROUND

The clustered regularly interspaced short palindromic repeats-Cas (CRISPR-Cas) systems, including type II Cas9 and type V Cas12 systems, which serve in the adaptive immunity of prokaryotes against viruses, have been developed into genome editing tools¹⁻³. Compared with type II systems, the type V systems including V-A to V-K showed more functional diversity^{4, 5}. Amongst them, Cas12i has a relatively smaller size (1033-1093 aa), compared to SpCas9 and Cas12a, and has a 5′-TTN protospacer adjacent motif (PAM) preference^{4, 6, 7}. Cas12i is characterized by the capability of autonomously processing precursor crRNA (pre-crRNA) to form short mature crRNA. Cas12i mediates cleavage of dsDNA with a single RuvC domain, by preferentially nicking the non-target strand and then cutting the target strand⁸⁻¹⁰. These intrinsic features of Cas12i enable multiplex high-fidelity genome editing. However, the previous Cas12i (Cas12i1 and Cas12i2) showed low editing efficiency which limits their utility for therapeutic gene editing. It is thus needed to develop CRISPR-Cas12i systems with higher efficiency for practical use.

Citation or identification of any document in this application is not an admission that such a document is available as prior art to the disclosure.

### SUMMARY

To address the limitations of previous Cas12i, the applicant screened ten Cas12i and found one, xCas12i (also referred to as “SiCas12i” herein), with robust high activity in HEK293T cells. Engineering of xCas12i by arginine substitutions at the PAM-interacting (PI), REC and RuvC domains led to the production of a variant, high-fidelity Cas12Max (hfCas12Max), with significantly elevated editing activity and minimal off-target cleavage efficiency. In addition, the applicant assessed the base editing efficiency of xCas12i-based base editor, and thus expanded the genome-editing toolbox. The applicant further demonstrated that hfCas12Max could be an effective genome-editing tool ex vivo and in vivo via ribonucleoprotein (RNP) and lipid nanoliposomes (LNP) respectively, suggesting the excellent potential for therapeutic genome editing applications.

It is against the above background that the disclosure provides certain advantages and advancements over the prior art.

Although the disclosure is not limited to specific advantages or functionalities, in one aspect, the disclosure provides a Cas12i polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the reference Cas12i polypeptide of SEQ ID NO: 1,
optionally wherein the Cas12i polypeptide has a function (e.g., a modified function that is either increased or decreased compared to that) of the reference Cas12i polypeptide
(e.g.,
(a) an ability to form a complex with a guide RNA capable of forming a complex with the reference Cas12i polypeptide; and/or,
(b) a spacer sequence-specific dsDNA cleavage activity).

In some embodiments, the Cas12i polypeptide has increased spacer sequence-specific dsDNA and/or ssDNA cleavage activity compared to that of the reference Cas12i polypeptide of SEQ ID NO: 1 when both used in combination with a same guide RNA, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more. In some embodiments, the Cas12i polypeptide comprises one or more mutations (such as, insertions, deletions, or substitutions) at one or more amino acids at one or more of the following positions of the amino acid sequence of the reference Cas12i polypeptide of SEQ ID NO: 1:
S118, D119, F121, W123, Q136, E138, E143, V146, S155, V158, E161, S162, T163, A165, N166, G178, D180, T185, K189, A193, D196, N199, N200, E202, L203, S221, V233, E235, N236, S241, N243, S245, K251, D255, L257, N273, D287, S295, V302, S332, E336, S338, V339, E362, D375, A377, N378, D381, T382, E385, D387, N390, E395, E396, Q398, N399, V400, D403, E406, Q407, V409, D411, C412, N416, N418, L440, L448, V451, Q455, E464, S806, S817, V818, S819, S832, M833, F835, T836, F837, C839, A840, E842, E843, K844, T846, N847, K848, N854, A856, S858, Q862, K863, Y865, L866, G868, K870, M871, D876, D877, V880, G883, K884, G886, K887, A888, A891, D892, M894, A900, K903, K904, N906, V910, M912, S913, C915, Y916, A918, M923, S925, H926, Q927, V931, M933, Q934, D935, K936, K937, T938, S939, V940, F945, M946, V948, N949, K950, D951, S952, D955, Y956, A959, G960, N966, S967, K968, S969, D970, A971, G972, S974, V975, Y976, Q979, A980, L982, H983, C985, E986, A987, G989, V990, S991, P992, E993, L994, V995, K996, N997, K998, K999, T1000, H1001, A1002, A1003, E1004, G1006, G1010, A1012, M1013, L1014, W1017, V1022, K1028, D1032, K1034, K1037, C1039, G1040, Q1045, H1047, C1063, and G1069; and optionally N243, E336, V880, G883, D892, and M923.

In some embodiments, the mutation is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)); and optionally Arginine (Arg/R).

In some embodiments, the substitution at N243 is a substitution with R, A, V, L, I, M, F, W, S, T, C, Y, N, Q, E, K, or H; and optionally R.

In some embodiments, the Cas12i polypeptide comprises substitution N243R in the amino acid sequence of the reference Cas12i polypeptide of SEQ ID NO: 1.

In some embodiments, the Cas12i polypeptide comprises, consists essentially of, or consists of the amino acid sequence of SEQ ID NO: 458, or an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 458.

In some embodiments, the Cas12i polypeptide comprises substitutions N243R and E336R in the amino acid sequence of the reference Cas12i polypeptide of SEQ ID NO: 1.

In some embodiments, the Cas12i polypeptide comprises, consists essentially of, or consists of the amino acid sequence of SEQ ID NO: 467, or an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 467.

In some embodiments, the Cas12i polypeptide has decreased spacer sequence-independent (off-target) dsDNA and/or ssDNA cleavage activity compared to that of the reference Cas12i polypeptide of SEQ ID NO: 1 when both used in combination with a same guide RNA, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the Cas12i polypeptide comprises one or more mutations (such as, insertions, deletions, or substitutions) at one or more amino acids at one or more of the following positions of the amino acid sequence of the reference Cas12i polypeptide of SEQ ID NO: 1: V880, G883, D892, and M923.

In some embodiments, the mutation is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)); and optionally Arginine (Arg/R).

In some embodiments, the Cas12i polypeptide comprises one or more substitutions selected from the group consisting of G883R, D892R, V880R, and M923R in the amino acid sequence of the reference Cas12i polypeptide of SEQ ID NO: 1.

In some embodiments, the Cas12i polypeptide comprises substitution N243R and one or more substitutions selected from the group consisting of G883R, D892R, V880R, and M923R in the amino acid sequence of the reference Cas12i polypeptide of SEQ ID NO: 1.

In some embodiments, the Cas12i polypeptide comprises substitutions N243R and E336R and one or more substitutions selected from the group consisting of G883R, D892R, V880R, and M923R in the amino acid sequence of the reference Cas12i polypeptide of SEQ ID NO: 1.

In some embodiments, the Cas12i polypeptide comprises substitutions N243R, E336R, and D892R in the amino acid sequence of the reference Cas12i polypeptide of SEQ ID NO: 1.

In some embodiments, the Cas12i polypeptide comprises, consists essentially of, or consists of the amino acid sequence of SEQ ID NO: 459, or an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 459.

In some embodiments, the Cas12i polypeptide has decreased spacer sequence-specific dsDNA and/or ssDNA cleavage activity compared to that of the reference Cas12i polypeptide of SEQ ID NO: 1 when both used in combination with a same guide RNA, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the Cas12i polypeptide is a dead Cas12i polypeptide having substantially no spacer sequence-specific dsDNA and/or ssDNA cleavage activity, e.g., having at most about 5%, 10%, 15%, 20%, 25%, 30%, 35%, 40%, 45%, or 50% of spacer sequence-specific dsDNA and/or ssDNA cleavage activity of the reference Cas12i polypeptide of SEQ ID NO: 1.

In some embodiments, the Cas12i polypeptide comprise one or more substitutions selected from the group consisting of D650A, D700A, E875A, and D1049A in the amino acid sequence of the reference Cas12i polypeptide of SEQ ID NO: 1.

In some embodiments, the Cas12i polypeptide further comprise one or more substitutions selected from the group consisting of D650A, D700A, E875A, and D1049A in the amino acid sequence of the reference Cas12i polypeptide of SEQ ID NO: 1.

In some embodiments, the Cas12i polypeptide comprises substitutions N243R, E336R, and D1049A in the amino acid sequence of the reference Cas12i polypeptide of SEQ ID NO: 1.

In some embodiments, the Cas12i polypeptide comprises, consists essentially of, or consists of the amino acid sequence of SEQ ID NO: 466, or an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 466.

In some embodiments, the Cas12i polypeptide comprises one or more mutations (such as, insertions, deletions, or substitutions) at one or more amino acids at one or more of the following positions of the amino acid sequence of the reference Cas12i polypeptide of SEQ ID NO: 1:
K109, L112, D125, 127, F144, L147, A148, L151, L157, V195, Y226, F252, 1258, M293, W305, A308, 1309, S312, A314, D315, V316, A318, L324, 1327, A348, L352, Y365, L372, L376, L379, L383, 1405, L424, I427, A436, F439, A443, V447, A457, H458, P459, T460, S463, S814, F859, A864, H867, Y977, S1031, A1053, and F1068.

In some embodiments, the mutation is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)); and optionally Arginine (Arg/R).

In some embodiments, the Cas12i polypeptide has (1) decreased spacer sequence-specific dsDNA cleavage activity compared to that of the reference Cas12i polypeptide of SEQ ID NO: 1 when both used in combination with a same guide RNA, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%; and (2) increased spacer sequence-specific ssDNA cleavage activity compared to that of the reference Cas12i polypeptide of SEQ ID NO: 1 when both used in combination with a same guide RNA, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more. In some embodiments, the Cas12i polypeptide is a Cas12i nickase having spacer sequence-specific ssDNA cleavage activity and having substantially no spacer sequence-specific dsDNA cleavage activity.

In some embodiments, the Cas12i nickase has nickase preference >1.0 and nickase activity >20% (as defined and measured according to the method in Example 12).

In some embodiments, the Cas12i polypeptide comprise one or more substitutions selected from the group consisting of W896R, W896P, W896K, S924F, S924D, S924E, S924H, and S925T in the amino acid sequence of the reference Cas12i polypeptide of SEQ ID NO: 1.

In some embodiments, the Cas12i polypeptide is capable of recognizing a target adjacent motif (TAM) immediately 5′ to the protospacer sequence on the non-target strand of a target dsDNA, and wherein the TAM is 5′-NTTN-3′, 5′-TN-3′, or 5′-TNN-3′, wherein N is A, T, G, or C.

In another aspect, the disclosure provides a fusion protein comprising the Cas12i polypeptide of the disclosure and a functional domain.

In some embodiments, the functional domain is fused N-terminally, C-terminally, or internally with respect to the Cas12i polypeptide.

In some embodiments, the functional domain is fused to the Cas12i polypeptide via a linker, e.g., a XTEN linker (SEQ ID NO: 442), a GS linker containing multiple glycine and serine residues, a GS linker containing multiple glycine and serine residues and a XTEN linker (SEQ ID NO: 442), a GS linker (SEQ ID NO: 465) containing multiple glycine and serine residues and a BP NLS (SEQ ID NO: 443).

In some embodiments, the functional domain is selected from the group consisting of a nuclear localization signal (NLS), a nuclear export signal (NES), a deaminase or a catalytic domain thereof, an uracil glycosylase inhibitor (UGI), an uracil glycosylase (UNG), a methylpurine glycosylase (MPG), a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease or a catalytic domain thereof, a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment thereof, and any combination thereof.

In some embodiments, the NLS comprises or is SV40 NLS (SEQ ID NO: 444), bpSV40 NLS (BP NLS, bpNLS, SEQ ID NO: 443 or 462), or NP NLS (Xenopus laevis Nucleoplasmin NLS, nucleoplasmin NLS, SEQ ID NO: 445).

In some embodiments, the functional domain comprises a deaminase or a catalytic domain thereof.

In some embodiments, the deaminase or catalytic domain thereof is an adenine deaminase (e.g., TadA, such as, TadA8e, TadA8.17, TadA8.20, TadA9) or a catalytic domain thereof, for example, TadA8e-V106W (SEQ ID NO: 439), TadA8e-W106V (SEQ ID NO: 461).

In some embodiments, the deaminase or catalytic domain thereof is a cytidine deaminase (e.g., APOBEC, such as, APOBEC3, for example, APOBEC3A, APOBEC3B, APOBEC3C; DddA) or a catalytic domain thereof, for example, hAPOBEC3-W104A (SEQ ID NO: 440).

In some embodiments, the functional domain comprises an uracil glycosylase inhibitor (UGI), for example, human UGI domain (SEQ ID NO: 441).

In some embodiments, the functional domain comprises an uracil glycosylase (UNG).

In some embodiments, the functional domain comprises a methylpurine glycosylase (MPG).

In some embodiments, the functional domain comprises a reverse transcriptase or a catalytic domain thereof.

In some embodiments, the functional domain comprises a methylase or a catalytic domain thereof.

In some embodiments, the functional domain comprises a transcription activating domain.

In yet another aspect, the disclosure provides a system (or composition) comprising:
(1) the Cas12i polypeptide of the disclosure or the fusion protein of the disclosure, or a polynucleotide encoding the Cas12i polypeptide or the fusion protein; and
(2) a guide RNA (also referred to as “CRISPR RNA” or “crRNA”) or a polynucleotide encoding the guide RNA, the guide RNA comprising:
(i) a direct repeat sequence capable of forming a complex with the Cas12i polypeptide or the fusion protein; and
(ii) a spacer sequence capable of hybridizing to a target sequence on a target strand of a target dsDNA, thereby guiding the complex to the target dsDNA;
optionally, wherein the direct repeat sequence is 5′ to the spacer sequence.

In some embodiments, the direct repeat sequence:
(1) is as set forth in any one of SEQ ID NOs: 11 and 451-457;
(2) comprises the polynucleotide sequence of any one of SEQ ID NOs: 11 and 451-457; or
(3) comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of any one of SEQ ID NOs: 11 and 451-457.

In some embodiments, the direct repeat sequence has substantially the same secondary structure as the secondary structure of any one of SEQ ID NOs: 11 and 451-457.

In some embodiments, the target sequence comprises, consists essentially of, or consists of at least about 16 contiguous nucleotides of a target gene, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous nucleotides of a target gene, or in a numerical range between any of two preceding values, e.g., from about 16 to about 50 contiguous nucleotides of a target gene.

In some embodiments, the target sequence is at least about 16 nucleotides in length, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length, or in a length of a numerical range between any of two preceding values, e.g., in a length of from about 16 to about 50 nucleotides.

In some embodiments, the spacer sequence is at least about 16 nucleotides in length, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length, or in a length of a numerical range between any of two preceding values, e.g., in a length of from about 16 to about 50 nucleotides.

In some embodiments, the guide RNA comprises a plurality (e.g., 2, 3, 4, 5 or more) of the spacer sequences capable of hybridizing to a plurality of the target sequences, respectively.

In some embodiments, the guide RNA comprises, in 5′ to 3′ orientation, the direct repeat sequence, the spacer sequence, the direct repeat sequence, and the spacer sequence.

In some embodiments, the spacer sequence comprises at least 16 contiguous nucleotides of any one of SEQ ID NOs: 82-119, 121-125, 130, 131-381, 382, 391, and 398-438.

In some embodiments, the dsDNA is within a cell.

In yet another aspect, the disclosure provides a method for modifying a target dsDNA, comprising contacting the target dsDNA with the system of the disclosure, wherein the spacer sequence is capable of hybridizing to a target sequence of a target strand of the target dsDNA, wherein the target sequence is modified by the complex.

In some embodiments, the target dsDNA is human TRAC gene.

In some embodiments, the spacer sequence comprises at least 10 contiguous nucleotides of any one of SEQ ID NOs: 123-125.

In yet another aspect, the disclosure provides a cell or a progeny thereof comprising the Cas12i polypeptide of the disclosure, the fusion protein of the disclosure, or the system of the disclosure.

In yet another aspect, the disclosure provides a modified cell or a progeny thereof, wherein the modified cell is modified by the method of the disclosure.

In some embodiments, the cell is in vivo, ex vivo, or in vitro.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is a cultured cell, an isolated primary cell, or a cell within a living organism.

In some embodiments, the cell is a T cell (such as, CAR-T cell), B cell, NK cell (such as, CAR-NK cell), or stem cell (such as, iPS cell, HSC cell).

In some embodiments, the cell is derived from or heterogenous to the subject.

In yet another aspect, the disclosure provides a host comprising the cell or progeny thereof of the disclosure.

In yet another aspect, the disclosure provides a (e.g., pharmaceutical) composition comprising the Cas12i polypeptide of the disclosure, the fusion protein of the disclosure, the system of the disclosure, or the cell or progeny thereof of the disclosure.

In yet another aspect, the disclosure provides a method for diagnosing, preventing, or treating a disease or disorder in a subject, comprising administering to the subject (e.g., an effective amount of) the system of the disclosure, the cell or progeny thereof of the disclosure, or the composition of the disclosure.

In some embodiments, the disease or disorder is a TTR-associated disease or disorder, e.g., ATTR.

In some embodiments, the spacer sequence comprises at least 10 contiguous nucleotides of SEQ ID NO: 107.

In some embodiments, the disease or disorder is a PCSK9-associated disease or disorder.

In some embodiments, the spacer sequence comprises at least 10 contiguous nucleotides of SEQ ID NO: 122.

In some aspects, the disclosure provides a Cas12i polypeptide:
(1) as set forth in any one of SEQ ID NOs: 1-3, 6, and 10;
(2) comprising the amino acid sequence of any one of SEQ ID NOs: 1-3, 6, and 10; or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 1-3, 6, and 10.

In some aspects, the disclosure provides a Cas12i polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the amino acid sequence of the reference Cas12i polypeptide of any one of SEQ ID NOs: 1-3, 6, and 10,
optionally wherein the Cas12i polypeptide has a function (e.g., a modified function that is either increased or decreased compared to that) of the reference Cas12i polypeptide (e.g.,
(a) an ability to form a complex with a guide RNA capable of forming a complex with the reference Cas12i polypeptide; and/or,
(b) a spacer sequence-specific dsDNA cleavage activity).

In some embodiment, the Cas12i polypeptide has increased spacer sequence-specific dsDNA and/or ssDNA cleavage activity compared to that of the reference Cas12i polypeptide of any one of SEQ ID NOs: 1-3, 6, and 10 when both used in combination with a same guide RNA, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiment, the Cas12i polypeptide has decreased spacer sequence-specific dsDNA and/or ssDNA cleavage activity compared to that of the reference Cas12i polypeptide of any one of SEQ ID NOs: 1-3, 6, and 10 when both used in combination with a same guide RNA, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiment, the Cas12i polypeptide is a dead Cas12i polypeptide having substantially no spacer sequence-specific dsDNA and/or ssDNA cleavage activity, e.g., having at most about 5%, 10%, 15%, 20%, 25%, 30%, 35%, 40%, 45%, or 50% of spacer sequence-specific dsDNA and/or ssDNA cleavage activity of the reference Cas12i polypeptide of any one of SEQ ID NOs: 1-3, 6, and 10.

In some embodiment, the Cas12i polypeptide comprise a substitution selected from the group consisting of D650A, D700A, E875A, and D1049A of SEQ ID NO: 1, or a combination thereof.

In some embodiment, the Cas12i polypeptide is a Cas12i nickase having spacer sequence-specific ssDNA cleavage activity.

In some embodiment, the Cas12i polypeptide is a Cas12i nickase having spacer sequence-specific ssDNA cleavage activity against the target strand of a target dsDNA.

In some embodiment, the Cas12i polypeptide is a Cas12i nickase having spacer sequence-specific ssDNA cleavage activity against the target strand of a target dsDNA, and having substantially no spacer sequence-specific dsDNA cleavage activity, e.g., having at most about 5%, 10%, 15%, 20%, 25%, 30%, 35%, 40%, 45%, or 50% of spacer sequence-specific dsDNA cleavage activity of the reference Cas 12i polypeptide of any one of SEQ ID NOS: 1-3, 6, and 10.

In some embodiment, the Cas12i polypeptide comprise a substitution selected from the group consisting of the mutant in Tables 11-14 of SEQ ID NO: 1, or a combination thereof.

In some embodiment, the Cas12i polypeptide is not any one of SEQ ID NOs: 1-3, 6, and 10.

In some embodiment, the Cas12i polypeptide has decreased spacer sequence-independent (off-target) dsDNA and/or ssDNA cleavage activity compared to that of the reference Cas12i polypeptide of any one of SEQ ID NOs: 1-3, 6, and 10 when both used in combination with a same guide RNA, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiment, the Cas12i polypeptide comprises one or more mutations (such as, insertions, deletions, or substitutions) at one or more amino acids corresponding to one or more amino acids of the amino acid sequence of the reference Cas12i polypeptide of any one of SEQ ID NOs: 1-3, 6, and 10.

In some embodiment, the one or more mutations are within a domain corresponding to the PI domain, REC-I domain, and/or RuvC-II domain of the reference Cas 12i polypeptide of any one of SEQ ID NOs: 1-3, 6, and 10.

In some embodiment, the one or more mutations are within the PI domain at positions 173-291, the REC-I domain at positions 427-473, and/or RuvC-II domain at positions 800-1082 of the reference Cas12i polypeptide of SEQ ID NO: 1.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of any one of SEQ ID NOS: 1-3, 6, and 10:

any one of positions 1 to the end of the reference Cas12i polypeptide of any one of SEQ ID NOs: 1-3, 6, and 10, e.g., 1080, such as, position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1: any one of positions 1 to 1080, such as, position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1: K109, N110, Y111, L112, M113, S114, N115, 1116, D117, S118, D119, F121, V122, W123, V124, D125, C126, 127, K128, F129, A130, K131, D132, F133, A134, Y135, Q136, M137, E138, L139, G140, F141, H142, E143, F144, T145, V146, L147, A148, E149, T150, L151, L152, A153, N154, S155, 1156, L157, V158, L159, N160, E161, S162, T163, K164, A165, N166, W167, A168, W169, G170, T171, V172, S173, A174, L175, Y176, G177, G178, G179, D180, K181, E182, D183, S184, T185, L186, K187, S188, K189, 1190, L191, L192, A193, F194, V195, D196, A197, L198, N199, N200, H201, E202, L203, K204, T205, K206, E208, 1209, L210, N211, Q212, V213, C214, E215, S216, L217, K218, Y219, Q220, S221, Y222, Q223, D224, M225, Y226, V227, D228, F229, S231, V232, V233, D234, E235, N236, G237, N238, K239, K240, S241, P242, N243, G244, S245, M246, P247, I248, V249, T250, K251, F252, E253, T254, D255, D256, L257, 1258, S259, D260, N261, Q262, K264, A265, M266, 1267, S268, N269, F270, T271, K272, N273, A274, A275, A276, K277, A278, A279, K280, K281, P282, I283, P284, Y285, L286, D287, 288, L289, K290, E291, M293, V294, S295, L296, C297, D298, Y300, N301, V302, Y303, A304, W305, A306, A307, A308, 1309, T310, N311, S312, N313, A314, D315, V316, T317, A318, N320, T321, L324, T325, F326, 1327, G328, E329, Q330, N331, S332, K335, E336, L337, S338, V339, L340, Q341, T342, T343, T344, N345, E346, K347, A348, K349, D350, 1351, L352, N353, K354, N356, D357, N358, L359, 1360, Q361, E362, V363, Y365, T366, P367, A368, K370, H371, L372, G373, D375, L376, A377, N378, L379, F380, D381, T382, L383, K384, E385, K386, D387, 1388, N389, N390, 1391, E392, N393, E394, E395, E396, K397, Q398, N399, V400, 1401, N402, D403, C404, 1405, E406, Q407, Y408, V409, D410, D411, C412, L415, N416, N418, P419, 1420, A421, A422, L423, L424, K425, H426, 1427, S428, Y430, Y431, E432, D433, F434, S435, A436, K437, N438, F439, L440, D441, G442, A443, K444, L445, N446, V447, L448, T449, E450, V451, V452, N453, Q455, K456, A457, H458, P459, T460, 1461, W462, S463, E464, 1800, S801, L802, K803, M804, 1805, S806, D807, F808, K809, G810, V811, V812, Q813, S814, Y815, F816, S817, V818, S819, G820, C821, V822, D823, D824, A825, S826, K827, K828, A829, H830, D831, S832, M833, L834, F835, T836, F837, M838, C839, A840, A841, E842, E843, K844, T846, N847, K848, E850, E851, K852, T853, N854, A856, A857, S858, F859, 1860, L861, Q862, K863, A864, Y865, L866, H867, G868, C869, K870, M871, 1872, V873, C874, E875, D876, D877, L878, P879, V880, A881, D882, G883, K884, T885, G886, K887, A888, Q889, N890, A891, D892, M894, D895, W896, C897, A898, A900, L901, A902, K903, K904, V905, N906, D907, G908, C909, V910, A911, M912, S913, 1914, C915, Y916, A918, P920, A921, Y922, M923, S924, S925, H926, Q927, D928, P929, F930, V931, H932, M933, Q934, D935, K936, K937, T938, S939, V940, L941, P943, F945, M946, E947, V948, N949, K950, D951, S952, 1953, D955, Y956, H957, V958, A959, G960, L961, L965, N966, S967, K968, S969, D970, A971, G972, T973, S974, V975, Y976, Y977, Q979, A980, A981, L982, H983, F984, C985, E986, A987, L988, G989, V990, S991, P992, E993, L994, V995, K996, N997, K998, K999, T1000, H1001, A1002, A1003, E1004, L1005, G1006, M1009, G1010, S1011, A1012, M1013, L1014, M1015, P1016, W1017, G1019, G1020, V1022, Y1023, I1024, A1025, S1026, K1027, K1028, L1029, T1030, S1031, D1032, A1033, K1034, S1035, V1036, K1037, Y1038, C1039, G1040, E1041, D1042, M1043, W1044, Q1045, Y1046, H1047, A1048, D1049, E1050, I1051, A1052, A1053, V1054, N1055, I1056, A1057, M1058, Y1059, E1060, V1061, C1062, C1063, Q1064, T1065, G1066, A1067, F1068, G1069, K1070, K1071, Q1072, K1073, K1074, S1075, D1076, E1077, L1078, P1079, and G1080.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1: S118, D119, F121, W123, Q136, E138, E143, V146, S155, V158, E161, S162, T163, A165, N166, G178, D180, T185, K189, A193, D196, N199, N200, E202, L203, S221, V233, E235, N236, S241, N243, S245, K251, D255, L257, N273, D287, S295, V302, S332, E336, S338, V339, E362, D375, A377, N378, D381, T382, E385, D387, N390, E395, E396, Q398, N399, V400, D403, E406, Q407, V409, D411, C412, N416, N418, L440, L448, V451, Q455, E464, S806, S817, V818, S819, S832, M833, F835, T836, F837, C839, A840, E842, E843, K844, T846, N847, K848, N854, A856, S858, Q862, K863, Y865, L866, G868, K870, M871, D876, D877, V880, G883, K884, G886, K887, A888, A891, D892, M894, A900, K903, K904, N906, V910, M912, S913, C915, Y916, A918, M923, S925, H926, Q927, V931, M933, Q934, D935, K936, K937, T938, S939, V940, F945, M946, V948, N949, K950, D951, S952, D955, Y956, A959, G960, N966, S967, K968, S969, D970, A971, G972, S974, V975, Y976, Q979, A980, L982, H983, C985, E986, A987, G989, V990, S991, P992, E993, L994, V995, K996, N997, K998, K999, T1000, H1001, A1002, A1003, E1004, G1006, G1010, A1012, M1013, L1014, W1017, V1022, K1028, D1032, K1034, K1037, C1039, G1040, Q1045, H1047, C1063, and G1069.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1: N243, E336, V880, G883, D892, and M923.

In some embodiment, the one or more mutation is a substitution with R.

In some embodiment, the Cas12i polypeptide further comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1: V880, G883, D892, and M923.

In some embodiment, the one or more mutation is a substitution with R.

In some embodiment, the Cas12i polypeptide comprises one or more mutations (such as, insertions, deletions, or substitutions) at one or more amino acids corresponding to one or more amino acids at one or more of the following positions of the amino acid sequence of the reference Cas12i polypeptide of SEQ ID NO: 1:
K109, L112, D125, 127, F144, L147, A148, L151, L157, V195, Y226, F252, 1258, M293, W305, A308, 1309, S312, A314, D315, V316, A318, L324, 1327, A348, L352, Y365, L372, L376, L379, L383, 1405, L424, 1427, A436, F439, A443, V447, A457, H458, P459, T460, S463, S814, F859, A864, H867, Y977, S1031, A1053, and F1068.

In some embodiment, the one or more mutation is a substitution with R:

In some embodiment, the substitution at N243 is a substitution with R, A, V, L, I, M, F, W, S, T, C, Y, N, Q, E, K, or H.

In some embodiment, the mutation is a substitution.

In some embodiment, the substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)).

In some embodiment, the substitution is a substitution with a positively charged amino acid residue, such as, Arginine (R).

In some embodiment, the substitution is a substitution with a non-polar amino acid residue, such as, Alanine (A).

In some embodiment, the Cas12i polypeptide comprises a substitution corresponding to any one of the mutants in Table 6, or a combination thereof, and wherein the amino acid location is relative to SEQ ID NO: 1.

In some embodiment, the Cas12i polypeptide comprises a substitution corresponding to any one of the mutants in Table 6 with increased spacer sequence-specific dsDNA cleavage activity compared to that of the reference Cas12i polypeptide of SEQ ID NO: 1 when both used in combination with a same guide RNA, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more, or a combination thereof, and wherein the amino acid location is relative to SEQ ID NO: 1.

In some embodiment, the Cas12i polypeptide is xCas12i-N243R mutant (SEQ ID NO: 458).

In some embodiment, the Cas12i polypeptide comprises a substitution corresponding to any one of the mutants in Table 8, or a combination thereof, and wherein the amino acid location is relative to SEQ ID NO: 1.

In some embodiment, the Cas12i polypeptide is xCas12i-N243R+E336R+D892R mutant (SEQ ID NO: 459) (SEQ ID NO: 459).

In some embodiment, the Cas12i polypeptide is xCas12i-N243R+E336R+G883R mutant (SEQ ID NO: 460).

In some aspect, the disclosure provides a Cas12i polypeptide:
(1) as set forth in the amino acid sequence of xCas12i-N243R mutant (SEQ ID NO: 458);
(2) comprising the amino acid sequence of xCas12i-N243R mutant (SEQ ID NO: 458); or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of xCas12i-N243R mutant (SEQ ID NO: 458).

In some aspect, the disclosure provides a Cas12i polypeptide:
(1) as set forth in the amino acid sequence of xCas12i-N243R+E336R+D892R mutant (SEQ ID NO: 459);
(2) comprising the amino acid sequence of xCas12i-N243R+E336R+D892R mutant (SEQ ID NO: 459); or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of xCas12i-N243R+E336R+D892R mutant (SEQ ID NO: 459).

In some aspect, the disclosure provides a Cas12i polypeptide:
(1) as set forth in the amino acid sequence of xCas12i-N243R+E336R+G883R mutant (SEQ ID NO: 460);
(2) comprising the amino acid sequence of xCas12i-N243R+E336R+G883R mutant (SEQ ID NO: 460); or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of xCas12i-N243R+E336R+G883R mutant (SEQ ID NO: 460).

In some embodiment, the Cas12i polypeptide is capable of recognizing a target adjacent motif (TAM) immediately 5′ to the protospacer sequence on the non-target strand of a target dsDNA, and wherein the TAM is 5′-NTTN-3′, wherein N is A, T, G, or C.

In some embodiment, the Cas12i polypeptide further comprises a functional domain associated with the Cas12i polypeptide.

In some embodiment, the functional domain has transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

In some aspect, the disclosure provides a fusion protein comprising the Cas12i polypeptide of the disclosure and a functional domain.

In some embodiments, the functional domain is fused N-terminally, C-terminally, or internally with respect to the Cas12i polypeptide.

In some embodiments, the functional domain is fused to the Cas12i polypeptide via a linker, e.g., a XTEN linker (SEQ ID NO: 442), a GS linker containing multiple glycine and serine residues, a GS linker containing multiple glycine and serine residues and a XTEN linker (SEQ ID NO: 442), a GS linker containing multiple glycine and serine residues and a BP NLS (SEQ ID NO: 443).

In some embodiments, the functional domain is selected from the group consisting of a nuclear localization signal (NLS), a nuclear export signal (NES), a deaminase or a catalytic domain thereof, an uracil glycosylase inhibitor (UGI), an uracil glycosylase (UNG), a methylpurine glycosylase (MPG), a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease or a catalytic domain thereof, a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment thereof, and any combination thereof.

In some embodiments, the NLS comprises or is SV40 NLS (SEQ ID NO: 444), bpSV40 NLS (BP NLS, bpNLS, SEQ ID NO: 443), or NP NLS (Xenopus laevis Nucleoplasmin NLS, nucleoplasmin NLS, SEQ ID NO: 445).

In some embodiments, the functional domain comprises a deaminase or a catalytic domain thereof.

In some embodiments, the deaminase or catalytic domain thereof is an adenine deaminase (e.g., TadA, such as, TadA8e, TadA8.17, TadA8.20, TadA9) or a catalytic domain thereof.

In some embodiments, the deaminase or catalytic domain thereof is a cytidine deaminase (e.g., APOBEC, such as, APOBEC3, for example, APOBEC3A, APOBEC3B, APOBEC3C; DddA) or a catalytic domain thereof.

In some embodiments, the functional domain comprises an uracil glycosylase inhibitor (UGI).

In some embodiments, the functional domain comprises an uracil glycosylase (UNG).

In some embodiments, the functional domain comprises a methylpurine glycosylase (MPG).

In some embodiments, the adenine deaminase domain is a wild type TadA or a variant thereof
(1) as set forth in SEQ ID NO: 439;
(2) comprising the amino acid sequence of SEQ ID NO: 439; or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 439.

In some embodiments, the adenine deaminase domain is TadA8e-V106W of SEQ ID NO: 439 or TadA8e.

In some embodiments, the UGI domain
(1) is as set forth in SEQ ID NO: 441;
(2) comprises the amino acid sequence of SEQ ID NO: 441; or
(3) comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 441.

In some embodiments, the cytidine deaminase domain is an APOBEC3 or a variant thereof
(1) as set forth in SEQ ID NO: 440;
(2) comprising the amino acid sequence of SEQ ID NO: 440; or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 440.

In some embodiments, the cytidine deaminase domain is human APOBEC3-W104A of SEQ ID NO: 440.

In some embodiments, the functional domain comprises a reverse transcriptase or a catalytic domain thereof.

In some embodiments, the functional domain comprises a methylase or a catalytic domain thereof.

In some embodiments, the functional domain comprises a transcription activating domain,

In some embodiments, the functional domain comprises an exonuclease or a catalytic domain thereof, such as, T5 exonuclease (T5E) (SEQ ID NO: 449).

In some embodiments, the exonuclease is N-terminally or C-terminally fused to the Cas12i polypeptide.

In some embodiments, the exonuclease is C-terminally fused to the Cas12i polypeptide.

In some embodiments, the T5 exonuclease
(1) is as set forth in SEQ ID NO: 449;
(2) comprises the amino acid sequence of SEQ ID NO: 449; or
(3) comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 449.

In some aspect, the disclosure provides a fusion protein comprising:
(1) an Cas12i polypeptide; and
(2) an adenine deaminase domain.

In some embodiments, the adenine deaminase domain is an adenine deaminase (e.g., TadA, such as, TadA8e, TadA8.17, TadA8.20, TadA9) or a catalytic domain thereof.

In some embodiments, the adenine deaminase domain is a wild type TadA or a variant thereof
(1) as set forth in SEQ ID NO: 439;
(2) comprising the amino acid sequence of SEQ ID NO: 439; or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 439.

In some embodiments, the adenine deaminase domain is TadA8e-V106W of SEQ ID NO: 439 or TadA8e.

In some aspect, the disclosure provides a fusion protein comprising:
(1) an Cas12i polypeptide; and
(2) a cytidine deaminase domain.

In some embodiments, the fusion protein further comprises an uracil glycosylase inhibitor (UGI) domain.

In some embodiments, the UGI domain
(1) is as set forth in SEQ ID NO: 441;
(2) comprises the amino acid sequence of SEQ ID NO: 441; or
(3) comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 441.

In some embodiments, the cytidine deaminase domain is a cytidine deaminase (e.g., APOBEC (apolipoprotein B mRNA-editing catalytic polypeptide-like), such as, APOBEC3, for example, APOBEC3A, APOBEC3B, APOBEC3C; DddA) or a catalytic domain thereof.

In some embodiments, the cytidine deaminase domain is an APOBEC3 or a variant thereof
(1) as set forth in SEQ ID NO: 440;
(2) comprising the amino acid sequence of SEQ ID NO: 440; or
(3) comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 440.

In some embodiments, the cytidine deaminase domain is human APOBEC3-W104A of SEQ ID NO: 440.

In some embodiments, the fusion protein comprises the amino acid sequence of SEQ ID NO: 85 or 184.

In some aspect, the disclosure provides a fusion protein comprising:
(1) an Cas12i polypeptide; and
(2) a non-LTR retrotransposon domain.

In some aspect, the disclosure provides a fusion protein comprising:
(1) an Cas12i polypeptide; and
(2) a transcription activating domain.

In some embodiments, the Cas12i polypeptide is the Cas12i polypeptide of the disclosure.

In some embodiments, the adenine deaminase domain is N-terminally or C-terminally fused to the Cas12i polypeptide.

In some embodiments, the cytidine deaminase domain is N-terminally or C-terminally fused to the Cas12i polypeptide.

In some embodiments, the uracil glycosylase inhibitor domain is N-terminally or C-terminally fused to the Cas12i polypeptide.

In some embodiments, the uracil glycosylase inhibitor domain is N-terminally or C-terminally fused to the cytidine deaminase domain.

In some embodiments, the non-LTR retrotransposon domain is N-terminally or C-terminally fused to the Cas12i polypeptide.

In some embodiments, the fusion protein comprises one, two, three, or more UGI domain.

In some embodiments, the fusion protein comprises one, two, three, or more UGI domain in tandem via a linker or not.

In some embodiments, the fusion protein comprises one, two, three, four, or more NLS and/or NES.

In some embodiments, the fusion protein comprises a NLS or a NES at the N-terminus and/or C-terminus of the Cas12i polypeptide.

In some embodiments, the fusion protein comprises a NLS or a NES at the N-terminus and/or C-terminus of the adenine deaminase domain.

In some embodiments, the fusion protein comprises a NLS or a NES at the N-terminus and/or C-terminus of the cytidine deaminase domain.

In some embodiments, the fusion protein comprises a NLS or a NES at the N-terminus and/or C-terminus of the UGI domain.

In some embodiments, the fusion protein comprises a NLS or a NES at the N-terminus and/or C-terminus of the reverse transcriptase domain.

In some embodiments, the fusion protein comprises a NLS or a NES at the N-terminus and/or C-terminus of the non-LTR retrotransposon domain.

In some embodiments, the fusion is via a linker.

In some embodiments, the linker is a GS linker, a XTEN linker (SEQ ID NO: 442), a XTEN-containing linker, a NLS or NES-containing linker, a XTEN-containing GS linker, a NLS or NES-containing GS linker.

In some embodiments, the fusion protein comprises an inducible element, e.g., an inducible polypeptide.

In some embodiments, the NLS comprises or is SV40 NLS (SEQ ID NO: 444), bpSV40 NLS (BP NLS, bpNLS, SEQ ID NO: 443), or NP NLS (Xenopus laevis Nucleoplasmin NLS, nucleoplasmin NLS, SEQ ID NO: 445).

In some aspect, the disclosure provides a vector, wherein the vector is an AAV vector genome comprising:
(1) a polynucleotide encoding a fusion protein comprising of the disclosure operably linked to a promoter; and
(2) a polynucleotide encoding a guide RNA operably linked to a promoter, the guide RNA comprising:
(i) a direct repeat sequence capable of forming a complex with the Cas12i polypeptide or the fusion protein; and
(ii) a spacer sequence capable of hybridizing to a target sequence on a target strand of a target dsDNA, thereby guiding the complex to the target dsDNA.

In some embodiments, the fusion protein has increased efficiency (e.g., base editing efficiency, methylation efficiency, transcription activating efficiency) compared to that of an otherwise identical control fusion protein or control conjugate or control fusion protein comprising the reference polypeptide of any one of SEQ ID NOs: 1-3, 6, and 10, e.g., an increase in efficiency by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some aspect, the disclosure provides a guide RNA comprising:
(1) a direct repeat sequence capable of forming a complex with an Cas12i polypeptide or a fusion protein comprising the Cas12i polypeptide and a functional domain; and
(2) a spacer sequence capable of hybridizing to a target sequence on a target strand of a target dsDNA, thereby guiding the complex to the target dsDNA.

In some embodiments, the direct repeat sequence is 5′ to the spacer sequence.

In some embodiments, the guide RNA further comprises an aptamer.

In some embodiments, the guide RNA further comprises an extension to add an RNA template.

In some embodiments, the guide RNA further comprises a donor sequence for insertion into the target dsDNA.

In some embodiments, the direct repeat sequence:
(1) is as set forth in any one of SEQ ID NOs: 11-13, 16, 20, and 451-457;
(2) comprises the polynucleotide sequence of any one of SEQ ID NOs: 11-13, 16, 20, and 451-457; or
(3) comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of any one of SEQ ID NOs: 11-13, 16, 20, and 451-457.

In some embodiments, the direct repeat sequence is a direct repeat sequence comprising a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%) and less than 100% to the polynucleotide sequence of any one of SEQ ID NOs: 11-13, 16, 20, and 451-457.

In some embodiments, the direct repeat sequence has substantially the same secondary structure as the secondary structure of any one of SEQ ID NOs: 11-13, 16, 20, and 451-457.

In some embodiments, the direct repeat sequence is not any one of SEQ ID NOs: 11-13, 16, and 20.

In some embodiments, when the guide RNA is used in combination with an Cas12i polypeptide (e.g., the Cas12i polypeptide of the disclosure), an increased spacer sequence-specific dsDNA and/or ssDNA cleavage activity is exhibited compared with that of an otherwise identical control guide RNA comprising any one of SEQ ID NOS: 11-13, 16, 20, and 451-457 used in combination with the Cas12i polypeptide, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiments, when the guide RNA is used in combination with an Cas12i polypeptide (e.g., the Cas12i polypeptide of the disclosure), an decreased spacer sequence-specific dsDNA and/or ssDNA cleavage activity is exhibited compared with that of an otherwise identical control guide RNA comprising any one of SEQ ID NOs: 11-13, 16, 20, and 451-457 used in combination with the Cas12i polypeptide, e.g., an decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, when the guide RNA is used in combination with a fusion protein comprising an Cas12i polypeptide (e.g., the Cas12i polypeptide of the disclosure) and a functional domain (e.g., a functional domain of the disclosure) (e.g., a fusion protein of the disclosure), an increased efficiency (e.g., base editing efficiency, methylation efficiency, transcription activating efficiency) is exhibited compared to that of an otherwise identical control guide RNA comprising any one of SEQ ID NOs: 11-13, 16, 20, and 451-457 used in combination with the fusion protein, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiments, the direct repeat sequence comprises one or more mutations (such as, insertions, deletions, or substitutions) at one or more nucleotides corresponding to one or more nucleotides of the polynucleotide sequence of any one of SEQ ID NOs: 11-13, 16, 20, and 451-457.

In some embodiments, the one or more mutations are within a stem-loop region corresponding to the stem-loop region (e.g., R1 region, R2 region, R3 region, R4 region) of the polynucleotide sequence of any one of SEQ ID NOs: 11-13, 16, 20, and 451-457.

In some embodiments, the direct repeat sequence comprises one or more mutations (such as, insertions, deletions, or substitutions) at one or more nucleotides corresponding to one or more nucleotides at one or more of the following positions of the polynucleotide sequence of any one of SEQ ID NOs: 11-13, 16, 20, and 451-457: any one of positions 1 to the end of any one of SEQ ID NOs: 11-13, 16, 20, and 451-457, e.g., 36, such as, position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36.

In some embodiments, the direct repeat sequence comprises one or more mutations (such as, insertions, deletions, or substitutions) at one or more nucleotides corresponding to one or more nucleotides at one or more of the following positions of the polynucleotide sequence of SEQ ID NO: 11:
any one of positions 1 to 36, such as, position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36.

In some embodiments, the mutation is a deletion.

In some embodiments, the mutation is a substitution.

In some embodiments, the mutation is a substitution with A, U, G, or C.

In some embodiments, the direct repeat sequence comprises a deletion.

In some embodiments, the deletion is within a stem-loop region (e.g., R1 region, R2 region, R3 region, R4 region, R5 region) of the direct repeat sequence.

In some embodiments, the deletion comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

In some embodiments, the stem-loop region comprising the deletion retains at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs.

In some embodiments, the stem-loop region comprising the deletion retains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs.

In some embodiments, the stem-loop region comprising the deletion contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 non-A-U or non-G-C mismatches.

In some embodiments, the direct repeat sequence comprises a substitution of one or more thermodynamically unstable base pairs with one or more G-C or C-G base pairs.

In some embodiments, the thermodynamically unstable base pair is a A-U or U-A base pair, a A-G or G-A base pair, or a U-G or G-U base pair.

In some embodiments, the thermodynamically unstable base pair is within the stem of a stem-loop region of the direct repeat sequence.

In some embodiments, the thermodynamically unstable base pair is the 1st, 2nd, 3rd, 4th, 5th, 6th, 7th, 8th, 9th, 10th, 11th, 12th, 13th, 14th, 15th, 16th, 17th, 18th, 19th, 20th, 21th, 22th, 23th, 24th, 25th, 26th, 27th, 28th, 29th, or 30th base pair starting from and including the base pair shared by both the stem and the loop of the stem-loop region.

In some embodiments, the direct repeat sequence
(1) is as set forth in any one of SEQ ID NOs: 451-457;
(2) comprises the polynucleotide sequence of any one of SEQ ID NOs: 451-457; or
(3) comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of any one of SEQ ID NOs: 451-457.

In some embodiments, the target sequence comprises, consists essentially of, or consists of at least about 16 contiguous nucleotides of a target gene, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous nucleotides of a target gene, or in a numerical range between any of two preceding values, e.g., from about 16 to about 50 contiguous nucleotides of a target gene.

In some embodiments, the target sequence is at least about 16 nucleotides in length, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length, or in a length of a numerical range between any of two preceding values, e.g., in a length of from about 16 to about 50 nucleotides.

In some embodiments, the protospacer sequence comprises, consists essentially of, or consists of at least about 16 contiguous nucleotides of a target gene, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 contiguous nucleotides of a target gene, or in a numerical range between any of two preceding values, e.g., from about 16 to about 50 contiguous nucleotides of a target gene.

In some embodiments, the protospacer sequence is at least about 16 nucleotides in length, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length, or in a length of a numerical range between any of two preceding values, e.g., in a length of from about 16 to about 50 nucleotides.

In some embodiments, the target sequence comprises a protospacer adjacent motif (PAM) sequence 5′ to the target sequence.

In some embodiments, the target sequence comprises a protospacer adjacent motif (PAM) sequence 5′ to the protospacer sequence reverse complementary to the target sequence.

In some embodiments, the spacer sequence is at least about 16 nucleotides in length, e.g., about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides in length, or in a length of a numerical range between any of two preceding values, e.g., in a length of from about 16 to about 50 nucleotides.

In some embodiments, the spacer sequence is about 90 to 100% complementary to the target sequence, and/or contains no more than 1, 2, 3, 4, or 5 mismatches to the target sequence.

In some embodiments, the guide RNA comprises a plurality (e.g., 2, 3, 4, 5 or more) of spacer sequences capable of hybridizing to a plurality of target sequences, respectively.

In some embodiments, the plurality of target sequences are on a same polynucleotide, or on separate polynucleotides.

In some embodiments, the spacer sequence comprises at least 16 contiguous nucleotides of any one of SEQ ID NOs: 82-125, 130, 131-381, 382, 391, 398-438.

In some embodiments, the dsDNA is within a cell.

In some aspect, the disclosure provides a polynucleotide encoding the Cas12i polypeptide or the fusion protein of the disclosure.

In some aspect, the disclosure provides a polynucleotide encoding the guide RNA of the disclosure.

In some embodiments, the polynucleotide is codon optimized for expression in eukaryotic (e.g., mammalian, such as, human) cells.

In some embodiments, the polynucleotide is a polydeoxyribonucleotide or a polyribonucleotide.

In some embodiments, one or more of the nucleotides of the polynucleotide is modified.

In some aspect, the disclosure provides a system or composition comprising:
(1) an Cas12i polypeptide or a fusion protein comprising the Cas12i polypeptide and a functional domain, or a polynucleotide encoding the Cas12i polypeptide or the fusion protein; and
(2) a guide RNA (also referred to as “CRISPR RNA” or “crRNA”) or a polynucleotide encoding the guide RNA, the guide RNA comprising:
(i) a direct repeat sequence capable of forming a complex with the Cas12i polypeptide or the fusion protein; and
(ii) a spacer sequence capable of hybridizing to a target sequence on a target strand of a target dsDNA, thereby guiding the complex to the target dsDNA.

In some embodiments, the system or composition is a non-naturally occurring, engineered system or composition.

In some embodiments, the Cas12i polypeptide or the fusion protein is the Cas12i polypeptide or the fusion protein of the disclosure.

In some embodiments, the guide RNA is the guide RNA of the disclosure.

In some embodiments, the direct repeat sequence is the direct repeat sequence of the disclosure.

In some embodiments, the spacer sequence is the spacer sequence of the disclosure.

In some embodiments, the system or composition further comprises an inducible system, such as, TMP, DOX, Degron.

In some embodiments, the inducible system comprises an inducing agent capable of activating the fusion protein comprising an inducible element.

In some embodiments, the inducible system comprises an inducing agent capable of activating the expression of the Cas12i polypeptide or the fusion protein comprising an inducible element.

In some embodiments, the system or composition comprises an activator capable of activating the fusion protein comprising a transcription activating domain.

In some embodiments, the coding sequence is a DNA coding sequence or an RNA coding sequence.

In some embodiments, the system or composition further comprises a serine or tyrosine recombinase.

In some embodiments, the system or composition further comprises a donor construct comprising a donor polynucleotide for insertion into the target dsDNA and located between two binding elements capable of forming a complex with the non-LTR retrotransposon protein.

In some embodiments, the Cas12i polypeptide is fused to the N-terminus of the non-LTR retrotransposon protein.

In some embodiments, the Cas12i polypeptide is a nickase.

In some embodiments, the guide RNA guides the fusion protein to a target sequence 5′ of the targeted insertion site, and wherein the Cas12i polypeptide generates a double-strand break at the targeted insertion site.

In some embodiments, the guide RNA guides the fusion protein to a target sequence 5′ or 3′ of the targeted insertion site, and wherein the Cas12i polypeptide generates a double-strand break at the targeted insertion site.

In some embodiments, the donor polynucleotide further comprises a polymerase processing element to facilitate 5′ or 3′ end processing of the donor polynucleotide sequence.

In some embodiments, the donor polynucleotide further comprises a homology region to the target sequence on the 5′ end of the donor construct, the 3′ end of the donor construct, or both.

In some embodiments, the homology region is from 8 to 25 base pairs.

In some aspect, the disclosure provides a vector comprising the polynucleotide encoding the Cas12i polypeptide or the fusion protein of the disclosure.

In some embodiments, the polynucleotide is operably linked to a promoter.

In some aspect, the disclosure provides a vector comprising the polynucleotide encoding the guide RNA of the disclosure.

In some embodiments, the polynucleotide is operably linked to a promoter.

In some aspect, the disclosure provides a vector comprising the polynucleotide encoding the Cas12i polypeptide or the fusion protein of the disclosure and the polynucleotide encoding the guide RNA of the disclosure.

In some embodiments, the polynucleotide encoding the Cas12i polypeptide or the fusion protein of the disclosure and the polynucleotide encoding the guide RNA of the disclosure are operably linked to a same promoter.

In some embodiments, the polynucleotide encoding the Cas12i polypeptide or the fusion protein of the disclosure and the polynucleotide encoding the guide RNA of the disclosure are each operably linked to a promoter.

In some embodiments, the promoter is selected from the group consisting of a ubiquitous promoter, a tissue-specific promoter, a cell-type specific promoter, a constitutive promoter, and an inducible promoter.

In some embodiments, the promoter comprises or is a promoter selected from the group consisting of: a (human) U6 promoter (such as SEQ ID NO: 446), an elongation factor 1α short (EFS) promoter, a (human) Cbh promoter, a MHCK7 promoter, a Cba promoter, a pol I promoter, a pol II promoter, a pol III promoter, a T7 promoter, a H1 promoter, a retroviral Rous sarcoma virus LTR promoter, a (human) cytomegalovirus (CMV) promoter (such as SEQ ID NO: 447), a SV40 promoter, a dihydrofolate reductase promoter, a β-actin promoter, a β glucuronidase (GUSB) promoter, a cytomegalovirus (CMV) immediate-early (Ie) enhancer and/or promoter, a chicken β-actin (CBA) promoter or derivative thereof such as a CAG promoter (such as SEQ ID NO: 500), CB promoter, a (human) elongation factor 1α-subunit (EF1α) promoter, a ubiquitin C (UBC) promoter, a prion promoter, a neuron-specific enolase (NSE) promoter, a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding polypeptide 2 (MeCP2) promoter, a Ca2+/calmodulin-dependent polypeptide kinase II (CaMKII) promoter, a metabotropic glutamate receptor 2 (mGluR2) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic polypeptide (GFAP) promoter, and a myelin basic polypeptide (MBP) promoter.

In some embodiments, the polynucleotide encoding the Cas12i polypeptide or the fusion protein of the disclosure is 5′ or 3′ to the polynucleotide encoding the guide RNA of the disclosure.

In some embodiments, the vector is a plasmid.

In some embodiments, the vector is a viral vector.

In some embodiments, the vector is a retroviral vector, a phage vector, an adenoviral vector, a herpes simplex viral (HSV) vector, an AAV vector, or a lentiviral vector.

In some embodiments, the AAV vector is a DNA-encapsidated AAV vector or a RNA-encapsidated AAV vector.

In some embodiments, the AAV vector comprises a capsid with a serotype of AAV1, AAV2, AAV3, AAV3A, AAV3B, AAV4, AAV5, AAV6, AAV7, AAVrh74, AAV8, AAV9, AAV10, AAV11, AAV12, AAV13, AAV-DJ, AAV.PHP.eB, a member of the Clade to which any of the AAV1-AAV13 belong, a functional truncated variant thereof, or a functional mutant thereof.

In some aspect, the disclosure provides a recombinant AAV (rAAV) particle comprising the vector of the disclosure.

In some embodiments, the rAAV particle comprises a capsid with a serotype of AAV1, AAV2, AAV3, AAV3A, AAV3B, AAV4, AAV5, AAV6, AAV7, AAVrh74, AAV8, AAV9, AAV10, AAV11, AAV12, AAV13, AAV-DJ, AAV.PHP.eB, a member of the Clade to which any of the AAV1-AAV13 belong, a functional truncated variant thereof, or a functional mutant thereof, encapsidating the vector.

In some aspect, the disclosure provides a lipid nanoparticle (LNP) comprising the polynucleotide encoding the Cas12i polypeptide or the fusion protein of the disclosure and the guide RNA of the disclosure.

In some embodiments, the polynucleotide encoding the Cas12i polypeptide or the fusion protein of the disclosure is in form of a mRNA.

In some embodiments, the polynucleotide encoding the Cas12i polypeptide or the fusion protein comprise a 5′ UTR.

In some embodiments, the polynucleotide encoding the Cas12i polypeptide or the fusion protein comprise a 3′ polyA tail.

In some aspect, the disclosure provides a method for modifying a target dsDNA, comprising contacting the target dsDNA with the system, vector, rAAV particle, or LNP of the disclosure, wherein the spacer sequence is capable of hybridizing to a target sequence of a target strand of the target dsDNA, wherein the target sequence is modified by the complex.

In some aspect, the disclosure provides use of the system, vector, rAAV particle, or LNP of the disclosure in the manufacture of an agent for modifying a target dsDNA, wherein the spacer sequence is capable of hybridizing to a target sequence of a target strand of the target dsDNA, wherein the target sequence is modified by the complex.

In some aspect, the disclosure provides the system, vector, rAAV particle, or LNP of the disclosure, for use in modifying a target dsDNA, wherein the spacer sequence is capable of hybridizing to a target sequence of a target strand of the target dsDNA, wherein the target sequence is modified by the complex.

In some embodiments, the target dsDNA is human TRAC gene.

In some embodiments, the spacer sequence comprises at least 16 contiguous nucleotides of any one of SEQ ID NOs: 123-125.

In some aspect, the disclosure provides a cell or a progeny thereof comprising the Cas12i polypeptide, the fusion protein, the guide RNA, the system, the polynucleotide, the vector, the rAAV particle, and/or the LNP of the disclosure.

In some aspect, the disclosure provides a modified cell or a progeny thereof, wherein the modified cell is modified by the method of the disclosure.

In some embodiments, the cell is in vivo, ex vivo, or in vitro.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is a cultured cell, an isolated primary cell, or a cell within a living organism.

In some embodiments, the cell is a T cell (such as, CAR-T cell), B cell, NK cell (such as, CAR-NK cell), or stem cell (such as, iPS cell, HSC cell).

In some embodiments, the cell is derived from or heterogenous to the subject.

In some aspect, the disclosure provides a host comprising the cell or progeny thereof of the disclosure.

In some embodiments, the host is a non-human animal or a plant.

In some embodiments, the non-human animal is an animal (e.g., rodent or non-human primate) model for a human genetic disorder.

In some aspect, the disclosure provides a (e.g., pharmaceutical) composition comprising the Cas12i polypeptide, the fusion protein, the guide RNA, the polynucleotide, the system, the vector, the rAAV particle, the LNP, and/or the cell or progeny thereof of the disclosure.

In some embodiments, the composition comprises a pharmaceutically acceptable excipient.

In some embodiments, the composition is formulated for delivery by nanoparticles, e.g., lipid nanopaticles, liposomes, exosomes, microvesicles, nucleic acid (e.g., DNA) nanoassemblies, a gene gun, or an implantable device.

In some aspect, the disclosure provides a delivery system comprising:
(1) a delivery vehicle, and
(2) the Cas12i polypeptide, the fusion protein, the guide RNA, the polynucleotide, the system, the vector, the rAAV particle, the LNP, the cell or progeny thereof, and/or the composition of the disclosure.

In some embodiments, the delivery vehicle is a nanoparticle, e.g., a lipid nanopaticle, a liposome, an exosome, a microvesicle, a nucleic acid (e.g., DNA) nanoassembly, a gene-gun, or an implantable device.

In some aspect, the disclosure provides a kit comprising the Cas12i polypeptide, the fusion protein, the guide RNA, the polynucleotide, the system, the vector, the rAAV particle, the LNP, the cell or progeny thereof, the composition, and/or the delivery system of the disclosure.

In some embodiments, the kit further comprising an instruction for modifying a target dsDNA.

In some aspect, the disclosure provides a method for diagnosing, preventing, or treating a disease or disorder in a subject, comprising administering to the subject (e.g., an effective amount of) the system, the vector, the rAAV particle, the LNP, the cell or progeny thereof, the composition, the delivery system, and/or the kit of the disclosure.

In some aspect, the disclosure provides use of (e.g., an effective amount of) the system, the vector, the rAAV particle, the LNP, the cell or progeny thereof, the composition, the delivery system, and/or the kit of the disclosure in the manufacture of a medicament or kit for diagnosing, preventing, or treating a disease or disorder in a subject.

In some aspect, the disclosure provides (e.g., an effective amount of) the system, the vector, the rAAV particle, the LNP, the cell or progeny thereof, the composition, the delivery system, and/or the kit of the disclosure, for use in diagnosing, preventing, or treating a disease or disorder in a subject.

In some embodiments, the disease or disorder is associated with an aberration of a target dsDNA in the subject.

In some embodiments, the spacer sequence is capable of hybridizing to a target sequence of a target strand of the target dsDNA, wherein the aberration of the target dsDNA is modified by the complex.

In some embodiments, the method or use further comprises administering to the subject an effective amount of a homologous recombination donor template comprising a donor sequence for insertion into a target dsDNA, wherein the insertion of the donor sequence corrects the aberration of the target dsDNA.

In some embodiments, the disease or disorder is prevented or treated by the modified cell or progeny thereof.

In some embodiments, the disease or disorder is a TTR-associated disease or disorder, e.g., ATTR.

In some embodiments, the spacer sequence comprises at least 16 contiguous nucleotides of SEQ ID NO: 107. In some embodiments, the disease or disorder is a PCSK9-associated disease or disorder.

In some embodiments, the spacer sequence comprises at least 16 contiguous nucleotides of SEQ ID NO: 122.

In some embodiments, the system further comprises a homologous recombination donor template comprising a donor sequence for insertion into a target dsDNA.

In some embodiments, said guiding the complex to the target dsDNA results in binding of the complex to the target dsDNA.

In some embodiments, said guiding the complex to the target dsDNA results in a modification of the target dsDNA.

In some embodiments, the modification of the target dsDNA comprises a double strand break (DSB) of the target dsDNA.

In some embodiments, the DSB results in generation of a deletion and/or insertion mutation (Indel mutation).

In some embodiments, the Indel mutation modifies the transcription and/or expression of the target dsDNA.

In some embodiments, a donor DNA template is inserted at the site of the DSB.

In some embodiments, the modification of the target dsDNA comprises a single strand break (SSB) of the target sequence of the target strand of the target dsDNA.

In some embodiments, the modification of the dsDNA comprises a substitution of one or more nucleotides of the protospacer sequence reverse complementary to the target sequence.

In some embodiments, the substitution is an A-to-T substitution, an A-to-G substitution, an A-to-C substitution, a C-to-A substitution, a C-to-T substitution, a C-to-G substitution, a T-to-A substitution, a T-to-G substitution, a T-to-C substitution, a G-to-A substitution, a G-to-T substitution, and/or a G-to-C substitution.

In some embodiments, the modification of the dsDNA comprises a single strand break (SSB) of the non-target strand of the target dsDNA.

In some embodiments, the modification of the dsDNA comprises an insertion, a deletion, and/or a substitution of one or more nucleotides of the non-target strand.

In some embodiments, the modification: a. introduces one or more base edits; b. corrects or introduces a premature stop codon; c. disrupts a splice site; d. inserts or restores a splice site; e. inserts a gene or gene fragment at one or both alleles of the target polynucleotide; or f. a combination thereof.

In some embodiments, the complex directs the reverse transcriptase domain to the target sequence, and the reverse transcriptase facilitates insertion of the donor sequence from the guide RNA into the target dsDNA.

In some embodiments, the insertion of the donor sequence: a. introduces one or more base edits; b. corrects or introduces a premature stop codon; c. disrupts a splice site; d. inserts or restores a splice site; e. inserts a gene or gene fragment at one or both alleles of the target polynucleotide; or, f. a combination thereof.

In some embodiments, the complex directs the non-LTR retrotransposon protein to the target sequence, and the non-LTR retrotransposon protein facilitates insertion of the donor polynucleotide sequence from the donor construct into the target dsDNA.

In some embodiments, the insertion of the donor sequence: a. introduces one or more base edits; b. corrects or introduces a premature stop codon; c. disrupts a splice site; d. inserts or restores a splice site; e. inserts a gene or gene fragment at one or both alleles of the target polynucleotide; or f. a combination thereof.

In some embodiments, said guiding the complex to the target dsDNA results in a modification of the transcription of the target dsDNA.

In some embodiments, the modification of the transcription is upregulated transcription, downregulated transcription, activated transcription, or inhibited transcription.

In some embodiments, the modification of the target dsDNA comprises methylation or demethylation of one or more nucleotides of the target dsDNA.

These and other aspects, objects, features, and advantages of the example embodiments will become apparent to those having ordinary skill in the art upon consideration of the following detailed description of illustrated example embodiments.

It should be understood that any one embodiment of the disclosure described herein, including those described only in the examples or claims, or only in one aspects/sections below, can be combined with any other one or more embodiments of the disclosure, unless explicitly disclaimed or improper.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 shows that hfCas12Max, an engineered variant of xCas12i, mediated high-efficient and -specificity genome editing, and dCas12i base editor exhibited high base editing activity in mammalian cells. A, xCas12i mediated EGFP activation efficiency determined by flow cytometry. NC represents non-specific (non-targeting) control. B, Schematics of protein engineering strategy for mutants with high efficiency and high fidelity using an activatable EGFP reporter screening system with on-targeted and off-targeted crRNA. C-D, Cas12Max exhibited significantly increased cleavage activity than xCas12i at reporter plasmids (C) or various genomic (D) target sites. Each dot represents the mean indel frequency at one targeted site (n=3). E, NGS analysis showed that hfCas12Max retained comparable activity at TTR.2-ON targets and almost no at 6 OT sites, to Cas12Max. F, Both Cas12Max and hfCas12Max exhibited a broader PAM recognition profile than other Cas proteins, including 5′-TN and 5′-TNN PAM. G, Comparison of indel activity from Cas12Max, hfCas12Max, LbCas12a, Ultra AsCas12a, SpCas9 and KKH-saCas9 at TTR locus. hfCas12Max retained the comparable activity of Cas12Max, and higher gene-editing efficiency than other Cas proteins. Each dot represents one of three repeats of single target site. H, Schematics of different versions of dxCas12i adenine base editors. I, Comparison of A-to-G editing frequency and product purity at the KLF4 site from TadA8e.1-dxCas12i-v1.2, v2.2 and v4.3, v4.3 showed a high editing activity of 80%. TadA8e-dxCas12i-v4.3, named as ABE-dCas12Max. TadA8e.1 represents TadA8e V106W. J, Schematics of different versions of dxCas12i cytosine base editors. K, Comparison of C-to-T editing frequency and product purity at the DYRK1A site from hA3A.1-dxCas12i,-v1.2 v2.2 and v3.1, v3.1 showed a high editing activity of 50%. hA3A.1-dxCas12i-v3.1, named as CBE-dCas12Max. hA3A.1 represents human APOBEC3A W104A.

FIG. 2 shows that hfCas12Max mediates high-efficiency gene editing ex vivo and in vivo. A, Schematics of hfCas12Max gene editing in primary human cells. B, Viability and indel activity of human CD3+ T cells following delivery of hfCas12Max RNPs with three different TRAC targeted crRNAs at 1.6 μM and 3.2 μM respectively (n=2 or 3). NC represents blank control, untreated with RNP. C, Representative flow cytometric analysis of edited CD3+ T cell 5 days after RNP delivery. NC represents blank control, untreated with RNP. D, Schematics of in vivo non-liposome delivery containing IVT-mRNA, LNP packaging process. E, Editing efficiency of LNP packaging with hfCas12Max mRNA and targeted Ttr crRNA at increased concentrations in N2a cells (n=8). F, Schematics of Ttr locus. G, Indel rates of LNP packaging with hfCas12Max mRNA and targeted Ttr crRNA at three dose (0.1, 0.3 and 0.5 mpk) in C57 mouse (n=6). H, The A to G editing percentage of LNP packaging with dCas12i-ABE mRNA and targeted Ttr crRNA at 3 mpk in C57 mouse (n=2).

FIG. 3 shows screen for functional Cas12i in HEK293T cells. A, Transfection of plasmids coding Cas12i and crRNA mediate EGFP activation. B, Five of ten Cas12i nuclease mediated EGFP-activated efficiency in HEK293T cells.

FIG. 4 shows identification and characterization of type V-I systems. A, Nuclease domain organization of SpCas9, LbCas12a, and xCas12i. B, Effective spacer sequence length for xCas12i. C, PAM scope comparison of LbCas12a, and xCas12i. xCas12i exhibited a higher dsDNA cleavage activity at 5′-TTN PAM than Cas12a. D, Flow diagram for detection of genome cleavage activity by transfection of an all-in-one plasmid containing xCas12i and targeted gRNA into HEK293T cells, followed by FACS and NGS analysis. E-F, xCas12i mediated robust genome cleavage (up to 90%) at the Ttr locus in N2a cells and TTR and PCSK9 in HEK293T cells.

FIG. 5 shows screen for engineered xCas12i mutants with increased dsDNA cleavage activity. A, The relative dsDNA cleavage activity of over 500 rationally engineered xCas12i mutants. v1.1 represents xCas12i with N243R, named as Cas12Max.

FIG. 6 shows other mutants mediated high-efficiency editing. A, Of the saturated mutants of N243, N243R increased the EGFP-activated fluorescent most. B-C, xCas12i mutant with N243R increased 1.2, 5, 20-fold activity at DMD.1, DMD.2 and DMD.3 locus. D, Both Cas12Max (xCas12i-N243R) and Cas12Max-E336R elevated EGFP-activated fluorescent at different PAM recognition sites.

FIG. 7 shows that Cas12Max induced off-target dsDNA cleavage activity at sites with mismatches using the reporter system (A) and targeted deep sequence (B).

FIG. 8 shows that hfCas12Max mediates high-efficiency and -specificity editing. A, Rational protein engineering screen of over 200 mutants for highly-fidelity Cas12Max. Four mutants show significantly decreased activity at both OT (off-target) sites and retains at ON.1 (on-target) site. B, Different versions of xCas12i mutants. C, v6.3 reduced off-target at OT.1, OT.2 and OT.3 sites and retained indel activity at TTR-ON targets, compared to v1.1-Cas12Max. D, v6.3 exhibited comparable indel activity at DMD.1, DMD.2, and higher at DMD.3 locus, than v1.1-Cas12Max. v1.1, named as Cas12Max. v6.3, named as hfCas12Max.

FIG. 9 shows comparison of the gene-editing efficiency of hfCas12Max with LbCas12a, Ultra AsCas12a, SpCas9 and KKH-saCas9 at TTR locus.

FIG. 10 shows that hfCas12Max mediated the high-efficient and -specific editing. A-B, Off-target efficiency of hfCas12Max, LbCas12a, and UltraAsCas12a at in-silico predicted off-target sites, determined by targeted deep sequencing. Sequences of on-target and predicted off-target sites are shown, PAM sequences are in blue and mismatched bases are in red.

FIG. 11 shows conserved cleavage sites of Cas12i. A, Sequence alignment of xCas12i, Cas12i1 and Cas12i2 shows that D650, D700, E875 and D1049 are conserved cleavage sites at RuvC domain. B, Introducing point mutations of D650A, E875A, and D1049A result in abolished activity of xCas12i.

FIG. 12 and FIG. 13 shows engineering for high-efficiency dxCas12i-ABE. FIG. 12 and FIG. 13A, Engineering schematic of TadA8e.1-dxCas12i. Four parts for engineering are indicated. FIG. 13B, TadA8e.1-dxCas12i-v1.2 and v1.3 exhibits significantly increased A-to-G editing activity among various variants at KLKF4 site of genome.

FIG. 13C, Increased A-to-G editing activity of TadA8e-dxCas12i-v2.2 by combining v1.2 and v1.3. FIG. 13D, Unchanged or even decreased editing activity from various dCas12-ABEs carrying different NLS at N-terminal.

FIG. 13E, Increased A-to-G editing activity of TadA8e-dxCas12i-v4.3 by combining v2.2, changed-NLS linker and high-activity Tade8e.

FIG. 14 shows other strategies for high-efficiency dxCas12i-ABE. A, Schematics of different versions of dxCas12i adenine base editors. B, dxCas12i-ABE-N by TadA at the C-terminus of dCas12 slightly increased editing activity.

FIG. 15 shows comparison of editing frequencies induced by various dCas12-ABEs at different genomic target sites. A-B, Comparison of A-to-G editing frequencies induced by indicated TadA8e.1-dxCas12i-v1.2, v2.2, and TadA8e.1-dLbCas12a at PCSK9 and TTR genomic locus.

FIG. 16 shows characterization of dxCas12i-ABE in HEK293T cells. A-C, dCas12Max-ABE base editing of each target sites with TTN (A), ATN (B), and CTN (C) PAM. D, dCas12Max-ABE base editing product purity of each target sites with TTN PAM of A. Target sites are indicated, with sequences of each target protospacer and PAM listed in Supplementary Table 4.

FIG. 17 shows comparison of editing frequencies induced by various dCas12-CBEs at different genomic target sites. A-B, Comparison of C-to-T editing frequencies and product purity induced by indicated hA3A.1-dxCas12i, v1.2, v2.2, and hA3A.1-dCas12a at DYRK1A and SITE4 genomic locus. hA3A.1 represents human APOBEC3A-W104A.

FIG. 18 shows that hfCas12Max mediates high editing efficiency in HEK293 cells. A-C, Unchanged viability and proliferation and increasing indel activity of HEK293 cells following delivery of hfCas12Max RNPs with targeted TTR or TRAC crRNA at increasing concentration (n=1).

FIG. 19 shows that hfCas12Max mediates high editing efficiency in mouse blastocyst. A, Schematics of hfCas12Max gene editing in mouse blastocyst. hfCas12Max mRNA and targeted Ttr crRNA were injected into mouse zygotes, and the injected zygotes were cultured into blastocyst stage for genotyping analysis by targeted deep sequencing. B, Indel rates of hfCas12Max targeted Ttr.3 and Ttr.12 in mouse blastocyst (n=12).

FIG. 20 shows interaction of CRISPR-Cas12i system and a target dsDNA.

FIG. 21 shows the dsDNA cleavage activity of xCas12i when using various DR sequence variant.

FIG. 22 shows the secondary structures of direct repeat sequences of the guide RNAs of the disclosure.

The figures herein are for illustrative purposes only and are not necessarily drawn to scale.

### DETAILED DESCRIPTION OF THE EXAMPLE EMBODIMENTS

### Overview

In this study, the applicant demonstrate that the Type V-I Cas12i system enables versatile and efficient genome editing in mammalian cells. The applicant found a Cas12i, xCas12i (also referred to as “SiCas12i” herein), that shows high editing efficiency at TTN-PAM sites. By semi-rational design and protein engineering of its PI, REC, RuvC domains, the applicant obtained a high-efficiency, high-fidelity variant, hfCas12Max, which contains N243R, E336R, and D892R substitutions. In agreement with the hypothesis that introducing arginine at key sites could strengthen the binding between Cas and DNA, the introduction of N243R in the PI domain and E336R at REC domain significantly increased editing activity and expanded PAM recognition. Interestingly, D892R or G883R substitutions in the RuvC domain reduced off-target and retained on-target cleavage activity, whereas, alanine substitutions^{28, 29}, which has been used to reduce off-target activity, did not (FIG. S6C). The D892R substituted hfCas12Max was obviously more sensitive to mismatch, which suggests that D892R or G883R improved sgRNA binding specificity. According to sequence alignment and predicted structure of xCas12i to Cas12i2, asparagine 892 is located on NUC domain, together with RuvC domain to forming a cleft, in which crRNA: DNA heteroduplex was located. The variant with D892R did not alter the on-target but eliminated off-target activity, probably due to arginine substitution of asparagine affecting the binding of non-target crRNA. Our data suggests that a semi-rational engineering strategy with arginine substitutions based on the EGFP-activated reporter system could be used as a general approach to improve the activity of CRISPR editing tools.

Through engineering, the Cas12i system of the disclosure has achieved high editing activity, high specificity and a broad PAM range, comparable to SpCas9, and better than other Cas12 systems. Given its smaller size, short crRNA guide, and self-processing features^{4, 8, 10}, the Type V-I Cas12i system is suitable for in vivo multiplexed gene editing applications, including AAV30 or LNP^{12, 13}. Indeed, the data of the disclosure indicates Type V-I Cas12i system mediates the robust ex vivo or in vivo genome-editing efficiencies via ribonucleoprotein (RNP) delivery and lipid nanoliposomes (LNP) delivery respectively, demonstrating the great potential for therapeutic genome editing applications.

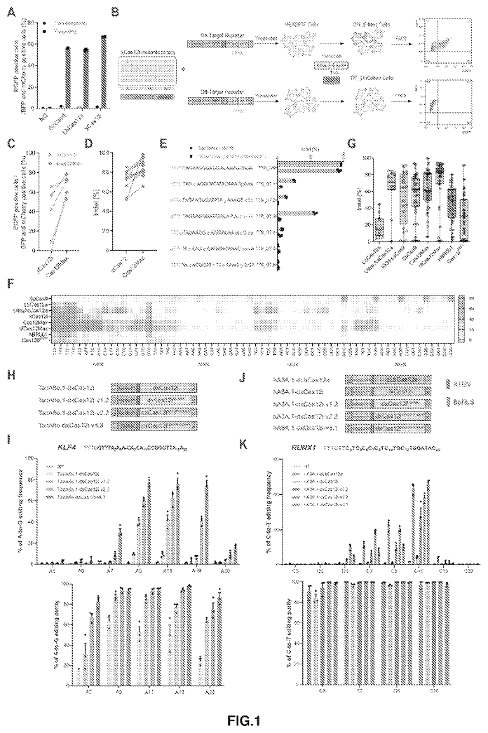

In addition, the applicant has confirmed that the Type V-I Cas12i system can be used in base editing applications. For base editor, the dCas12i system shows high A-to-G editing at A9-A11 sites even A19 of KLF locus, and C-to-T editing at A7-A10 sites, which is similar to the dCas12a system but is distinct from the dCas9/nCas9 system. Comparable to dCas12a, dCas12i-BE exhibited higher base editing activity at KLF4, PCSK9 and DYRK1A loci (FIG. 1K, FIG. S13A, FIG. S15A), suggesting it may have more potential as a base editor. This suggests that the dCas12i system is useful for broad genome engineering applications, including epigenome editing, genome activation, and chromatin imaging^{1, 31-34}.

In summary, the Cas12i system described here, which has robust editing activity and high specificity, is a versatile platform for genome editing or base editing in mammalian cells and could be useful in the future for in vivo or ex vivo therapeutic applications.

### General Definitions

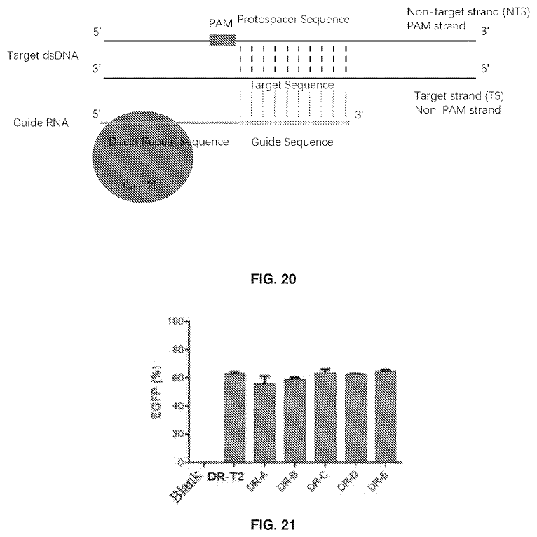

Cas12i is a programmable RNA-guided dsDNA endonuclease that may generate a double-strand break (DSB) on a target dsDNA as guided by a programmable RNA referred to as guide RNA (gRNA) comprising a spacer sequence and a direct repeat sequence. Without wishing to be bound by theory, it is believed that the direct repeat sequence is responsible for forming a complex with Cas12i and the spacer sequence is responsible for hybridizing to a target sequence of a target dsDNA, thereby guiding the complex of the gRNA and the Cas12i to the target dsDNA. Referring to FIG. 20, a target dsDNA is depicted to comprise a 5′ to 3′ upside strand and a 3′ to 5′ downside strand. A guide RNA is depicted to comprise a spacer sequence in green and a direct repeat sequence in orange. The spacer sequence is designed to hybridize to a part of the downside strand, and so the spacer sequence “targets” the part of the downside strand. And thus, the downside strand is referred to as a “target DNA strand” or a “target strand (TS)” of the target dsDNA, while the upside strand is referred to as a “non-target DNA strand” or a “non-target strand (NTS)” of the target dsDNA. The part of the target strand based on which the spacer sequence is designed and to which the spacer sequence may hybridize is referred to as a “target sequence”, while the corresponding part of the part on the non-target strand is referred to as the “reverse complementary sequence of the target sequence” or “reverse complementary sequence” or “protospacer sequence”. In case of any conflict with elsewhere of the disclosure, the definitions in this paragraph shall prevail.

Unless otherwise specifically indicated, the invention will be practiced using conventional methods of chemistry, biochemistry, organic chemistry, molecular biology, microbiology, recombinant DNA technology, genetics, immunology, cell biology, stem cell protocols, cell culture, and transgenic biology in the art, many of which are described below for illustrative purposes. Such technologies are well described in the literature.

All publications, patents and patent applications cited herein are incorporated herein by reference in their entirety. Unless otherwise specified, all technical and scientific terms used herein have the meaning commonly understood by one of ordinary skill in the art to which this invention belongs. For the purposes of the invention, the following terms are defined to conform to the meanings commonly understood in the art.

The articles “a/an” and “the” are used herein to refer to one or more than one (i.e., at least one) grammatical object of the article. For example, “element” means one element or more than one element.

The use of alternatives (e.g., “or”) is to be understood to mean either, both, or any combination thereof.

The term “and/or” should be understood to mean either or both of the alternatives.

As used herein, the term “about” or “approximately” refers to an amount, level, value, quantity, frequency, percentage, dimension, size, mass, weight, or length that is changed by up to 15%, 10%, 9%, 8%, 7%, 6%, 5%, 4%, 3%, 2%, or 1% as compared to the reference amount, level, value, quantity, frequency, percentage, dimension, size, mass, weight, or length. In one embodiment, the term “about” or “approximately” refers to a range of amount, level, value, quantity, frequency, percentage, dimension, size, mass, weight, or length that is ±15%, ±10%, ±9%, ±8%, ±7%, ±6%, ±5%, ±4%, ±3%, ±2%, or ±1% around the reference amount, level, value, frequency, frequency, percentage, scale, size, weight, quantity, weight, or length.

As used herein, the term “substantially/essentially” refers to a degree, amount, level, value, quantity, frequency, percentage, dimension, size, mass, weight, or length that is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% or more of the reference degree, amount, level, value, quantity, frequency, percentage, dimension, size, mass, weight, or length.

A numerical range includes the end values of the range, and each specific value within the range, for example, “16 to 100 nucleotides” includes 16 and 100, and each specific value between 16 and 100.

Throughout this specification, the terms “comprise”, “include”, “contain”, and “have” are to be understood as implying that a stated step or element or a group of steps or elements is included, but not excluding any other step or element or group of steps or elements, unless the context requires otherwise. In certain embodiments, the terms “comprise”, “include”, “contain”, and “have” are used synonymously.

“Consist of” means including but limited to any element after the phrase “consist of”. Thus, the phrase “consist of” indicates that the listed elements are required or mandatory, and that no other elements can be present.

“Consist essentially of” is intended to include any element listed after the phrase “consist essentially of” and is limited to other elements that do not interfere with or contribute to the activities or actions specified in the disclosure of the listed elements. Thus, the phrase “consist essentially of” is intended to indicate that the listed elements are required or mandatory, but no other elements are optional, and may or may not be present depending on whether they affect the activities or actions of the listed elements.

Throughout the specification, reference to “one embodiment”, “embodiment”, “a specific embodiment”, “a related embodiment”, “an embodiment”, “another embodiment” or “a further embodiment” or a combination thereof means that specific features, structures, or characteristics described in connection with the embodiment are included in at least one embodiment of the invention. Accordingly, the appearances of the foregoing phrases in various places throughout the specification are not necessarily all referring to the same embodiments. Furthermore, specific features, structures, or characteristics may be combined in any suitable manner in one or more embodiments.

“Sequence identity” between two polypeptides or nucleic acid sequences refers to the percentage of the number of identical residues between the sequences relative to the total number of the residues, and the calculation of the total number of residues is determined based on types of mutations. Types of mutations include insertion (extension) at either end or both ends of a sequence, deletions (truncations) at either end or both ends of a sequence, substitutions/replacements of one or more amino acids/nucleotides, insertions within a sequence, deletions within a sequence. Taking polypeptide as an example (the same for nucleotide), if the mutation type is one or more of the following: replacement/substitution of one or more amino acids/nucleotides, insertion within a sequence, and deletion within a sequence, then the number of residues of the larger molecule in the compared molecules is taken as the total number of residues. If the mutation type also includes an insertion (extension) at either end or both ends of the sequence or a deletion (truncation) at either end or both ends of the sequence, the number of amino acids inserted or deleted at either end or both ends (e.g., less than 20 inserted or deleted at both ends) is not counted in the total number of residues. In calculating the percentage of identity, the sequences being compared are aligned in a manner that produces the largest match between the sequences, and the gaps (if present) in the alignment are resolved by a particular algorithm.

Conservative substitutions of non-critical amino acids may be made without affecting the normal functions of the protein. Conservative substitutions refer to the substitution of amino acids with chemically or functionally similar amino acids. Conservative substitution tables that provide similar amino acids are well known in the art. For example, in some embodiments, the amino acid groups provided below are considered to be mutual conservative substitutions.

In certain embodiments, selected groups of amino acids considered as mutual conservative substitutions are as follows:

| | |
|---|---|
| Acidic residues | D and E |
| Basic residues | K, R and H |
| Hydrophilic uncharged residues | S, T, N, and Q |
| Aliphatic uncharged residues | G, A, V, L and I |
| Nonpolar uncharged residues | C, M and P |
| Aromatic residues | F, Y and W |

In certain embodiments, other selected groups of amino acids considered as mutual conservative substitutions are as follows:

| | |
|---|---|
| Group 1 | A, S and T |
| Group 2 | D and E |
| Group 3 | N and Q |
| Group 4 | R and K |
| Group 5 | I, L and M |
| Group 6 | F, Y and W |

In certain embodiments, other selected groups of amino acids considered as mutual conservative substitutions are as follows:

| | |
|---|---|
| Group A | A and G |
| Group B | D and E |
| Group C | N and Q |
| Group D | R, K and H |
| Group E | I, L, M, V |
| Group F | F, Y and W |
| Group G | S and T |
| Group H | C and M |

The term “amino acid” means twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V).

As used herein, the term “Cas12i protein” is used in its broadest sense and includes parental or reference Cas12i proteins (e.g., Cas12i protein comprising any of SEQ ID NOs: 1-10), derivatives or variants thereof, and functional fragments such as oligonucleotide-binding fragments thereof.

As used herein, the term “crRNA” is used interchangeably with guide molecule, gRNA, and guide RNA, and refers to nucleic acid-based molecules, which include but are not limited to RNA-based molecules capable of forming complexes with CRISPR-Cas proteins (e.g., any of Cas12i proteins described herein) (e.g., via direct repeat, DR), and comprises sequences (e.g., spacers) that are sufficiently complementary to a target nucleic acid sequence to hybridize to the target nucleic acid sequence and guide sequence-specific binding of the complex to the target nucleic acid sequence.

As used herein, the term “CRISPR array” refers to a nucleic acid (e.g., DNA) fragment comprising CRISPR repeats and spacers, which begins from the first nucleotide of the first CRISPR repeat and ends at the last nucleotide of the last (terminal) CRISPR repeat. Typically, each spacer in the CRISPR array is located between two repeats. As used herein, the term “CRISPR repeat” or “CRISPR direct repeat” or “direct repeat” refers to a plurality of short direct repeat sequences that exhibit very little or no sequence variation in a CRISPR array. Appropriately, V-I direct repeats may form a stem-loop structure.

“Stem-loop structure” refers to a nucleic acid having a secondary structure including a nucleotide region known or predicted to form a double strand (stem) connected on one side by a region (loop) which is mainly a single-stranded nucleotide. The terms “hairpin” and “fold-back” structures are also used herein to refer to stem-loop structures. Such structures are well known in the art and these terms are used in accordance with their well-known meanings in the art. As known in the art, the stem-loop structure does not require accurate base pairing. Thus, the stem may include one or more base mismatches. Alternatively, the base pairing may be accurate, i.e., no mismatch is included.

As use herein, target nucleic acid is used interchangeably with target sequence or target nucleic acid sequence to refer to a specific nucleic acid comprising a nucleic acid sequence complementary to all or part of a spacer in a crRNA. In some examples, the target nucleic acid comprises a gene or a sequence within the gene. In some examples, the target nucleic acid comprises a non-coding region (e.g., a promoter). In some examples, the target nucleic acid is single-stranded. In some examples, the target nucleic acid is double-stranded.

As used herein, “donor template nucleic acid” or “donor template” is used interchangeably to refer to a nucleic acid molecule that can be used by one or more cell proteins to alter the structure of a target nucleic acid after the CRISPR enzyme described herein alters the target nucleic acid. In some examples, the donor template nucleic acid is a double-stranded nucleic acid. In some examples, the donor template nucleic acid is a single-stranded nucleic acid. In some examples, the donor template nucleic acid is linear. In some examples, the donor template nucleic acid is circular (e.g., plasmid). In some examples, the donor template nucleic acid is an exogenous nucleic acid molecule. In some examples, the donor template nucleic acid is an endogenous nucleic acid molecule (e.g., chromosome).

The target nucleic acid should be associated with PAM (protospacer adjacent motif), that is, short sequences recognized by the CRISPR complex. Depending on the nature of the CRISPR-Cas protein, the target sequence should be selected such that its complementary sequence (the complementary sequence of the target sequence) in the DNA duplex is upstream or downstream of PAM. In an embodiment of the invention, the complementary sequence of the target sequence is downstream or 3′ of PAM. The requirements for exact sequence and length of PAM vary depending on the Cas12i protein used.

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

As use herein, the term “cleavage” refers to DNA breakage in a target nucleic acid produced by a nuclease of the CRISPR system described herein. In some examples, the cleavage is double-stranded DNA breakage. In some examples, the cleavage is single-stranded DNA breakage.

As used herein, the meanings of “cleaving target nucleic acid” or “modifying target nucleic acid” may overlap. Modifying a target nucleic acid includes not only modification of a mononucleotide but also insertion or deletion of a nucleic acid fragment.

### Cas12i Proteins

The present application provides Cas12i proteins, such as those of SEQ ID NOs: 1-10, which have single-stranded or double-stranded DNA cleavage activity. The Cas12i proteins described herein have less than about 50% sequence identity to other known Cas12i, are smaller and have better delivery efficiency than other Cas such as Cas9 or Cas12. In some embodiments, the Cas12i protein comprises a sequence of any of SEQ ID NOs: 1-10, such as any of SEQ ID NOs: 1-3, 6, and 10, or SEQ ID NO: 1. In some embodiments, the Cas12i protein is isolated. In some embodiments, the Cas12i protein is engineered. In some embodiments, the Cas12i protein is man-made.

Cas12i proteins described herein, such as SiCas12i, Si2Cas12i, WiCas12i, and SaCas12i, have excellent cleavage activity for exogenous or endogenous genes in vitro or at the cellular level, comparable to or even better than the cleavage activity of SpCas9, LbCas12a, and Cas12i.3. The cleavage activity of Cas12i proteins described herein, such as SiCas12i, Si2Cas12i, WiCas12i, and SaCas12i, for specific target sequences of exogenous or endogenous genes can be greater than about any of 15%, 20%, 30%, 40%, 50%, 60%, 70%, 80%, 90%, 95% or even greater than 99% at the cellular level. Generally speaking, the cleavage activity of Cas12i proteins described herein for specific target sequences of exogenous or endogenous genes at the cellular level is superior to that of Cas12i.3. The cleavage activity of SiCas12i for exogenous or endogenous genes in vitro or at the cellular level is comparable to, or even better than that of SpCas9 or LbCas12a, and significantly better than that of Cas12i.3. Its cleavage activity for specific target sequences of exogenous or endogenous genes at the cellular level may be greater than about any of 15%, 20%, 30%, 40%, 50%, 60%, 70%, 80%, 90%, 95% or even greater than 99%. In general, the cleavage activity of SiCas12i for specific target sequences of exogenous or endogenous genes at the cellular level is significantly superior to that of Cas12i.3.

The above Cas12i proteins may also comprise amino acid mutations that do not substantially affect (e.g., affect no more than about any of 5%, 4%, 3%, 2%, 1%, or smaller) the catalytic activity (endonuclease cleavage activity) or nucleic acid binding function of the Cas12i.

In some embodiments, the Cas12i proteins of the disclosure (including variants, dCas, nickases, etc.), such as SiCas12i, comprise one or more nuclear localization sequences (NLSs) at its N-terminus and/or C-terminus, preferably one NLS at its N-terminus and one NLS at C-terminus. In some embodiments, the NLS is an SV40 NLS (e.g., as set forth in SEQ ID NO: 444), preferably when the Cas12i protein is used for cleavage. In some embodiments, the NLS is a BP NLS, such as shown in SEQ ID NO: 443, preferably when the Cas12i protein is used for base editing, more preferably the Cas12i protein is fused at its N-terminus a BP NLS of SEQ ID NO: 443, and fused at its C-terminus a BP NLS of SEQ ID NO: 443.

### Cas12i Protein Variants

The present invention also provides variants of any of the Cas12i proteins described herein, such as Cas12i variants with at least about 80% (e.g., at least about any of 81%, 82%, 83%, 84%, 85%, 86%, 87%, 88%, 89%, 90%, 91%, 92%, 93%, 94%, 95%, 96%, 97%, 98%, 99%, 99.5% or higher) but less than 100% identical sequence to any of SEQ ID NOs: 1-10 (preferably, SEQ ID NOs: 1-3, 6, and 10, more preferably, SEQ ID NO: 1). In some embodiments, the Cas12i variant comprises one or more substitutions, insertions, deletions, or truncations relative to the amino acid sequence of a reference Cas12i protein (e.g., a Cas12i protein comprising the amino acid sequence of any one of SEQ ID NOs: 1-10).

As used herein, “variant” refers to a polynucleotide or a polypeptide that differs from a reference (e.g., parental) polynucleotide or polypeptide, respectively, but retains the necessary properties. A typical variant of a polynucleotide differs in nucleic acid sequence from a reference polynucleotide. Nucleotide changes may or may not alter the amino acid sequence of the polypeptide encoded by the reference polynucleotide. Nucleotide changes can result in amino acid substitutions, additions, deletions, or truncations in the polypeptide encoded by the reference polynucleotide. A typical variant of a polypeptide differs in amino acid sequence from a reference polypeptide. Typically, this difference is limited such that the sequences of the reference and variant polypeptides are generally very similar and identical in many regions. The amino acid sequences of the variant polypeptide and the reference polypeptide may differ by any combination of one or more of substitutions, additions, deletions, or truncations. A substituted or inserted amino acid residue may or may not be an amino acid residue encoded by the genetic code. Variants of a polynucleotide or polypeptide may be naturally occurring (such as allelic variants), or may be non-naturally occurring. Non-naturally occurring variants of polynucleotides and polypeptides can be prepared by mutagenesis techniques, by direct synthesis, or by other recombinant methods known to those of skill in the art.

As used herein, the term “wild-type” has the meaning commonly understood by those skilled in the art and means the typical form of an organism, strain, gene or trait. It can be isolated from resources in nature and has not been deliberately decorated.

As used herein, the terms “non-naturally occurring” and “engineered” are used interchangeably and refer to artificial involvement. When these terms are used to describe a nucleic acid molecule or polypeptide, it is meant that the nucleic acid molecule or polypeptide is at least substantially free of at least one other component with which it is naturally associated or occurs in nature.

In some embodiments, the Cas12i variant is isolated. In some embodiments, the Cas12i variant is engineered or non-naturally occurring. In some embodiments, the Cas12i variant is artificially synthesized. In some embodiments, the Cas12i variant has one or more amino acid mutations (e.g., insertions, deletions, or substitutions) in one or more domains relative to a reference Cas12i protein (e.g., the parental Cas12i protein), such as PI domain, Helical domain, RuvC domain, WED domain, Nuc domain, etc.

In some embodiments, the Cas12i variant is a variant relative to SiCas12i (SEQ ID NO: 1). This means that the Cas12i variant (e.g., a variant of Si2Cas12i) in its original sequence (e.g., Si2Cas12i, SEQ ID NO: 2) and the original SiCas12i (SEQ ID NO: 1) can be aligned, and the one or more positions with amino acid mutations (such as insertions, deletions or substitutions) can be identified. In some embodiments, the Cas12i variant is an engineered SiCas12i.

In some embodiments, the Cas12i variant (e.g., a SiCas12i variant) has a higher spacer-specific endonuclease cleavage activity against a target sequence of a target DNA that is complementary to the guide sequence, compared to the corresponding reference Cas12i protein (e.g., Cas12i protein comprising any of SEQ ID NOs: 1-10), such as at least about 1.2-fold (e.g., at least about any of 1.3, 1.4, 1.5, 1.6, 1.7, 1.8, 1.9, 2, 2.5, 3, 3.5, 4, 5, 10, 20, 50-fold, or higher) higher than the corresponding reference Cas12i protein.

In some embodiments, the original reference Cas12i protein (e.g., Cas12i protein comprising any of SEQ ID NOs: 1-10) has a higher spacer-specific endonuclease cleavage activity against a target sequence of a target DNA that is complementary to the guide sequence, compared to the corresponding Cas12i variant (e.g., SiCas12i variant), such as at least about 1.2-fold (e.g., at least about any of 1.3, 1.4, 1.5, 1.6, 1.7, 1.8, 1.9, 2, 2.5, 3, 3.5, 4, 5, 10, 20, 50-fold, or higher) higher than the Cas12i variant.

In some embodiments, the spacer-specific endonuclease cleavage activity of the Cas12i variant (e.g., a SiCas12i variant) against a target sequence of a target DNA that is complementary to a guide sequence is the same as or not significantly different from (e.g., within about 1.2-fold) that of the corresponding original Cas12i protein (e.g., Cas12i protein comprising any of SEQ ID NOs: 1-10). For example, in some embodiments, the Cas12i variant has the same spacer-specific endonuclease cleavage activity against the target sequence of the target DNA that is complementary to the guide sequence as the corresponding original Cas12i protein. In some embodiments, the Cas12i variant has a spacer-specific endonuclease cleavage activity against a target sequence of a target DNA that is complementary to a guide sequence of no more than about 1.2-fold higher than the corresponding original Cas12i protein (e.g., less than or equal to about any of 1.2, 1.19, 1.15, 1.1, 1.01, 1.001-fold, etc.). In some embodiments, the spacer-specific endonuclease cleavage activity of the original Cas12i protein against a target sequence of a target DNA that is complementary to the guide sequence is no more than about 1.2-fold higher than that of the corresponding Cas12i variant (e.g., less than or equal to about any of 1.2, 1.19, 1.15, 1.1, 1.01, 1.001-fold, etc.).

Cas12i Proteins Substantially Lacking Catalytic Activity (dCas12i)

The present invention also provides dead Cas12i (dCas12i) proteins lacking or substantially lacking catalytic activity. For example, in some embodiments, the dCas12i protein retains less than about 50% (e.g., less than about any of 40%, 35%, 30%, 27.5%, 25%, 22.5%, 20%, 17.5%, 15%, 12.5%, 10%, 7.5%, 5%, 4%, 3%, 2.5%, 2%, 1% or less) spacer-specific endonuclease cleavage activity of the corresponding parental Cas12i protein (e.g., Cas12i protein comprising any of SEQ ID NOs: 1-10) for a target sequence of a target DNA that is complementary to a guide sequence. In some embodiments, the dCas12i protein comprises one or more amino acid substitutions in the RuvC domain (e.g., RuvC domain of a Cas12i protein comprising any of SEQ ID NOs: 1-10), resulting in substantial lack of catalytic activity. In some embodiments, the DNA cleavage activity of dCas12i is zero or negligible compared to the non-mutated Cas12i form. In some embodiments, the dCas12i is a Cas12i protein without catalytic activity, which contains mutation(s) in the RuvC domain that allow for formation of a CRISPR complex and successful binding to a target nucleic acid while not allowing for successful nuclease activity (catalytic/cleavage activity).

In some embodiments, the dCas12i is a dSiCas12i substantial lacking catalytic activity. In some embodiments, the dSiCas12i comprises one or more substitutions at amino acid residues 650, 700, 875, and/or 1049 relative to SEQ ID NO: 1. In some embodiments, the dSiCas12i comprises one or more substitutions selected from the group consisting of D700A, D700V, D650A, D650V, E875A, E875V, D1049A, and D1049V relative to SEQ ID NO: 1. In one embodiment, the dSiCas12i comprises the amino acid sequence of any of dSiCas12i-D700A, dSiCas12i-D650A, dSiCas12i-E857A, and dSiCas12i-D1049A, respectively. In some embodiments, the dSiCas12i comprises one or more substitutions selected from the group consisting of D650A, D700A, E875A, D1049A, D650A+D700A, D700A+E875A, D700A+D1049A, D650A+E875A, D650A+D1049A, E875A+D1049A, D650A+D700A+E875A, D650A+D700A+D1049A, D650A+E875A+D1049A, D700A+E875A+D1049A, and D650A+D700A+E875A+D1049A, relative to SEQ ID NO: 1. In addition, the dCas12i may contain mutations other than those previously described that do not substantially affect (e.g., affect no more than about any of 5%, 4%, 3%, 2%, 1%, or smaller) the catalytic activity or nucleic acid binding function of the dCas12i protein. The dCas12i protein, which substantially lacks catalytic activity, can be used as a DNA-binding protein.

In some embodiments, the dCas12i described herein can be fused with an adenosine deaminase (ADA) or a cytidine deaminase (CDA), or a catalytic domain thereof, to achieve single-base editing. In some embodiments, the single-base editing efficiency of a fusion protein comprising any of the dCas12i proteins described herein and an ADA or a CDA (or catalytic domain thereof) is at least about 10% higher (e.g., at least about any of 20%, 30%, 40%, 50%, 60%, 70%, 80% 90%, 100%, 120%, 150%, 200%, 500%, 1000%, or higher) than that of a fusion protein comprising a dCas12i not from present invention and a sane ADA or CDA (or catalytic domain thereof).

The number of amino acids in a full-length sequence of any of the Cas12i or dCas12i proteins described above is remarkably less than that of Cas12 proteins of other types, and their smaller molecular size facilitates the subsequent assembly and delivery of the Cas system in vivo.

In some embodiments, the adenosine deaminase is TadA8e, such as TadA8e comprising the sequence of SEQ ID NO: 439 or 461.

In some embodiments, the C′ terminus of a deaminase, such as adenosine deaminase, is fused to the N′ terminus of a dCas12i via an optional peptide linker, such as a peptide linker comprising SEQ ID NO: 442. In some embodiments, the N′ terminus of a deaminase, such as adenosine deaminase, is fused to the C′ terminus of a dCas12i via an optional peptide linker, such as a peptide linker comprising SEQ ID NO: 442. In some embodiments, there is provided a fusion protein comprising dSiCas12i and an adenosine deaminase (e.g., TadA8e), such as fusion protein TadA8e-dSiCas12i-D1049A, or fusion protein TadA8e-dSiCas12i-E875A.

Unless otherwise specified, “Cas12i,” or “Cas12i protein” described herein include any Cas12i protein described in the disclosure and its variants (such as mutants), derivatives (such as Cas12i fusion proteins), as well as dCas12i proteins substantially lacking catalytic activity and derivatives thereof (such as dCas12i fusion proteins, such as dCas12i-TadA). The present invention also provides nucleotide sequences encoding any of the Cas12i proteins and variants and derivatives thereof, such as the polynucleotide sequences of any of SEQ ID NOs: 21-40.

CRISPR (crRNA) or Guide RNA (gRNA)

Typically, crRNAs (exchangeable with guide RNA/gRNA) described herein comprise, consist essentially of, or consist of a direct repeat (DR) and a spacer. In some embodiments, the crRNA comprises, consists essentially of, or consists of a DR linked to a spacer. In some embodiments, the crRNA comprises a DR, a spacer, and a DR (DR-spacer-DR). This is a typical configuration of a pre-crRNA. In some embodiments, the crRNA comprises a DR, a spacer, a DR, and a spacer (DR-spacer-DR-spacer). In some embodiments, the crRNA comprises two or more DRs and two or more spacers. In some embodiments, the crRNA comprises a truncated DR, and a spacer. This is typical for processed or mature crRNAs. In some embodiments, the CRISPR-Cas12i effector protein forms a complex with the crRNA, and the spacer directs the complex to a target nucleic acid that is complementary to the spacer for sequence-specific binding.

In some embodiments, the CRISPR-Cas12i system described herein comprises one or more crRNAs (e.g., 1, 2, 3, 4, 5, 10, 15, or more), or nucleic acids encoding thereof. In some embodiments, the two or more crRNAs target different target sites, e.g., 2 target sites of the same target DNA or gene, or 2 target sites of 2 different target DNA or genes.

The sequences and lengths of the crRNAs described herein can be optimized. In some embodiments, the optimal length of the crRNA can be determined by identifying the processed form of the crRNA or by empirical length studies of the crRNA. In some embodiments, the crRNA comprises base modifications.

### Direct Repeat (DR)

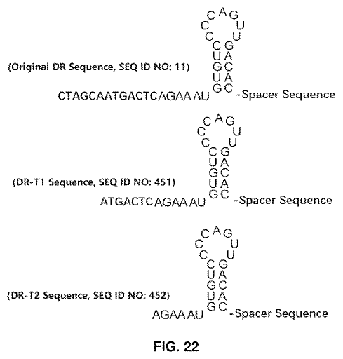

SEQ ID NOs: 11-20 exemplifies DR sequences of corresponding Cas12i protein of the disclosure. For example, the DR sequence corresponding to SiCas12i (or a variant or derivative thereof, or dSiCas12i or a fusion protein thereof) may comprise the nucleotide sequence set forth in SEQ ID NO: 11 or a functional variant thereof. Any DR sequence that can mediate the binding of the Cas12i protein described herein to the corresponding crRNA can be used in the disclosure. In some embodiments, the DR comprises the RNA sequence of any one of SEQ ID NOs: 11-20 and 451-457. In some embodiments, the DR is a “functional variant” of any of the RNA sequences of SEQ ID NOs: 11-20, such as a “functionally truncated version,” “functionally extended version,” or “functionally replacement version.” For example, DR sequence of SEQ ID NO: 451 or 452 is a part of SEQ ID NO: 11 (truncated version), it still has DR function, as demonstrated in Example, and is therefore a functional variant, or a functionally truncated DR variant. A “functional variant” of a DR is a 5′ and/or 3′ extended (functionally extended version) or truncated (functionally truncated version) variant of a reference DR (e.g., a parental DR), or comprises one or more insertions, deletions, and/or substitutions (functional replacement version) of one or more nucleotides relative to the reference DR (e.g., a parental DR), while still retaining at least about 20% (such as at least about any of 30%, 40%, 50%, 60%, 60%, 70%, 80%, 90%, 95%, or higher) functionality of the reference DR, i.e., the function to mediate the binding of a Cas12i protein to the corresponding crRNA. DR functional variants typically retain stem-loop-like secondary structure or portions thereof available for Cas12i protein binding. As shown in FIGS. 21 and 22, DR-T2 (SEQ ID NO: 452) is one of the functionally truncated versions of the DR shown in SEQ ID NO: 11. In some embodiments, the DR or functional variant thereof comprises a stem-loop-like secondary structure or portion thereof available for binding by the Cas12i protein. In some embodiments, the DR or functional variant thereof comprises at least two (e.g., 2, 3, 4, 5 or more) stem-loop-like secondary structures or portions thereof available for binding by the Cas 12i protein.

In some embodiments, the DR or functional variant thereof comprises at least about 16 nucleotides (nt), such as 16, 17, 18, 19, 20, 21, 22, 23, 24, 25, 26, 27, 28, 29, 30, 31, 32, 33, 34, 35, 36, 37, 38, 39, 40 or more nucleotides. In some embodiments, the DR comprises about 20nt to about 40nt, such as about 20nt to about 30nt, about 22nt to about 40nt, about 23nt to about 38nt, about 23nt to about 36nt, or about 30nt to about 40nt. In some embodiments, the DR comprises 22nt, 23nt, or 24nt. In some embodiments, the DR comprises 35nt, 36nt, or 37nt.

In some embodiments, the DR sequence comprises a stem-loop structure near the 3′ end (immediately adjacent to the spacer sequence). “Stem-loop structure” refers to a nucleic acid having a secondary structure that includes regions of nucleotides known or predicted to form a double-strand (stem) portion and connected at one end by a linking region (loop) of substantially single-stranded nucleotides. The term “hairpin” structure is also used herein to refer to stem-loop structures. Such structures are well known in the art, and these terms are used in accordance with their commonly known meanings in the art. Stem-loop structures do not require precise base pairing. Thus, the stem may comprise one or more base mismatches. Alternatively, base pairing may be exact, i.e., not including any mismatches.

The crRNA of the disclosure comprises a DR comprising a stem-loop structure near the 3′ end of the DR sequence. The DR stem-loop structure of SiCas12i is exemplified in FIG. 11. In some embodiments, the stem contained in the DR consists of 5 pairs of complementary bases that hybridize to each other, and the loop length is 6, 7, 8, or 9 nucleotides. In some embodiments, the loop length is 7 nucleotides. In some embodiments, the stem can comprise at least 2, at least 3, at least 4, or at least 5 base pairs. In some embodiments, the DR comprises two complementary stretches of nucleotides about 5 nucleotides in length separated by about 7 nucleotides. In some embodiments, the stem-loop structure comprises a first stem nucleotide chain of 5 nucleotides in length; a second stem nucleotide chain of 5 nucleotides in length, wherein the first and the second stem nucleotide chains can hybridize to each other; and a cyclic nucleotide chain arranged between the first and second stem nucleotide chains, wherein the cyclic nucleotide chain comprises 6, 7 or 8 nucleotides.

As used herein, the secondary structure of two or more crRNAs are substantially identical or not substantially different means that these crRNAs contain stems and/or loops differing by no more than 1, 2, or 3 nucleotides in length; in terms of nucleotide type (A, U, G, or C), the nucleotide sequences of these crRNAs when compared by sequence alignment differ by no more than 1, 2, 3, 4, 5, 6, 7 or 8 nucleotides. In some embodiments, the secondary structure of two or more crRNAs are substantially identical or not substantially different means that the crRNAs contain stems that differ by at most one pair of complementary bases, and/or loops that differ by at most one nucleotide in length, and/or contain stems with same length but with mismatched bases. In some embodiments, the stem-loop structure comprises 5′-X₁X₂X₃X₄X₅NNNnNNNX₆X₇X₈X₉X₁₀-3′, wherein X₁, X₂, X₃, X₄, X₅, X₆, X₇, X₈, X₉, and X₁₀ can be any base, n can be any base or deletion, and N can be any base; wherein X₁X₂X₃X₄X₅ and X₆X₇X₈X₉X₁₀ can hybridize to each other to form a stem and make NNNnNNN form a loop. In some embodiments, the stem-loop structure comprises the sequence of any one of SEQ ID NOs: 453-457.

In some embodiments, the DR sequence that can direct any of the Cas12i of the invention to the target site comprises one or more nucleotide changes selected from the group consisting of nucleotide additions, insertions, deletions, and substitutions that do not result in substantial differences in secondary structure compared to DR sequence set forth in any of SEQ ID NOs: 11-20 and 451-457 or functionally truncated version thereof.

### Spacer

In some embodiments, the length of the spacer sequence is at least about 16 nucleotides, preferably about 16 to about 100 nucleotides, more preferably about 16 to about 50 nucleotides (e.g., about any of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides). In some embodiments, the spacer is about 16 to about 27 nucleotides, such as any of about 17 to about 24 nucleotides, about 18 to about 24 nucleotides, or about 18 to about 22 nucleotides.

In some embodiments, the spacer is at least about 70% (e.g., at least about any of 75%, 80%, 85%, 90%, 90%, 91%, 92%, 93%, 94%, 95%, 96%, 97%, 98%, 99% or 100%) complementary to the target sequence. In some embodiments, there are at least about 15 (e.g., at least about any of 16, 17, 18, 19, 20, 21, 22, 23, 24, 25, 26, 27, 28, 29, 30, 35, 40, 45, 50 or more) between the spacer sequence and the target sequence of the target nucleic acid (e.g., DNA).

Complete complementarity is not required for spacers, provided that there is sufficient complementarity for the crRNA to function (i.e., directing Cas12i protein to the target site). The cleavage efficiency by Cas12i mediated by the crRNA can be adjusted by introducing one or more mismatches (e.g., 1 or 2 mismatches between the spacer sequence and the target sequence, including the positions along the mismatches of the spacer/target sequence). Mismatches, such as double mismatches, have greater impact on cleavage efficiency when they are located more central to the spacer (i.e., not at the 3′ or 5′ end of the spacer). Thus, by choosing the position of mismatches along the spacer sequence, the cleavage efficiency of Cas12i can be tuned. For example, if less than 100% cleavage of the target sequence is desired (e.g., in a population of cells), 1 or 2 mismatches between the spacer sequence and the target sequence can be introduced into the spacer sequence.

### PAM

In some embodiments, the Cas12i protein of the disclosure can recognize PAM (protospacer adjacent motif, protospacer adjacent motif) to act on the target sequence. In some embodiments, the PAM comprises or consists of 5′-NTTN-3′ (wherein N is A, T, G, or C). In some embodiments, the PAM comprises or consists of 5′-TTC-3′, 5′-TTA-3′, 5′-TTT-3′, 5′-TTG-3′, 5′-ATA-3′, or 5′-ATG-3′. In some embodiments, the PAM comprises or consists of 5′-TTC-3′.

### Collateral Activity

The Cas12i protein may have collateral activity, that is, under certain conditions, the activated Cas12i protein remains active after binding to the target sequence and continues to non-specifically cleave non-target oligonucleotides. This collateral activity enables detection of the presence of specific target oligonucleotides using the Cas12i system. In one embodiment, the Cas12i system is engineered to non-specifically cleave ssDNA or transcript. In certain embodiments, Cas12i is transiently or stably provided or expressed in an in vitro system or cell and is targeted or triggered to non-specifically cleave cellular nucleic acids, such as ssDNA, such as viral ssDNA. In some embodiments, the Cas12i protein described herein is modified to reduce (e.g., reduce at least about any of 10%, 20%, 30%, 40%, 50%, 60%, 70%, 80%, 90%, 95%, or higher) or eliminate spacer non-specific endonuclease cleavage activity. In some embodiments, the Cas12i protein described herein substantially lacks (e.g., lacks at lease about any of 50%, 60%, 70%, 80%, 90%, 95%, or 100%) spacer non-specific endonuclease collateral activity of the parental/reference Cas12i protein (e.g., Cas12i protein of any of SEQ ID NOs: 1-10) against a non-target DNA.

The collateral activity has recently been used in a highly sensitive and specific nucleic acid detection platform known as SHERLOCK which can be used in many clinical diagnostics (Gootenberg, J. S. et al., Nucleic acid detection with CRISPR-Cas13a/C2c2. Science 356, 438-442 (2017)).

### Reporter Nucleic Acid

A “reporter nucleic acid” refers to a molecule that can be cleaved or otherwise deactivated by the activated CRISPR system protein as described herein. The reporter nucleic acid comprises a nucleic acid element cleavable by the CRISPR protein. Cleavage of the nucleic acid element releases an agent or produces a conformational change allowing for the generation of a detectable signal. The reporter nucleic acid prevents the generation or detection of a positive detectable signal prior to cleavage or when the reporter nucleic acid is in an “active” state. It will be appreciated that in certain exemplary embodiments, minimal background signals may be generated in the presence of the active reporter nucleic acid. The positive detectable signal may be any signal that may be detected using optical, fluorescent, chemiluminescent, electrochemical or other detection methods known in the art. For example, in certain embodiments, a first signal (i.e., a negative detectable signal) may be detected when a reporter nucleic acid is present, and then it is converted to a second signal (e.g., a positive detectable signal) when the target molecule is detected and the reporter nucleic acid is cleaved or deactivated by the activated CRISPR protein.

### Functional Domains

Functional domains are used in their broadest sense and include proteins such as enzymes or factors themselves or specific functional fragments (domains) thereof.

A Cas12i protein (e.g., dCas12i) is associated with one or more functional domains selected from the group consisting of a deaminase (e.g., adenosine deaminase or cytidine deaminase) catalytic domain, a DNA methylation catalytic domain, a DNA demethylation catalytic domain, a histone residue modification domain, a nuclease catalytic domain, a fluorescent protein, a transcription modification factor (e.g., a transcription activation catalytic domain, a transcription inhibition catalytic domain), a nuclear localization signal (NLS), nuclear export signal (NES), a light gating factor, a chemical inducible factor, or a chromatin visualization factor; preferably, the functional domain is selected from the group consisting of an adenosine deaminase catalytic domain or cytidine deaminase catalytic domain.

In some embodiments, the functional domain may be a transcription activation domain. In some embodiments, the functional domain is a transcription repression domain. In some embodiments, the functional domain is an epigenetic modification domain such that an epigenetic modification enzyme is provided. In some embodiments, the functional domain is an activation domain. In some embodiments, the Cas12i protein is associated with one or more functional domains; and the Cas12i protein contains one or more mutations within the RuvC domain, and the resulting CRISPR complex can deliver epigenetic modifiers, or transcript or translate activation or repression signals.

In some embodiments, the functional domain exhibits activity to modify a target DNA or proteins associated with the target DNA, wherein the activity is one or more selected from the group consisting of nuclease activity (e.g., HNH nuclease, RuvC nuclease, Trex1 nuclease, Trex2 nuclease), methylation activity, demethylation activity, DNA repair activity, DNA damage activity, deamination activity, dismutase activity, alkylation activity, depurination activity, oxidation activity, pyrimidine dimer formation activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, transcription inhibition activity, and transcription activation activity. Target DNA associated proteins include, but not limited to, proteins that can bind to target DNA, or proteins that can bind to proteins bound to target DNA, such as histones, transcription factors, Mediator, etc.

The functional domain may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g., photo-inducible). When more than one functional domain is included, the functional domains may be the same or different.

### Base Editing

In certain exemplary embodiments, Cas12i (e.g., dCas12i) may be fused to adenosine deaminase or cytidine deaminase for base editing purposes.

### Adenosine Deaminase

As used herein, the term “adenosine deaminase” or “adenosine deaminase protein” refers to a protein, polypeptide, or one or more functional domains of a protein or polypeptide that can catalyze hydrolytic deamination reaction to convert adenine (or the adenine portion of a molecule) to hypoxanthine (or the hypoxanthine portion of a molecule), as shown below. In some embodiments, the adenine-containing molecule is adenosine (A) and the hypoxanthine-containing molecule is inosine (I). The adenine-containing molecule may be deoxyribonucleic acid (DNA) or ribonucleic acid (RNA).

According to the present disclosure, adenosine deaminases that can be used in combination with the present disclosure include, but are not limited to, enzyme family members referred to as adenosine deaminase acting on RNA (ADAR), enzyme family members referred to as adenosine deaminase acting on tRNA (ADAT), and other family members comprising adenosine deaminase domain (ADAD). According to the present disclosure, the adenosine deaminase is capable of targeting adenine in RNA/DNA and RNA duplexes. In fact, Zheng et al. (Nucleic Acids Res. 2017, 45 (6): 3369-3377) demonstrated that ADAR can edit adenosine to inosine in RNA/DNA and RNA/RNA duplexes. In specific embodiments, adenosine deaminase has been modified to increase its ability to edit DNA in the RNA/DNA heteroduplex of the RNA duplex, as described in detail below.

In some embodiments, the adenosine deaminase is derived from one or more metazoan species, including but not limited to mammals, birds, frogs, squid, fish, flies, and worms. In some embodiments, the adenosine deaminase is human, squid, or drosophila adenosine deaminase.

In some embodiments, the adenosine deaminase is human ADAR, including hADAR1, hADAR2, and hADAR3. In some embodiments, the adenosine deaminase is Caenorhabditis elegans ADAR protein, including ADR-1 and ADR-2. In some embodiments, the adenosine deaminase is drosophila ADAR protein, including dAdar. In some embodiments, the adenosine deaminase is squid (Loligo pealeii) ADAR protein, including sqADAR2a and sqADAR2b. In some embodiments, adenosine deaminase is human ADAT protein. In some embodiments, the adenosine deaminase is drosophila ADAT protein. In some embodiments, the adenosine deaminase is human ADAD protein, including TENR (hADAD1) and TENRL (hADAD2).

In some embodiments, the adenosine deaminase is TadA protein, such as E. coli TadA. See Kim et al., Biochemistry 45:6407-6416 (2006); Wolf et al., EMBO J. 21:3841-3851 (2002). In some embodiments, the adenosine deaminase is mouse ADA. See Grunebaum et al., Curr. Opin. Allergy Clin. Immunol. 13:630-638 (2013). In some embodiments, the adenosine deaminase is human ADAT2. See Fukui et al., J. Nucleic Acids 2010:260512 (2010). In some embodiments, the deaminase (e.g., adenosine or cytidine deaminase) is one or more of those described in: Cox et al., Science. Nov. 24, 2017; 358 (6366): 1019-1027; Komore et al., Nature. May 19, 2016; 533 (7603): 420-4; and Gaudelli et al., Nature. Nov. 23, 2017; 551 (7681): 464-471.

In some embodiments, the adenosine deaminase protein recognizes one or more target adenosine residues in a double-stranded nucleic acid substrate and converts them to inosine residues. In some embodiments, the double-stranded nucleic acid substrate is an RNA-DNA heteroduplex. In some embodiments, the adenosine deaminase protein recognizes a binding window on a double-stranded substrate. In some embodiments, the binding window comprises at least one target adenosine residue. In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp or 100 bp.

In some embodiments, the adenosine deaminase protein comprises one or more deaminase domains. Without wishing to be bound by a particular theory, it is contemplated that the deaminase domain is used to recognize one or more target adenosine (A) residues contained in a double-stranded nucleic acid substrate and convert them to inosine (I) residues. In some embodiments, the deaminase domain comprises an active center. In some embodiments, the active center comprises zinc ions. In some embodiments, during A-I editing, the base pair at the target adenosine residue is destroyed and the target adenosine residue is “flipped” out of the double helix to become accessible by the adenosine deaminase. In some embodiments, amino acid residues in or near the active center interact with one or more nucleotides 5′ of the target adenosine residue. In some embodiments, amino acid residues in or near the active center interact with one or more nucleotides 3′ of the target adenosine residue. In some embodiments, amino acid residues in or near the active center further interact with nucleotides complementary to the target adenosine residues on the opposite chain. In some embodiments, the amino acid residue forms a hydrogen bond with the 2′ hydroxyl group of the nucleotide.

In some embodiments, the adenosine deaminase comprises human ADAR2 whole protein (hADAR2) or deaminase domain (hADAR2-D) thereof. In some embodiments, the adenosine deaminase is a member of the ADAR family homologous to hADAR2 or hADAR2-D.

In particular, in some embodiments, the homologous ADAR protein is human ADAR1 (hADAR1) or deaminase domain (hADAR1-D) thereof. In some embodiments, glycine 1007 of hADAR1-D corresponds to glycine 487hADAR2-D, and glutamic acid 1008 of hADAR1-D corresponds to glutamic acid 488 of hADAR2-D.

In some embodiments, the adenosine deaminase comprises the wild-type amino acid sequence of hADAR2-D. In some embodiments, the adenosine deaminase comprises one or more mutations in the hADAR2-D sequence such that the editing efficiency and/or substrate editing preference of hADAR2-D are changed as desired.

In some embodiments, the adenosine deaminase is TadA8e, such as TadA8e comprising the sequence of SEQ ID NO: 439 or 461. In some embodiments, the Cas12i protein described herein (e.g., dCas12i) is fused to TadA8e or functional fragment thereof (i.e., capable of A-to-I single base editing). In some embodiments, the ABE of the disclosure comprises the amino acid sequence of SEQ ID NO: 463, which is dCas12Max-ABE.

### Cytidine Deaminase

In some embodiments, the deaminase is cytidine deaminase. As used herein, the term “cytidine deaminase” or “cytidine deaminase protein” refers to a protein, polypeptide, or one or more functional domains of a protein or polypeptide that can catalyze hydrolytic deamination reaction to convert cytosine (or the cytosine portion of a molecule) to uracil (or the uracil portion of a molecule), as shown below. In some embodiments, the cytosine-containing molecule is cytidine (C) and the uracil-containing molecule is uridine (U). The cytosine-containing molecule may be deoxyribonucleic acid (DNA) or ribonucleic acid (RNA).

According to the present disclosure, cytidine deaminases that can be used in combination with the present disclosure include, but are not limited to, members of an enzyme family known as apolipoprotein B mRNA editing complex (APOBEC) family deaminases, activation-induced deaminase (AID), or cytidine deaminase 1 (CDA1), and in specific embodiments, the deaminase in APOBEC1 deaminases, APOBEC2 deaminases, APOBEC3A deaminases, APOBEC3B deaminases, APOBEC3C deaminases and APOBEC3D deaminases, APOBEC3E deaminases, APOBEC3F deaminases, APOBEC3G deaminases, APOBEC3H deaminases or APOBEC4 deaminases.

In the methods and systems of the invention, the cytidine deaminase is capable of targeting cytosines in a DNA single strand. In certain exemplary embodiments, the cytidine deaminase can edit on a single strand present outside of the binding component, e.g., bind to Cas13. In other exemplary embodiments, the cytidine deaminase may edit at localized bubbles, such as those formed at target editing sites but with guide sequence mismatching. In certain exemplary embodiments, the cytidine deaminase may comprise mutations that contribute to focus activity, such as those described in Kim et al., Nature Biotechnology (2017) 35 (4): 371-377 (doi: 10.1038/nbt.3803).

In some embodiments, the cytidine deaminase is derived from one or more metazoan species, including but not limited to mammals, birds, frogs, squid, fish, flies, and worms. In some embodiments, the cytidine deaminase is human, primate, bovine, canine, rat, or mouse cytidine deaminase.

In some embodiments, the cytidine deaminase is human APOBEC, including hAPOBEC1 or hAPOBEC3. In some embodiments, the cytidine deaminase is human AID.

In some embodiments, the cytidine deaminase protein recognizes one or more target cytosine residues in a single-stranded bubble of a RNA duplex and converts them to uracil residues. In some embodiments, the cytidine deaminase protein recognizes a binding window on a single-stranded bubble of an RNA duplex. In some embodiments, the binding window comprises at least one target cytosine residue. In some embodiments, the binding window is in the range of about 3 bp to about 100 bp. In some embodiments, the binding window is in the range of about 5 bp to about 50 bp. In some embodiments, the binding window is in the range of about 10 bp to about 30 bp. In some embodiments, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp or 100 bp.

In some embodiments, the cytidine deaminase protein comprises one or more deaminase domains. Without wishing to be bound by theory, it is contemplated that deaminase domains are used to recognize one or more target cytosine (C) residues contained in a single-stranded bubble of a RNA duplex and convert them to uracil (U) residues. In some embodiments, the deaminase domain comprises an active center. In some embodiments, the active center comprises zinc ions. In some embodiments, amino acid residues in or near the active center interact with one or more nucleotides at 5′ of the target cytosine residue. In some embodiments, amino acid residues in or near the active center interact with one or more nucleotides at 3′ of the target cytosine residue.

In some embodiments, the cytidine deaminase comprises human APOBEC1 whole protein (hAPOBEC1) or its deaminase domain (hAPOBEC1-D) or its C-terminal truncated form (hAPOBEC-T). In some embodiments, the cytidine deaminase is a member of the APOBEC family homologous to hAPOBEC1, hAPOBEC-D, or hAPOBEC-T. In some embodiments, the cytidine deaminase comprises human AID1 whole protein (hAID) or its deaminase domain (hAID-D) or its C-terminal truncated form (hAID-T). In some embodiments, the cytidine deaminase is a member of the AID family homologous to hAID, hAID-D, or hAID-T. In some embodiments, hAID-T is hAID with the C-terminus truncated by about 20 amino acids.

In some embodiments, the cytidine deaminase comprises the wild-type amino acid sequence of cytosine deaminase. In some embodiments, the cytidine deaminase comprises one or more mutations in the cytosine deaminase sequence such that the editing efficiency and/or substrate editing preference of the cytosine deaminase are changed as desired. In some embodiments, the CBE of the disclosure comprises the amino acid sequence of SEQ ID NO: 464, which is dCas12Max-CBE.

As used herein, “associated” is used in its broadest sense and encompasses both the case where two functional modules form a fusion protein directly or indirectly (via a linker) and the case where two functional modules are each independently bonded together by covalent bonds (e.g., disulfide bond) or non-covalent bonds.

The term “vector” refers to a nucleic acid molecule capable of transporting another nucleic acid attached thereto. It is a replicon, such as a plasmid, phage, or cosmid, into which another DNA segment can be inserted to effect replication of the inserted segment. Typically, the vector is capable of replication when combined with suitable control elements.

In some cases, the vector system comprises a single vector. Alternatively, the vector system comprises a plurality of vectors. The vector may be a viral vector.

The vector includes, but are not limited to, a single-stranded, double-stranded or partially double-stranded nucleic acid molecule; a nucleic acid molecule comprising one or more free ends, or without a free end (e. g., circular); a nucleic acid molecule comprising DNA, RNA or both; and other polynucleotide variants known in the art. One type of vector is “plasmid”, which refers to a circular double-stranded DNA ring into which other DNA segments can be inserted, for example by standard molecular cloning techniques. Another type of vector is viral vector in which a viral-derived DNA or RNA sequence is present for packaging into a virus (e.g., retrovirus, replication-defective retrovirus, adenovirus, replication-defective adenovirus, and adeno-associated virus). The viral vector also comprises a polynucleotide carried by the virus for transfection into a host cell. Certain vectors are capable of autonomous replication in the host cells into which they are introduced (e.g., bacterial vectors having origins of bacterial replication and episomal mammalian vectors). After these vectors are introduced into the host cells, other vectors (e.g., non-episomal mammalian vectors) are integrated into the genomes of the host cells for replication with the host genomes. In addition, certain vectors are capable of guiding expression of genes operably linked thereto. Such vectors are referred to herein as “expression vectors”. Vectors expressed in eukaryotic cells and vectors resulting in expression in eukaryotic cells may be referred to herein as “eukaryotic expression vectors”. Common expression vectors useful in recombinant DNA techniques are usually in the forms of plasmids.

The recombinant expression vector may comprise the nucleic acid of the invention in a form suitable for expression in a host cell, which means that the recombinant expression vector comprises one or more regulatory elements that can be selected according to the host cell to be used for expression, and the nucleic acid is operably linked to a nucleic acid sequence to be expressed. Within recombinant expression vectors, “operably linked” is intended to mean that the nucleotide sequence of interest is linked to a regulatory element in a manner that allows expression of the nucleotide sequence (e.g., in an in vitro transcription/translation system or in a host cell when the vector is introduced into the host cell). Advantageous vectors include lentiviruses and adeno-associated viruses, and the type of these vectors may also be selected to target specific types of cells.

The term “regulatory element” is intended to include promoters, enhancers, internal ribosome entry sites (IRES), and other expression control elements (e.g., transcription termination signals such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990) (1990). Regulatory elements include those that guide constitutive expression of nucleotide sequences in many types of host cells and those that guide expression of nucleotide sequences only in certain host cells (e.g., tissue-specific regulatory sequences). Tissue-specific promoters may guide expression primarily in desired target tissues such as muscle, neuron, bone, skin, blood, particular organs (e. g., liver, pancreas) or particular cell types (e.g., lymphocytes). Regulatory elements may also guide expression in a time-dependent manner, e.g., in a cell cycle dependent or developmental stage dependent manner, which may or may not be tissue or cell type specific.

In some embodiments, the vector encodes a Cas12i protein comprising one or more nuclear localization sequences (NLSs), e.g., about or greater than about 1, 2, 3, 4, 5, 6, 7, 8, 9, 10 or more NLSs. More specifically, the vector comprises one or more NLSs that are not naturally occurring in the Cas12i protein. Most particularly, the NLS is present in 5′ and/or 3′ of the vector for the Cas12i protein sequence. In some embodiments, the protein targeting RNA comprises about or greater than about 1, 2, 3, 4, 5, 6, 7, 8, 9, 10 or more NLSs at or near the amino terminus and about or greater than about 1, 2, 3, 4, 5, 6, 7, 8, 9, 10 or more NLSs at or near the carboxyl terminus, or a combination of these (e.g., 0 or at least one or more NLSs at the amino terminus and 0 or one or more NLSs at the carboxyl terminus). When more than one NLSs are present, each of them may be selected independently of the others such that a single NLS may be present in more than one copies and/or in combination with one or more other NLSs in one or more copies. In some embodiments, NLS is considered to be near the N-terminus or C-terminus when its nearest amino acid is within about 1, 2, 3, 4, 5, 10, 15, 20, 25, 30, 40, 50, or more amino acids along the polypeptide chain from the N-terminus or C-terminus.

“Codon optimization” refers to a method of modifying a nucleic acid sequence in a target host cell to enhance expression by replacing at least one codon (e.g., about or greater than about 1, 2, 3, 4, 5, 10, 15, 20, 25, 50, or more codons) of a natural sequence with a codon that is more frequently or most frequently used in the gene of the host cell while maintaining the natural amino acid sequence. A variety of species show particular bias towards certain codons for particular amino acids. Codon bias (the difference in codon usage among organisms) is generally related to the translation efficiency of messenger RNA (mRNA), which in turn is thought to depend, inter alia, on the characteristics of the translated codons and the availability of specific transfer RNA (tRNA) molecules. The dominance of the selected tRNA in the cell generally reflects the codons most commonly used in peptide synthesis. Thus, genes can be tailored to optimize gene expression in a given organism based on codon optimization. Codon usage tables are readily available, for example, in the “codon usage database” in www.kazusa.orjp/codon/, and may be modified in a number of ways. See Nakamura, Y., et al. “Codon usage tabulated from the international DNA Sequence databases: status for the year 2000” Nucl. Acids Res. 28:292 (2000). Computerized algorithms for codon optimization of specific sequences for expression in specific host cells are also available, such as Gene Forge (Aptagen; Jacobus, PA). In some embodiments, one or more codons (e.g., 1, 2, 3, 4, 5, 10, 15, 20, 25, 50 or more or all codons) in a sequence encoding the Cas protein targeting DNA/RNA correspond to the codons most commonly used for particular amino acids. For codon usage in yeast, reference can be made to the online saccharomyces genome database available from www.yeastgenome.org/community/codon_usage.shtml, or Codon selection in yeast, Bennetzen and Hall, J Biol Chem. Mar. 25 1982; 257 (6): 3026-31. For codon usage in plants including algae, see Codon usage in higher plants, green algae, and cyanobacteria, Campbell and Gowri, Plant Physiol., January 1990; 92 (1): 1-11.; and Codon usage in plant genes, Murray et al., Nucleic Acids Res. Jan. 25, 1989; 17 (2): 477-98; or Selection on the codon bias of chloroplast and cyanelle genes in different plant and algal lineages, Morton BR, J Mol Evol. April 1998; 46 (4): 449-59.

### Delivery System

In some embodiments, the components of the CRISPR-Cas system may be delivered in various forms, such as a combination of DNA/RNA or RNA/RNA or protein RNA. For example, the Cas12i protein may be delivered as a polynucleotide encoding DNA or a polynucleotide encoding RNA or as a protein. The guide may be delivered as a polynucleotide encoding DNA or RNA. All possible combinations are contemplated, including mixed delivery forms.

In some aspects, the invention provides a method for delivering one or more polynucleotides, such as one or more vectors, one or more transcripts thereof, and/or one or more proteins transcribed therefrom as described herein, to host cells.

In some embodiments, one or more vectors that drive expression of one or more elements of the nucleic acid targeting system are introduced into host cells such that expression of elements of the nucleic acid targeting system guides formation of the nucleic acid targeting complex at one or more target sites. For example, the nucleic acid encoding effector enzymes and the nucleic acid encoding guide RNAs may each be operably linked to separate regulatory elements on separate vectors. The RNA of the nucleic acid targeting system can be delivered to a transgenic nucleic acid targeting effector protein animal or mammal, e.g., an animal or mammal that constitutively or inductively or conditionally expresses the nucleic acid targeting effector protein; or an animal or mammal that otherwise expresses the nucleic acid targeting effector protein or has cells containing the nucleic acid targeting effector protein, for example, by administering thereto one or more vectors encoding and expressing the in vivo nucleic acid targeting effector protein in advance. Alternatively, two or more elements regulated by the same or different regulatory elements may be combined in a single vector, while one or more additional vectors provide any components of the nucleic acid targeting system not contained in the first vector. The elements of the nucleic acid targeting system combined in the single vector may be arranged in any suitable orientation, for example, one element is positioned 5′ (“upstream”) relative to the second element or 3′ (“downstream”) relative to the second element. The coding sequence of one element may be on the same or opposite chain of the coding sequence of the second element and oriented in the same or opposite direction. In some embodiments, a single promoter drives the expression of transcripts encoding the nucleic acid targeting effector protein and the nucleic acid targeting guide RNA, and the transcripts are embedded into one or more intron sequences (e.g., each in a separate intron, two or more in at least one intron, or all in a single intron). In some embodiments, the nucleic acid targeting effector protein and the nucleic acid targeting guide RNA may be operably linked to the same promoter and expressed from the same promoter. Delivery vehicles, vectors, particles, nanoparticles, formulations and components thereof for expressing one or more elements of the nucleic acid targeting system are as used in the previous documents such as WO 2014/093622 (PCT/US2013/074667; the content of which is incorporated herein by reference in its entirety). In some embodiments, the vector comprises one or more insertion sites, such as a restriction endonuclease recognition sequence (also referred to as a “cloning site”). In some embodiments, one or more insertion sites (e.g., about or greater than about 1, 2, 3, 4, 5, 6, 7, 8, 9, 10 or more insertion sites) are located upstream and/or downstream of one or more sequence elements of one or more vectors. When a plurality of different guide sequences are used, a single expression construct may be used to target nucleic acids to various corresponding target sequences within active target cells. For example, a single vector may comprise about or greater than about 1, 2, 3, 4, 5, 6, 7, 8, 9, 10, 15, 20 or more guide sequences. In some embodiments, about or greater than about 1, 2, 3, 4, 5, 6, 7, 8, 9, 10 or more such vectors containing guide sequences may be provided and optionally delivered to the cells. In some embodiments, the vector comprises a regulatory element operably linked to an enzyme coding sequence encoding the nucleic acid targeting effector protein. The nucleic acid targeting effector protein or one or more nucleic acid targeting guide RNAs may be delivered separately; and advantageously at least one of these is delivered via a particle complex. The nucleic acid targeting effector protein mRNA may be delivered prior to the nucleic acid targeting guide RNA to allow time for expression of the nucleic acid targeting effector protein. The nucleic acid targeting effector protein mRNA may be administered 1-12 h (preferably about 2-6 h) prior to administration of the nucleic acid targeting guide RNA. Alternatively, the nucleic acid targeting effector protein mRNA and the nucleic acid targeting guide RNA may be administered together. Advantageously, the second boosted dose of guide RNA may be administered 1-12 h (preferably about 2-6 h) after the initial administration of the nucleic acid targeting effector protein mRNA+guide RNA. The additional administration of the nucleic acid targeting effector protein mRNA and/or guide RNA may be useful to achieve the most effective level of genomic modification.

Conventional viral and non-viral based gene transfer methods can be used to introduce nucleic acids into mammalian cells or target tissues. Such methods can be used to administer nucleic acids encoding the components of a nucleic acid targeting system to cells in culture or in a host organism. A non-viral vector delivery system comprises DNA plasmids, RNA (e.g., transcripts of vectors as described herein), naked nucleic acids, and nucleic acids complexed with a delivery vehicle such as liposome. Viral vector delivery systems comprise DNA and RNA viruses that have episomal or integrated genomes upon delivery to cells. For a review of gene therapy procedures, see Anderson, Science 256:808-813 (1992); Nabel and Felgner, TIBTECH 11:211-217 (1993); Mitani and Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer and Perricaudet, British Medical Bulletin 51 (1): 31-44 (1995); Haddada et al., Current Topics in Microbiology and Immunology, Doerfler and Böhm (eds.) (1995); and Yu et al., Gene Therapy 1:13-26 (1994). Non-viral delivery methods for nucleic acids include lipid transfection, nuclear transfection, microinjection, biolistics, virosomes, liposomes, immunoliposomes, polycations or lipids: nucleic acid conjugates, naked DNA, artificial virosomes, and reagent-enhanced DNA uptake. Lipid transfection is described, for example, in U.S. Pat. Nos. 5,049,386, 4,946,787; and 4,897,355, and lipid transfection reagents are commercially available (e.g., Transfectam™ and Lipofectin™). Cationic and neutral lipids suitable for effective receptor recognition lipid transfection for polynucleotides include those in Felgner, WO 91/17424; WO 91/16024, which can be delivered to cells (e.g., in vitro or ex vivo administration) or target tissues (e.g., in vivo administration).

Plasmid delivery involves cloning the guide RNA into a plasmid expressing the CRISPR-Cas protein and transfecting DNA in cell culture. The plasmid backbone is commercially available and does not require specific equipment. Advantageously, they are modularized, and can carry CRISPR-Cas coding sequences of different sizes, including sequences encoding larger-sized protein, as well as selection markers. Also, plasmids are advantageous in that they ensure transient but continuous expression. However, the delivery of plasmids is not direct, usually leading to low in vivo efficiency. Continuous expression may also be disadvantageous in that it can increase off-target editing. In addition, excessive accumulation of CRISPR-Cas proteins may be toxic to cells. Finally, plasmids always have the risk of random integration of dsDNA into the host genome, more particularly considering the risk of double-stranded breakage (on-target and off-target).

The preparation of lipid: nucleic acid complexes (including targeting liposomes, such as immunolipid complexes) are well known to those skilled in the art (see, for example, Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028 and 4,946,787), as will be discussed in more detail below.

The use of RNA or DNA virus-based systems to deliver nucleic acids takes advantage of a highly evolved process of targeting viruses to specific cells in vivo and transporting viral payloads to the nuclei. The viral vectors may be administered directly to a patient (in vivo) or they may be used to treat cells in vitro, and the modified cells may optionally be administered to a patient (ex vivo). Conventional virus-based systems may include retrovirus, lentivirus, adenovirus, adeno-associated virus and herpes simplex virus vectors for gene transfer. Integration into the host genome by retroviral, lentiviral and adeno-associated virus gene transfer methods often results in long-term expression of the inserted transgene. In addition, high transduction efficiency has been observed in many different cell types and target tissues.

The tropism of a retrovirus can be altered by incorporation of a foreign envelope protein to expand the potential target population of target cells. Lentiviral vectors are retroviral vectors that can transduce or infect non-dividing cells and generally produce high viral titers. Therefore, the choice of a retroviral gene transfer system will depend on the target tissue. Retroviral vectors consist of cis-acting long terminal repeats with a packaging capacity up to 6-10 kb of foreign sequences. The minimal cis-acting LTR is sufficient to replicate and package the vector, which is then used to integrate therapeutic genes into target cells to provide permanent transgene expression. Widely used retroviral vectors include vectors based on murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenovirus-based systems may be used. Adenovirus-based vectors provide high transduction efficiency in many cell types and do not require cell division. With such vectors, high titers and expression levels have been achieved. The vector can be mass produced in a relatively simple system. Adeno-associated virus (“AAV”) vectors can also be used to transduce cells with target nucleic acids, e.g., in the in vitro production of nucleic acids and peptides, as well as in in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors is described in numerous publications, including U.S. Pat. No. 5,173,414; Tratschin et al., Mol. Cell. Biol. 5:3251-3260 (1985); Tratschin et al., Mol. Cell. Biol. 4:2072-2081 (1984); Hermonat and Muzyczka, PNAS 81:6466-6470 (1984); and Samulski et al., J. Virol. 63:03822-3828 (1989).

The invention provides AAV comprising or consisting essentially of an exogenous nucleic acid molecule encoding a CRISPR system, e.g., a plurality of cassettes comprising or consisting of a first cassette comprising or consisting essentially of a promoter, a nucleic acid molecule encoding a CRISPR associated (Cas) protein (putative nuclease or helicase protein), e.g., Cas12i and a terminator, and one or more, advantageously up to the packaging size limit of the vector, for example five cassettes in total (including the first cassette) comprising or consisting essentially of a promoter, a nucleic acid molecule encoding guide RNA (gRNA) and a terminator (for example, each cassette is schematically represented as promoter-gRNA1-terminator, promoter-gRNA2-terminator . . . promoter-gRNA (N)-terminator, where N is the upper limit of the package size limits of the insertable vectors), or two or more individual rAAVs, wherein each rAAV contains one or more cassettes of the CRISPR system, for example, a first rAAV contains a first cassette comprising or consisting essentially of a promoter, a Cas-encoding nucleic acid molecule such as Cas (Cas12i) and a terminator, and a second rAAV contains one or more cassettes, each cassette comprising or consisting essentially of a promoter, a nucleic acid molecule encoding guide RNA (gRNA) and a terminator (e.g., each cassette is schematically represented as promoter-gRNA1-terminator, promoter-gRNA2-terminator . . . promoter-gRNA (N)-terminator, where N is the upper limit of the package size limits of the insertable vectors). Alternatively, a single crRNA/gRNA array can be used for multiplex gene editing, since Cas12i can process its own crRNA/gRNA. Thus, rather than comprising a plurality of cassettes to deliver gRNA, rAAV can contain a single cassette comprising or consisting essentially of a promoter, a plurality of crRNA/gRNA, and a terminator (e.g., schematically represented as promoter-gRNA1-gRNA2 . . . gRNA (N)-terminator, where N is the upper limit of the package size limits of the insertable vector). See Zetsche et al., Nature Biotechnology 35, 31-34 (2017), which is incorporated herein by reference in its entirety. Since rAAV is a DNA virus, the nucleic acid molecule in the discussion herein with respect to AAV or rAAV is advantageously DNA. In some embodiments, the promoter is advantageously human synaptophysin I promoter (hSyn). Other methods for delivering nucleic acids to cells are known to those skilled in the art. See, for example, US20030087817, which is incorporate herein by reference.

In another embodiment, cocal vesiculovirus enveloped pseudoretrovirus vector particles are considered (see, for example, U.S. Patent Publication No. 20120164118 assigned to Fred Hutchinson Cancer Research Center). Cocal virus belongs to the genus vesiculovirus and is the pathogen of vesicular stomatitis in mammals. The cocal virus was originally isolated from mites in Trinidad (Jonkers et al., Am. J. Vet. Res. 25:236-242 (1964)), and cocal virus infections have been identified in insects, cattle, and horses in Trinidad, Brazil, and Argentina. Many vesicular viruses that infect mammals have been isolated from naturally infected arthropods, suggesting that they are vector-borne. Antibodies to vesicular viruses are widely available in rural areas where the viruses are obtained locally and in laboratories; their infections in humans usually cause flu-like symptoms. The envelope glycoprotein of cocal virus shares 71.5% identity to VSV-G Indiana at the amino acid level, and phylogenetic comparison of the vesicular virus envelope gene shows that cocal virus is serologically distinct from, but most closely related to, the VSV-G Indiana strain of vesicular virus. Jonkers et al., Am. J. Vet. Res. 25:236-242 (1964) and Travassos da Rosa et al., AM. J. Tropical Med. & Hygiene 33:999-1006 (1984). Cocal vesicular virus envelope pseudoretrovirus vector particles may include, for example, lentivirus, alpha retrovirus, beta retrovirus, gamma retrovirus, delta retrovirus and epsilon retrovirus vector particles, which may comprise retrovirus Gag, Pol and/or one or more helper proteins and cocal vesicular virus envelope proteins. In certain aspects of these embodiments, the Gag, Pol and helper proteins are lentiviruses and/or gamma retroviruses.

In some embodiments, host cells are transiently or non-transiently transfected with one or more vectors described herein. In some embodiments, when the cells are naturally present in the subject, the cells are transfected, and optionally reintroduced therein. In some embodiments, the transfected cells are taken from a subject. In some embodiments, the cells are derived from cells from a subject, such as cell lines. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelium, BALB/3T3 mouse embryonic fibroblasts, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cell, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cell, K562 cell, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell line, Peer, PNT-1A/PNT 2, RenCa, RIN-5F, RMA/RMAS, Saos-2 cell, Sf-9, SkBr3, T2, T-47D, T84, THP1 cell line, U373, U87, U937, VCaP, Vero cell, WM39, WT-49, X63, YAC-1, YAR and transgenic varieties thereof. Cell lines may be obtained from a variety of sources known to those skilled in the art (see, for example, the American Type Culture Collection (ATCC) (Manassas, Va.)).

In particular embodiments, the transient expression and/or presence of one or more components of an AD-functionalized CRISPR system may be of interest, for example, to reduce off-target effects. In some embodiments, cells transfected with one or more vectors described herein are used to establish novel cell lines comprising one or more vector derived sequences. In some embodiments, cells transiently transfected (e.g., transiently transfected with one or more vectors, or transfected with RNA) with components of the AD-functionalized CRISPR system as described herein and modified by the activity of the CRISPR complex are used to establish new cell lines comprising cells containing the modifications but lacking any other exogenous sequence. In some embodiments, cells transiently or non-transiently transfected with one or more vectors described herein, or cell lines derived from such cells, are used to evaluate one or more test compounds.

In some embodiments, direct introduction of RNA and/or protein into host cells is contemplated. For example, the CRISPR-Cas protein may be delivered as encoded mRNA along with guide RNA from in vitro transcription. Such methods may reduce and ensure the action time of the CRISPR-Cas protein and further prevent long-term expression of the components of the CRISPR system.

In some embodiments, the RNA molecules of the invention are delivered as liposomes or lipofectin formulations and the like, and may be prepared by methods well known to those skilled in the art. Such methods are described, for example, in U.S. Pat. Nos. 5,593,972, 5,589,466 and 5,580,859, which are incorporated herein by reference in their entirety. Delivery systems specifically designed to enhance and improve the delivery of siRNA into mammalian cells have been developed (see, e.g., Shen et al., FEBS Let. 2003, 539:111-114; Xia et al., Nat. Biotech. 2002, 20:1006-1010; Reich et al., Mol. Vision. 2003, 9:210-216; Sorensen et al., J. Mol. Biol. 2003, 327:761-766; Lewis et al., Nat. Gen. 2002, 32:107-108; and Simeoni et al., NAR 2003, 31, 11:2717-2724) and may be applied to the invention. siRNA have recently been successfully used to inhibit gene expression in primates (see, for example, Tolentino et al., Retina 24 (4): 660), which can also be applied to the invention.

In fact, RNA delivery is a useful method of delivery in vivo. Cas12i, adenosine deaminase, and guide RNA may be delivered to cells using liposomes or particles. Thus, the delivery of CRISPR-Cas proteins (e.g., Cas12i), the delivery of adenosine deaminase (which may be fused to CRISPR-Cas proteins or adaptor proteins) and/or the delivery of RNA of the invention may be in the form of RNA and via microvesicles, liposomes or particles or nanoparticles. For example, Cas12i mRNA, adenosine deaminase mRNA, and guide RNA may be packaged into liposome particles for delivery in vivo. Liposome transfection reagents, such as lipofectamine from Life Technologies and other reagents on the market, can efficiently deliver RNA molecules into the liver. In some embodiments, the lipid nanoparticle (LNP) comprises ALC-0315: Cholesterol: PEG-DMG: DOPE at a molar ratio of 50 mM:50 mM:10 mM:20 mM. In some embodiments, the LNP encapsulates both Cas12i and its corresponding crRNA (e.g., SiCas12i: crRNA with a weight ratio of 1:1), or nucleic acid(s) encoding thereof. In some embodiments, the LNP comprising Cas12i and/or crRNA (or nucleic acid(s) encoding thereof) is administered to an individual (e.g., human) by intravenous infusion.

Delivery of RNA also preferably includes RNA delivery via particles (Cho, S., Goldberg, M., Son, S., Xu, Q., Yang, F., Mei, Y., Bogatyrev, S., Langer, R., and Anderson, D., Lipid-like nanoparticles for small interfering RNA delivery to endothelial cells, Advanced Functional Materials, 19:3112-3118, 2010) or via exosomes (Schroeder, A., Levins, C., Cortez, C., Langer, R., and Anderson, D., Lipid-based nanotherapeutics for siRNA delivery, Journal of Internal Medicine, 267:9-21, 2010, PMID: 20059641). In fact, exosomes have been shown to be particularly useful in delivering siRNA, and this system is somewhat similar to the CRISPR system. For example, El-Andaloussi S et al. (“Exosome-mediated delivery of siRNA in vitro and in vivo.” Nat Protoc. December 2012; 7 (12): 2112-26. doi: 10.1038/nprot.2012.131. Electronically published on Nov. 15, 2012) describes how exosomes can become promising tools for drug delivery across different biological barriers and for in vitro and in vivo delivery of siRNA. Their method involves generating targeting exosomes by transfecting an expression vector comprising an exosome protein fused to a peptide ligand. The exosome is then purified and characterized from the transfected cell supernatant, and the RNA is loaded into the exosome. Delivery or administration according to the invention may be performed using exosomes, particularly (but not limited to) the brain. Vitamin E (a-tocopherol) can be conjugated with CRISPR Cas and delivered to the brain along with high-density lipoprotein (HDL), for example, in a manner similar to that of Uno et al. (HUMAN GENE THERAPY 22:711-719 (June 2011)) for delivery of short interfering RNA (siRNA) to the brain. Infusion to mice is performed via an Osmotic micro-pump (Model 1007D; Alzet, Cupertino, CA) filled with phosphate buffered saline (PBS) or free TocsiBACE or Toc-siBACE/HDL and connected to brain infusion kit 3 (Alzet). A brain infusion cannula is placed approximately 0.5 mm posterior to the anterior fontanel at the midline for infusion into the dorsal side of the third ventricle. Uno et al. found that Toc-siRNA containing HDL as low as 3 nmol could induce the target reduction considerably by the same ICV infusion method. In the invention, for humans, similar doses of CRISPR Cas conjugated to a-tocopherol and co-administered with brain-targeted HDL may be considered, for example, about 3 nmol to about 3 μmol of brain-targeted CRISPR Cas may be considered. Zou et al. (HUMAN GENE THERAPY 22:465-475 (April 2011)) describes a lentivirus-mediated delivery method of short hairpin RNA targeting PKCγ for in vivo gene silencing in the spinal cords of rats. Zou et al. administered approximately 10 μl of recombinant lentivirus through an intrathecal catheter with a titer of 1×10″ transducing units (TU)/ml. In the invention, for humans, a similar dose of CRISPR Cas expressed in a brain-targeted lentivirus vector may be considered, for example, about 10-50 ml of brain-targeted CRISPR Cas in a lentivirus with a titer of 1×10⁹ transduced units (TU)/ml may be considered.

Other suitable modifications and variations of the methods of the invention described herein will be apparent to those skilled in the art and may be made using suitable equivalents without departing from the scope of the invention or the embodiments disclosed herein.

Further embodiments are illustrated in the following Examples which are given for illustrative purposes only and are not intended to limit the scope of the disclosure.

### EXAMPLES

### Material and Methods

Unless otherwise specified, the experimental methods used in the Examples are conventional.

Unless otherwise specified, the materials, reagents, etc., used in the Examples are commercially available.

Unless otherwise specified, the following materials and experimental methods were used in the Examples.

### Plasmid Vector Construction.

Human codon-optimized Cas12i, TadA8e and human APOBEC3A genes were synthesized by the GenScript Co., Ltd., and cloned to generate pCAG_NLS-Cas12i-NLS_pA_pU6_BpiI_pCMV_mCherry_pA by Gibson Assembly. crRNA oligos were synthesized by HuaGene Co., Ltd., annealed and ligated into BpiI site to produce the pCAG_NLS-Cas12i-NLS_pA_pU6_crRNA_pCMV_mCherry_pA.

### Cell Culture, Transfection, and Flow Cytometry Analysis.

The mammalian cell lines used in this study were HEK293T and N2A. Cells were cultured in Dulbecco's modified Eagle's medium (DMEM) supplemented with 10% FBS, penicillin/streptomycin and GlutMAX. Transfections were performed using Polyetherimide (PEI). For variant screening, HEK293T cells were cultured in 24-well plates, and after 12 hours 2 μg of the plasmids (1 μg of an expression plasmid and 1 μg of a reporter plasmid) were transfected into these cells with 4 μL PEI. 48 hours after transfection, BFP, mCherry, and EGFP fluorescence were analyzed using a Beckman CytoFlex flow-cytometer. For assay of mutations in target sites of endogenous genes, 1 μg of expression plasmid was transfected into HEK293T or N2A cells, which were then sorted using a BD FACS Aria III, BD LSRFortessa X-20 flow cytometer, 48 hours after transfection.

### Detection of Gene Editing Frequency.

Six thousand sorted cells were lysed in 20 μl of lysis buffer (Vazyme). Targeted sequence primers were synthesized and used in nested PCR amplification by Phanta Max Super-Fidelity DNA Polymerase (Vazyme). Targeted deep sequence analysis was used to determine indel frequencies. A-to-G or C-to-T editing frequencies were calculated by targeted deep sequence analysis or Sanger sequencing and EditR. A-to-G editing purity were calculated as A-to-G editing efficiency/(A-to-T editing efficiency+A-to-C editing efficiency+A-to-G editing efficiency). C-to-T editing purity were calculated as C-to-T editing efficiency/(C-to-A editing efficiency+C-to-G editing efficiency+C-to-T editing efficiency).

### PEM-seq.

PEM-seq in HEK293 cells was performed as previously described²³. Briefly, all-in-one plasmids containing LbCas12a, Ultra-AsCas12a, hfCas12Max, ABR001 or Cas12i2HiFi with targeting TTR.2 crRNA were transfected into HEK293 cells by PEI respectively, and after 48 hrs, positive cells were harvested for DNA extraction. The 20 μg genomic DNA was fragmented with a peak length of 300-700 bp by Covaris sonication. DNA fragments was tagged with biotin by a one-round biotinylated primer extension at 5′-end, and then primer removal by AMPure XP beads and purified by streptavidin beads. The single-stranded DNA on streptavidin beads is ligased with a bridge adapter containing 14-bp RMB, and PCR product was performed nested PCR for enriching DNA fragment containing the bait DSB and tagged with illumine adapter sequences. The prepared sequencing library was sequencing on an Hi-seq 2500, with a 2×150 bp.

### RNP Delivery and Ex Vivo Editing.

RNP was complexed by mixing purified hfCas12Max proteins with chemically synthesized RNA oligonucleotides (Genscript) at a 1:2 molar ratio in 1×PBS. RNP was incubated at room temperature for >15 min prior to electroporation with Lonza® 4D-Nucleofector™. 0.2× 106 cells were resuspended in 20 μL of Lonza buffer and mixed with 5 μL RNP with different concentrations electroporated according to Lonza specifications. HEK293 or CD3+ T cells were harvested 72 hrs post-electroporation for targeted deep sequence analysis.

### LNP Delivery and In Vivo Editing.

LNPs were formulated with ALC0315, cholesterol, DMG-PEG2k, DSPC in 100% ethanol, carrying in vitro transcription (IVT) mRNA and chemically synthesized RNA oligonucleotides (Genscript) with a 1:1 weight ratio. LNPs were formed according to the manufacturer's protocol, by microfluidic mixing the lipid with RNA solutions using a Precision Nano-systems NanoAssemblr Benchtop Instrument. LNPs diluted in PBS were transfected into N2a cells at 0.1, 0.3, 0.5, 1 μg RNA, or delivered into C57 mouse with different dose by through tail intravenous injection. Cells were harvested 48 hrs post-transfection for lysis and targeted deep sequence analysis. For in vivo editing, liver tissue was collected from the left or median lateral lobe of each mouse 7 days post-injection for DNA extraction and targeted deep sequence analysis.

### Zygote Injection and Embryo Culturing.

Super ovulated female C57 mice (7-8 weeks old) by injecting 5 IU of pregnant mare serum gonadotropin (PMSG), followed by 5 IU of human chorionic gonadotropin (hCG) 48 hrs later were mated to B6D2F1 males, and fertilized embryos were collected from oviducts 20 hrs post hCG injection. For zygote injection, hfCas12Max mRNA (100 ng/μL) and sgRNA (100 ng/μL) were mixed and injected into the cytoplasm of fertilized eggs in a droplet of HEPES-CZB medium containing 5 mg/ml cytochalasin B (CB) using a FemtoJet microinjector (Eppendorf) with constant flow settings. The injected zygotes were cultured in KSOM medium with amino acids at 37° C. under 5% CO₂ in air to blastocysts and harvested for targeted deep sequence analysis.

### Example 1 Identification of Cas12i Proteins and Evaluation of their dsDNA Cleavage Activity

In order to identify more Cas12i proteins, the applicant developed and employed a bioinformatics pipeline to annotate Cas12i proteins, CRISPR arrays, DR sequences, and predicted PAM preferences, and identified 10 Cas12i proteins and associated sequences in Table 1 below.

TABLE 1

| | | SEQ ID NO: | | | |
|---|---|---|---|---|---|
| | | | | Codon- | |
| | | Cas12i | Corres- | optimized | |
| Name | Name | amino | ponding | Cas12i | Cas12i |
| Cas12i | Cas12i | acid | DR | coding | coding |
| protein | protein | sequence | sequence | sequences | sequence |
| SiCas12i | Cas12i12 | 1 | 11 | 21 | 31 |
| | (xCas12i) | | | | |
| Si2Cas12i | Cas12i3 | 2 | 12 | 22 | 32 |
| WiCas12i | Cas12i7 | 3 | 13 | 23 | 33 |
| Wi2Cas12i | Cas1218 | 4 | 14 | 24 | 34 |
| Wi3Cas12i | Cas12i9 | 5 | 15 | 25 | 35 |
| SaCas12i | Cas12i11 | 6 | 16 | 26 | 36 |
| Sa2Cas12i | Cas12i4 | 7 | 17 | 27 | 37 |
| Sa3Cas12i | Cas1215 | 8 | 18 | 28 | 38 |
| WaCas12i | Cas12i6 | 9 | 19 | 29 | 39 |
| Wa2Cas12i | Cas12i10 | 10 | 20 | 30 | 40 |

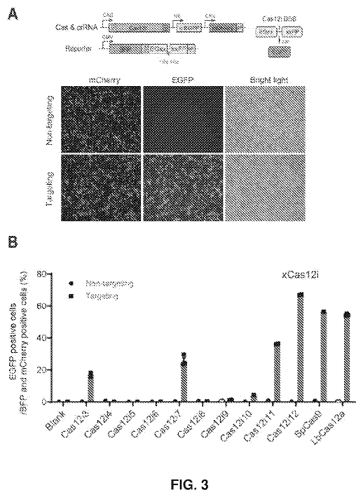

To evaluate the dsDNA cleavage activity of these Cas12i proteins in mammalian cells, the applicant designed a dual plasmid fluorescent reporter system, which detected the increased enhanced green fluorescent protein (EGFP) signal intensity activated by Cas-mediated dsDNA cleavage or double strand breaks (FIG. 3A). This system relied on the co-transfection of an expression plasmid encoding mCherry, a nuclear localization signal (NLS)-tagged Cas protein, and a guide RNA (gRNA, or crRNA), and a reporter plasmid encoding BFP and activatable EGxxFP cassette, which is EGxx-target site-xxFP11. EGFP activation was carried out by Cas mediated DSB and single-strand annealing (SSA)-mediated repair.

Specifically, referring to FIG. 3A, the reporter plasmid comprised a polynucleotide encoding, from 5′ to 3′, BFP-P2A-activatable EGxxxxFP (SEQ ID NO: 41) (EGxx-insertion sequence (SEQ ID NO: 42) (containing, from 5′ to 3′, a protospacer adjacent motif (PAM)) of [symbol] for Cas12i protein, a protospacer sequence (SEQ ID NO: 43) (which is the reverse complementary sequence of a target sequence (SEQ ID NO: 44)), and a protospacer adjacent motif (PAM)) of [symbol] for Cas9 protein-xxFP), followed by a bGH polyA (SEQ ID NO: 448) coding sequence, operably linked to human CMV promoter (SEQ ID NO: 447). The protospacer sequence (SEQ ID NO: 43) contained a premature stop codon [symbol] that prevented the expression of EGFP and hence emission of green fluorescent signals. The BFP coding sequence expresses BFP to indicate the successful transfection of the reporter plasmid into host cells through blue fluorescence.

Most of the known Cas12i proteins recognize a 5′-T-rich PAM in dsDNA, while Cas9 recognizes a 3′-G-rich PAM in dsDNA. The co-existence of the 5′ PAM of [symbol] for Cas12i protein and the 3′ PAM of [symbol] for Cas9 protein flanking the protospacer sequence (SEQ ID NO: 43) allows the simultaneous evaluation and comparison of dsDNA cleavage activity of Cas12i protein and Cas9 protein.

SEQ ID NO: 41
Activatable EGxxxxFP coding sequence,
atgagcgagctgattaaggagaacatgcacatgaagctgtatatggagggcaccgtggacaaccatcacttcaagtgcacatccgagggcgaaggc
aagccctacgagggcacccagaccatgagaatcaaggtggtcgagggcggccctctccccttcgccttcgacatcctggctactagcttcctcta
cggcagcaagaccttcatcaaccacacccagggcatccccgacttcttcaagcagtccttccctgagggcttcacatgggagagagtca
ccacatacgaggacgggggcgtgctgaccgctacccaggacaccagcctccaggacggctgcctcatctacaacgtcaagatcagaggggtgaac
ttcacatccaacggccctgtgatgcagaagaaaacactcggctgggaggccttcaccgagacactgtaccccgctgacggcggcctggaaggcaga
aacgacatggccctgaagctcgtggggggagccatctgatcgcaaacatcaagaccacatatagatccaagaaacccgctaagaacctcaagatgcc
tggcgtctactatgtggactacagactggaaagaatcaaggaggccaacaacgagacatacgtcgagcagcacgaggtggcagtggccagatactgc
gacctccctagcaaactggggcacaagctgaatgaattcgagggcaggggcagcctgctgacctgcggcgacgtggaggagaaccccggccccatgg
tgagcaagggcgaggagctgttcaccggggtggtgcccatcctggtcgagctggacggcgacgtaaacggccacaagttcagcgtgtccggcgaggg
cgagggcgatgccacctacggcaagctgaccctgaagttcatctgcaccaceggcaagctgcccgtgccctggcccaccctcgtgaccaccctgacc
tacggcgtgcagtgcttcagccgctaccccgaccacatgaagcagcacgacttcttcaagtccgccatgcccgaaggctacgtccaggagcgc
accatcttcttcaaggacgacggcaactacaagacccgcgccgaggtgaagttcgagggcgacaccctggtgaaccgcatcgagctgaagggc
atcgacttcaaggaggacggcaacatcctggggcacaagctggagtacaactacaacagccacaacgtctatatcatggccgacaagcagaag
aacggcatcaaggtgaacttcaag

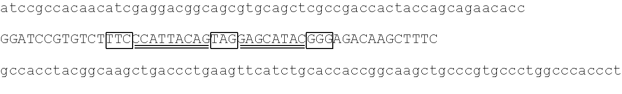

cgtgaccaccctgacctacggcgtgcagtgcttcagccgctaccccgaccacatgaagcagcacgacttcttcaagtccgccatgcccgaaggcta
cgtccaggagcgcaccatcttcttcaaggacgacggcaactacaagacccgcgccgaggtgaagttcgagggcgacaccctggtgaaccgcatcgag
ctgaagggcatcgacttcaaggaggacggcaacatcctggggcacaagctggagtacaactacaacagccacaacgtctatatcatggccgacaa
gcagaagaacggcatcaaggtgaacttcaagatccgccacaacatcgaggacggcagegtgcagctegccgaccactaccagcagaacacccccat
atggtcctgctggagttcgtgaccgccgccgggatcactctcggcatggacg
agctgtacaagtaa
SEQ ID NO: 42
Insertion sequence,

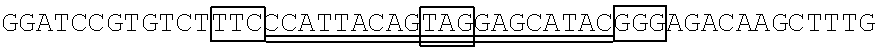

SEQ ID NO: 43
Protospacer sequence (Reverse complementary sequence of the target sequence), 20bp,

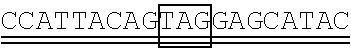

SEQ ID NO: 44
Target sequence, 20 nt,

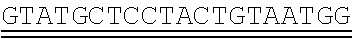

SEQ ID NO: 45
EGxxxxFP-targeting spacer sequence, 20 nt,

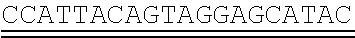

SEQ ID NO: 46
Non-targeting (“NT”) spacer sequence, 20 nt,
GGTCTTCGATAAGAAGACCT
PAM for Cas12i

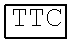

PAM for Cas9

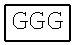

Also referring to FIG. 3A, the expression plasmid comprised from 5′ to 3′ i) a Cas12i coding sequence codon optimized for expression in mammalian cells (one of SEQ ID NOs: 31-40) encoding a Cas12i protein (one of SEQ ID NOs: 1-10) flanked by a SV40 NLS (SEQ ID NO: 444) coding sequence on its 5′ end and a NP NLS (SEQ ID NO: 445) coding sequence on its 3′ end, followed by a bGH polyA (SEQ ID NO: 448) coding sequence, operably linked to CAG promoter (SEQ ID NO: 500), ii) a sequence encoding a guide RNA (gRNA) in the configuration of 5′-DR sequence-spacer sequence-3′ operably linked to human U6 promoter (SEQ ID NO: 446); and iii) a coding sequence for mCherry followed by a bGH polyA (SEQ ID NO: 448) coding sequence operably linked to human CMV promoter (SEQ ID NO: 447). The mCherry coding sequence expresses mCherry to indicate the successful transfection of the expression plasmid into host cells through red fluorescence.

In the event that both the target sequence on the target strand and the protospacer sequence on the nontarget strand of the target dsDNA are successfully cleaved by a Cas12i protein guided by a gRNA to generate a double-strand break (DSB), the subsequent DNA repairing such as single-strand annealing (SSA)-mediated repair trigged by the DSB would restore the EGFP coding sequence to express EGFP with green fluorescence emission indicative of dsDNA cleavage activity.

For test groups, the spacer sequence comprised in the gRNA (“crEGFP”, one of SEQ ID NOs: 51-60) for use with each corresponding tested Cas12i protein (one of SEQ ID NOs: 1-10) is a EGxxxxFP-targeting spacer sequence (SEQ ID NO: 45) designed to target and hybridize to the target sequence (SEQ ID NO: 44), and the DR sequence in the gRNA (one of SEQ ID NOs: 51-60) is a DR sequence (one of SEQ ID NOs: 11-20) corresponding to each tested Cas12i protein (one of SEQ ID NOs: 1-10), as shown in Table 2.

TABLE 2

| | SEQ ID NO: | | |
|---|---|---|---|
| | DR | Spacer | Guide |
| Cas12i protein | sequence | sequence | RNA |
| SiCas12i (xCas12i) | 11 | 45 | 51 |
| Si2Cas12i | 12 | 45 | 52 |
| WiCas12i | 13 | 45 | 53 |
| Wi2Cas12i | 14 | 45 | 54 |
| Wi3Cas12i | 15 | 45 | 55 |
| SaCas12i | 16 | 45 | 56 |
| Sa2Cas12i | 17 | 45 | 57 |
| Sa3Cas12i | 18 | 45 | 58 |
| WaCas12i | 19 | 45 | 59 |
| Wa2Cas12i | 20 | 45 | 60 |

For negative control (“NT”) for each tested Cas12/9 protein (Cas12i, SpCas9, LbCas12a), a non-targeting spacer sequence (“NT”, SEQ ID NO: 46) incapable of hybridizing to the target sequence (SEQ ID NO: 44) was used in place of the EGxxxxFP-targeting spacer sequence (SEQ ID NO: 45), while the other elements of each tested CRISPR-Cas12/9 system remained.

For positive control, CRISPR-SpCas9 and CRISPR-LbCas12a systems each comprising a Cas protein and a guide RNA as shown in Table 3 below were used in place of the tested CRISPR-Cas12i systems in Tables 1 and 2 above, using the same EGxxxxFP-targeting spacer sequence (SEQ ID NO: 45). Note that the gRNA for the CRISPR-SpCas9 system was in the configuration of 5′-spacer sequence-scaffold sequence-3′, and the gRNA for the CRISPR-LbCas12a system was in the configuration of 5′-DR sequence-spacer sequence-3′.

TABLE 3

| Control | Control | |
|---|---|---|
| Cas | Cas amino | |
| protein | acid sequence | Guide RNA |
| SpCas9 | SEQ ID NO: 47 | SEQ ID NO: 48 |
| LbCas12a | SEQ ID NO: 49 | SEQ ID NO: 50 |

HEK293T cells were cultured in 24-well tissue culture plates according to standard methods for 12 hours, before the reporter and expression plasmids were co-transfected into the cells using standard polyethyleneimine (PEI) transfection. The transfected cells were then cultured at 37° C. under 5% CO₂ for 48 hours. Then the cultured cells were analyzed by flow cytometry for BFP, EGFP, and mCherry fluorescent signals. A “blank” control group was also set up, where only the reporter plasmid was transfected, and no expression plasmid was introduced.

The dsDNA cleavage activities of the tested Cas proteins were calculated as the percentage of EGFP positive cells in BFP & mCherry dual-positive cells (“EGFP⁺”, indicating dsDNA cleavage at the indicated target site on the reporter plasmid; “mCherry⁺ BFP⁺”, indicating successful co-transfection and co-expression of the expression and reporter plasmids). The higher the % EGFP⁺/mCherry⁺ BFP⁺ is, the higher the dsDNA cleavage activity would be.

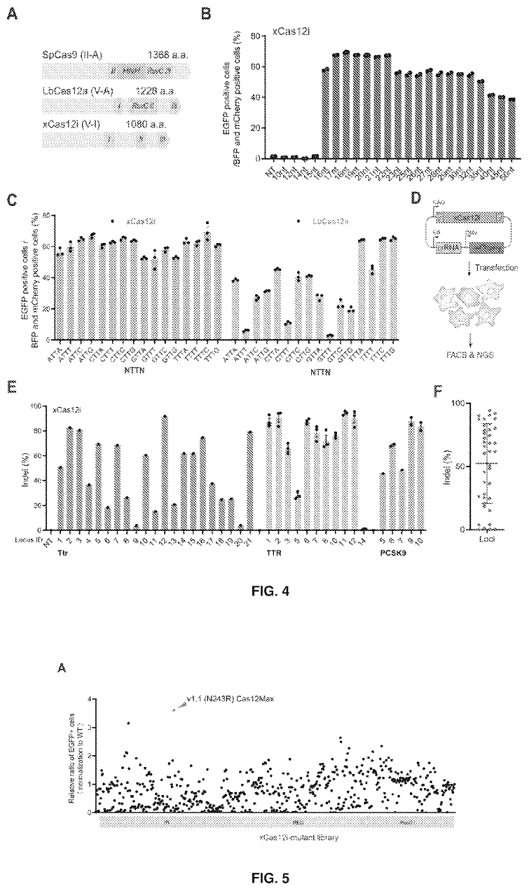

Using this dual plasmid fluorescent reporter system, it was observed that five Cas12i proteins (Cas12i3, Cas12i7, Cas12i10, Cas12i11, and Cas12i12 (also referred to as SiCas12i or xCas12i herein)) exhibited targeted gRNA induced significant activation of EGFP expression indicative of significant dsDNA cleavage (FIG. 1A, FIG. 3B), and among them, Cas12i12 even exhibited a higher dsDNA cleavage activity than both LbCas12a and SpCas9 as determined by Fluorescence Activated Cell Sorter (FACS) analysis (FIG. 1A, FIG. 3B). The xCas12i was smaller in size compared to SpCas9 and LbCas12a (FIG. 4A).

### Example 2 Evaluation of Effective Spacer Sequence Length for xCas12i

Using the dual plasmid fluorescent reporter system in Example 1, to test the effective spacer sequence length for xCas12i, 22 spacer sequences of different lengths ranging from 10 to 50 nt (SEQ ID NOs: 45 and 61-81 as shown in Table 4 below) were designed to target and hybridize to the reverse complementary sequence of a protospacer sequence (SEQ ID NO: 43, or one of SEQ ID NOs: 61-81) in the insertion sequence (SEQ ID NO: 42) of the GFxxxxFP reporter plasmid in Example 1, wherein the 20 nt spacer sequence in Table 4 is exactly the EGxxxxFP-targeting spacer sequence (SEQ ID NO: 45) in Example 1. To evaluate the additional spacer sequence lengths, the EGxxxxFP targeting spacer sequence (SEQ ID NO: 45) in the guide RNA encoded in the expression plasmid was replaced with the spacer sequence in respective length (one of SEQ ID NOs: 61-81) in Table 4, while the other elements of the dual plasmid fluorescent reporter system remained. To save drafting, the sequences in Table 4 refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NOs: 61-81 in the sequence listing are annotated as spacer sequences (RNA sequences).

TABLE 4

| | Protospacer sequence/Spacer sequence | SEQ ID NO: |
|---|---|---|
| 10-nt | CCATTACAGT | 61 |
| 12-nt | CCATTACAGTAG | 62 |
| 14-nt | CCATTACAGTAGGA | 63 |
| 15-nt | CCATTACAGTAGGAG | 64 |
| 16-nt | CCATTACAGTAGGAGC | 65 |
| 17-nt | CCATTACAGTAGGAGCA | 66 |
| 18-nt | CCATTACAGTAGGAGCAT | 67 |
| 19-nt | CCATTACAGTAGGAGCATA | 68 |
| 20-nt | CCATTACAGTAGGAGCATAC | 45 |
| 21-nt | CCATTACAGTAGGAGCATACG | 69 |
| 22-nt | CCATTACAGTAGGAGCATACGG | 70 |
| 23-nt | CCATTACAGTAGGAGCATACGGG | 71 |
| 24-nt | CCATTACAGTAGGAGCATACGGGA | 72 |
| 26-nt | CCATTACAGTAGGAGCATACGGGAGA | 73 |
| 27-nt | CCATTACAGTAGGAGCATACGGGAGAC | 74 |
| 28-nt | CCATTACAGTAGGAGCATACGGGAGACA | 75 |
| 30-nt | CCATTACAGTAGGAGCATACGGGAGACAAG | 76 |
| 32-nt | CCATTACAGTAGGAGCATACGGGAGACAAGCT | 77 |
| 35-nt | CCATTACAGTAGGAGCATACGGGAGACAAGCTTTG | 78 |
| 40-nt | CCATTACAGTAGGAGCATACGGGAGACAAGCTTTGGCCAC | 79 |
| 45-nt | CCATTACAGTAGGAGCATACGGGAGACAAGCTTTGGCCACCTACG | 80 |
| 50-nt | CCATTACAGTAGGAGCATACGGGAGACAAGCTTTGGCCACCTACGGCAAG | 81 |

By using the experimental procedure in Example 1, it was observed that a spacer sequence length range of at least 16 nucleotides is effective for xCas12i's activity, and among that range, 17-22 nt is optimal (FIG. 4B).

### Example 3 Evaluation of PAM Recognition for xCas12i

Considering the 5′-TTN PAM preference of Cas12i, the applicant performed a NTTN PAM identification assay (, wherein N is A, T, C, or G) using the dual plasmid fluorescent reporter system in Example 1, in which various 5′ PAM was used in place of the original 5′ PAM of [symbol] , while the other elements of the dual plasmid fluorescent reporter system remained.

By using the experimental procedure in Example 1, it was observed that xCas12i showed a consistently high frequency of EGFP activation at target sites with 5′-NTTN PAM sequences, wherein N is A, T, C, or G, while LbCas12a had comparable activity at just 5′-TTTN PAM, respectively (FIG. 4C), showing the much more broad PAM site recognition of xCas12i.

### Example 4 Effect of DR Sequence on xCas12i's dsDNA Cleavage Activity

To test whether the original direct repeat (DR) sequence (SEQ ID NO: 11, 36 nt) identified together with xCas12i could tolerate variation, the applicant truncated the original DR sequence to generate two functional fragments DR-T1 (30 nt) and DR-T2 (23 nt) of SEQ ID NOs: 451 and 452, respectively, without destroying the secondary structure of the original DR sequence, and then designed five DR variants of DR-T2 to generate DR-A, DR-B, DR-C, DR-D, and DR-E sequences of SEQ ID NOs: 453-457, respectively, each containing 5% to 30% mutations in the stem-loop regions without destroying the secondary structure of the original DR sequence. That is, the secondary structures of the 7 DR variants were substantially the same as that of the original DR sequence (FIG. 22).

SEQ ID NO: 451 DR-T1, 30 nt

ATGACTCAGAAATGTGTCCCCAGTTGACAC

SEQ ID NO: 452 DR-T2 sequence, 23 nt

AGAAATGTGTCCCCAGTTGACAC

SEQ ID NO: 453 DR-A sequence, 23 nt

AGAAATCCGTCCTTAGTTGACGG

SEQ ID NO: 454 DR-B sequence, 22 nt

AGACATGTGTCCCCAGTGACAC

SEQ ID NO: 455 DR-C sequence, 23 nt

AGAAATGTTTCCCCAGTTGAAAC

SEQ ID NO: 456 DR-D sequence, 23 nt

AGAAATGTGTTCCCAGTTAACAC

SEQ ID NO: 457 DR-E sequence, 23 nt

AGAAATTTGTCCCCAGTTGACAA

By using the dual plasmid fluorescent reporter system for xCas12i in Example 1 with the original DR sequence (SEQ ID NO: 11) replaced with each of the DR variants (DR-T1, DR-T2, DR-A, DR-B, DR-C, DR-D, and DR-E), while the other element of the reporter system remained, the results (FIG. 21) show that xCas12i still exhibited high dsDNA cleavage activity mediated by gRNAs with various DR sequence variants. It can be seen that under the condition that the secondary structure of the DR sequence is maintained (i.e., the secondary structures of the DR variants are substantially the same as that of the original DR sequence), the CRISPR-SiCas12i system tolerated mismatching or deletion on DR sequence without substantial loss of dsDNA cleavage activity, indicating wide adaptability to variations in the DR sequence. These data also demonstrated that the two functionally truncated versions of original xCas12i DR sequence of SEQ ID NO: 11 (36 nt), i.e., DR-T1 (SEQ ID NO: 451, 30 nt) and DR-T2 (SEQ ID NO: 452, 23 nt), could still mediate high dsDNA cleavage activity of xCas12i.

### Example 5 Evaluation of dsDNA Cleavage Activity of xCas12i at Endogenous Gene

To further verify the dsDNA cleavage activity of xCas12i at an endogenous gene (genome cleavage) in mammalian cells, the applicant transfected the expression plasmid (FIG. 3A, FIG. 4D) in Example 1 encoding NLS tagged xCas12i with gRNAs targeting 37 sites from human TTR¹² gene and human PCSK9¹³ gene in HEK293T (human embryonic kidney 293 cells) or mouse Ttr gene in N2a cells (Neuro2a cells, a fast-growing mouse neuroblastoma cell line). The EGxxxxFP targeting spacer sequence (SEQ ID NO: 45) in Example 1 was replaced with respective gene-targeting spacer sequence (SEQ ID NOs: 82-119 and 121-125 in Table 5), the DR-T1 sequence (SEQ ID NO: 451) was used in place of the original DR sequence (SEQ ID NO: 11) (and also in the Examples below unless otherwise specified), while the other elements of the CRISPR-xCas12i system in Example 1 remained. The dsDNA cleavage activity, i.e., indel (insertion and/or deletion) formation, at these loci was measured 48 hours after transfection using FACS and targeted deep sequencing. To save drafting, the sequences in Table 5 refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NOs: 82-119 and 121-125 in the sequence listing are annotated as DNA sequences.

It was observed that xCas12i mediated a high frequency, up to 90%, of indel formation at most sites from Ttr, TTR and PCSK9, with a mean indel formation rate of over 50% (FIG. 4E-F). These data indicate that xCas12i exhibits a robust genome editing efficiency in mammalian cells, suggesting that it has excellent potential for therapeutic genome editing applications.

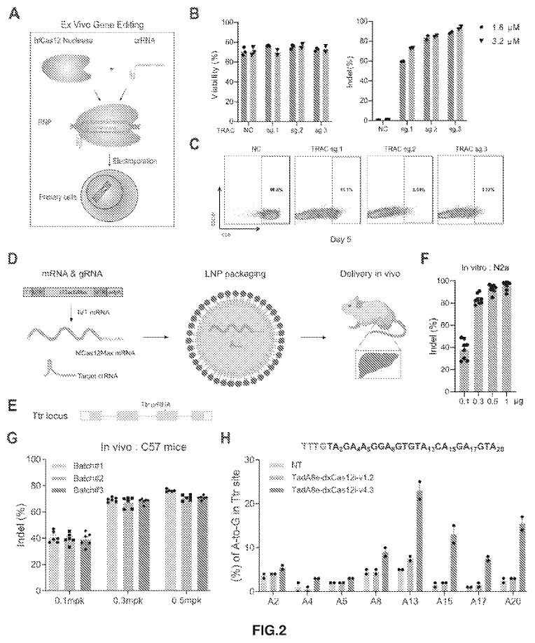

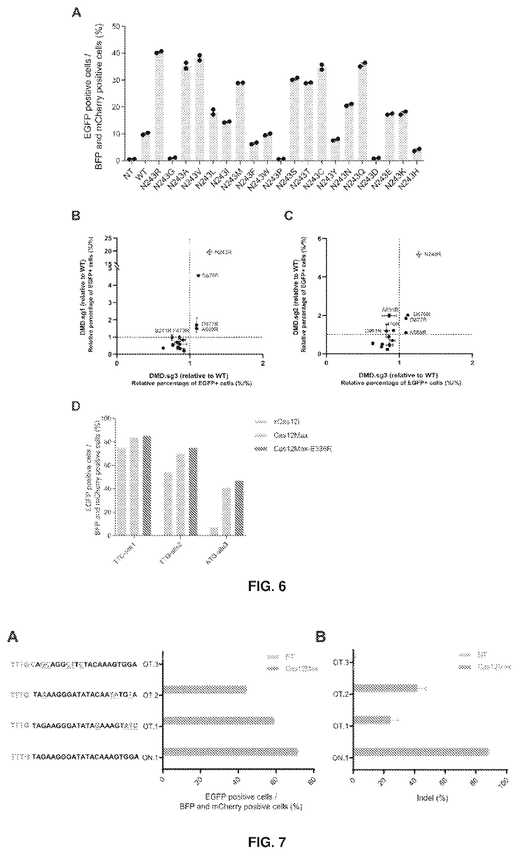

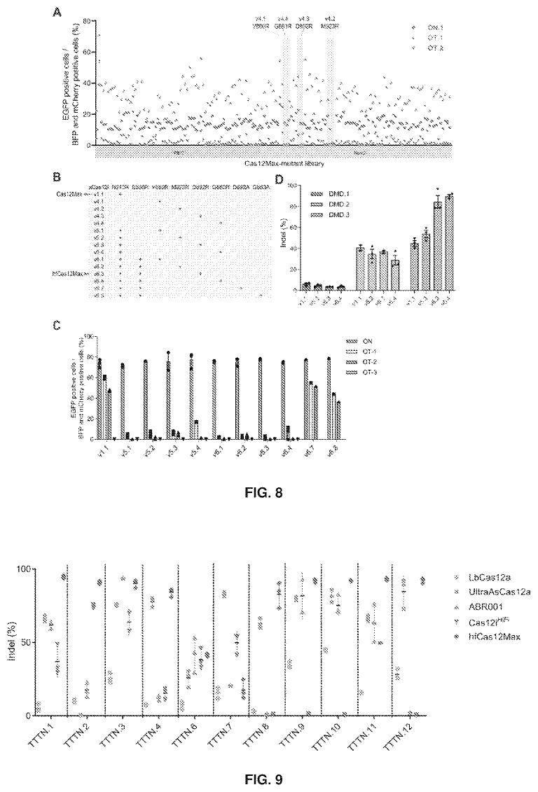

TABLE 5
Sequences for testing genome cleavage at target loci

| | | | | SEQ ID | |
|---|---|---|---|---|---|
| | | | | NO of | |
| | | | | protospacer/ | |
| Genomic | | | Protospacer sequence/ | spacer | |
| loci | Guide RNA | PAM | Spacer sequences | sequence | Figure |
| DMD | DMD_sg1 | TTTG | CAAAAACCCAAAATATTTTA | 82 | FIG. 1D, FIG. 6B-C, |
| | | | | | and FIG. 8D |
| | DMD_sg2 | TTTA | GCTCCTACTCAGACTGTTAC | 83 | FIG. 1D, FIG. 6B-C, |
| | | | | | and FIG. 8D |
| | DMD_sg3 | GTTG | TGTCACCAGAGTAACAGTC | 84 | FIG. 1D, FIG. 6B-C, |
| | | | | | and FIG. 8D |
| Ttr | Ttr_sg1 | TTTG | CCTCGCTGGACTGGTATTTG | 85 | FIG. 4E-F |
| | Ttr_sg2 | TTTG | TGTCTGAAGCTGGCCCCGCG | 86 | FIG. 1D and FIG. |
| | | | | | 4E-F |
| | Ttr_sg3 | CTTC | CCTTCGACTCTTCCTCCTTTG | 87 | FIG. 1D and FIG. |
| | | | | | 4E-F, 19B |
| | Ttr_sg4 | CTTC | CTCCTTTGCCTCGCTGGACTG | 88 | FIG. 4E-F |
| | Ttr_sg5 | TTTG | ACCATCAGAGGACATTTGGA | 89 | FIG. 4E-F |
| | Ttr_sg6 | TTTG | GATTCTCCAGCACCCTGGGC | 90 | FIG. 4E-F |
| | Ttr_sg7 | TTTA | CAGCCACGTCTACAGCAGGG | 91 | FIG. 4E-F |
| | Ttr_sg8 | TTTT | ACAGCCACGTCTACAGCAGG | 92 | FIG. 4E-F |
| | Ttr_sg9 | TTTT | GAACACTTTTACAGCCACGT | 93 | FIG. 4E-F |
| | Ttr_sg10 | GTTC | AAAAAGACCTCTGAGGGATC | 94 | FIG. 4E-F |
| | Ttr_sg11 | TTTG | AACACTTTTACAGCCACGTC | 95 | FIG. 4E-F |
| | Ttr_sg12 | TTTG | TAGAAGGAGTGTACAGAGTA | 96 | FIG. 1D, FIG. 2F-H |
| | | | | | and FIG. 4E-F, 19B |
| | Ttr_sg13 | CTTG | GCATTTCCCCGTTCCATGAA | 97 | FIG. 4E-F |
| | Ttr_sg14 | CTTC | TCATCTGTGGTGAGCCCGTG | 98 | FIG. 4E-F |
| | Ttr_sg15 | TTTG | GTGTCCAGTTCTACTCTGTA | 99 | FIG. 4E-F |
| | Ttr_sg16 | CTTC | CAGTACGATTTGGTGTCCAG | 100 | FIG. 4E-F |
| | Ttr_sg17 | CTTC | TACAAACTTCTCATCTGTGG | 101 | FIG. 4E-F |
| | Ttr_sg18 | TTTT | CACAGCCAACGACTCTGGCC | 102 | FIG. 4E-F |
| | Ttr_sg19 | TTTC | ACAGCCAACGACTCTGGCCA | 103 | FIG. 4E-F |
| | Ttr_sg20 | GTTG | CTGACGACAGCCGTGGTGCTG | 104 | FIG. 4E-F |
| | | | T | | |
| | Ttr_sg21 | GTTC | AAAAAGACCTCTGAGGGATCC | 105 | FIG. 4E-F |
| | | | T | | |
| TTR | TTR_sg1 | GTTC | AGAAAGGCTGCTGATGACACC | 106 | FIG. 1D and FIG. |
| | | | T | | 4E-F |
| | TTR_sg2 | TTTG | TAGAAGGGATATACAAAGTGG | 107 | FIG. 1D and FIG. |
| | | | A | | 4E-F, 16 |
| | TTR_sg3 | ATTC | CACCACGGCTGTCGTCACCAA | 108 | FIG. 4E-F |
| | | | T | | |
| | TTR_sg5 | TTTG | AATCCAAGTGTCCTCTGATGGT | 109 | FIG. 4E-F |
| | TTR_sg6 | TTTC | AATGTGGCCGTGCATGTGTTCA | 110 | FIG. 4E-F |
| | TTR_sg7 | GTTC | TAGATGCTGTCCGAGGCAGTC | 111 | FIG. 4E-F |
| | | | C | | |
| | TTR_sg8 | ATTC | GCATGGGCTCACAACTGAGGA | 112 | FIG. 4E-F |
| | | | G | | |
| | TTR_sg10 | TTTG | TATACAAAGTGGAAATAGACA | 113 | FIG. 4E-F |
| | | | C | | |
| | TTR_sg11 | CTTA | CTGGAAGGCACTTGGCATCTC | 114 | FIG. 1D and FIG. |
| | | | C | | 4E-F |
| | TTR_sg12 | CTTG | GCATCTCCCCATTCCATGAGCA | 115 | FIG. 1D and FIG. |
| | | | | | 4E-F |
| | TTR_sg14 | ATTC | ACAGCCAACGACTCCGGCCCC | 116 | FIG. 4E-F |
| | | | C | | |
| PCSK9 | PCSK9_sg5 | GTTG | CCTGGCACCTACGTGGTGG | 117 | FIG. 4E-F |
| | PCSK9_sg6 | CTTC | CATGGCCTTCTTCCTGGC | 118 | FIG. 1D and FIG. |
| | | | | | 4E-F |
| | PCSK9_sg7 | CTTC | TTCCTGGCTTCCTGGTGAAG | 119 | FIG. 4E-F |
| | PCSK9_sg9 | CTTG | AAGTTGCCCCATGTCGACTA | 121 | FIG. 1D and FIG. |
| | | | | | 4E-F |
| | PCSK9_sg10 | TTTG | CCCAGAGCATCCCGTGGAAC | 122 | FIG. 1D and FIG. |

4E-F

| | | | | | |
|---|---|---|---|---|---|
| TRAC | TRAC_sg1 | TTTA | CAGATACGAACCTAAACTTT | 123 | FIG. 2B-C |
| | TRAC_sg2 | TTTA | GAGTCTCTCAGCTGGTACAC | 124 | FIG. 2B-C |
| | TRAC_sg3 | TTTG | TCTGTGATATACACATCAGA | 125 | FIG. 2B-C |

### Example 6 Development of xCas12i Mutants and Evaluation of their dsDNA Cleavage Activity

To vary xCas12i's dsDNA cleavage activity and expand its scope of PAM site recognition, the applicant engineered xCas12i protein via mutagenesis and screened for mutants with higher dsDNA cleavage activity and broader PAM using a dual plasmid fluorescent reporter system similar to the dual plasmid fluorescent reporter system in Example 1, except that the EGxxxxFP-targeting guide RNA (SEQ ID NO: 51; “crON”) coding sequence operably linked to U6 promoter was not located on the expression plasmid together with the xCas12i (or its mutant) coding sequence (SEQ ID NO: 31) but located on the reporter plasmid together with the BFP-P2A-EGxxxxFP coding sequence (SEQ ID NO: 41) (referring to “On-Target Reporter” in FIG. 1B). Combined with predictive structural analysis of xCas12i, the applicant performed an arginine (R) scanning mutagenesis approach in the domains including PI domain (amino acid residue position 173-291), REC-I domain (amino acid residue position 427-473), and RuvC-II domain (amino acid residue position 800-1082) of xCas12i, generating a library of 599 xCas12i mutants with a single non-R amino acid substitution with R. The xCas12i (SEQ ID NO: 1) coding sequence on the expression plasmid was replaced with a sequence encoding each of the xCas12i mutants in Table 6, the DR-T1 sequence (SEQ ID NO: 451) was used in place of the original DR sequence (SEQ ID NO: 11), while the other elements of the reporter system remained. The applicant then individually transfected the expression plasmid and the reporter plasmid into HEK293T cells and analyzed them by FACS (FIG. 1B).

For negative control (“NT”), a non-targeting spacer sequence (“NT”, SEQ ID NO: 46) incapable of hybridizing to the target sequence (SEQ ID NO: 44) was used in place of the EGxxxxFP-targeting spacer sequence (SEQ ID NO: 45) and used in combination with xCas12i (SEQ ID NO: 1), while the other elements of the reporter system remained. For positive control (“WT”), the original xCas12i (SEQ ID NO: 1) was used.

TABLE 6
Mutants of xCas12i and dsDNA cleavage activity thereof

| | dsDNA | | dsDNA | | dsDNA | | dsDNA |
|---|---|---|---|---|---|---|---|
| | cleavage | | cleavage | | cleavage | | cleavage |
| Mutant | activity | Mutant | activity | Mutant | activity | Mutant | activity |
| K109R | 0.034 | K264R | 0.671 | S428R | 0.075 | M923R | 1.044 |
| N110R | 0.778 | A265R | 0.725 | Y430R | 0.359 | S924R | 0.351 |
| Y111R | 0.634 | M266R | 0.250 | Y431R | 0.856 | S925R | 1.276 |
| L112R | 0.041 | I267R | 0.933 | E432R | 0.670 | H926R | 1.440 |
| M113R | 0.062 | S268R | 0.959 | D433R | 0.605 | Q927R | 1.933 |
| S114R | 0.837 | N269R | 0.401 | F434R | 0.161 | D928R | 0.164 |
| N115R | 0.312 | F270R | 0.131 | S435R | 0.981 | P929R | 0.179 |
| I116R | 0.836 | T271R | 0.450 | A436R | 0.033 | F930R | 0.203 |
| D117R | 0.499 | K272R | 0.383 | K437R | 0.880 | V931R | 1.547 |
| S118R | 1.481 | N273R | 1.652 | N438R | 0.309 | H932R | 0.229 |
| D119R | 1.337 | A274R | 0.207 | F439R | 0.010 | M933R | 1.827 |
| F121R | 1.356 | A275R | 0.713 | L440R | 1.379 | Q934R | 2.147 |
| V122R | 0.737 | A276R | 0.309 | D441R | 0.671 | D935R | 1.413 |
| W123R | 1.010 | K277R | 0.282 | G442R | 0.051 | K936R | 1.489 |
| V124R | 0.119 | A278R | 0.471 | A443R | 0.033 | K937R | 1.442 |
| D125R | 0.040 | A279R | 0.683 | K444R | 0.547 | T938R | 1.452 |
| C126R | 0.051 | K280R | 0.556 | L445R | 0.107 | S939R | 1.413 |
| R127R | 0.025 | K281R | 0.671 | N446R | 0.410 | V940R | 1.333 |
| K128R | 0.844 | P282R | 0.575 | V447R | 0.004 | L941R | 0.988 |
| F129R | 0.802 | I283R | 0.390 | L448R | 1.369 | P943R | 0.812 |
| A130R | 0.064 | P284R | 0.274 | T449R | 0.514 | F945R | 1.055 |
| K131R | 0.728 | Y285R | 0.287 | E450R | 0.887 | M946R | 1.207 |
| D132R | 0.839 | L286R | 0.745 | V451R | 1.883 | E947R | 0.885 |
| F133R | 0.990 | D287R | 1.084 | V452R | 0.735 | V948R | 1.231 |
| A134R | 0.076 | R288R | 0.386 | N453R | 0.895 | N949R | 1.893 |
| Y135R | 0.863 | L289R | 0.400 | Q455R | 1.190 | K950R | 1.640 |
| Q136R | 1.067 | K290R | 0.403 | K456R | 0.887 | D951R | 2.347 |
| M137R | 0.128 | E291R | 0.363 | A457R | 0.004 | S952R | 1.500 |
| E138R | 1.010 | M293R | 0.019 | H458R | 0.008 | I953R | 0.382 |
| L139R | 0.194 | V294R | 0.665 | P459R | 0.008 | D955R | 1.221 |
| G140R | 0.957 | S295R | 1.172 | T460R | 0.009 | Y956R | 1.768 |
| F141R | 0.429 | L296R | 0.752 | I461R | 0.801 | H957R | 0.681 |
| H142R | 0.941 | C297R | 0.061 | W462R | 0.358 | V958R | 0.541 |
| E143R | 1.240 | D298R | 0.719 | S463R | 0.020 | A959R | 1.635 |
| F144R | 0.007 | Y300R | 0.168 | E464R | 1.127 | G960R | 1.840 |
| T145R | 0.951 | N301R | 0.359 | I800R | 0.596 | L961R | 0.152 |
| V146R | 1.106 | V302R | 1.517 | S801R | 0.204 | L965R | 0.443 |
| L147R | 0.038 | Y303R | 0.324 | L802R | 0.398 | N966R | 1.933 |
| A148R | 0.013 | A304R | 0.067 | K803R | 0.436 | S967R | 1.529 |
| E149R | 0.319 | W305R | 0.026 | M804R | 0.130 | K968R | 1.241 |
| T150R | 0.686 | A306R | 0.187 | I805R | 0.325 | S969R | 1.548 |
| L151R | 0.038 | A307R | 0.265 | S806R | 1.214 | D970R | 1.451 |
| L152R | 0.097 | A308R | 0.030 | D807R | 0.899 | A971R | 1.848 |
| A153R | 1.000 | I309R | 0.009 | F808R | 0.261 | G972R | 1.152 |
| N154R | 0.307 | T310R | 0.163 | K809R | 0.905 | T973R | 0.641 |
| S155R | 1.577 | N311R | 0.120 | G810R | 0.954 | S974R | 1.180 |
| I156R | 0.531 | S312R | 0.037 | V811R | 0.178 | V975R | 1.097 |
| L157R | 0.041 | N313R | 0.246 | V812R | 0.187 | Y976R | 1.148 |
| V158R | 1.990 | A314R | 0.030 | Q813R | 0.161 | Y977R | 0.007 |
| L159R | 0.085 | D315R | 0.046 | S814R | 0.023 | Q979R | 1.421 |
| N160R | 0.860 | V316R | 0.007 | Y815R | 0.284 | A980R | 1.057 |
| E161R | 2.115 | T317R | 0.143 | F816R | 0.299 | A981R | 0.341 |
| S162R | 2.096 | A318R | 0.037 | S817R | 1.290 | L982R | 1.146 |
| T163R | 1.054 | N320R | 0.098 | V818R | 1.410 | H983R | 1.372 |
| K164R | 0.760 | T321R | 0.156 | S819R | 1.130 | F984R | 0.580 |
| A165R | 3.151 | L324R | 0.035 | G820R | 0.407 | C985R | 1.076 |
| N166R | 1.548 | T325R | 0.209 | C821R | 0.801 | E986R | 1.137 |
| W167R | 0.775 | F326R | 0.183 | V822R | 0.699 | A987R | 1.220 |
| A168R | 0.058 | I327R | 0.031 | D823R | 0.911 | L988R | 0.954 |
| W169R | 0.161 | G328R | 0.879 | D824R | 0.939 | G989R | 1.420 |
| G170R | 0.572 | E329R | 0.249 | A825R | 0.884 | V990R | 1.094 |
| T171R | 0.211 | Q330R | 0.159 | S826R | 0.707 | S991R | 1.211 |
| V172R | 0.564 | N331R | 0.538 | K827R | 0.654 | P992R | 1.128 |
| S173R | 0.202 | S332R | 1.136 | K828R | 0.917 | E993R | 1.154 |
| A174R | 0.398 | K335R | 0.577 | A829R | 0.954 | L994R | 1.148 |
| L175R | 0.170 | E336R | 1.463 | H830R | 0.593 | V995R | 1.109 |
| Y176R | 0.215 | L337R | 0.613 | D831R | 0.318 | K996R | 1.038 |
| G177R | 0.135 | S338R | 1.505 | S832R | 1.010 | N997R | 1.211 |
| G178R | 1.920 | V339R | 1.183 | M833R | 1.088 | K998R | 1.171 |
| G179R | 0.737 | L340R | 0.419 | L834R | 0.835 | K999R | 1.348 |
| D180R | 1.025 | Q341R | 0.766 | F835R | 1.280 | T1000R | 1.128 |
| K181R | 0.172 | T342R | 0.322 | T836R | 1.402 | H1001R | 1.209 |
| E182R | 0.235 | T343R | 0.710 | F837R | 1.270 | A1002R | 1.171 |
| D183R | 0.279 | T344R | 0.646 | M838R | 0.961 | A1003R | 1.241 |
| S184R | 0.987 | N345R | 0.218 | C839R | 1.700 | E1004R | 1.460 |
| T185R | 1.685 | E346R | 0.554 | A840R | 1.412 | L1005R | 0.665 |
| L186R | 0.641 | K347R | 0.684 | A841R | 0.245 | G1006R | 1.031 |
| K187R | 0.193 | A348R | 0.048 | E842R | 1.540 | M1009R | 0.980 |
| S188R | 0.234 | K349R | 0.461 | E843R | 1.710 | G1010R | 1.172 |
| K189R | 1.010 | D350R | 0.474 | K844R | 1.520 | S1011R | 0.558 |
| I190R | 0.070 | I351R | 0.146 | T846R | 1.620 | A1012R | 1.098 |
| L191R | 0.118 | L352R | 0.023 | N847R | 1.180 | M1013R | 1.207 |
| L192R | 0.910 | N353R | 0.553 | K848R | 1.230 | L1014R | 1.044 |
| A193R | 1.566 | K354R | 0.681 | E850R | 0.867 | M1015R | 0.535 |
| F194R | 0.194 | N356R | 0.542 | E851R | 0.977 | P1016R | 0.088 |
| V195R | 0.019 | D357R | 0.472 | K852R | 0.337 | W1017R | 1.744 |
| D196R | 1.317 | N358R | 0.554 | T853R | 0.928 | G1019R | 0.387 |
| A197R | 0.791 | L359R | 0.398 | N854R | 1.031 | G1020R | 0.396 |
| L198R | 0.204 | I360R | 0.580 | A856R | 1.262 | V1022R | 1.260 |
| N199R | 1.354 | Q361R | 0.676 | A857R | 0.384 | Y1023R | 0.814 |
| N200R | 1.417 | E362R | 1.430 | S858R | 1.117 | I1024R | 0.296 |
| H201R | 0.183 | V363R | 0.696 | F859R | 0.000 | A1025R | 0.062 |
| E202R | 1.102 | Y365R | 0.016 | I860R | 0.146 | S1026R | 0.971 |
| L203R | 1.344 | T366R | 0.973 | L861R | 0.770 | K1027R | 0.978 |
| K204R | 0.817 | P367R | 0.195 | Q862R | 1.882 | K1028R | 1.550 |
| T205R | 0.973 | A368R | 0.709 | K863R | 1.427 | L1029R | 0.444 |
| K206R | 0.871 | K370R | 0.648 | A864R | 0.000 | T1030R | 0.824 |
| E208R | 0.279 | H371R | 0.068 | Y865R | 1.179 | S1031R | 0.000 |
| I209R | 0.108 | L372R | 0.006 | L866R | 1.417 | D1032R | 1.230 |
| L210R | 0.346 | G373R | 0.430 | H867R | 0.000 | A1033R | 0.563 |
| N211R | 0.499 | D375R | 1.408 | G868R | 1.613 | K1034R | 1.301 |
| Q212R | 0.650 | L376R | 0.006 | C869R | 0.131 | S1035R | 0.790 |
| V213R | 0.114 | A377R | 1.097 | K870R | 1.510 | V1036R | 0.627 |
| C214R | 0.166 | N378R | 1.113 | M871R | 1.334 | K1037R | 1.750 |
| E215R | 0.329 | L379R | 0.008 | I872R | 0.163 | Y1038R | 0.666 |
| S216R | 0.591 | F380R | 0.087 | V873R | 0.306 | C1039R | 1.430 |
| L217R | 0.465 | D381R | 1.502 | C874R | 0.519 | G1040R | 1.077 |
| K218R | 0.294 | T382R | 1.517 | E875R | 0.100 | E1041R | 0.920 |
| Y219R | 0.375 | L383R | 0.006 | D876R | 2.637 | D1042R | 0.928 |
| Q220R | 0.371 | K384R | 0.941 | D877R | 2.492 | M1043R | 0.930 |
| S221R | 1.150 | E385R | 1.424 | L878R | 0.132 | W1044R | 0.870 |
| Y222R | 0.417 | K386R | 0.980 | P879R | 0.132 | Q1045R | 1.560 |
| Q223R | 0.574 | D387R | 1.050 | V880R | 1.458 | Y1046R | 0.708 |
| D224R | 0.301 | I388R | 0.317 | A881R | 0.236 | H1047R | 1.430 |
| M225R | 0.099 | N389R | 0.895 | D882R | 0.356 | A1048R | 0.739 |
| Y226R | 0.000 | N390R | 1.066 | G883R | 1.303 | D1049R | 0.699 |
| V227R | 0.177 | I391R | 0.685 | K884R | 1.624 | E1050R | 0.788 |
| D228R | 0.168 | E392R | 0.996 | T885R | 0.464 | I1051R | 0.678 |
| F229R | 0.190 | N393R | 0.662 | G886R | 1.856 | A1052R | 0.114 |
| S231R | 0.284 | E394R | 0.871 | K887R | 1.606 | A1053R | 0.035 |
| V232R | 0.559 | E395R | 1.144 | A888R | 2.077 | V1054R | 0.122 |
| V233R | 1.253 | E396R | 1.214 | Q889R | 0.720 | N1055R | 0.108 |
| D234R | 0.217 | K397R | 0.918 | N890R | 0.151 | I1056R | 0.078 |
| E235R | 1.727 | Q398R | 1.043 | A891R | 2.265 | A1057R | 0.285 |
| N236R | 1.242 | N399R | 1.050 | D892R | 1.417 | M1058R | 0.354 |
| G237R | 0.470 | V400R | 1.222 | M894R | 1.386 | Y1059R | 0.762 |
| N238R | 0.069 | I401R | 0.754 | D895R | 0.539 | E1060R | 0.623 |
| K239R | 0.988 | N402R | 0.934 | W896R | 0.265 | V1061R | 0.947 |
| K240R | 0.908 | D403R | 1.712 | C897R | 0.873 | C1062R | 0.699 |
| S241R | 1.828 | C404R | 0.689 | A898R | 0.192 | C1063R | 1.137 |
| P242R | 0.167 | I405R | 0.048 | A900R | 1.324 | Q1064R | 0.948 |
| N243R | 3.606 | E406R | 1.758 | L901R | 0.376 | T1065R | 0.781 |
| G244R | 0.060 | Q407R | 1.735 | A902R | 0.621 | G1066R | 0.906 |
| S245R | 1.293 | Y408R | 0.064 | K903R | 1.115 | A1067R | 0.994 |
| M246R | 0.124 | V409R | 1.004 | K904R | 1.106 | F1068R | 0.010 |
| P247R | 0.240 | D410R | 0.771 | V905R | 0.203 | G1069R | 1.067 |
| I248R | 0.962 | D411R | 1.447 | N906R | 1.606 | K1070R | 0.969 |
| V249R | 0.114 | C412R | 1.852 | D907R | 0.238 | K1071R | 0.833 |
| T250R | 0.140 | L415R | 0.650 | G908R | 0.244 | Q1072R | 0.879 |
| K251R | 1.434 | N416R | 1.541 | C909R | 0.499 | K1073R | 0.464 |
| F252R | 0.009 | N418R | 1.292 | V910R | 1.406 | K1074R | 0.286 |
| E253R | 0.321 | P419R | 0.171 | A911R | 0.222 | S1075R | 0.971 |
| T254R | 0.927 | I420R | 0.058 | M912R | 1.106 | D1076R | 0.777 |
| D255R | 1.182 | A421R | 0.910 | S913R | 1.471 | E1077R | 0.709 |
| D256R | 0.595 | A422R | 0.674 | I914R | 1.000 | L1078R | 0.915 |
| L257R | 1.162 | L423R | 0.092 | C915R | 1.663 | P1079R | 0.860 |
| I258R | 0.044 | L424R | 0.013 | Y916R | 1.356 | G1080R | 0.996 |
| S259R | 0.531 | K425R | 0.745 | A918R | 1.882 | WT | 1.000 |
| D260R | 0.293 | H426R | 0.742 | P920R | 0.831 | NT | 0.0084 |
| N261R | 0.484 | I427R | 0.005 | A921R | 0.338 | | |
| Q262R | 0.498 | | | Y922R | 0.446 | | |

Based on the fluorescence intensity of cells with activated EGFP, it was observed that 192 xCas12i mutants showed an increased dsDNA cleavage activity relative to xCas12i (WT; SEQ ID NO: 1) (FIG. 5A, Table 6), and among them, one mutant, xCas12i-N243R, referred to as Cas12Max, showed about 3.6-fold improvement (FIG. 5A). In addition, 51 xCas12i mutants has no more than 5% dsDNA cleavage activity relative to WT xCas12i (SEQ ID NO: 1) (FIG. 5A, Table 6).

The applicant then performed saturation mutagenesis of N243 and observed that the mutation to R indeed showed the highest dsDNA cleavage activity (FIG. 6A).

The applicant next targeted DMD or Ttr sites using the fluorescent reporter system (replacing the insertion sequence (SEQ ID NO: 42) with an insertion sequence containing DMD or Ttr protospacer and corresponding 5′ PAM as listed in Table 5) and observed that Cas12Max displayed a markedly increased frequency of EGFP activation, relative to xCas12i (WT) (FIG. 1C, FIG. 6B-C).

To further test the efficacy of Cas12Max in targeting genomic loci, the applicant designed a total of eight gRNAs to target sites TTR and PCSK9 in HEK293T cells and three more to target Ttr in N2a cells (Table 5), in which DR-T2 (SEQ ID NO: 452) was used. Consistent with the previous results, Cas12Max exhibited a significantly increased frequency of indels compared to WT xCas12i (FIG. 1D).

### Example 7 Further Development of Mutants Based on Cas12Max and Evaluation of their Off-Target dsDNA Cleavage Activity

To examine the specificity of Cas12Max, the applicant transfected a construct designed to express it with a gRNA targeting TTR¹² (with TTR-targeting (on-target) spacer sequence of SEQ ID NO: 130), and performed indel frequency analysis of on- and off-target (OT) sites predicted by Cas-OFFinder¹⁷.

TABLE 7

| | Off-target protospacer sequence | SEQ ID NO: |
|---|---|---|
| | (with 5′ PAM of TTTG) | |
| TTR off-target.3 (OT.3) | CAGCAGGCTTCTACAAAGTGGA | 127 |
| TTR off-target.2 (OT.2) | TAAAAGGGATATACAATATGTA | 128 |
| TTR off-target.1 (OT.1) | TAGAAGGGATATAGAAAGTATC | 129 |
| | On-target protospacer sequence/spacer sequence | |
| | (with 5′ PAM of TTTG) | |
| TTR on-target.1 (ON.1) | TAGAAGGGATATACAAAGTGGA | 130 |

A dual plasmid fluorescent reporter system for evaluation of off-target dsDNA cleavage activity (off-target reporter system; referring to “Off-Target Reporter” in FIG. 1B) was established, which was similar to the dual plasmid fluorescent reporter system in Example 6 for evaluation of (on-target) dsDNA cleavage activity, except that the insertion sequence of the EGxxxxFP coding sequence contains an TTR off-target protospacer sequence (one of SEQ ID NOs: 127-129) containing one or more mismatches (bold, underlined) with a TTR-targeting spacer sequence (SEQ ID NO: 130) in the gRNA, rather than containing a TTR on-target protospacer sequence (SEQ ID NO: 130; which is the same as SEQ ID NO: 107 in Example 5); DR-T1 sequence (SEQ ID NO: 451) was used. To save drafting, the on-target protospacer sequence/spacer sequence in Table 7 refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NO: 130 in the sequence listing is annotated as a DNA sequence.

Using the off-target reporter system (FIG. 7A) or targeted deep sequence analysis on endogenous gene (FIG. 7B), the applicant observed that Cas12Max efficiently edited the target site (“ON.1”), while resulting in indel formation at 2 (“OT.1” and “OT.2”) of 3 predicted off-target sites (“OT.1”, “OT.2”, and “OT.3”), indicating off-target dsDNA cleavage activity.

To eliminate the off-target activity of Cas12Max, the applicant selected those mutants in Example 5 with a single mutation in the REC and RuvC domains¹⁸ and undiminished on-target cleavage activity (comparable to WT), and then tested their off-target dsDNA cleavage activity by using two off-target reporter systems above with TTR OT1 and OT2, respectively (FIG. 1B). It was observed that four xCas12i mutants (xCas12i-V880R (v4.1), xCas12i-M923R (v4.2), xCas12i-D892R (v4.3), and xCas12i-G883R (v4.4)) maintained a high level of on-target dsDNA cleavage activity and showed substantially no off-target dsDNA cleavage activity at both TTR OT1 and OT2 (FIG. 8A).

The applicant further combined one or more of these four amino acid substitutions with N243R or N243R+E336R (FIG. 8B) and it was observed that the variant v6.3 (N243R+E336R+D892R) showed the lowest off-target EGFP activation at OT.1 and OT.2 sites and high on-target at the ON.1 site (FIG. 8B-C). Targeted deep sequencing analysis of endogenous TTR.2 site and its off-target sites in HEK293T showed that v6.3 (N243R+E336R+D892R) significantly reduced off-target indel frequencies at six OT sites and retained on-target indel frequency at ON site, compared to Cas12Max (FIG. 1E). In addition, relative to Cas12Max (v1.1), v6.3 (N243R+E336R+D892R) retained comparable or even higher on-target activity at DMD.1, DMD.2 and DMD.3 sites (FIG. 8D). Therefore, the applicant named v6.3 as high-fidelity Cas12Max (hfCas12Max).

TABLE 8

| Mutant | Version | ON | OT-1 | OT-2 | OT-3 |
|---|---|---|---|---|---|
| N243R | v1.1 | 73.80 | 60.17 | 47.50 | 0.11 |
| N243R + V880R | v5.1 | 71.60 | 3.82 | 0.24 | 0.15 |
| N243R + M923R | v5.2 | 76.10 | 4.90 | 0.92 | 0.15 |
| N243R + D892R | v5.3 | 75.85 | 6.66 | 5.46 | 0.21 |
| N243R + G883R | v5.4 | 77.30 | 16.80 | 1.36 | 0.15 |
| N243R + E336R + | v6.1 | 75.70 | 2.04 | 0.44 | 0.15 |
| V880R | | | | | |
| N243R + E336R + | v6.2 | 75.57 | 2.41 | 2.90 | 0.05 |
| M923R | | | | | |
| N243R + E336R + | v6.3 | 77.73 | 1.55 | 0.25 | 0.13 |
| D892R | | | | | |
| (hfCas12Max) | | | | | |
| N243R + E336R + | v6.4 | 74.75 | 6.65 | 0.64 | 0.03 |
| G883R | | | | | |
| N243R + E336R + | v6.7 | 77.30 | 54.80 | 51.50 | |
| D892A | | | | | |
| N243R + E336R + | v6.8 | 78.50 | 44.00 | 36.40 | |
| G883A | | | | | |
| NT | | 0.028 | 0.048 | 0.067 | 0.014 |

Additionally, to investigate hfCas12Max's PAM preference, the applicant performed a 5′-NNN PAM recognition assay by designing reporter plasmids with the same target sequence but different PAM, similar to Example 3. Besides showing a consistent or higher cleavage activity at sites with a 5′-TTN PAM, hfCas12Max and Cas12Max showed a similarly high cleavage activity for targets with 5′-TNN, 5′-ATN, 5′-GTN, and 5′-CTN PAM sites, compared with the commonly used Cas12″, 19 (LbCas12a, Ultra-AsCas12a) and recently reported improved Cas12i220, 21 (ABR001, Cas12i2H″) (FIG. 1F). Taken together, these results demonstrate that hfCas12Max exhibits high-efficiency editing activity with highly flexible 5′-TN or 5′-TNN PAM recognition.

### Example 8 Verification and Comparison of hfCas12Max's On- and Off-Target dsDNA Cleavage Activity at TTR Gene

To comprehensively evaluate the performance of hfCas12Max in human cells, the applicant designed large number of target sites in the exons of TTR for various Cas nucleases. DR-T2 (SEQ ID NO: 452) was used in this and subsequent Example unless otherwise specified.

In total, editing activity was monitored at 43 sites for hfCas12Max with 5′ TTN PAMs, 43 sites for ABR001 (engineered Cas12i2 from Arbor Biotech) with TTN PAMs, 43 sites for Cas 12i2^{Hifi} (from Prof. L I Wei) with TTN PAMs, 45 sites for SpCas9 with NGG PAMs, 12 sites for LbCas12a with TTTN PAMs, 12 sites for Ultra AsCas12a with TTTN PAMs, and 20 sites for KKH-saCas9 with NNNRRT PAMs (Table 9). Indel analysis showed that hfCas12Max exhibited a higher average on-target dsDNA cleavage activity than all the other Cas nucleases and Cas12Max (FIG. 1G, FIG. 9). To save drafting, the sequences in Table 9 refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NOs: 131-381 in the sequence listing are annotated as protospacer/spacer sequences (DNA).

TABLE 9
Sequences for testing genome cleavage at target loci (FIG. 1G, FIG. 9)

| | | | | | SEQ ID |
|---|---|---|---|---|---|
| | | | | | NO of |
| | | | | | protospacer |
| | | | | | sequence/ |
| Genomic | | | | Protospacer sequence/ | spacer |
| loci | Cas | SITE | 5′/3′PAM | Spacer Sequence | sequence |
| TTR | LbCas12a | TTTN.1 | TTTG | TGTCTGAGGCTGGCCCTACGGTG | 131 |
| | | TTTN.2 | TTTG | ACCATCAGAGGACACTTGGATTC | 132 |
| | | TTTN.3 | TTTC | TGAACACATGCACGGCCACATTG | 133 |
| | | TTTN.4 | TTTG | CCTCTGGGTAAGTTGCCAAAGAA | 134 |
| | | TTTN.5 | TTTG | GCAACTTACCCAGAGGCAAATGG | 135 |
| | | TTTN.6 | TTTC | ACACCTTATAGGAAAACCAGTGA | 136 |
| | | TTTN.7 | TTTC | CTATAAGGTGTGAAAGTCTGGAT | 137 |
| | | TTTN.8 | TTTT | CCTATAAGGTGTGAAAGTCTGGA | 138 |
| | | TTTN.9 | TTTG | TAGAAGGGATATACAAAGTGGAA | 139 |
| | | TTTN.10 | TTTG | TATATCCCTTCTACAAATTCCTC | 140 |
| | | TTTN.11 | TTTC | CACTTTGTATATCCCTTCTACAA | 141 |
| | | TTTN.12 | TTTG | GTGTCTATTTCCACTTTGTATAT | 142 |
| | UltraCas12a | TTTN.1 | TTTG | TGTCTGAGGCTGGCCCTACGGTG | 143 |
| | | TTTN.2 | TTTG | ACCATCAGAGGACACTTGGATTC | 144 |
| | | TTTN.3 | TTTC | TGAACACATGCACGGCCACATTG | 145 |
| | | TTTN.4 | TTTG | CCTCTGGGTAAGTTGCCAAAGAA | 146 |
| | | TTTN.5 | TTTG | GCAACTTACCCAGAGGCAAATGG | 147 |
| | | TTTN.6 | TTTC | ACACCTTATAGGAAAACCAGTGA | 148 |
| | | TTTN.7 | TTTC | CTATAAGGTGTGAAAGTCTGGAT | 149 |
| | | TTTN.8 | TTTT | CCTATAAGGTGTGAAAGTCTGGA | 150 |
| | | TTTN.9 | TTTG | TAGAAGGGATATACAAAGTGGAA | 151 |
| | | TTTN.10 | TTTG | TATATCCCTTCTACAAATTCCTC | 152 |
| | | TTTN.11 | TTTC | CACTTTGTATATCCCTTCTACAA | 153 |
| | | TTTN.12 | TTTG | GTGTCTATTTCCACTTTGTATAT | 154 |
| | KKH-SaCas9 | NNGRRT.1 | ACAGAT | CCACCTATGAGAGAAGACAG | 155 |
| | | NNGRRT.2 | AGGAAT | GGCTGTCGTCACCAATCCCA | 156 |
| | | NNGRRT.3 | AGGAGT | GACGACAGCCGTGGTGGAAT | 157 |
| | | NNGRRT.4 | ATTGAT | CTGAACACATGCACGGCCAC | 158 |
| | | NNGRRT.5 | CCAAGT | CACCCAGGGCACCGGTGAAT | 159 |
| | | NNGRRT.6 | CGGAGT | AATGGTGTAGCGGCGGGGGC | 160 |
| | | NNGRRT.7 | GCAAAT | CTTTGGCAACTTACCCAGAG | 161 |
| | | NNGRRT.8 | GTGAGT | TGTCTGAGGCTGGCCCTACG | 162 |
| | | NNGRRT.9 | TACGGT | TTTGTGTCTGAGGCTGGCCC | 163 |
| | | NNGRRT.10 | TGGAAT | ATTGGTGACGACAGCCGTGG | 164 |
| | | NNGRRT.11 | TGGGAT | AGGAGAAGTCCCTCATTCCT | 165 |
| | | NNGRRT.12 | TTTGGT | CCAAGTGCCTTCCAGTAAGA | 166 |
| | SpCas9 | NGG.1 | AGG | ACACAAATACCAGTCCAGCA | 167 |
| | | NGG.2 | AGG | CCAGTCCAGCAAGGCAGAGG | 168 |
| | | NGG.3 | AGG | GAAGTCCACTCATTCTTGGC | 169 |
| | | NGG.4 | AGG | AAAGTTCTAGATGCTGTCCG | 170 |
| | | NGG.5 | AGG | CCCAGAGGCAAATGGCTCCC | 171 |
| | | NGG.6 | AGG | TTCTTTGGCAACTTACCCAG | 172 |
| | | NGG.7 | AGG | ACTGAGGAGGAATTTGTAGA | 173 |
| | | NGG.8 | AGG | CCCATTCCATGAGCATGCAG | 174 |
| | | NGG.9 | AGG | GCATGGGCTCACAACTGAGG | 175 |
| | | NGG.10 | AGG | AATAGGAGTAGGGGCTCAGC | 176 |
| | | NGG.11 | AGG | GACGACAGCCGTGGTGGAAT | 177 |
| | | NGG.12 | AGG | GGCTGTCGTCACCAATCCCA | 178 |
| | | NGG.13 | AGG | GTCACCAATCCCAAGGAATG | 179 |
| | | NGG.14 | CGG | TGTGTCTGAGGCTGGCCCTA | 180 |
| | | NGG.15 | CGG | AGCCTTTCTGAACACATGCA | 181 |
| | | NGG.16 | CGG | CAGAGGACACTTGGATTCAC | 182 |
| | | NGG.17 | CGG | CATTGATGGCAGGACTGCCT | 183 |
| | | NGG.18 | CGG | CTTCTCTACACCCAGGGCAC | 184 |
| | | NGG.19 | CGG | AATGGTGTAGCGGCGGGGGC | 185 |
| | | NGG.20 | CGG | CCCCTACTCCTATTCCACCA | 186 |
| | | NGG.21 | CGG | GCAGGGCGGCAATGGTGTAG | 187 |
| | | NGG.22 | CGG | GGAGTAGGGGCTCAGCAGGG | 188 |
| | | NGG.23 | CGG | GTATTCACAGCCAACGACTC | 189 |
| | | NGG.24 | GGG | TCACAGAAACACTCACCGTA | 190 |
| | | NGG.25 | GGG | AAAGGCTGCTGATGACACCT | 191 |
| | | NGG.26 | GGG | CTTGGATTCACCGGTGCCCT | 192 |
| | | NGG.27 | GGG | GCCGTGGTGGAATAGGAGTA | 193 |
| | | NGG.28 | GGG | GCGGCAATGGTGTAGCGGCG | 194 |
| | | NGG.29 | GGG | GGAGAAGTCCCTCATTCCTT | 195 |
| | | NGG.30 | GGG | GGCGGCAATGGTGTAGCGGC | 196 |
| | | NGG.31 | GGG | TCACCAATCCCAAGGAATGA | 197 |
| | | NGG.32 | TGG | GCAACTTACCCAGAGGCAAA | 198 |
| | | NGG.33 | TGG | AAGTGCCTTCCAGTAAGATT | 199 |
| | | NGG.34 | TGG | ACCTCTGCATGCTCATGGAA | 200 |
| | | NGG.35 | TGG | TACTCACCTCTGCATGCTCA | 201 |
| | | NGG.36 | TGG | TGTAGAAGGGATATACAAAG | 202 |
| | | NGG.37 | TGG | AGGAGAAGTCCCTCATTCCT | 203 |
| | | NGG.38 | TGG | ATTGGTGACGACAGCCGTGG | 204 |
| | | NGG.39 | TGG | GCGGCGGGGGCCGGAGTCGT | 205 |
| | | NGG.40 | TGG | GGGATTGGTGACGACAGCCG | 206 |
| | | NGG.41 | TGG | GGGGCTCAGCAGGGCGGCAA | 207 |
| | Cas12Max | TTTN.1 | TTTG | TGTCTGAGGCTGGCCCTACGGTG | 208 |
| | | TTTN.2 | TTTG | ACCATCAGAGGACACTTGGATTC | 209 |
| | | TTTN.3 | TTTC | TGAACACATGCACGGCCACATTG | 210 |
| | | TTTN.4 | TTTG | CCTCTGGGTAAGTTGCCAAAGAA | 211 |
| | | TTTN.5 | TTTG | GCAACTTACCCAGAGGCAAATGG | 212 |
| | | TTTN.6 | TTTC | ACACCTTATAGGAAAACCAGTGA | 213 |
| | | TTTN.7 | TTTC | CTATAAGGTGTGAAAGTCTGGAT | 214 |
| | | TTTN.8 | TTTT | CCTATAAGGTGTGAAAGTCTGGA | 215 |
| | | TTTN.9 | TTTG | TAGAAGGGATATACAAAGTGGAA | 216 |
| | | TTTN.10 | TTTG | TATATCCCTTCTACAAATTCCTC | 217 |
| | | TTTN.11 | TTTC | CACTTTGTATATCCCTTCTACAA | 218 |
| | | TTTN.12 | TTTG | GTGTCTATTTCCACTTTGTATAT | 219 |
| | | VTTN.1 | CTTA | CTGGAAGGCACTTGGCATCT | 220 |
| | | VTTN.2 | CTTA | TAGGAAAACCAGTGAGTCTG | 221 |
| | | VTTN.3 | CTTC | TCATCGTCTGCTCCTCCTCT | 222 |
| | | VTTN.4 | ATTC | TTGGCAGGATGGCTTCTCAT | 223 |
| | | VTTN.5 | ATTC | ACCGGTGCCCTGGGTGTAGA | 224 |
| | | VTTN.6 | GTTC | AGAAAGGCTGCTGATGACAC | 225 |
| | | VTTN.7 | GTTC | TAGATGCTGTCCGAGGCAGT | 226 |
| | | VTTN.8 | CTTC | TCTACACCCAGGGCACCGGT | 227 |
| | | VTTN.9 | GTTC | TTTGGCAACTTACCCAGAGG | 228 |
| | | VTTN.10 | CTTC | CAGTAAGATTTGGTGTCTAT | 229 |
| | | VTTN.11 | ATTC | CATGAGCATGCAGAGGTGAG | 230 |
| | | VTTN.12 | ATTC | CTCCTCAGTTGTGAGCCCAT | 231 |
| | | VTTN.13 | CTTC | TACAAATTCCTCCTCAGTTG | 232 |
| | | VTTN.14 | ATTC | ACAGCCAACGACTCCGGCCC | 233 |
| | | VTTN.15 | ATTC | CACCACGGCTGTCGTCACCA | 234 |
| | | VTTN.16 | ATTC | CTTGGGATTGGTGACGACAG | 235 |
| | | VTTN.17 | CTTC | TCTCATAGGTGGTATTCACA | 236 |
| | | VTTN.18 | CTTG | CTGGACTGGTATTTGTGTCT | 237 |
| | | VTTN.19 | CTTG | GCAGGATGGCTTCTCATCGT | 238 |
| | | VTTN.20 | ATTG | ATGGCAGGACTGCCTCGGAC | 239 |
| | | VTTN.21 | CTTG | GATTCACCGGTGCCCTGGGT | 240 |
| | | VTTN.22 | CTTG | GCATCTCCCCATTCCATGAG | 241 |
| | | VTTN.23 | GTTG | TGAGCCCATGCAGCTCTCCA | 242 |
| | | VTTN.24 | ATTG | CCGCCCTGCTGAGCCCCTAC | 243 |
| | | VTTN.25 | GTTG | GCTGTGAATACCACCTATGA | 244 |
| | | VTTN.26 | CTTG | GGATTGGTGACGACAGCCGT | 245 |
| | | VTTN.27 | ATTG | GTGACGACAGCCGTGGTGGA | 246 |
| | | VTTN.28 | ATTT | GTGTCTGAGGCTGGCCCTAC | 247 |
| | | VTTN.29 | CTTT | GACCATCAGAGGACACTTGG | 248 |
| | | VTTN.30 | ATTT | GCCTCTGGGTAAGTTGCCAA | 249 |
| | | VTTN.31 | CTTT | GGCAACTTACCCAGAGGCAA | 250 |
| | | VTTN.32 | ATTT | GGTGTCTATTTCCACTTTGT | 251 |
| | | VTTN.33 | CTTT | GTATATCCCTTCTACAAATT | 252 |
| | hfCas12Max | TTTN.1 | TTTG | TGTCTGAGGCTGGCCCTACGGTG | 253 |
| | | TTTN.2 | TTTG | ACCATCAGAGGACACTTGGATTC | 254 |
| | | TTTN.3 | TTTC | TGAACACATGCACGGCCACATTG | 255 |
| | | TTTN.4 | TTTG | CCTCTGGGTAAGTTGCCAAAGAA | 256 |
| | | TTTN.6 | TTTC | ACACCTTATAGGAAAACCAGTGA | 257 |
| | | TTTN.7 | TTTC | CTATAAGGTGTGAAAGTCTGGAT | 258 |
| | | TTTN.8 | TTTT | CCTATAAGGTGTGAAAGTCTGGA | 259 |
| | | TTTN.9 | TTTG | TAGAAGGGATATACAAAGTGGAA | 260 |
| | | TTTN.10 | TTTG | TATATCCCTTCTACAAATTCCTC | 261 |
| | | TTTN.11 | TTTC | CACTTTGTATATCCCTTCTACAA | 262 |
| | | TTTN.12 | TTTG | GTGTCTATTTCCACTTTGTATAT | 263 |
| | | VTTN.1 | CTTA | CTGGAAGGCACTTGGCATCT | 264 |
| | | VTTN.2 | CTTA | TAGGAAAACCAGTGAGTCTG | 265 |
| | | VTTN.3 | CTTC | TCATCGTCTGCTCCTCCTCT | 266 |
| | | VTTN.4 | ATTC | TTGGCAGGATGGCTTCTCAT | 267 |
| | | VTTN.5 | ATTC | ACCGGTGCCCTGGGTGTAGA | 268 |
| | | VTTN.6 | GTTC | AGAAAGGCTGCTGATGACAC | 269 |
| | | VTTN.7 | GTTC | TAGATGCTGTCCGAGGCAGT | 270 |
| | | VTTN.9 | GTTC | TTTGGCAACTTACCCAGAGG | 271 |
| | | VTTN.10 | CTTC | CAGTAAGATTTGGTGTCTAT | 272 |
| | | VTTN.11 | ATTC | CATGAGCATGCAGAGGTGAG | 273 |
| | | VTTN.12 | ATTC | CTCCTCAGTIGTGAGCCCAT | 274 |
| | | VTTN.13 | CTTC | TACAAATTCCTCCTCAGTTG | 275 |
| | | VTTN.14 | ATTC | ACAGCCAACGACTCCGGCCC | 276 |
| | | VTTN.15 | ATTC | CACCACGGCTGTCGTCACCA | 277 |
| | | VTTN.16 | ATTC | CTTGGGATTGGTGACGACAG | 278 |
| | | VTTN.17 | CTTC | TCTCATAGGTGGTATTCACA | 279 |
| | | VTTN.18 | CTTG | CTGGACTGGTATTTGTGTCT | 280 |
| | | VTTN.19 | CTTG | GCAGGATGGCTTCTCATCGT | 281 |
| | | VTTN.20 | ATTG | ATGGCAGGACTGCCTCGGAC | 282 |
| | | VTTN.21 | CTTG | GATTCACCGGTGCCCTGGGT | 283 |
| | | VTTN.22 | CTTG | GCATCTCCCCATTCCATGAG | 284 |
| | | VTTN.23 | GTTG | TGAGCCCATGCAGCTCTCCA | 285 |
| | | VTTN.24 | ATTG | CCGCCCTGCTGAGCCCCTAC | 286 |
| | | VTTN.25 | GTTG | GCTGTGAATACCACCTATGA | 287 |
| | | VTTN.26 | CTTG | GGATTGGTGACGACAGCCGT | 288 |
| | | VTTN.27 | ATTG | GTGACGACAGCCGTGGTGGA | 289 |
| | | VTTN.28 | ATTT | GTGTCTGAGGCTGGCCCTAC | 290 |
| | | VTTN.29 | CTTT | GACCATCAGAGGACACTTGG | 291 |
| | | VTTN.30 | ATTT | GCCTCTGGGTAAGTTGCCAA | 292 |
| | | VTTN.31 | CTTT | GGCAACTTACCCAGAGGCAA | 293 |
| | | VTTN.32 | ATTT | GGTGTCTATTTCCACTTTGT | 294 |
| | | VTTN.33 | CTTT | GTATATCCCTTCTACAAATT | 295 |
| | ABR001 | TTTN.1 | TTTG | TGTCTGAGGCTGGCCCTACGGTG | 296 |
| | | TTTN.2 | TTTG | ACCATCAGAGGACACTTGGATTC | 297 |
| | | TTTN.3 | TTTC | TGAACACATGCACGGCCACATTG | 298 |
| | | TTTN.4 | TTTG | CCTCTGGGTAAGTTGCCAAAGAA | 299 |
| | | TTTN.6 | TTTC | ACACCTTATAGGAAAACCAGTGA | 300 |
| | | TTTN.7 | TTTC | CTATAAGGTGTGAAAGTCTGGAT | 301 |
| | | TTTN.8 | TTTT | CCTATAAGGTGTGAAAGTCTGGA | 302 |
| | | TTTN.9 | TTTG | TAGAAGGGATATACAAAGTGGAA | 303 |
| | | TTTN.10 | TTTG | TATATCCCTTCTACAAATTCCTC | 304 |
| | | TTTN.11 | TTTC | CACTTTGTATATCCCTTCTACAA | 305 |
| | | TTTN.12 | TTTG | GTGTCTATTTCCACTTTGTATAT | 306 |
| | | VTTN.1 | CTTA | CTGGAAGGCACTTGGCATCT | 307 |
| | | VTTN.2 | CTTA | TAGGAAAACCAGTGAGTCTG | 308 |
| | | VTTN.3 | CTTC | TCATCGTCTGCTCCTCCTCT | 309 |
| | | VTTN.4 | ATTC | TTGGCAGGATGGCTTCTCAT | 310 |
| | | VTTN.5 | ATTC | ACCGGTGCCCTGGGTGTAGA | 311 |
| | | VTTN.6 | GTTC | AGAAAGGCTGCTGATGACAC | 312 |
| | | VTTN.7 | GTTC | TAGATGCTGTCCGAGGCAGT | 313 |
| | | VTTN.9 | GTTC | TTTGGCAACTTACCCAGAGG | 314 |
| | | VTTN.10 | CTTC | CAGTAAGATTTGGTGTCTAT | 315 |
| | | VTTN.11 | ATTC | CATGAGCATGCAGAGGTGAG | 316 |
| | | VTTN.12 | ATTC | CTCCTCAGTTGTGAGCCCAT | 317 |
| | | VTTN.13 | CTTC | TACAAATTCCTCCTCAGTTG | 318 |
| | | VTTN.14 | ATTC | ACAGCCAACGACTCCGGCCC | 319 |
| | | VTTN.15 | ATTC | CACCACGGCTGTCGTCACCA | 320 |
| | | VTTN.16 | ATTC | CTTGGGATTGGTGACGACAG | 321 |
| | | VTTN.17 | CTTC | TCTCATAGGTGGTATTCACA | 322 |
| | | VTTN.18 | CTTG | CTGGACTGGTATTTGTGTCT | 323 |
| | | VTTN.19 | CTTG | GCAGGATGGCTTCTCATCGT | 324 |
| | | VTTN.20 | ATTG | ATGGCAGGACTGCCTCGGAC | 325 |
| | | VTTN.21 | CTTG | GATTCACCGGTGCCCTGGGT | 326 |
| | | VTTN.22 | CTTG | GCATCTCCCCATTCCATGAG | 327 |
| | | VTTN.23 | GTTG | TGAGCCCATGCAGCTCTCCA | 328 |
| | | VTTN.24 | ATTG | CCGCCCTGCTGAGCCCCTAC | 329 |
| | | VTTN.25 | GTTG | GCTGTGAATACCACCTATGA | 330 |
| | | VTTN.26 | CTTG | GGATTGGTGACGACAGCCGT | 331 |
| | | VTTN.27 | ATTG | GTGACGACAGCCGTGGTGGA | 332 |
| | | VTTN.28 | ATTT | GTGTCTGAGGCTGGCCCTAC | 333 |
| | | VTTN.29 | CTTT | GACCATCAGAGGACACTTGG | 334 |
| | | VTTN.30 | ATTT | GCCTCTGGGTAAGTTGCCAA | 335 |
| | | VTTN.31 | CTTT | GGCAACTTACCCAGAGGCAA | 336 |
| | | VTTN.32 | ATTT | GGTGTCTATTTCCACTTTGT | 337 |
| | | VTTN.33 | CTTT | GTATATCCCTTCTACAAATT | 338 |
| | Cas12i2^{HiFi} | TTTN.1 | TTTG | TGTCTGAGGCTGGCCCTACGGTG | 339 |
| | | TTTN.2 | TTTG | ACCATCAGAGGACACTTGGATTC | 340 |
| | | TTTN.3 | TTTC | TGAACACATGCACGGCCACATTG | 341 |
| | | TTTN.4 | TTTG | CCTCTGGGTAAGTTGCCAAAGAA | 342 |
| | | TTTN.6 | TTTC | ACACCTTATAGGAAAACCAGTGA | 343 |
| | | TTTN.7 | TTTC | CTATAAGGTGTGAAAGTCTGGAT | 344 |
| | | TTTN.8 | TTTT | CCTATAAGGTGTGAAAGTCTGGA | 345 |
| | | TTTN.9 | TTTG | TAGAAGGGATATACAAAGTGGAA | 346 |
| | | TTTN.10 | TTTG | TATATCCCTTCTACAAATTCCTC | 347 |
| | | TTTN.11 | TTTC | CACTTTGTATATCCCTTCTACAA | 348 |
| | | TTTN.12 | TTTG | GTGTCTATTTCCACTTTGTATAT | 349 |
| | | VTTN.1 | CTTA | CTGGAAGGCACTTGGCATCT | 350 |
| | | VTTN.2 | CTTA | TAGGAAAACCAGTGAGTCTG | 351 |
| | | VTTN.3 | CTTC | TCATCGTCTGCTCCTCCTCT | 352 |
| | | VTTN.4 | ATTC | TTGGCAGGATGGCTTCTCAT | 353 |
| | | VTTN.5 | ATTC | ACCGGTGCCCTGGGTGTAGA | 354 |
| | | VTTN.6 | GTTC | AGAAAGGCTGCTGATGACAC | 355 |
| | | VTTN.7 | GTTC | TAGATGCTGTCCGAGGCAGT | 356 |
| | | VTTN.9 | GTTC | TTTGGCAACTTACCCAGAGG | 357 |
| | | VTTN.10 | CTTC | CAGTAAGATTTGGTGTCTAT | 358 |
| | | VTTN.11 | ATTC | CATGAGCATGCAGAGGTGAG | 359 |
| | | VTTN.12 | ATTC | CTCCTCAGTTGTGAGCCCAT | 360 |
| | | VTTN.13 | CTTC | TACAAATTCCTCCTCAGTTG | 361 |
| | | VTTN.14 | ATTC | ACAGCCAACGACTCCGGCCC | 362 |
| | | VTTN.15 | ATTC | CACCACGGCTGTCGTCACCA | 363 |
| | | VTTN.16 | ATTC | CTTGGGATTGGTGACGACAG | 364 |
| | | VTTN.17 | CTTC | TCTCATAGGTGGTATTCACA | 365 |
| | | VTTN.18 | CTTG | CTGGACTGGTATTTGTGTCT | 366 |
| | | VTTN.19 | CTTG | GCAGGATGGCTTCTCATCGT | 367 |
| | | VTTN.20 | ATTG | ATGGCAGGACTGCCTCGGAC | 368 |
| | | VTTN.21 | CTTG | GATTCACCGGTGCCCTGGGT | 369 |
| | | VTTN.22 | CTTG | GCATCTCCCCATTCCATGAG | 370 |
| | | VTTN.23 | GTTG | TGAGCCCATGCAGCTCTCCA | 371 |
| | | VTTN.24 | ATTG | CCGCCCTGCTGAGCCCCTAC | 372 |
| | | VTTN.25 | GTTG | GCTGTGAATACCACCTATGA | 373 |
| | | VTTN.26 | CTTG | GGATTGGTGACGACAGCCGT | 374 |
| | | VTTN.27 | ATTG | GTGACGACAGCCGTGGTGGA | 375 |
| | | VTTN.28 | ATTT | GTGTCTGAGGCTGGCCCTAC | 376 |
| | | VTTN.29 | CTTT | GACCATCAGAGGACACTTGG | 377 |
| | | VTTN.30 | ATTT | GCCTCTGGGTAAGTTGCCAA | 378 |
| | | VTTN.31 | CTTT | GGCAACTTACCCAGAGGCAA | 379 |
| | | VTTN.32 | ATTT | GGTGTCTATTTCCACTTTGT | 380 |
| | | VTTN.33 | CTTT | GTATATCCCTTCTACAAATT | 381 |

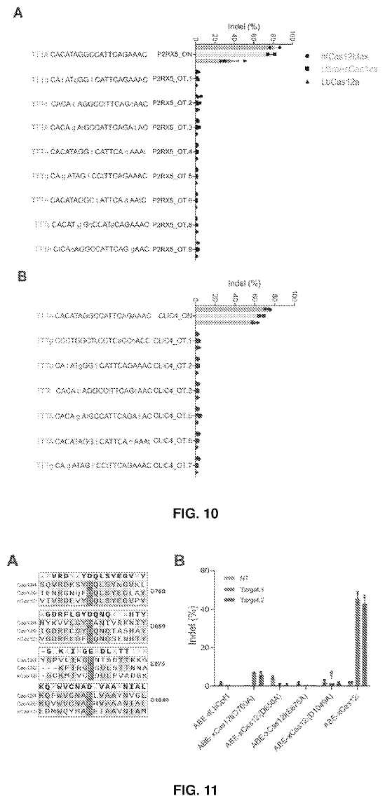

To further evaluate the specificity of hfCas12Max on endogenous genes in human cells, the applicant determined indel frequencies of P2RX5 and NLRC4 on-target and their corresponding in silico predicted off-target sites “2. Targeted deep sequence analysis showed that hfCas12Max had a higher on-target editing efficiency and similarly almost no indel activity at potential off target sites, compared to Ultra AsCas12a and LbCas12a (FIG. 10A-B; protospacer sequences/spacer sequences of SEQ ID NOs: 382-390 from upside to downside in FIG. 10A; protospacer sequences/spacer sequences of SEQ ID NOs: 391-397 from upside to downside in FIG. 10B). To save drafting, the sequences in black in FIGS. 10A and 10B refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NOs: 382-397 in the sequence listing are annotated as protospacer/spacer sequences (DNA).

To sufficiently detect off-target of hfCas12Max and to compare to other Cas proteins, the applicant used PEM-seq²³ to quantify germline events (uncut or perfect rejoining) and editing events including indels and translocations events of TTR.2 libraries. Overall, these results demonstrate that hfCas12Max has high efficiency and specificity and is superior to SpCas9 and other Cas12 nucleases.

### Example 9 Development and Evaluation of Base Editor Based on Dead xCas12i

The applicant further explored the base editing of xCas12i by generating a nuclease-deactivated xCas12i mutant (dead xCas12i, dxCas12i). This was done by introducing single mutations (D650A, D700A, E875A, or D1049A) in the conserved active site of xCas12i based on alignment to Cas12i1⁸ and Cas12i2¹⁰ (FIG. 11A). The dsDNA cleavage activity (Indel %) of each of the four dxCas12i mutants was measured in comparison to dead LbCpf1 (dLbCpf1-D832A) and xCas12i (WT), with N-terminally fusion of TadA8e^{V106W} (SEQ ID NO: 439, TadA8e.1), and the results confirmed that all the four dxCas12i mutants had none or little dsDNA cleavage activity (FIG. 11B). xCas12i-D1049A had the lowest overall dsDNA cleavage activity and thus used in further base editor designs.

### Dxcas12i Mutants:

xCas12i-D650A
xCas12i-D700A
xCas12i-E875A
xCas12i-D1049A

Then, dxCas12i-D1049A was C-terminally fused to TadA8e^{V106W} (SEQ ID NO: 439, TadA8e.1) via a GS linker containing a XTEN linker (SEQ ID NO: 442) or a GS linker containing a BP NLS (SEQ ID NO: 443) to form an adenine base editor (ABE) TadA8e.1-dxCas12i, and dxCas12i-D1049A was C-terminally fused to human APOBEC3A^{W104A} (SEQ ID NO: 440, hA3A.1) via a GS linker containing a XTEN linker (SEQ ID NO: 442) or a GS linker containing a BP NLS (SEQ ID NO: 443), and one UGI (SEQ ID NO: 441), to form a cytidine base editor (CBE) hA3A.1-dxCas12i24-26 ‘(FIGS. 1H and 1J). For the adenine base editor, it contained a N-terminal SV40 NLS (SEQ ID NO: 444) and a C-terminal BP NLS (SEQ ID NO: 443) flanking the fusion of the deaminase and dCas. For the cytidine base editor, it contained a N-terminal BP NLS (SEQ ID NO: 443) and a C-terminal BP NLS (SEQ ID NO: 443) flanking the fusion of the deaminase and dCas.

TadA8e V106W, SEQ ID NO: 439

SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGWRNSKRGAAGSLMNVLNYPGMNHRVEITEGILADECAALLCD
FYRMPRQVFNAQKKAQSSIN

TadA8e W106V, SEQ ID NO: 461

SEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNSKRGAAGSLMNVLNYPGMNHRVEITEGILADECAALLCDF
YRMPRQVFNAQKKAQSSIN

hAPOBEC3W104A, SEQ ID NO: 440

MEASPASGPRHLMDPHIFTSNFNNGIGRHKTYLCYEVERLDNGTSVKMD
QHRGFLHNQAKNLLCGFYGRHAELRFLDLVPSLQLDPAQIYRVTWFISY
SPCFSAGCAGEVRAFLQENTHVRLRIFAARIFDYDPLYKEALQMLRDAG
AQVSIMTYDEFKHCWDTFVDHQGCPFQPWDGLDEHSQALSGRLRAILQN
QGN

UGI, SEQ ID NO: 441

TNLSDIIEKETGKQLVIQESILMLPEEVEEVIGNKPESDILVHTAYDES
TDENVMLLTSDAPEYKPWALVIQDSNGENKIKML

XTEN linker, SEQ ID NO: 442

SGSETPGTSESATPES

bpNLS (also known as BP NLS or bpSV40 NLS) 1, (doi: 10.1038/nature20565.), SEQ ID NO: 443

KRTADGSEFESPKKKRKV

bpNLS 2, SEQ ID NO: 462

KRTADGSESEPKKKRKV

SV40 NLS, from Betapolyomavirus macacae, SEQ ID NO: 444

PKKKRKV

NP NLS (also known as Xenopus laevis Nucleoplasmin NLS or nucleoplasmin NLS), (doi: 10.1126/science.abj6856.), also a bipartite NLS, SEQ ID NO: 445

KRPAATKKAGQAKKKK

human U6 promoter, 241 bp, SEQ ID NO: 446

gagggcctatttcccatgattccttcatatttgcatatacgatacaagg
ctgttagagagataattggaattaatttgactgtaaacacaaagatatt
agtacaaaatacgtgacgtagaaagtaataatttcttgggtagtttgca
gttttaaaattatgttttaaaatggactatcatatgcttaccgtaactt
gaaagtatttcgatttcttggctttatatatcttgtggaaaggac

human CMV promoter, 204 bp, SEQ ID NO: 447

gtgatgcggttttggcagtacatcaatgggcgtggatagcggtttgact
cacggggatttccaagtctccaccccattgacgtcaatgggagtttgtt
ttggcaccaaaatcaacgggactttccaaaatgtcgtaacaactccgcc
ccattgacgcaaatgggcggtaggcgtgtacggtgggaggtctatataa
gcagagct

bGH polyA signal, 208 bp, SEQ ID NO: 448

ctgtgccttctagttgccagccatctgttgtttgcccctcccccgtgcc
ttccttgaccctggaaggtgccactcccactgtcctttcctaataaaat
gaggaaattgcatcgcattgtctgagtaggtgtcattctattctggggg
gtggggggggcaggacagcaagggggaggattgggaagagaatagcagg
catgctgggga

T5 EXO, SEQ ID NO: 449

MSKSWGKFIEEEEAEMASRRNLMIVDGTNLGFRFKHNNSKKPFASSYVS
TIQSLAKSYSARTTIVLGDKGKSVFRLEHLPEYKGNRDEKYAQRTEEEK
ALDEQFFEYLKDAFELCKTTFPTFTIRGVEADDMAAYIVKLIGHLYDHV
WLISTDGDWDTLLTDKVSRFSFTTRREYHLRDMYEHHNVDDVEQFISLK
AIMGDLGDNIRGVEGIGAKRGYNIIREFGNVLDIIDQLPLPGKQKYIQN
LNASEELLFRNLILVDLPTYCVDAIAAVGQDVLDKFTKDILEIAEQ

CAG promoter (human CMV enhancer+chicken β-actin promoter) (containing a hybrid intron), SEQ ID NO: 450

cgttacataacttacggtaaatggcccgcctggctgaccgcccaacgacccccgcccattgacgtcaat
agtaacgccaatagggactttccattgacgtcaatgggtggagtatttacggtaaactgcccacttggc
agtacatcaagtgtatcatatgccaagtacgccccctattgacgtcaatgacggtaaatggcccgcctg
gcattGtgcccagtacatgaccttatgggactttcctacttggcagtacatctacgtattagtcatcgc
tattaccatggtcgaggtgagccccacgttctgcttcactctccccatctcccccccctccccaccccc
aattttgtatttatttattttttaattattttgtgcagcgatgggggcggggggggggggggggcgcgc
gccaggcggggcggggcggggcgaggggggggcggggcgaggcggagaggtgcggcggcagccaatcag
agcggcgcgctccgaaagtttccttttatggcgaggcggcggcggcggcggccctataaaaagcgaagc
gcgcggggggggagtcgctgcgacgctgccttcgccccgtgccccgctccgccgccgcctcgcgccgcc
cgccccggctctgactgaccgcgttactcccacaggtgagcggggggacggcccttctcctccgggctg
taattagctgagcaagaggtaagggtttaagggatggttggttggtggggtattaatgtttaattacct
ggagcacctgcctgaaatcactttttttcag

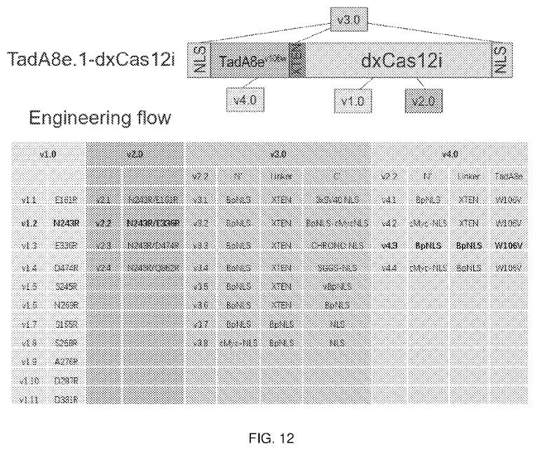

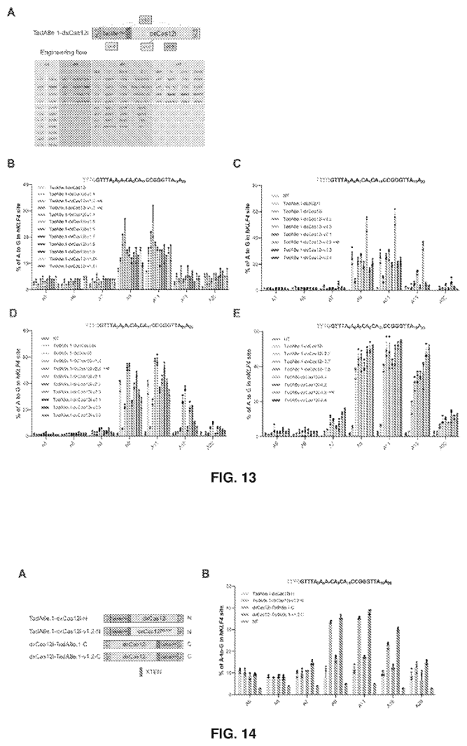

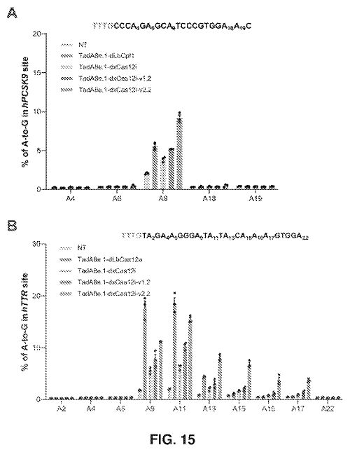

The initial versions of ABE and CBE showed low base editing activity with frequencies of about 8% A-to-G and about 2% C-to-T, respectively (FIG. 11, 1K). To address this, the applicant conducted a series of designs, including introduction of single and combined mutations for high cleavage activity into the PI and Rec domains of dxCas12i (FIG. 12 and FIG. 13A), which resulted in significantly increased A-to-G editing activity. Among the improved variants, TadA8e.1-dxCas12i-v2.2 (D1049A+N243R+E336R) achieved 50% activity at A9 and A11 sites of the KLF4 locus, markedly higher than the 30% activity of TadA8e.1-dLbCas12a (FIG. 11, FIG. 13B-C). At target sites within PCSK9 and TTR, TadA8e.1-dxCas12i-v2.2 showed a similarly increased efficiency to mediate A-to-G transitions, and higher than TadA8e.1-dLbCas12a at PCSK9 site (FIG. 15). To test whether the orientation of deaminase fusion affects the base editing efficiency, the applicant constructed dxCas12i-ABE by fusing the TadA8e.1 to N or C terminus of dxCas12i, and found that TadA8e.1 at C terminus of dxCas12i showed slightly higher activity than N terminus (FIG. 14). The applicant then further engineered the NLS, linker, and TadA8e.1 protein (return back to TadA8e) (FIG. 13A) to produce v3.1-v3.8 and v4.1-v4.4, where TadA8e-dxCas12i-v4.3 exhibited a nearly 80% A-to-G editing efficiency and >95% editing purity, while the editing activities of other dxCas12i-ABE versions were unchanged (FIG. 1H-I, FIG. 13D-E). The applicant named TadA8e-dxCas12i-v4.3 as dCas12Max-ABE (SEQ ID NO: 463), which contains, from N-terminal to C-terminal, Methionine (M), bpNLS 1 (SEQ ID NO: 443), TadA8e-W106V (SEQ ID NO: 461), a bpNLS 1-containing GS linker (SEQ ID NO: 465), xCas12i-N243R+E336R+D1049A (SEQ ID NO: 466), and npNLS (SEQ ID NO: 445).

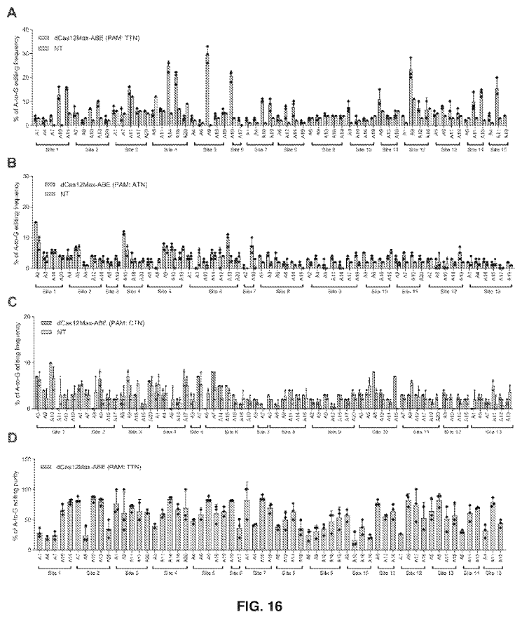

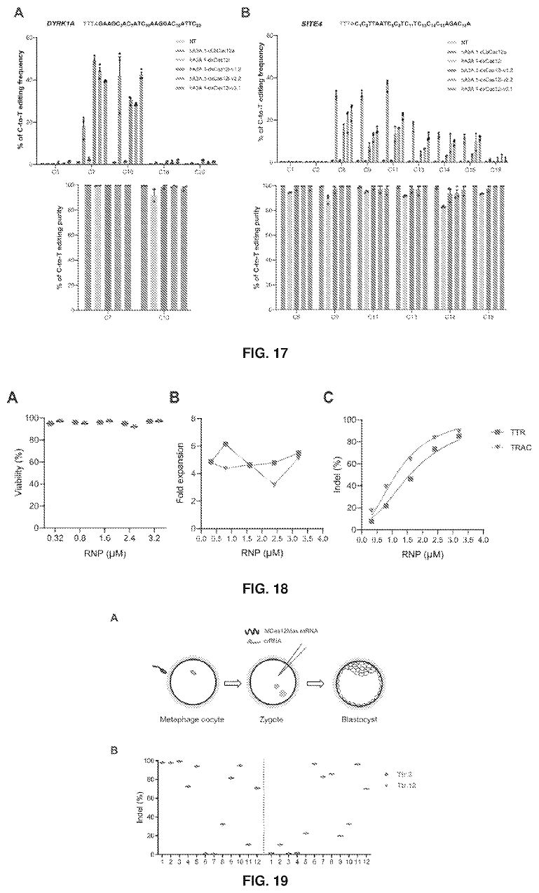

To further characterize the base editing activity of dCas12Max-ABE, the applicant performed 21 sites with TTN PAM, 13 sites with ATN PAMs and 13 sites with CTN PAMs (Table 10). It was observed that dCas12Max-ABE exhibited significant A-to-G activity at sites with TTN PAM (FIG. 16).

In addition, hA3A.1-dxCas12i-v1.2 (N243R), hA3A.1-dxCas12i-v2.2 (N243R+E336R), and hA3A.1-dxCas12i-v3.1 (N243R+E336R-bpNLS) showed consistently elevated C-to-T editing efficiency along with >95% editing purity, at C7 and C10 sites of RUNX1, DYRK1A, and SITE4 locus, even higher than hA3A.1-dLbCas12a at RUNX1 and DYRK1A (FIG. 1J-K). The applicant named hA3A.1-dxCas12i-v3.1 (N243R+E336R-bpNLS) as dCas12Max-CBE (SEQ ID NO: 464), which contains, from N-terminal to C-terminal, Methionine (M), bpNLS 1 (SEQ ID NO: 443), hAPOBEC3W104A (SEQ ID NO: 440), bpNLS 1-containing GS linker (SEQ ID NO: 465), xCas12i-N243R+E336R+D1049A (SEQ ID NO: 466), a short GS linker, SV40 NLS (SEQ ID NO: 444), a short GS linker, UGI (SEQ ID NO: 441), a short GS linker, and bpNLS 2 (SEQ ID NO: 462).

These results together demonstrate that both the engineered dxCas12i-based ABE and CBE exhibit high base editing activity in mammalian cells. To save drafting, the sequences in Table 10 refer to both the protospacer sequence (a DNA sequence) and the spacer sequence (an RNA sequence) with any “T” in the sequence when referring to a protospacer sequences standing for “T” and when referring to a such spacer sequence standing for “U”, although the assigned SEQ ID NOs: 398-438 in the sequence listing are annotated as protospacer/spacer sequences (DNA).

TABLE 10
Sequence of target loci for A to G frequency at different sites (FIG. 16)

| | | | | | SEQ ID NO |
|---|---|---|---|---|---|
| | | | | | of |
| Genomic | | | | | Protospacer/ |
| loci | ABE | SITE | 5′/3′PAM | Protospacer/Spacer Sequence | Spacer Sequence |
| TTR | TTN | site 1 | CTTC | AGCACCACCACGTAGGTGCC | 398 |
| | | site2 | CTTC | CTGGTGAAGATGAGTGGCGA | 399 |
| | | site3 | CTTG | AAGTTGCCCCATGTCGACTA | 400 |
| | | site4 | GTTG | CCCCATGTCGACTACATCGA | 401 |
| | | site5 | TTTG | CCCAGAGCATCCCGTGGAAC | 402 |
| | | site6 | TTTC | CCGGTGGTCACTCTGTATGC | 403 |
| | | site7 | GTTG | AGCACGCGCAGGCTGCGCAT | 404 |
| | | site8 | GTTA | GCGGCACCCTCATAGGTGAG | 405 |
| | | site9 | GTTG | GGGCCACCAATGCCCAGGAC | 406 |
| | | site10 | ATTG | GTGGCCCCAACTGTGATGAC | 407 |
| | | site11 | ATTG | GTGCCTCCAGCGACTGCAGC | 408 |
| | | site12 | ATTC | ACCCCTGCACCAGGCATTGC | 409 |
| | | site13 | GTTC | CCTGAGGACCAGCGGGTACT | 410 |
| | | site14 | GTTG | GTGGCAGTGGACACGGGTCC | 411 |
| | | site15 | GTTG | TCTACGGCGTAGGCCCCCAG | 412 |
| | ATN | site1 | AATC | CAAGTGTCCTCTGATGGTCA | 413 |
| | | site2 | GATG | GTCAAAGTTCTAGATGCTGT | 414 |
| | | site3 | GATG | CTGTCCGAGGCAGTCCTGCC | 415 |
| | | site4 | AATG | TGGCCGTGCATGTGTTCAGA | 416 |
| | | site5 | CATG | TGTTCAGAAAGGCTGCTGAT | 417 |
| | | site6 | GATG | ACACCTGGGAGCCATTTGCC | 418 |
| | | site7 | GATT | CACCGGTGCCCTGGGTGTAG | 419 |
| | | site8 | CATC | AGAGGACACTTGGATTCACC | 420 |
| | | site9 | CATC | TAGAACTTTGACCATCAGAG | 421 |
| | | site10 | GATG | GCAGGACTGCCTCGGACAGC | 422 |
| | | site11 | CATT | GATGGCAGGACTGCCTCGGA | 423 |
| | | site12 | CATG | CACGGCCACATTGATGGCAG | 424 |
| | | site13 | CATC | AGCAGCCTTTCTGAACACAT | 425 |
| | CTN | site1 | CCTC | TGATGGTCAAAGTTCTAGAT | 426 |
| | | site2 | TCTG | ATGGTCAAAGTTCTAGATGC | 427 |
| | | site3 | GCTG | TCCGAGGCAGTCCTGCCATC | 428 |
| | | site4 | GCTG | ATGACACCTGGGAGCCATTT | 429 |
| | | site5 | CCTG | GGAGCCATTTGCCTCTGGGT | 430 |
| | | site6 | CCTC | TGGGTAAGTTGCCAAAGAAC | 431 |
| | | site7 | ACTT | GGATTCACCGGTGCCCTGGG | 432 |
| | | site8 | ACTT | TGACCATCAGAGGACACTTG | 433 |
| | | site9 | TCTA | GAACTTTGACCATCAGAGGA | 434 |
| | | site10 | CCTC | GGACAGCATCTAGAACTTTG | 435 |
| | | site11 | ACTG | CCTCGGACAGCATCTAGAAC | 436 |
| | | site12 | GCTC | CCAGGTGTCATCAGCAGCCT | 437 |
| | | site13 | ACTT | ACCCAGAGGCAAATGGCTCC | 438 |

### Example 10 Evaluation of RNP Delivery of hfCas12Max in T Cells

To explore the therapeutic potential application of hfCas12Max, the applicant delivered hfCas12Max RNP targeting TRAC in CD3+ T cells¹⁹ (FIG. 2A). Beforehand, the applicant tested hfCas12Max RNP targeting TTR and TRAC in HEK293 cells, and it was found that gene editing efficiency was increased following increasing dose of RNPs, with unaffected cellular viability and proliferation (FIG. 18A-C). The applicant achieved about 90% dsDNA cleavage activity and >95% viability at 3.2 μM dose for TRAC (FIG. 18A-C) in HEK293 cells. Three guides were designed to target TRAC (Table 5), and both TRAC sg.2 and sg.3 generated ˜90% editing at both 1.6 and 3.2 μM dose along with ˜80% viability (FIG. 2B) in CD3+ T cells. Flow cytometric analysis showed that TRAC expression was detected to be reduced to a level of 2-3% in CD3+ T cells post 5 days post electroporation treated with RNPs targeting sg.2 or sg.3, compared to 96.6% with untreated cells (FIG. 2C). The guide RNA used in this Example was in the configuration of 5’ DR-T1-spacer sequence-DR-T2-spacer sequence-3′.

### Example 11 Evaluation of LNP Delivery of hfCas12Max In Vivo

To assess the feasibility of hfCas12Max or its base editor of in vivo gene editing, the applicant delivered a guide RNA and a mRNA encoding hfCas12Max or its base editor by LNP packaging to the liver of C57 mouse via tail intravenous injection²⁷ (FIG. 2D). The applicant targeted the exon 3 in the murine transthyretin (Ttr) gene (Ttr_sg12 in Table 5) by gene editing (dsDNA cleavage) and base editing (FIG. 2E). Robust editing efficiencies were detected at four concentration and nearly 100% at 1 μg dose in N2a cells (FIG. 2F). Similarly, targeted deep sequence analysis indicated that the editing efficiencies of murine liver were approximately 70% at the dose of 0.3 and 0.5 milligrams per kilogram (mpk), equivalent to saturation (FIG. 2G). Further, through the LNP packaging delivery, TadA8e-dxCas12i-v4.3 (dCas12Max-ABE) achieved approximately 25% A-to-G efficiency at A13 in Ttr locus in murine liver at 3 mpk dose (FIG. 2H). The guide RNA used in this Example was in the configuration of 5′ DR-T1-spacer sequence-DR-T2-spacer sequence-3′.

In addition, the applicant injected hfCas12Max mRNA with two gRNAs (Ttr_sg3 and 12 in Table 5) targeting Ttr gene into murine zygotes, which were cultured to blastocyst stage for genotyping analysis (FIG. 19A). Targeted deep sequence analysis showed that most zygotes were edited and some up to 100% (FIG. 19B). These results indicate that hfCas12Max mediates robust ex vivo and in vivo gene editing, showing significant potential for disease modeling and therapies.

Mis-folding and aggregation of transthyretin (TTR) is associated with amyloid diseases, including transthyretin-related wild-type amyloidosis (ATTRwt), transthyretin-related hereditary amyloidosis (ATTRm), familial amyloid polyneuropathy (FAP), and familial amyloid cardiomyopathy (FAC). Gene silencing of TTR to reduce TTR protein production may have therapeutic effects in TTR-associated amyloid diseases. The high-efficiency cleavage of TTR target sites in mice in this Example demonstrates that the CRISPR-Cas12i system of the disclosure has very promising prospects for the treatment of TTR-related amyloid diseases, such as ATTR (e.g., ATTRwt or ATTRm).

### Example 12: Screening of xCas12i Mutant with Nickase Activity

To screen xCas12i mutant with nickase activity (i.e., having ssDNA cleavage activity and substantially lacking dsDNA cleavage activity), xCas12i mutant in Tables 11-14 were designed and tested for their nickase activity and dsDNA cleavage activity, by using the reporter system for dsDNA cleavage activity in Example 1 and a reporter system for nickase activity established based on the reporter system for dsDNA cleavage activity in Example 1 wherein the insertion sequence was replaced with an insertion sequence containing, from 5′ to 3′, a 5′ PAM, a protospacer sequence (SEQ ID NO: 43), a linker, a target sequence (SEQ ID NO: 44), and a reverse complementary sequence of the 5′ PAM.

When the xCas12i mutant has only nickase activity, it does not generate green fluorescence with the reporter system for dsDNA cleavage activity but can generate green fluorescence with the reporter system for nickase activity. When the xCas12i mutant has dsDNA cleavage activity, it can generate green fluorescence with both the reporter systems for nickase activity and dsDNA cleavage activity. So the reporter system for nickase activity indicates the sum of the dsDNA cleavage activity and nickase activity. The nickase activity is calculated as green fluorescence from the reporter system for nickase activity minus green fluorescence from the reporter system for dsDNA cleavage activity. Nickase preference was calculated as nickase activity/dsDNA cleavage activity.

It was observed that xCas12i-W896R, xCas12i-S924R, and xCas12i-S925R exhibited significant nickase activity relative to xCas12i (WT) and substantially lacked dsDNA cleavage activity compared with xCas12i (WT).

TABLE 11

| | Nickase | | Nickase |
|---|---|---|---|
| | (ssDNA | dsDNA | activity/ |
| | cleavage) | cleavage | dsDNA |
| | activity | activity | cleavage |
| Mutant | (%) | (%) | activity |
| NT | 0.000 | 0.020 | 0.000 |
| Blank | 0.000 | 0.020 | 0.000 |
| xCas12i | −0.300 | 76.100 | −0.004 |
| xCas12i-W896R | 30.130 | 4.970 | 6.062 |
| xCas12i-S924R | 22.300 | 26.800 | 0.832 |
| xCas12i-S925R | 6.650 | 5.350 | 1.243 |

Further mutagenesis was conducted at W896, S924, or S925 of xCas12i to generate the mutants in Tables 12-14. It was observed that eight xCas12i mutants, W896R, W896P, W896K, S924F, S924D, S924E, S924H, and S925T, achieved both significant nickase preference (Nickase activity/dsDNA cleavage activity >1.0) and high nickase activity (higher than 20%).

TABLE 12
xCas12i-W896 mutants

| | Nickase | | Nickase |
|---|---|---|---|
| | (SSDNA | dsDNA | activity/ |
| | cleavage) | cleavage | dsDNA |
| | activity | activity | cleavage |
| Mutant | (%) | (%) | activity |
| W896G | −3.100 | 72.900 | −0.043 |
| W896A | 6.500 | 75.700 | 0.086 |
| W896V | −0.300 | 64.300 | −0.005 |
| W896L | 13.900 | 61.300 | 0.227 |
| W896I | −0.600 | 74.700 | −0.008 |
| W896M | 0.500 | 76.800 | 0.007 |
| W896F | 5.800 | 74.100 | 0.078 |
| W896W | −0.400 | 80.300 | −0.005 |
| W896P | 32.170 | 8.030 | 4.006 |
| W896S | 0.000 | 72.000 | 0.000 |
| W896T | 0.600 | 67.200 | 0.009 |
| W896C | 2.200 | 72.800 | 0.030 |
| W896Y | 2.300 | 67.700 | 0.034 |
| W896N | 0.700 | 63.700 | 0.011 |
| W896Q | 1.500 | 69.800 | 0.021 |
| W896D | −1.900 | 49.200 | −0.039 |
| W896E | 11.900 | 58.400 | 0.204 |
| W896K | 37.500 | 14.700 | 2.551 |
| W896H | 3.100 | 68.000 | 0.046 |

TABLE 13
xCas12i-S924 mutants

| | | | Nickase |
|---|---|---|---|
| | Nickase | dsDNA | activity/ |
| | (SSDNA | cleavage | dsDNA |
| | cleavage) | activity | cleavage |
| Mutant | activity (%) | (%) | activity |
| S924G | 0.100 | 70.900 | 0.001 |
| S924A | 18.000 | 53.400 | 0.337 |
| S924V | 11.100 | 53.500 | 0.207 |
| S924L | 2.800 | 54.500 | 0.051 |
| S924I | 14.900 | 41.800 | 0.356 |
| S924M | 8.100 | 49.600 | 0.163 |
| S924F | 26.600 | 15.500 | 1.716 |
| S924W | 3.530 | 8.670 | 0.407 |
| S924P | 15.500 | 10.100 | 1.535 |
| S924S | −5.000 | 82.200 | −0.061 |
| S924T | 2.800 | 78.200 | 0.036 |
| S924C | 2.700 | 70.700 | 0.038 |
| S924Y | 11.000 | 11.000 | 1.000 |
| S924N | 8.400 | 71.800 | 0.117 |
| S924Q | 23.400 | 29.200 | 0.801 |
| S924D | 29.000 | 12.700 | 2.283 |
| S924E | 22.800 | 15.400 | 1.481 |
| S924K | 14.600 | 41.600 | 0.351 |
| S924H | 36.000 | 25.300 | 1.423 |

TABLE 14
xCas12i-S925 mutants

| | | | Nickase |
|---|---|---|---|
| | Nickase | dsDNA | activity/ |
| | (SSDNA | cleavage | dsDNA |
| | cleavage) | activity | cleavage |
| Mutant | activity (%) | (%) | activity |
| S925G | 28.700 | 40.900 | 0.702 |
| S925A | −0.600 | 12.700 | −0.047 |
| S925V | 3.000 | 3.560 | 0.843 |
| S925L | 6.650 | 5.750 | 1.157 |
| S925I | 9.000 | 5.800 | 1.552 |
| S925M | 5.350 | 5.150 | 1.039 |
| S925F | 7.530 | 6.870 | 1.096 |
| S925W | 3.330 | 9.770 | 0.341 |
| S925P | 4.700 | 9.700 | 0.485 |
| S925S | −0.300 | 76.300 | −0.004 |
| S925T | 32.000 | 21.200 | 1.509 |
| S925C | 7.600 | 8.000 | 0.950 |
| S925Y | 7.780 | 5.820 | 1.337 |
| S925N | 1.300 | 12.300 | 0.106 |
| S925Q | 6.230 | 5.970 | 1.044 |
| S925D | 9.320 | 6.180 | 1.508 |
| S925E | 11.690 | 6.610 | 1.769 |
| S925K | 6.700 | 10.800 | 0.620 |
| S925H | 6.100 | 10.600 | 0.575 |

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### REFERENCES

1. Anzalone, A. V., Koblan, L. W. & Liu, D. R. Genome editing with CRISPR-Cas nucleases, base editors, transposases and prime editors. Nat Biotechnol 38, 824-844 (2020).
2. Doudna, J. A. The promise and challenge of therapeutic genome editing. Nature 578, 229-236 (2020).
3. Makarova, K. S. et al. Evolutionary classification of CRISPR-Cas systems: a burst of class 2 and derived variants. Nat Rev Microbiol 18, 67-83 (2020).
4. Yan, W. X. et al. Functionally diverse type V CRISPR-Cas systems. Science 363, 88-91 (2019).
5. Kleinstiver, B. P. et al. Genome-wide specificities of CRISPR-Cas Cpf1 nucleases in human cells. Nat Biotechnol 34, 869-+ (2016).
6. Cong, L. et al. Multiplex Genome Engineering Using CRISPR/Cas Systems. Science 339, 819-823 (2013).
7. Zetsche, B. et al. Cpf1 is a single RNA-guided endonuclease of a class 2 CRISPR-Cas system. Cell 163, 759-771 (2015).
8. Zhang, B. et al. Mechanistic insights into the R-loop formation and cleavage in CRISPR-Cas12i1. Nat Commun 12, 3476 (2021).
9. Zhang, H., Li, Z., Xiao, R. & Chang, L. Mechanisms for target recognition and cleavage by the Cas12i RNA-guided endonuclease. Nat Struct Mol Biol 27, 1069-1076 (2020).
10. Huang, X. et al. Structural basis for two metal-ion catalysis of DNA cleavage by Cas12i2. Nat Commun 11, 5241 (2020).
11. Yang, Y. et al. Highly Efficient and Rapid Detection of the Cleavage Activity of Cas9/gRNA via a Fluorescent Reporter. Appl Biochem Biotechnol 180, 655-667 (2016).
12. Gillmore, J. D. et al. CRISPR-Cas9 In Vivo Gene Editing for Transthyretin Amyloidosis. N Engl J Med 385, 493-502 (2021).
13. Musunuru, K. et al. In vivo CRISPR base editing of PCSK9 durably lowers cholesterol in primates. Nature 593, 429-434 (2021).
14. Strecker, J. et al. Engineering of CRISPR-Cas12b for human genome editing. Nat Commun 10, 212 (2019).
15. Kleinstiver, B. P. et al. Engineered CRISPR-Cas12a variants with increased activities and improved targeting ranges for gene, epigenetic and base editing. Nat Biotechnol 37, 276-282 (2019).
16. Xu, X. et al. Engineered miniature CRISPR-Cas system for mammalian genome regulation and editing. Mol Cell 81, 4333-4345 e4334 (2021).
17. Bae, S., Park, J. & Kim, J. S. Cas-OFFinder: a fast and versatile algorithm that searches for potential off-target sites of Cas9 RNA-guided endonucleases. Bioinformatics 30, 1473-1475 (2014).
18. Yuen, C. T. L. et al. High-fidelity KKH variant of Staphylococcus aureus Cas9 nucleases with improved base mismatch discrimination. Nucleic Acids Res 50, 1650-1660 (2022).
19. Zhang, L. et al. AsCas12a ultra nuclease facilitates the rapid generation of therapeutic cell medicines. Nat Commun 12, 3908 (2021).
20. McGaw, C. et al. Engineered Cas12i2 is a versatile high-efficiency platform for therapeutic genome editing. Nat Commun 13, 2833 (2022).
21. Chen, Y. et al. Synergistic engineering of CRISPR-Cas nucleases enables robust mammalian genome editing. Innovation (Camb) 3, 100264 (2022).
22. Kim, D. Y. et al. Efficient CRISPR editing with a hypercompact Cas12f1 and engineered guide RNAs delivered by adeno-associated virus. Nat Biotechnol 40, 94-102 (2022).
23. Yin, J. et al. Optimizing genome editing strategy by primer-extension-mediated sequencing. Cell Discov 5, 18 (2019).
24. Wang, X. et al. Cas12a Base Editors Induce Efficient and Specific Editing with Low DNA Damage Response. Cell Rep 31, 107723 (2020).
25. Richter, M. F. et al. Phage-assisted evolution of an adenine base editor with improved Cas domain compatibility and activity. Nat Biotechnol 38, 883-891 (2020).
26. Li, X. et al. Base editing with a Cpf1-cytidine deaminase fusion. Nat Biotechnol 36, 324-327 (2018).
27. Finn, J. D. et al. A Single Administration of CRISPR/Cas9 Lipid Nanoparticles Achieves Robust and Persistent In Vivo Genome Editing. Cell Rep 22, 2227-2235 (2018).
28. Bravo, J. P. K. et al. Structural basis for mismatch surveillance by CRISPR-Cas9. Nature 603, 343-347 (2022).
29. Kleinstiver, B. P. et al. High-fidelity CRISPR-Cas9 nucleases with no detectable genome-wide off-target effects. Nature 529, 490-495 (2016).
30. Wang, D., Zhang, F. & Gao, G. CRISPR-Based Therapeutic Genome Editing: Strategies and In Vivo Delivery by AAV Vectors. Cell 181, 136-150 (2020).
31. Wang, H. et al. CRISPR-Mediated Programmable 3D Genome Positioning and Nuclear Organization. Cell 175, 1405-1417 e1414 (2018).
32. Konermann, S. et al. Genome-scale transcriptional activation by an engineered CRISPR-Cas9 complex. Nature 517, 583-588 (2015).
33. Nakamura, M., Gao, Y., Dominguez, A. A. & Qi, L. S. CRISPR technologies for precise epigenome editing. Nat Cell Biol 23, 11-22 (2021).
34. Fellmann, C., Gowen, B. G., Lin, P. C., Doudna, J. A. & Corn, J. E. Cornerstones of CRISPR-Cas in drug discovery and therapy. Nat Rev Drug Discov 16, 89-100 (2017).

1. A Cas12i polypeptide comprising an amino acid sequence having from about 80% to less than 100% sequence identity to the amino acid sequence of a reference Cas12i polypeptide of SEQ ID NO: 1.

2. (canceled)

3. The Cas12i polypeptide of claim 1, wherein the Cas12i polypeptide comprises a mutation at one or more positions selected from the group consisting of N243, V880, G883, D892, and M923, and wherein the position is relative to SEQ ID NO: 1.

4-5. (canceled)

6. The Cas12i polypeptide of claim 3, wherein the Cas12i polypeptide comprises an N243R substitution.

7. The Cas12i polypeptide of claim 6, wherein the Cas12i polypeptide comprises the amino acid sequence of SEQ ID NO: 458, or an amino acid sequence having at least about 80% sequence identity to the amino acid sequence of SEQ ID NO: 458.

8-12. (canceled)

13. The Cas12i polypeptide of claim 3, wherein the Cas12i polypeptide comprises one or more substitutions selected from the group consisting of G883R, D892R, V880R, and M923R.

14-20. (canceled)

21. The Cas12i polypeptide of claim 1, wherein the Cas12i polypeptide comprises one or more substitutions selected from the group consisting of D650A, D700A, E875A, and D1049A, and wherein the amino acid position is relative to SEQ ID NO: 1.

22. The Cas12i polypeptide of claim 21, wherein the Cas12i polypeptide comprises substitutions N243R and D1049A.

23-26. (canceled)

27. The Cas 12i polypeptide of claim 1, wherein the Cas 12i polypeptide is a Cas12i nickase having spacer sequence-specific ssDNA cleavage activity and having substantially no spacer sequence-specific dsDNA cleavage activity.

28. (canceled)

29. The Cas12i polypeptide of claim 27, wherein the Cas12i polypeptide comprise one or more substitutions selected from the group consisting of W896R, W896P, W896K, S924F, S924D, S924E, S924H, and S925T, and wherein the amino acid position is relative to SEQ ID NO: 1.

30. (canceled)

31. A fusion protein comprising the Cas 12i polypeptide of claim 1 and a functional domain.

32-44. (canceled)

45. A system comprising:
(1) a Cas12i polypeptide comprising an amino acid sequence having at least 80% sequence identity to the amino acid sequence of a reference Cas12i polypeptide of SEQ ID NO: 1 or a fusion protein comprising the Cas12i polypeptide and a functional domain, or a polynucleotide encoding the Cas 12i polypeptide or the fusion protein; and
(2) a guide RNA or a polynucleotide encoding the guide RNA, the guide RNA comprising:
(i) a direct repeat sequence capable of forming a complex with the Cas 12i polypeptide or the fusion protein; and
(ii) a spacer sequence capable of hybridizing to a target sequence on a target strand of a target dsDNA, thereby guiding the complex to the target dsDNA.

46-54. (canceled)

55. A method for modifying a target dsDNA, comprising contacting the target dsDNA with the system of claim 45, wherein the spacer sequence is capable of hybridizing to a target sequence of a target strand of the target dsDNA, wherein the target sequence is modified by the complex.

56-58. (canceled)

59. A modified cell or a progeny thereof, wherein the modified cell is modified by the method of claim 55.

60-64. (canceled)

65. A host comprising the modified cell or progeny thereof of claim 59.

66. A composition comprising the Cas12i polypeptide of claim 1.

67. A method for diagnosing, preventing, or treating a disease or disorder in a subject, comprising administering to the subject the system of claim 45.

68-71. (canceled)
